(19) United States
(12) Patent Application Publication
(10) Pub. No.: US 2023/0416268 A1
(43) Pub. Date: Dec. 28, 2023

(54) PYRIMIDINONE-CONTAINING 17-BETA-HYDROXYSTEROID DEHYDROGENASE TYPE 13 INHIBITORS

(71) Applicant: ENANTA PHARMACEUTICALS, INC., WATERTOWN, MA (US)

(72) Inventors: Yat Sun Or, Waltham, MA (US); Joseph D. Panarese, Newton, MA (US); Guoqiang Wang, Belmont, MA (US); Jing He, Somerville, MA (US); Jiang Long, Wayland, MA (US); Jun Ma, Wayland, MA (US); Bin Wang, Newton, MA (US); Sourav Ghorai, Waltham, MA (US)

(21) Appl. No.: 18/139,506

(22) Filed: Apr. 26, 2023

Related U.S. Application Data
(60) Provisional application No. 63/335,840, filed on Apr. 28, 2022.

(51) Int. Cl. C07D 495/04

(52) U.S. Cl. CPC C07D 495/04

(57) ABSTRACT

The present invention provides compounds of Formula (I),

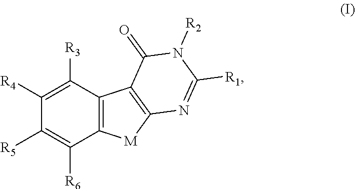

pharmaceutical compositions comprising these compounds and methods of using these compounds for treating a metabolic disease or liver condition. The present invention relates generally to compounds and pharmaceutical compositions useful as 17β-HSD13 inhibitors. Specifically, the present invention relates to compounds useful as inhibitors of 17β-HSD13 and methods for their preparation and use.

### RELATED APPLICATION

This application claims the benefit of U.S. Provisional Application No. 63/335,840, filed on Apr. 28, 2022. The entire teachings of the above application are incorporated herein by reference.

### TECHNICAL FIELD

The present invention relates generally to compounds and pharmaceutical compositions useful as 17β-HSD13 inhibitors. Specifically, the present invention relates to compounds useful as inhibitors of 17β-HSD13 and methods for their preparation and use.

### BACKGROUND OF THE INVENTION

17-Beta-hydroxysteroid dehydrogenases (17β-HSDs) are NADP or NAD⁺ dependent oxidoreductases that catalyze oxidation/reduction reactions of 17β-hydroxysteroids or 17-ketosteroids, respectively. For example, 17β-HSDs can catalyze the interconversion of androstenedione with testosterone, estrone with estradiol, or dehydroepiandrosterone (DHEA) with androstenediol. Of the fifteen 17β-HSDs that have been identified, all but one (17β-HSD type 5) are short-chain dehydrogenases/reductases (SDRs) (J. M. Day, et al., Endocrine-Related Cancer 2008, 15, 665-692).

More specifically, 17-Beta-hydroxysteroid dehydrogenase type 13 (17β-HSD13) is encoded by the HSD17B13 gene and is mainly expressed in the liver (S. Liu, et al., Acta Biochim. Pol. 2007, 54, 213-218). Moreover, 17β-HSD13 was identified as a lipid droplet associated protein and is up-regulated in mice and patients with nonalcoholic fatty liver disease (NAFLD) (Y. Horiguchi, et al., Biochem. Biophys. Res. Commun. 2008, 370, 235-238; W. Su, et al., Mol. Cell. Endocrinol. 2019, 489, 119-125). Further studies have shown that a 17β-HSD13 loss-of-function variant has been associated with a significantly reduced risk of NAFLD, cirrhosis associated with nonalcoholic steatohepatitis (NASH), alcoholic liver disease, alcoholic cirrhosis, hepatocellular carcinoma (HCC), NASH disease severity, ballooning degeneration, lobular inflammation, and fibrosis (N. S. Abul-Husn, et al., N. Engl. J. Med 2018, 378, 1096-1106; C. J. Pirola, et al., J. Lipid Res. 2019, 60, 176-185). This variant has also shown a reduction in liver damage among obese children (A. Di Sessa, et al., J. Pediatr. Gastroenterol. Nutr. 2020, 70, 371-374).

Recently small molecule compounds which act as 17β-HSD13 inhibitors have been published, WO 2023/023310, WO 2022/020714, WO 2022/020730, WO 2021/211974, WO 2021/003295A1. Other agents that act as 17β-HSD13 inhibitors have been disclosed in the following publications: WO 2021/211981, WO 2021/211959, WO 2020/132564, WO 2020/061177, WO 2019/075181, WO 2019/183164, WO 2019/183329, US 2019/0106749, and WO 2018/136758.

There is a need for the development of 17β-HSD13 inhibitors for the treatment and prevention of disease. The present invention has identified compounds which inhibit 17β-HSD13 as well as methods of using these compounds to treat disease.

### SUMMARY OF THE INVENTION

The present invention relates to compounds and pharmaceutical compositions useful as 17β-HSD13 inhibitors. Specifically, the present invention relates to compounds useful as inhibitors of 17β-HSD13 and methods for their preparation and use. In addition, the present invention includes the process for the preparation of the said compounds.

In its principal aspect, the present invention provides compounds represented by Formula (I), or a pharmaceutically acceptable salt or ester thereof:

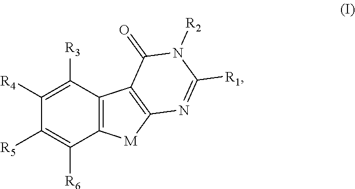

wherein,
M is S, SO, SO₂, O or NR₇;
R₁ and R₂ are each independently selected from the group consisting of:
   1) Hydrogen;
   2) Optionally substituted —C₁-C₈ alkyl;
   3) Optionally substituted —C₂-C₈ alkenyl;
   4) Optionally substituted —C₂-C₈ alkynyl;
   5) Optionally substituted —C₃-C₈ cycloalkyl;
   6) Optionally substituted aryl;
   7) Optionally substituted arylalkyl;
   8) Optionally substituted 3- to 8-membered heterocycloalkyl;
   9) Optionally substituted heteroaryl; and
   10) Optionally substituted heteroarylalkyl;
R₃, R₄, R₅, and R₆ are each independently selected from the group consisting of hydrogen, halogen, —CN, —OR₉, —SR₉, —C(O)R₇, —C(O)OR₇, —NR₇R₈, —C(O)NR₇R₈, optionally substituted —C₁-C₈ alkyl, optionally substituted aryl, and optionally substituted heteroaryl,
alternatively, R₅ and R₆ are taken together with the carbon atoms to which they are attached to form an optionally substituted carbocyclic or heterocyclic ring;
alternatively, R₄ and R₅ are taken together with the carbon atoms to which they are attached to form an optionally substituted carbocyclic or heterocyclic ring;
alternatively, R₃ and R₄ are taken together with the carbon atoms to which they are attached to form an optionally substituted carbocyclic or heterocyclic ring;
each R₇ and R₈ is independently selected from the group consisting of:
   1) Hydrogen;
   2) Optionally substituted —C₁-C₈ alkyl;
   3) Optionally substituted —C₂-C₈ alkenyl;
   4) Optionally substituted —C₂-C₈ alkynyl;
   5) Optionally substituted —C₃-C₈ cycloalkyl;
   6) Optionally substituted 3- to 8-membered heterocycloalkyl;
   7) Optionally substituted aryl;
   8) Optionally substituted arylalkyl;
   9) Optionally substituted heteroaryl; and
   10) Optionally substituted heteroarylalkyl;
alternatively, R₇ and R₈ are taken together with the nitrogen atom to which they are attached to form an optionally substituted heterocyclic ring;
R₉ is selected from the group consisting of:
   1) Hydrogen;
   2) Optionally substituted —C₁-C₈ alkyl;
   3) Optionally substituted —C₂-C₈ alkenyl;
   4) Optionally substituted —C₂-C₈ alkynyl;
   5) Optionally substituted —C₃-C₈ cycloalkyl;
   6) Optionally substituted 3- to 8-membered heterocycloalkyl;
   7) Optionally substituted aryl;
   8) Optionally substituted arylalkyl;
   9) Optionally substituted heteroaryl;
   10) Optionally substituted heteroarylalkyl;
   11) —C(O)R₁₁;
   12) —C(O)NR₁₁R₁₂;
   13) —C(O)OR₁₁;
   14) —P(O)(OR₁₃)₂; and
   15) —P(O)(OR₁₃)(NR₁₁R₁₂);
R₁₁ and R₁₂ are each independently selected from the group consisting of:
   1) Hydrogen;
   2) Optionally substituted —C₁-C₈ alkyl;
   3) Optionally substituted —C₂-C₈ alkenyl;
   4) Optionally substituted —C₂-C₈ alkynyl;
   5) Optionally substituted —C₃-C₈ cycloalkyl;
   6) Optionally substituted 3- to 8-membered heterocycloalkyl;
   7) Optionally substituted aryl;
   8) Optionally substituted arylalkyl;
   9) Optionally substituted heteroaryl; and
   10) Optionally substituted heteroarylalkyl;
R₁₃ is hydrogen, optionally substituted —C₁-C₈ alkyl, or Na⁺.

In certain embodiments, the present invention provides a pharmaceutical composition comprising a therapeutically effective amount of a compound or combination of compounds of the present invention, or a pharmaceutically acceptable salt, ester or combination thereof, in combination with a pharmaceutically acceptable carrier or excipient.

In certain embodiments, the present invention provides a method for the prevention or treatment of an 17β-HSD13 mediated disease or condition. The method comprises administering a therapeutically effective amount of a compound of Formula (I) or a pharmaceutically acceptable salt thereof. The present invention also provides the use of a compound of Formula (I), or a pharmaceutically acceptable salt thereof, in the manufacture of a medicament for the prevention or treatment of a 17β-HSD13 mediated disease or condition including, but not limited to: nonalcoholic fatty liver disease (NAFLD), nonalcoholic steatohepatitis (NASH), liver cirrhosis, liver fibrosis, hepatocellular carcinoma (HCC), and other metabolic disorders.

### DETAILED DESCRIPTION OF THE INVENTION

In one embodiment of the invention is a compound represented by Formula (I) as described above, or a pharmaceutically acceptable salt or ester thereof.

In certain embodiments of the compounds of Formula (I), R₃ is hydrogen or halogen.

In certain embodiments of the compounds of Formula (I), R₄ is hydrogen or halogen.

In certain embodiments of the compounds of Formula (I), R₅ is hydrogen or halogen.

In certain embodiments of the compounds of Formula (I), R₃ is hydrogen, R₄ is hydrogen, and R₅ is hydrogen.

In certain embodiments of the compounds of Formula (I), R₆ is —OR₉.

In certain embodiments of the compounds of Formula (I), R₆ is —OH.

In certain embodiments of the compounds of Formula (I), R₁ is optionally substituted aryl or optionally substituted heteroaryl.

In certain embodiments of the compounds of Formula (I), R₁ is optionally substituted heterocycloalkyl-C₁-C₆-alkyl.

In certain embodiments of the compounds of Formula (I), R₁ is optionally substituted —C₃-C₈ cycloalkyl or optionally substituted 3- to 8-membered heterocycloalkyl.

In certain embodiments of the compounds of Formula (I), R₁ is selected from the groups below, wherein each group is optionally substituted:

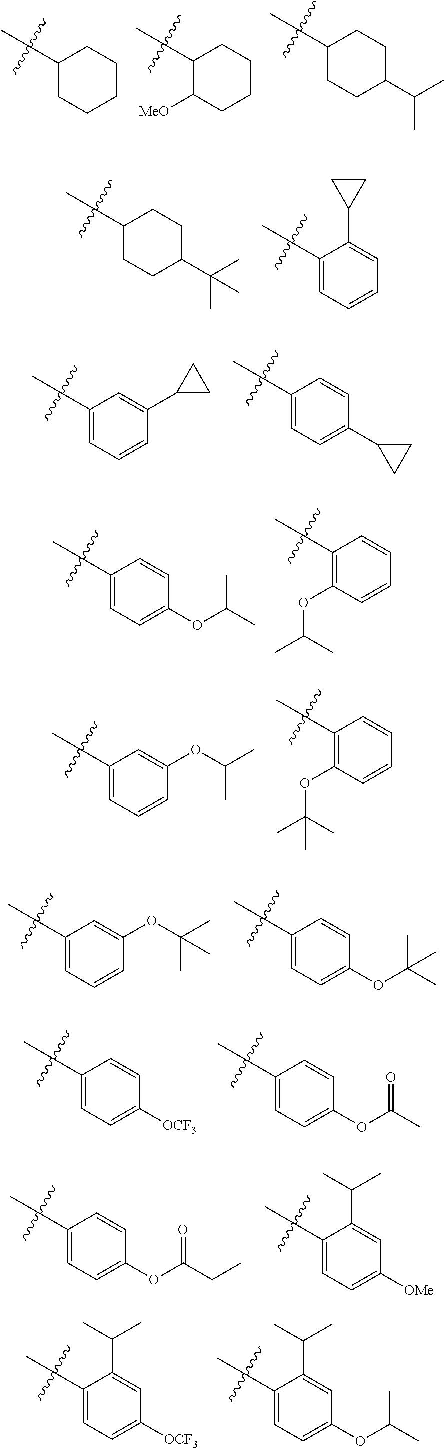

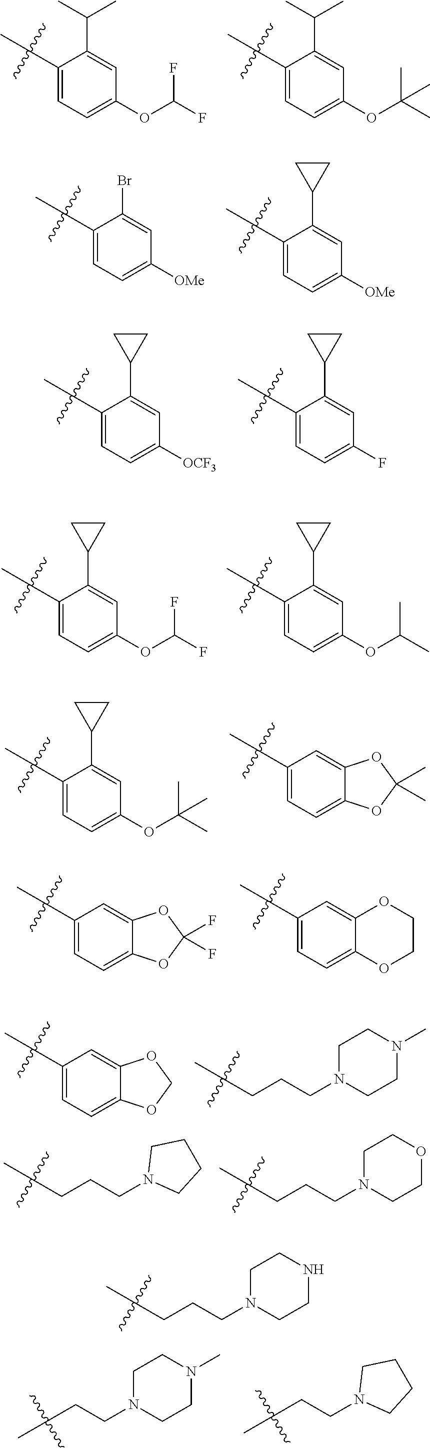

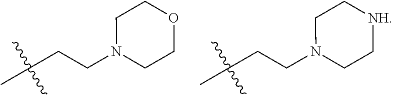 [SMILES: *CCN1CCOCC1 ; *CCN1CCNCC1]

In certain embodiments of the compounds of Formula (I), R₂ is optionally substituted —C₁-C₈ alkyl, preferably optionally substituted C₁-C₆-alkyl. Preferred substituents include halogen, C₃-C₆-cycloalkyl, hydroxy, amino, C₁-C₆-alkylamino, di(C₁-C₆-alkyl)amino, C₁-C₆-alkylNHC(O)—, di(C₁-C₆-alkyl)NC(O)— and —C(O)O—C₁-C₆-alkyl.

In certain embodiments of the compounds of Formula (I), R₂ is optionally substituted —C₂-C₈ alkenyl, preferably optionally substituted C₂-C₆-alkenyl. Preferred substituents include halogen, C₃-C₆-cycloalkyl, hydroxy, amino, C₁-C₆-alkylamino, di(C₁-C₆-alkyl)amino and —C(O)O—C₁-C₆-alkyl.

In certain embodiments of the compounds of Formula (I), R₂ is optionally substituted —C₃-C₈ cycloalkyl, preferably optionally substituted C₃-C₆-cycloalkyl. Preferred substituents include halogen, C₁-C₄-alkyl, and hydroxy.

In certain embodiments of the compounds of Formula (I), R₂ is optionally substituted arylalkyl or optionally substituted heteroarylalkyl.

In certain embodiments of the compounds of Formula (I), R₂ is optionally substituted aryl-C₁-C₆-alkyl, optionally substituted heteroaryl-C₁-C₆-alkyl or optionally substituted heterocyclyl-C₁-C₆-alkyl, preferably optionally substituted aryl-C₁-C₄-alkyl, optionally substituted heteroaryl-C₁-C₄-alkyl or optionally substituted heterocyclyl-C₁-C₄-alkyl. Preferred substituents include halogen, C₁-C₄-alkyl, and hydroxy.

In certain embodiments of the compounds of Formula (I), R₂ is optionally substituted aryl-C₂-C₆-alkenyl, optionally substituted heteroaryl-C₂-C₆-alkenyl or optionally substituted heterocyclyl-C₂-C₆-alkenyl, preferably optionally substituted aryl-C₂-C₄-alkenyl, optionally substituted heteroaryl-C₂-C₄-alkenyl or optionally substituted heterocyclyl-C₂-C₄-alkenyl. Preferred substituents include halogen, C₁-C₄-alkyl, and hydroxy.

In certain embodiments of the compounds of Formula (I), R₂ is optionally substituted —C₁-C₄-alkylN(R)—C₁-C₄-alkylaryl, optionally substituted —C₁-C₄-alkylN(R)—C₁-C₄-alkylheteroaryl or optionally substituted —C₁-C₄-alkylN(R)—C₁-C₄-alkylheterocyclyl, where R is H or C₁-C₄-alkyl. Preferred substituents include halogen, C₁-C₄-alkyl, and hydroxy.

In certain embodiments of the compounds of Formula (I), R₂ is —C₁-C₆-alkyl-L-R′, where L is —O—, —S—, —N(R)—, —N(R)C(O)—, —NRC(O)O—, or —N(R)SO₂—; R′ is optionally substituted C₁-C₆-alkyl, optionally substituted aryl, or optionally substituted heteroaryl; alternatively R, R′ and the nitrogen atom to which they are attached form an optionally substituted 3- to 8-membered heterocyclyl.

In certain embodiments of the compounds of Formula (I), R₂ is selected from the group below, wherein each is optionally substituted:

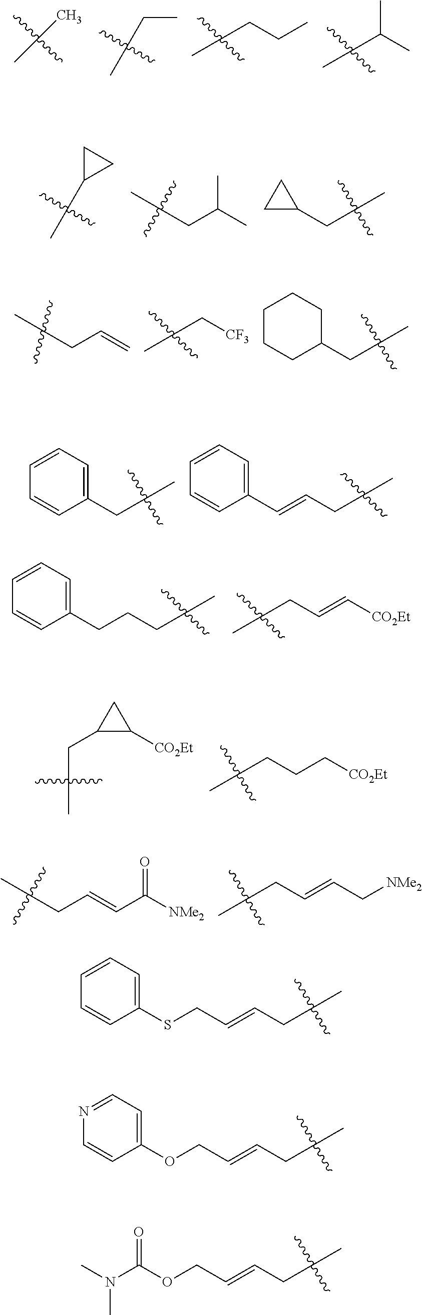

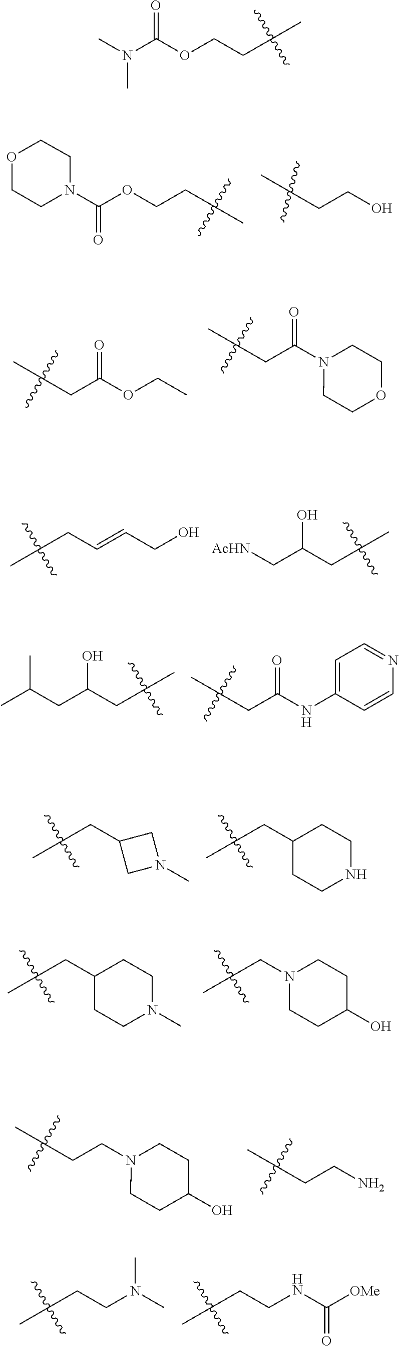

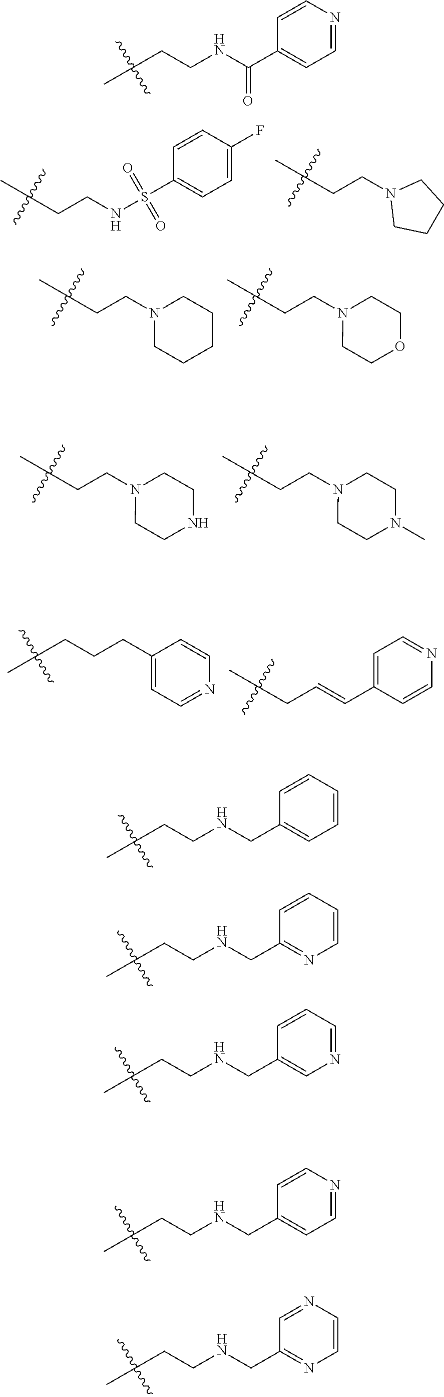

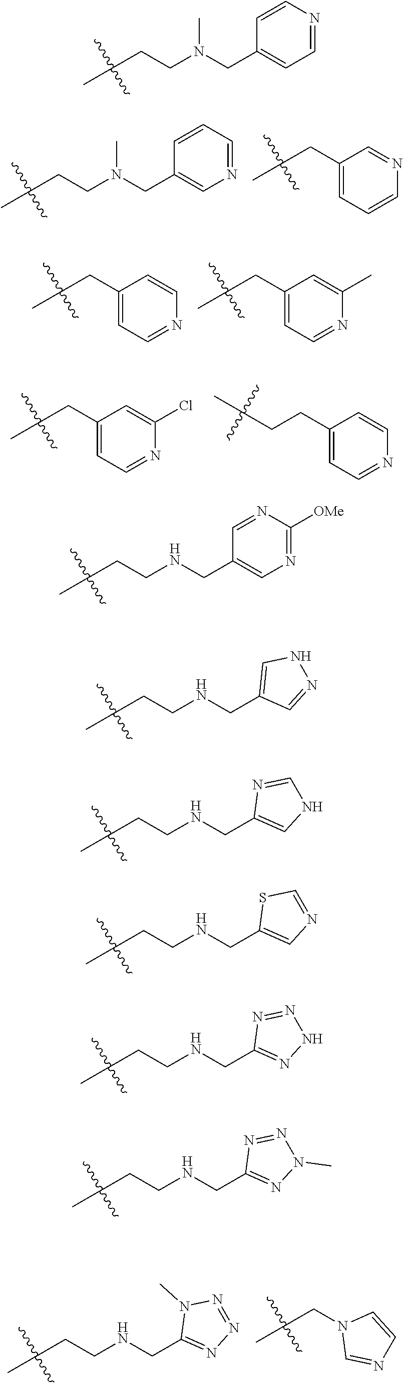

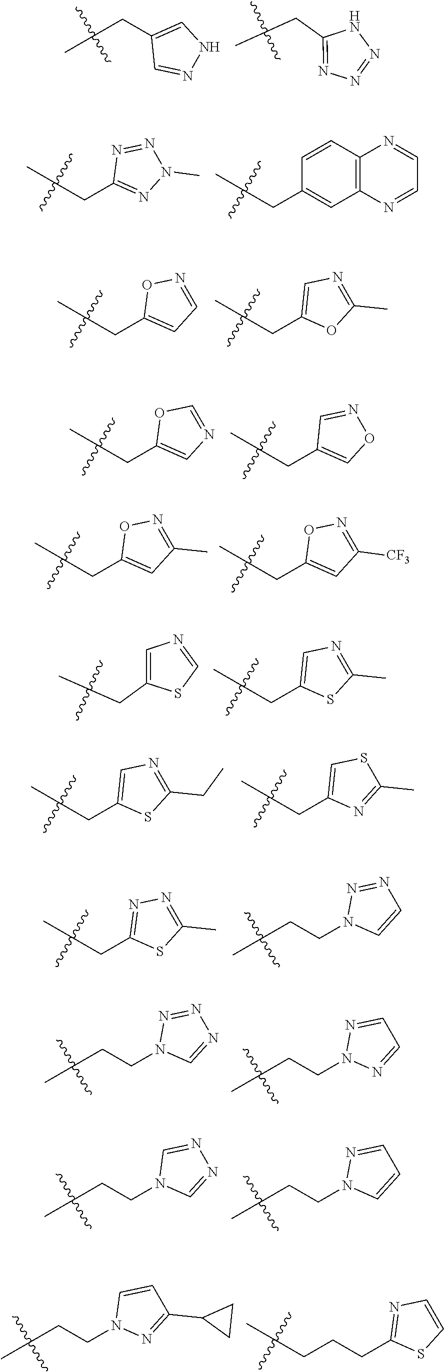

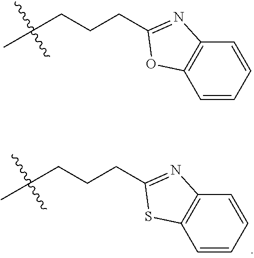 [SMILES: *CCCc1nc2ccccc2o1 ; *CCCc1nc2ccccc2s1]

In one embodiment, the present invention provides compounds represented by Formula (II) or (III), or a pharmaceutically acceptable salt or ester thereof:

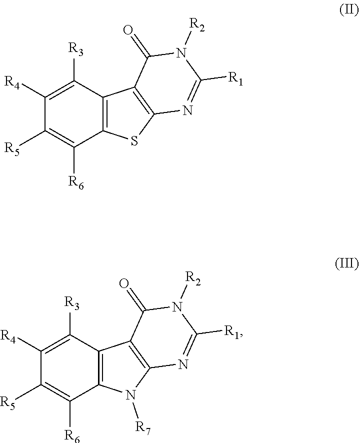

wherein, R₁, R₂, R₃, R₄, R₅, R₆, and R₇ are as previously defined.

In certain embodiments, the present invention provides compounds represented by Formula (IV) or (V), or a pharmaceutically acceptable salt or ester thereof:

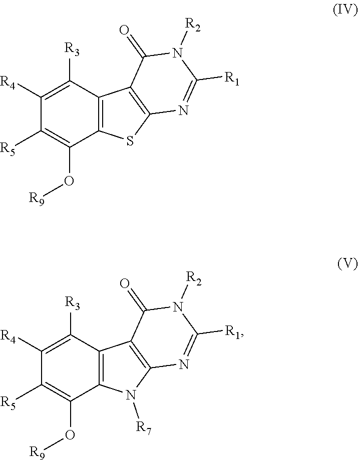

wherein, R₁, R₂, R₃, R₄, R₅, R₇, and R₉ are as previously defined.

In certain embodiments, the present invention provides compounds represented by Formula (IV) or (V), or a pharmaceutically acceptable salt or ester thereof, wherein R₉ is selected from the groups below, wherein each group is optionally substituted:

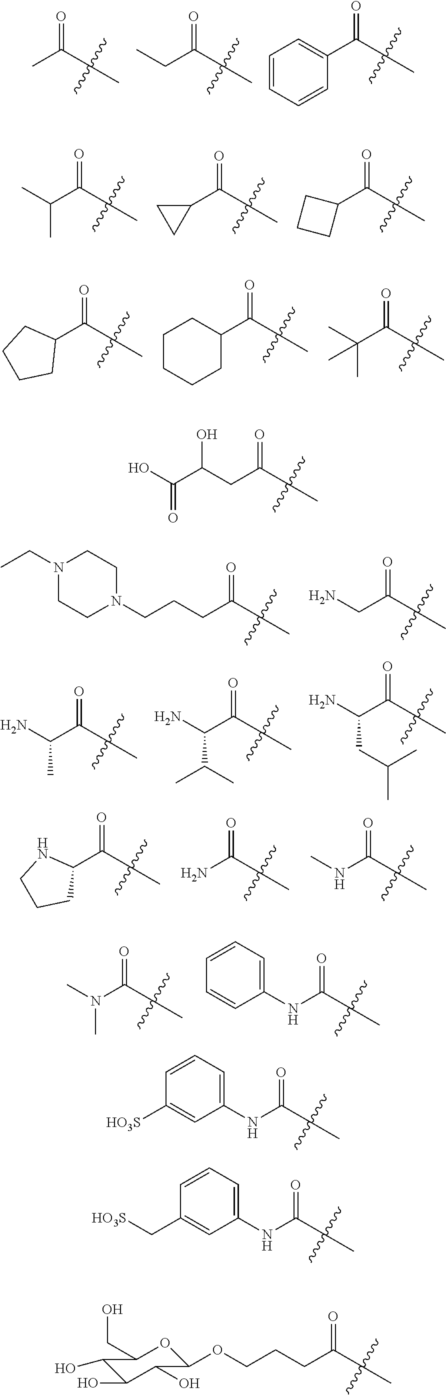

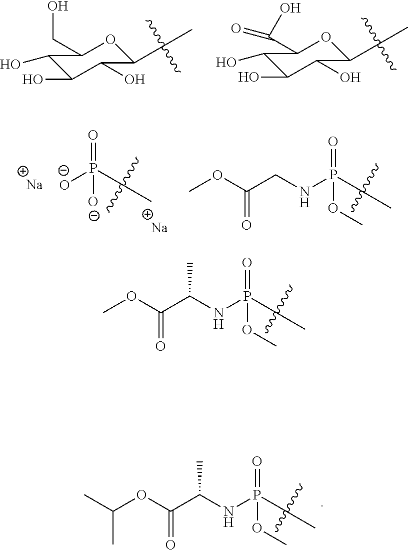

In one embodiment, the present invention provides compounds represented by Formula (VI) or (VII), or a pharmaceutically acceptable salt or ester thereof:

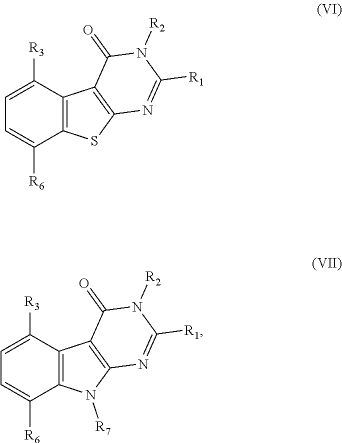

wherein, R₁, R₂, R₃, R₆, and R₇ are as previously defined.

In certain embodiments, the present invention provides compounds represented by Formula (VIII) or (IX), or a pharmaceutically acceptable salt or ester thereof:

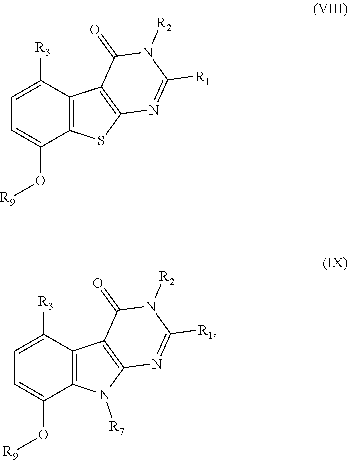

wherein, R₁, R₂, R₃, R₇, and R₉ are as previously defined.

In certain embodiments, the present invention provides compounds represented by Formula (X) or (XI), or a pharmaceutically acceptable salt or ester thereof:

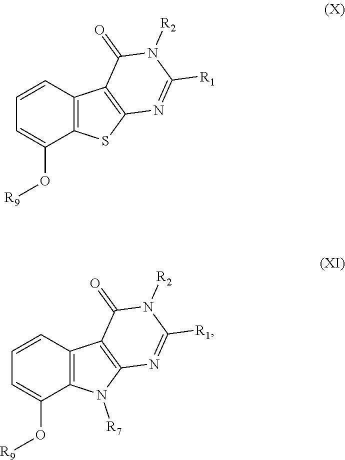

wherein, R₁, R₂, R₇, and R₉ are as previously defined. Preferably R₉ is hydrogen.

In certain embodiments, the present invention provides compounds represented by Formula (X) or (XI), or a pharmaceutically acceptable salt or ester thereof, wherein R₉ is selected from the group consisting of below, wherein each of them is optionally substituted:

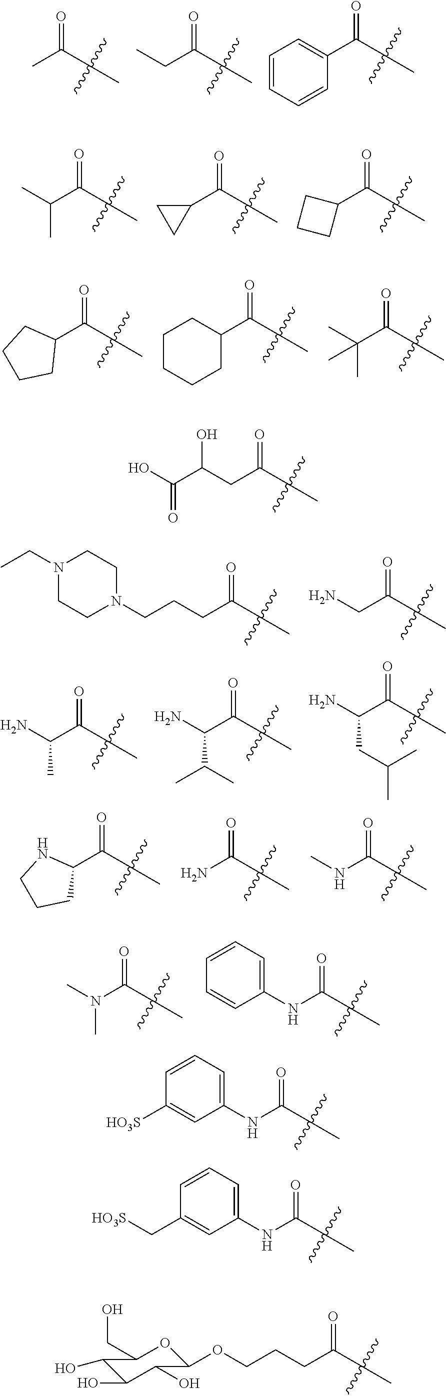

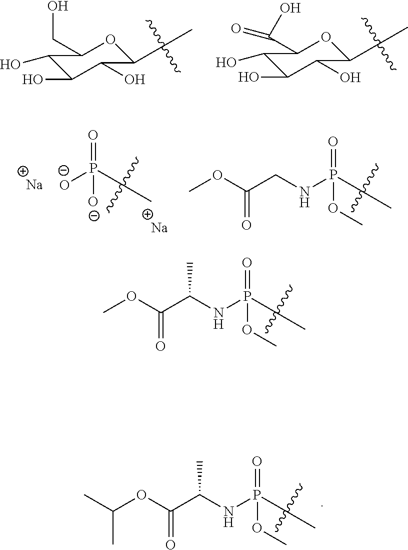

Representative compounds of the invention include, but are not limited to, the following compounds (Entry 1 to Entry 80 in Table 1) according to Formula (X), wherein R₉ is hydrogen, and R₁ and R₂ are delineated for each compound in Table 1,

TABLE 1

| Entry | R₁ | R₂ |
|---|---|---|
| 1 | 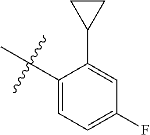 [SMILES: *c1ccc(F)cc1C1CC1] | 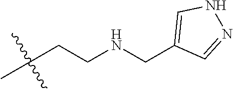 [SMILES: *CCNCc1cn[nH]c1] |
| 2 | 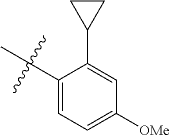 [SMILES: *c1ccc(OC)cc1C1CC1] | 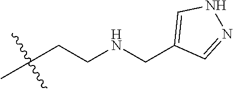 [SMILES: *CCNCc1cn[nH]c1] |
| 3 | 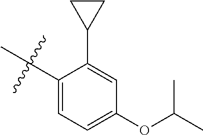 [SMILES: *c1ccc(OC(C)C)cc1C1CC1] | 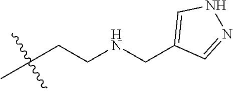 [SMILES: *CCNCc1cn[nH]c1] |
| 4 | 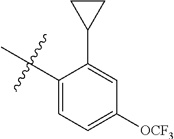 [SMILES: *c1ccc(OC(F)(F)F)cc1C1CC1] | 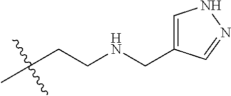 [SMILES: *CCNCc1cn[nH]c1] |
| 5 | 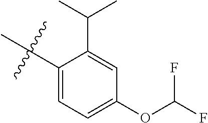 [SMILES: *c1ccc(OC(F)F)cc1C(C)C] | 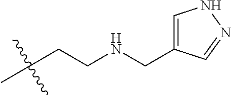 [SMILES: *CCNCc1cn[nH]c1] |
| 6 | 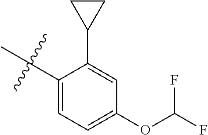 [SMILES: *c1ccc(OC(F)F)cc1C1CC1] | 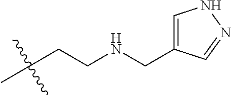 [SMILES: *CCNCc1cn[nH]c1] |
| 7 | 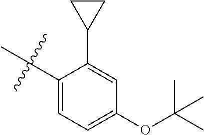 [SMILES: *c1ccc(OC(C)(C)C)cc1C1CC1] | 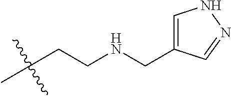 [SMILES: *CCNCc1cn[nH]c1] |
| 8 | 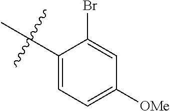 [SMILES: *c1ccc(OC)cc1Br] | 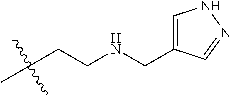 [SMILES: *CCNCc1cn[nH]c1] |
| 9 | 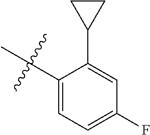 [SMILES: *c1ccc(F)cc1C1CC1] | 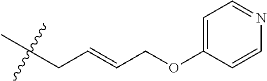 [SMILES: *C/C=C/COc1ccncc1] |
| 10 | 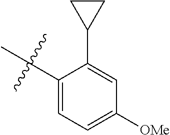 [SMILES: *c1ccc(OC)cc1C1CC1] | 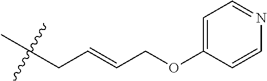 [SMILES: *C/C=C/COc1ccncc1] |
| 11 | 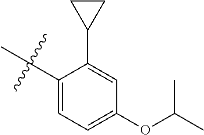 [SMILES: *c1ccc(OC(C)C)cc1C1CC1] | 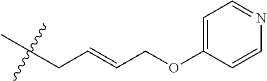 [SMILES: *C/C=C/COc1ccncc1] |
| 12 | 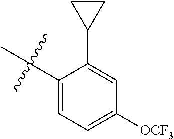 [SMILES: *c1ccc(OC(F)(F)F)cc1C1CC1] | 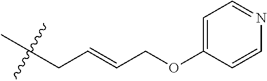 [SMILES: *C/C=C/COc1ccncc1] |
| 13 | 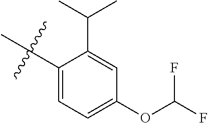 [SMILES: *c1ccc(OC(F)F)cc1C(C)C] | 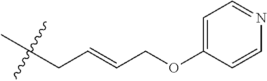 [SMILES: *C/C=C/COc1ccncc1] |
| 14 | 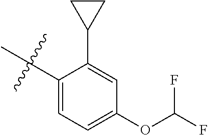 [SMILES: *c1ccc(OC(F)F)cc1C1CC1] | 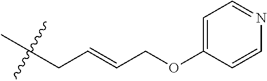 [SMILES: *C/C=C/COc1ccncc1] |
| 15 | 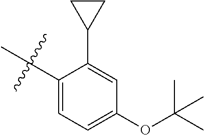 [SMILES: *c1ccc(OC(C)(C)C)cc1C1CC1] | 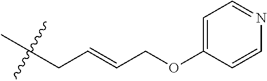 [SMILES: *C/C=C/COc1ccncc1] |
| 16 | 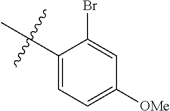 [SMILES: *c1ccc(OC)cc1Br] | 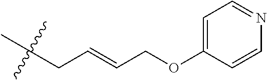 [SMILES: *C/C=C/COc1ccncc1] |
| 17 | 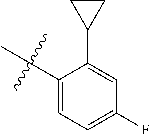 [SMILES: *c1ccc(F)cc1C1CC1] | 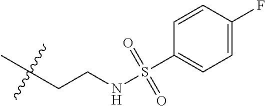 [SMILES: *CCNS(=O)(=O)c1ccc(F)cc1] |
| 18 | 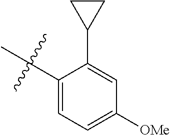 [SMILES: *c1ccc(OC)cc1C1CC1] | 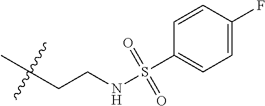 [SMILES: *CCNS(=O)(=O)c1ccc(F)cc1] |
| 19 | 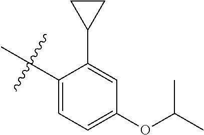 [SMILES: *c1ccc(OC(C)C)cc1C1CC1] | 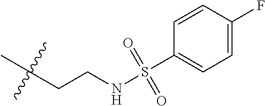 [SMILES: *CCNS(=O)(=O)c1ccc(F)cc1] |
| 20 | 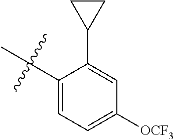 [SMILES: *c1ccc(OC(F)(F)F)cc1C1CC1] | 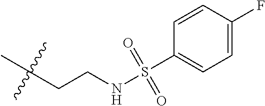 [SMILES: *CCNS(=O)(=O)c1ccc(F)cc1] |
| 21 | 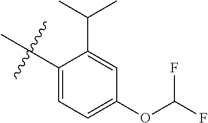 [SMILES: *c1ccc(OC(F)F)cc1C(C)C] | 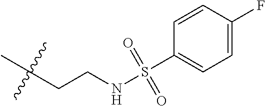 [SMILES: *CCNS(=O)(=O)c1ccc(F)cc1] |
| 22 | 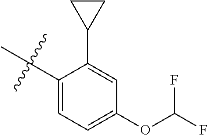 [SMILES: *c1ccc(OC(F)F)cc1C1CC1] | 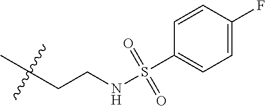 [SMILES: *CCNS(=O)(=O)c1ccc(F)cc1] |
| 23 | 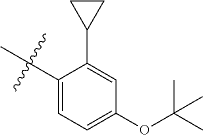 [SMILES: *c1ccc(OC(C)(C)C)cc1C1CC1] | 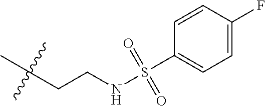 [SMILES: *CCNS(=O)(=O)c1ccc(F)cc1] |
| 24 | 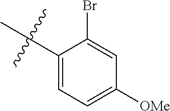 [SMILES: *c1ccc(OC)cc1Br] | 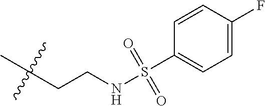 [SMILES: *CCNS(=O)(=O)c1ccc(F)cc1] |
| 25 | 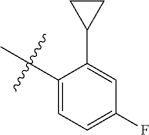 [SMILES: *c1ccc(F)cc1C1CC1] | 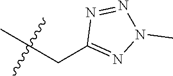 [SMILES: *Cc1nnn(C)n1] |
| 26 | 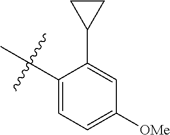 [SMILES: *c1ccc(OC)cc1C1CC1] | 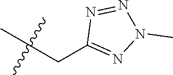 [SMILES: *Cc1nnn(C)n1] |
| 27 | 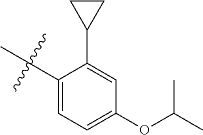 [SMILES: *c1ccc(OC(C)C)cc1C1CC1] | 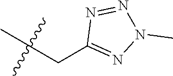 [SMILES: *Cc1nnn(C)n1] |
| 28 | 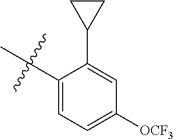 [SMILES: *c1ccc(OC(F)(F)F)cc1C1CC1] | 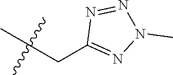 [SMILES: *Cc1nnn(C)n1] |
| 29 | 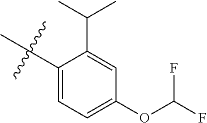 [SMILES: *c1ccc(OC(F)F)cc1C(C)C] | 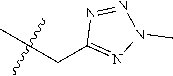 [SMILES: *Cc1nnn(C)n1] |
| 30 | 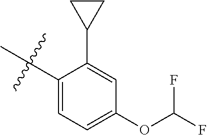 [SMILES: *c1ccc(OC(F)F)cc1C1CC1] | 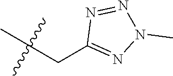 [SMILES: *Cc1nnn(C)n1] |
| 31 | 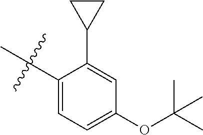 [SMILES: *c1ccc(OC(C)(C)C)cc1C1CC1] | 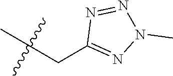 [SMILES: *Cc1nnn(C)n1] |
| 32 | 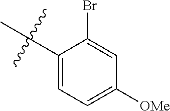 [SMILES: *c1ccc(OC)cc1Br] | 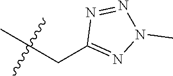 [SMILES: *Cc1nnn(C)n1] |
| 33 | 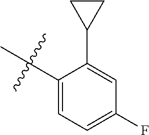 [SMILES: *c1ccc(F)cc1C1CC1] | 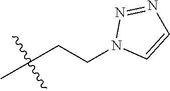 [SMILES: *CCn1ccnn1] |
| 34 | 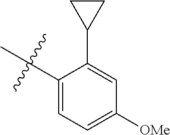 [SMILES: *c1ccc(OC)cc1C1CC1] | 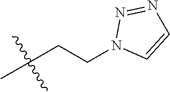 [SMILES: *CCn1ccnn1] |
| 35 | 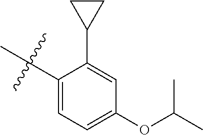 [SMILES: *c1ccc(OC(C)C)cc1C1CC1] | 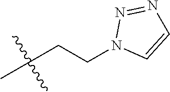 [SMILES: *CCn1ccnn1] |
| 36 | 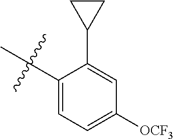 [SMILES: *c1ccc(OC(F)(F)F)cc1C1CC1] | 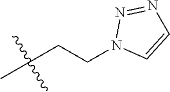 [SMILES: *CCn1ccnn1] |
| 37 | 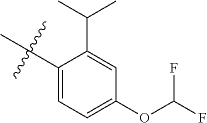 [SMILES: *c1ccc(OC(F)F)cc1C(C)C] | 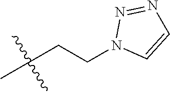 [SMILES: *CCn1ccnn1] |
| 38 | 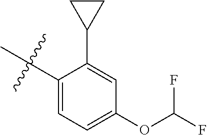 [SMILES: *c1ccc(OC(F)F)cc1C1CC1] | 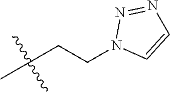 [SMILES: *CCn1ccnn1] |
| 39 | 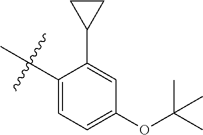 [SMILES: *c1ccc(OC(C)(C)C)cc1C1CC1] | 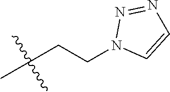 [SMILES: *CCn1ccnn1] |
| 40 | 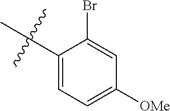 [SMILES: *c1ccc(OC)cc1Br] | 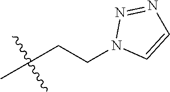 [SMILES: *CCn1ccnn1] |
| 41 | 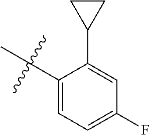 [SMILES: *c1ccc(F)cc1C1CC1] | 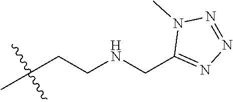 [SMILES: *CCNCc1nnnn1C] |
| 42 | 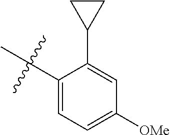 [SMILES: *c1ccc(OC)cc1C1CC1] | 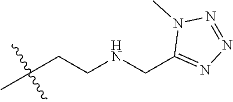 [SMILES: *CCNCc1nnnn1C] |
| 43 | 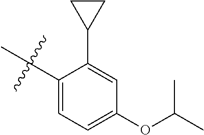 [SMILES: *c1ccc(OC(C)C)cc1C1CC1] | 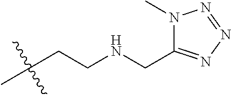 [SMILES: *CCNCc1nnnn1C] |
| 44 | 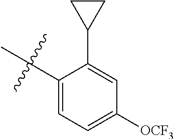 [SMILES: *c1ccc(OC(F)(F)F)cc1C1CC1] | 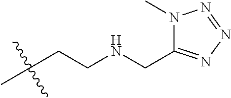 [SMILES: *CCNCc1nnnn1C] |
| 45 | 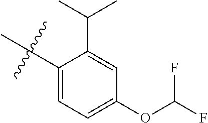 [SMILES: *c1ccc(OC(F)F)cc1C(C)C] | 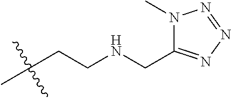 [SMILES: *CCNCc1nnnn1C] |
| 46 | 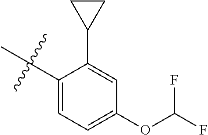 [SMILES: *c1ccc(OC(F)F)cc1C1CC1] | 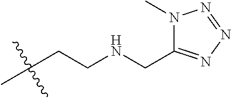 [SMILES: *CCNCc1nnnn1C] |
| 47 | 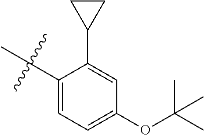 [SMILES: *c1ccc(OC(C)(C)C)cc1C1CC1] | 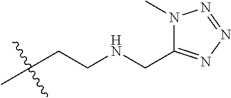 [SMILES: *CCNCc1nnnn1C] |
| 48 | 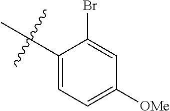 [SMILES: *c1ccc(OC)cc1Br] | 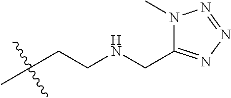 [SMILES: *CCNCc1nnnn1C] |
| 49 | 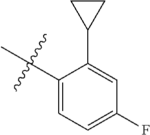 [SMILES: *c1ccc(F)cc1C1CC1] | 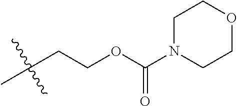 [SMILES: *CCOC(=O)N1CCOCC1] |
| 50 | 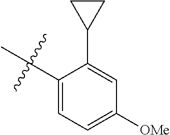 [SMILES: *c1ccc(OC)cc1C1CC1] | 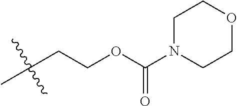 [SMILES: *CCOC(=O)N1CCOCC1] |
| 51 | 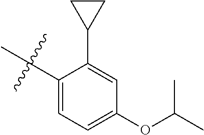 [SMILES: *c1ccc(OC(C)C)cc1C1CC1] | 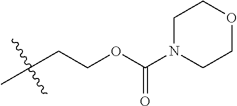 [SMILES: *CCOC(=O)N1CCOCC1] |
| 52 | 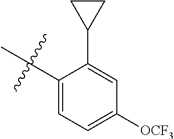 [SMILES: *c1ccc(OC(F)(F)F)cc1C1CC1] | 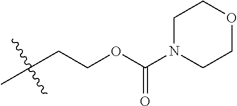 [SMILES: *CCOC(=O)N1CCOCC1] |
| 53 | 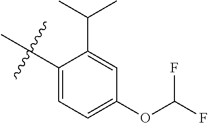 [SMILES: *c1ccc(OC(F)F)cc1C(C)C] | 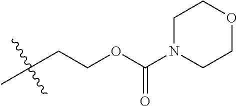 [SMILES: *CCOC(=O)N1CCOCC1] |
| 54 | 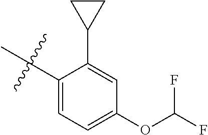 [SMILES: *c1ccc(OC(F)F)cc1C1CC1] | 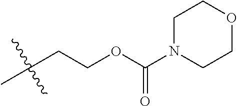 [SMILES: *CCOC(=O)N1CCOCC1] |
| 55 | 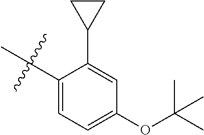 [SMILES: *c1ccc(OC(C)(C)C)cc1C1CC1] | 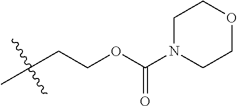 [SMILES: *CCOC(=O)N1CCOCC1] |
| 56 | 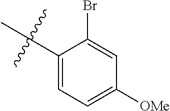 [SMILES: *c1ccc(OC)cc1Br] | 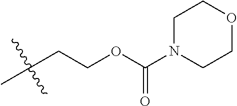 [SMILES: *CCOC(=O)N1CCOCC1] |
| 57 | 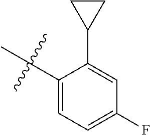 [SMILES: *c1ccc(F)cc1C1CC1] | 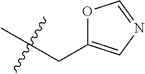 [SMILES: *Cc1cnco1] |
| 58 | 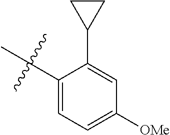 [SMILES: *c1ccc(OC)cc1C1CC1] | 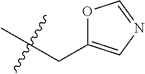 [SMILES: *Cc1cnco1] |
| 59 | 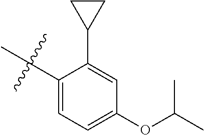 [SMILES: *c1ccc(OC(C)C)cc1C1CC1] | 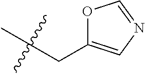 [SMILES: *Cc1cnco1] |
| 60 | 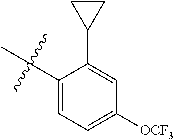 [SMILES: *c1ccc(OC(F)(F)F)cc1C1CC1] | 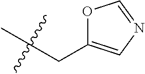 [SMILES: *Cc1cnco1] |
| 61 | 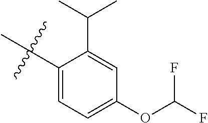 [SMILES: *c1ccc(OC(F)F)cc1C(C)C] | 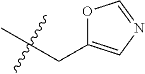 [SMILES: *Cc1cnco1] |
| 62 | 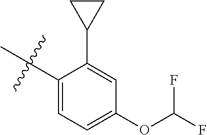 [SMILES: *c1ccc(OC(F)F)cc1C1CC1] | 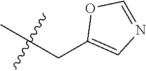 [SMILES: *Cc1cnco1] |
| 63 | 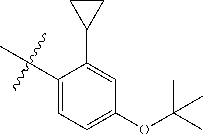 [SMILES: *c1ccc(OC(C)(C)C)cc1C1CC1] | 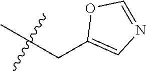 [SMILES: *Cc1cnco1] |
| 64 | 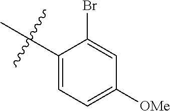 [SMILES: *c1ccc(OC)cc1Br] | 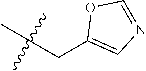 [SMILES: *Cc1cnco1] |
| 65 | 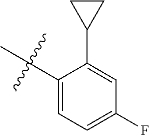 [SMILES: *c1ccc(F)cc1C1CC1] | 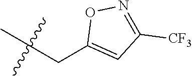 [SMILES: *Cc1cc(C(F)(F)F)no1] |
| 66 | 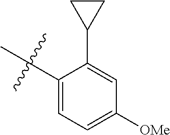 [SMILES: *c1ccc(OC)cc1C1CC1] | 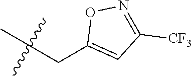 [SMILES: *Cc1cc(C(F)(F)F)no1] |
| 67 | 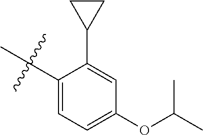 [SMILES: *c1ccc(OC(C)C)cc1C1CC1] | 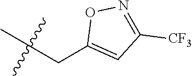 [SMILES: *Cc1cc(C(F)(F)F)no1] |
| 68 | 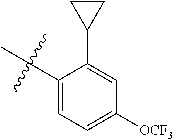 [SMILES: *c1ccc(OC(F)(F)F)cc1C1CC1] | 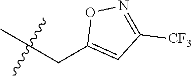 [SMILES: *Cc1cc(C(F)(F)F)no1] |
| 69 | 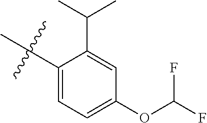 [SMILES: *c1ccc(OC(F)F)cc1C(C)C] | 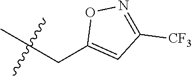 [SMILES: *Cc1cc(C(F)(F)F)no1] |
| 70 | 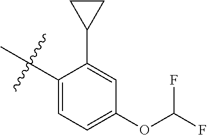 [SMILES: *c1ccc(OC(F)F)cc1C1CC1] | 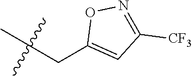 [SMILES: *Cc1cc(C(F)(F)F)no1] |
| 71 | 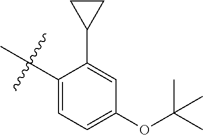 [SMILES: *c1ccc(OC(C)(C)C)cc1C1CC1] | 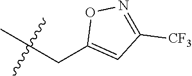 [SMILES: *Cc1cc(C(F)(F)F)no1] |
| 72 | 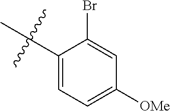 [SMILES: *c1ccc(OC)cc1Br] | 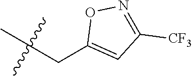 [SMILES: *Cc1cc(C(F)(F)F)no1] |
| 73 | 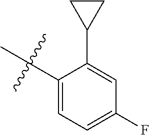 [SMILES: *c1ccc(F)cc1C1CC1] | 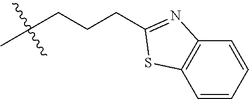 [SMILES: *CCCc1nc2ccccc2s1] |
| 74 | 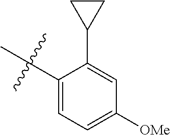 [SMILES: *c1ccc(OC)cc1C1CC1] | 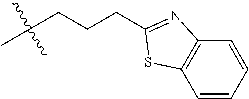 [SMILES: *CCCc1nc2ccccc2s1] |
| 75 | 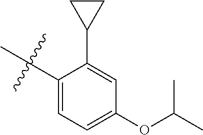 [SMILES: *c1ccc(OC(C)C)cc1C1CC1] | 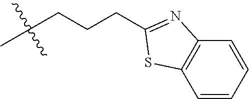 [SMILES: *CCCc1nc2ccccc2s1] |
| 76 | 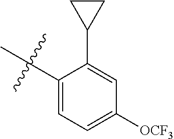 [SMILES: *c1ccc(OC(F)(F)F)cc1C1CC1] | 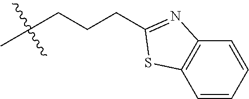 [SMILES: *CCCc1nc2ccccc2s1] |
| 77 | 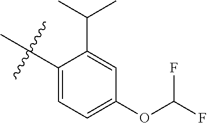 [SMILES: *c1ccc(OC(F)F)cc1C(C)C] | 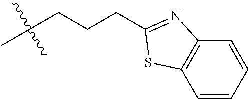 [SMILES: *CCCc1nc2ccccc2s1] |
| 78 | 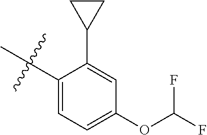 [SMILES: *c1ccc(OC(F)F)cc1C1CC1] | 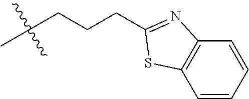 [SMILES: *CCCc1nc2ccccc2s1] |
| 79 | 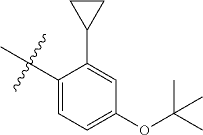 [SMILES: *c1ccc(OC(C)(C)C)cc1C1CC1] | 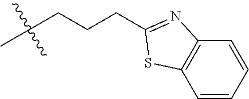 [SMILES: *CCCc1nc2ccccc2s1] |
| 80 | 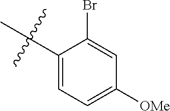 [SMILES: *c1ccc(OC)cc1Br] | 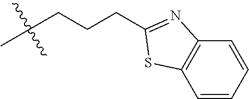 [SMILES: *CCCc1nc2ccccc2s1] |

Representative compounds of the invention include, but are not limited to, the following compounds (Entry 81 to Entry 230 in Table 2) according to Formula (X), wherein R₁, R₂ and R₉ are delineated for each compound in Table 2.

TABLE 2

| Entry | R₁ | R₂ | R₉ |
|---|---|---|---|
| 81 | 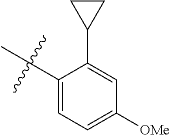 [SMILES: *c1ccc(OC)cc1C1CC1] | 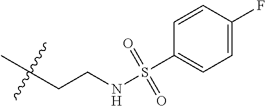 [SMILES: *CCNS(=O)(=O)c1ccc(F)cc1] | 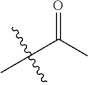 [SMILES: *C(C)=O] |
| 82 | 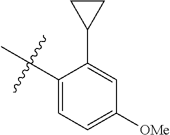 [SMILES: *c1ccc(OC)cc1C1CC1] | 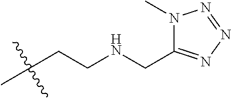 [SMILES: *CCNCc1nnnn1C] | 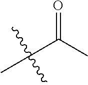 [SMILES: *C(C)=O] |
| 83 | 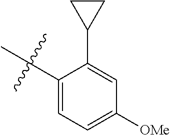 [SMILES: *c1ccc(OC)cc1C1CC1] | 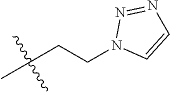 [SMILES: *CCn1ccnn1] | 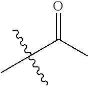 [SMILES: *C(C)=O] |
| 84 | 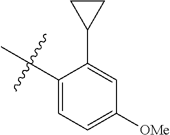 [SMILES: *c1ccc(OC)cc1C1CC1] | 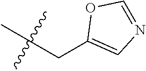 [SMILES: *Cc1cnco1] | 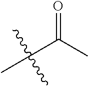 [SMILES: *C(C)=O] |
| 85 | 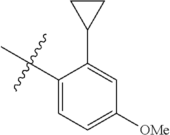 [SMILES: *c1ccc(OC)cc1C1CC1] | 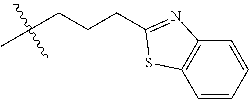 [SMILES: *CCCc1nc2ccccc2s1] | 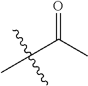 [SMILES: *C(C)=O] |
| 86 | 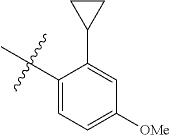 [SMILES: *c1ccc(OC)cc1C1CC1] | 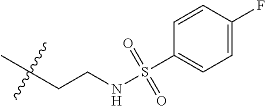 [SMILES: *CCNS(=O)(=O)c1ccc(F)cc1] | 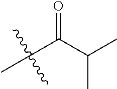 [SMILES: *C(=O)C(C)C] |
| 87 | 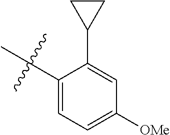 [SMILES: *c1ccc(OC)cc1C1CC1] | 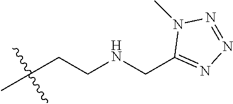 [SMILES: *CCNCc1nnnn1C] | 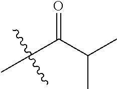 [SMILES: *C(=O)C(C)C] |
| 88 | 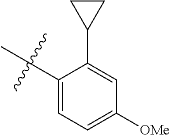 [SMILES: *c1ccc(OC)cc1C1CC1] | 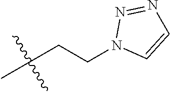 [SMILES: *CCn1ccnn1] | 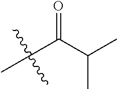 [SMILES: *C(=O)C(C)C] |
| 89 | 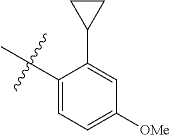 [SMILES: *c1ccc(OC)cc1C1CC1] | 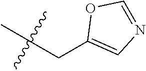 [SMILES: *Cc1cnco1] | 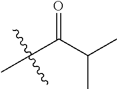 [SMILES: *C(=O)C(C)C] |
| 90 | 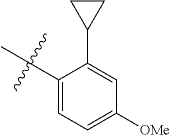 [SMILES: *c1ccc(OC)cc1C1CC1] | 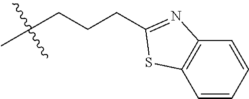 [SMILES: *CCCc1nc2ccccc2s1] | 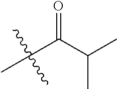 [SMILES: *C(=O)C(C)C] |
| 91 | 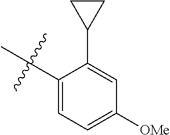 [SMILES: *c1ccc(OC)cc1C1CC1] | 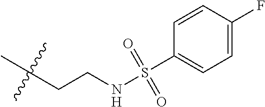 [SMILES: *CCNS(=O)(=O)c1ccc(F)cc1] | 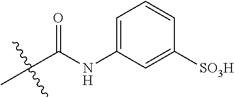 [SMILES: *C(=O)Nc1cccc(S(=O)(=O)O)c1] |
| 92 | 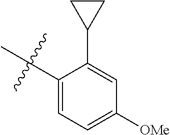 [SMILES: *c1ccc(OC)cc1C1CC1] | 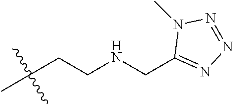 [SMILES: *CCNCc1nnnn1C] | 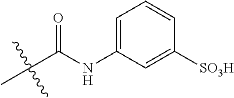 [SMILES: *C(=O)Nc1cccc(S(=O)(=O)O)c1] |
| 93 | 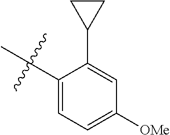 [SMILES: *c1ccc(OC)cc1C1CC1] | 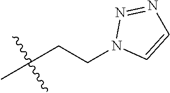 [SMILES: *CCn1ccnn1] | 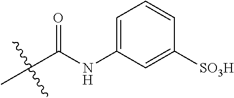 [SMILES: *C(=O)Nc1cccc(S(=O)(=O)O)c1] |
| 94 | 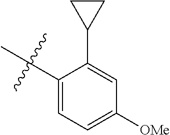 [SMILES: *c1ccc(OC)cc1C1CC1] | 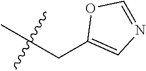 [SMILES: *Cc1cnco1] | 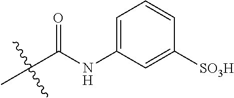 [SMILES: *C(=O)Nc1cccc(S(=O)(=O)O)c1] |
| 95 | 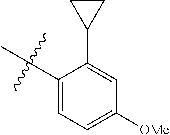 [SMILES: *c1ccc(OC)cc1C1CC1] | 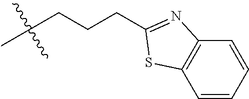 [SMILES: *CCCc1nc2ccccc2s1] | 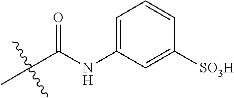 [SMILES: *C(=O)Nc1cccc(S(=O)(=O)O)c1] |
| 96 | 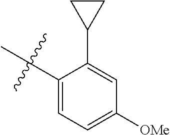 [SMILES: *c1ccc(OC)cc1C1CC1] | 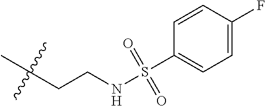 [SMILES: *CCNS(=O)(=O)c1ccc(F)cc1] | 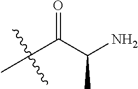 [SMILES: *C(=O)[C@H](C)N] |
| 97 | 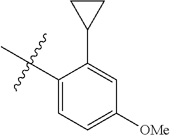 [SMILES: *c1ccc(OC)cc1C1CC1] | 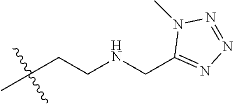 [SMILES: *CCNCc1nnnn1C] | 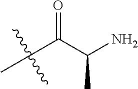 [SMILES: *C(=O)[C@H](C)N] |
| 98 | 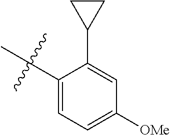 [SMILES: *c1ccc(OC)cc1C1CC1] | 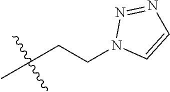 [SMILES: *CCn1ccnn1] | 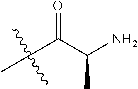 [SMILES: *C(=O)[C@H](C)N] |
| 99 | 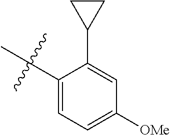 [SMILES: *c1ccc(OC)cc1C1CC1] | 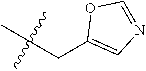 [SMILES: *Cc1cnco1] | 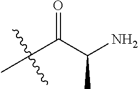 [SMILES: *C(=O)[C@H](C)N] |
| 100 | 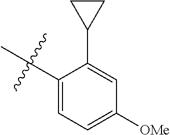 [SMILES: *c1ccc(OC)cc1C1CC1] | 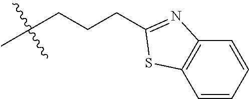 [SMILES: *CCCc1nc2ccccc2s1] | 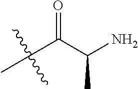 [SMILES: *C(=O)[C@H](C)N] |
| 101 | 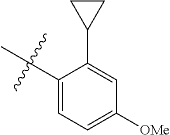 [SMILES: *c1ccc(OC)cc1C1CC1] | 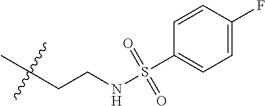 [SMILES: *CCNS(=O)(=O)c1ccc(F)cc1] | 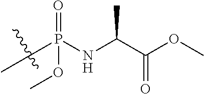 [SMILES: *P(=O)(N[C@@H](C)C(=O)OC)OC] |
| 102 | 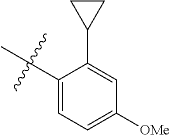 [SMILES: *c1ccc(OC)cc1C1CC1] | 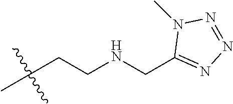 [SMILES: *CCNCc1nnnn1C] | 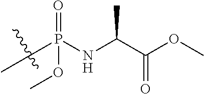 [SMILES: *P(=O)(N[C@@H](C)C(=O)OC)OC] |
| 103 | 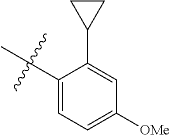 [SMILES: *c1ccc(OC)cc1C1CC1] | 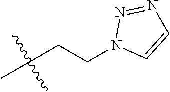 [SMILES: *CCn1ccnn1] | 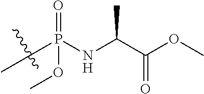 [SMILES: *P(=O)(N[C@@H](C)C(=O)OC)OC] |
| 104 | 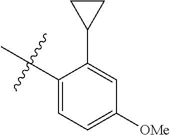 [SMILES: *c1ccc(OC)cc1C1CC1] | 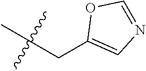 [SMILES: *Cc1cnco1] | 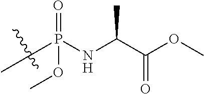 [SMILES: *P(=O)(N[C@@H](C)C(=O)OC)OC] |
| 105 | 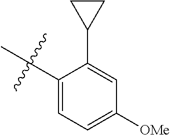 [SMILES: *c1ccc(OC)cc1C1CC1] | 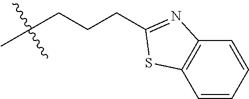 [SMILES: *CCCc1nc2ccccc2s1] | 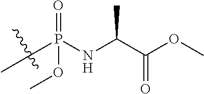 [SMILES: *P(=O)(N[C@@H](C)C(=O)OC)OC] |
| 106 | 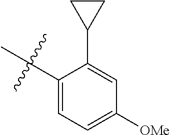 [SMILES: *c1ccc(OC)cc1C1CC1] | 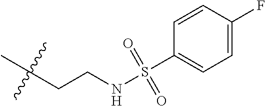 [SMILES: *CCNS(=O)(=O)c1ccc(F)cc1] | 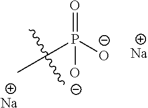 [SMILES: *P(=O)([O-])[O-] ; [Na+] ; [Na+]] |
| 107 | 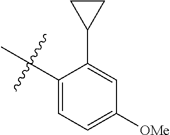 [SMILES: *c1ccc(OC)cc1C1CC1] | 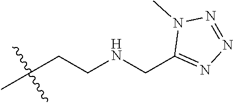 [SMILES: *CCNCc1nnnn1C] | 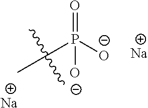 [SMILES: *P(=O)([O-])[O-] ; [Na+] ; [Na+]] |
| 108 | 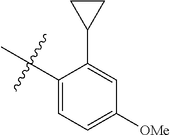 [SMILES: *c1ccc(OC)cc1C1CC1] | 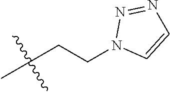 [SMILES: *CCn1ccnn1] | 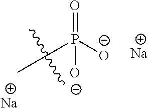 [SMILES: *P(=O)([O-])[O-] ; [Na+] ; [Na+]] |
| 109 | 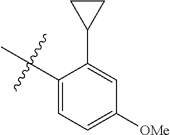 [SMILES: *c1ccc(OC)cc1C1CC1] | 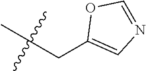 [SMILES: *Cc1cnco1] | 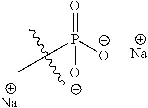 [SMILES: *P(=O)([O-])[O-] ; [Na+] ; [Na+]] |
| 110 | 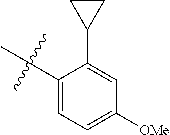 [SMILES: *c1ccc(OC)cc1C1CC1] | 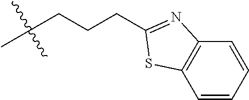 [SMILES: *CCCc1nc2ccccc2s1] | 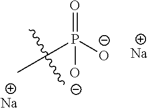 [SMILES: *P(=O)([O-])[O-] ; [Na+] ; [Na+]] |
| 111 | 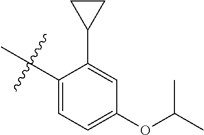 [SMILES: *c1ccc(OC(C)C)cc1C1CC1] | 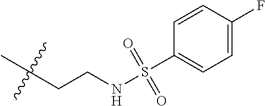 [SMILES: *CCNS(=O)(=O)c1ccc(F)cc1] | 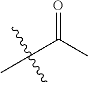 [SMILES: *C(C)=O] |
| 112 | 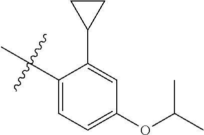 [SMILES: *c1ccc(OC(C)C)cc1C1CC1] | 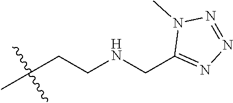 [SMILES: *CCNCc1nnnn1C] | 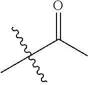 [SMILES: *C(C)=O] |
| 113 | 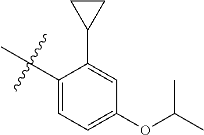 [SMILES: *c1ccc(OC(C)C)cc1C1CC1] | 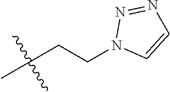 [SMILES: *CCn1ccnn1] | 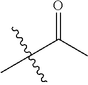 [SMILES: *C(C)=O] |
| 114 | 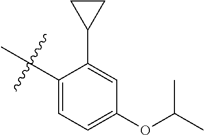 [SMILES: *c1ccc(OC(C)C)cc1C1CC1] | 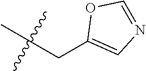 [SMILES: *Cc1cnco1] | 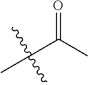 [SMILES: *C(C)=O] |
| 115 | 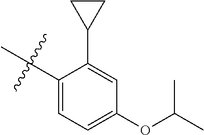 [SMILES: *c1ccc(OC(C)C)cc1C1CC1] | 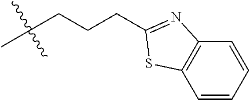 [SMILES: *CCCc1nc2ccccc2s1] | 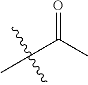 [SMILES: *C(C)=O] |
| 116 | 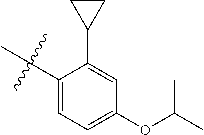 [SMILES: *c1ccc(OC(C)C)cc1C1CC1] | 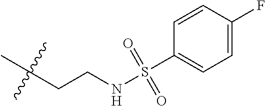 [SMILES: *CCNS(=O)(=O)c1ccc(F)cc1] | 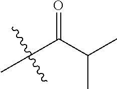 [SMILES: *C(=O)C(C)C] |
| 117 | 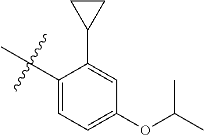 [SMILES: *c1ccc(OC(C)C)cc1C1CC1] | 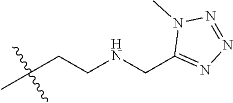 [SMILES: *CCNCc1nnnn1C] | 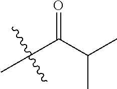 [SMILES: *C(=O)C(C)C] |
| 118 | 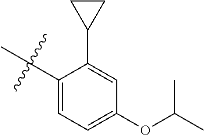 [SMILES: *c1ccc(OC(C)C)cc1C1CC1] | 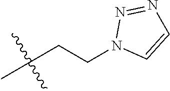 [SMILES: *CCn1ccnn1] | 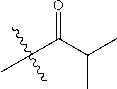 [SMILES: *C(=O)C(C)C] |
| 119 | 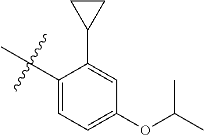 [SMILES: *c1ccc(OC(C)C)cc1C1CC1] | 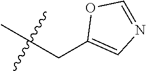 [SMILES: *Cc1cnco1] | 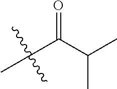 [SMILES: *C(=O)C(C)C] |
| 120 | 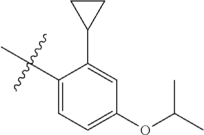 [SMILES: *c1ccc(OC(C)C)cc1C1CC1] | 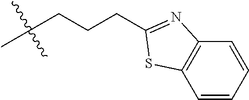 [SMILES: *CCCc1nc2ccccc2s1] | 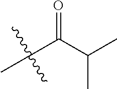 [SMILES: *C(=O)C(C)C] |
| 121 | 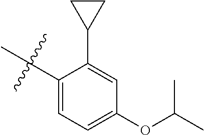 [SMILES: *c1ccc(OC(C)C)cc1C1CC1] | 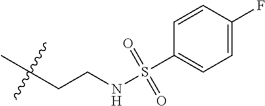 [SMILES: *CCNS(=O)(=O)c1ccc(F)cc1] | 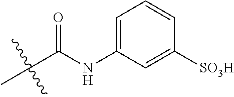 [SMILES: *C(=O)Nc1cccc(S(=O)(=O)O)c1] |
| 122 | 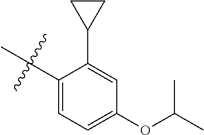 [SMILES: *c1ccc(OC(C)C)cc1C1CC1] | 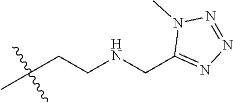 [SMILES: *CCNCc1nnnn1C] | 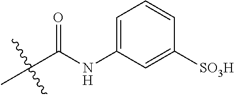 [SMILES: *C(=O)Nc1cccc(S(=O)(=O)O)c1] |
| 123 | 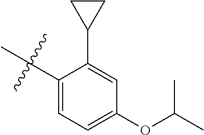 [SMILES: *c1ccc(OC(C)C)cc1C1CC1] | 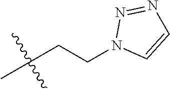 [SMILES: *CCn1ccnn1] | 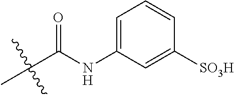 [SMILES: *C(=O)Nc1cccc(S(=O)(=O)O)c1] |
| 124 | 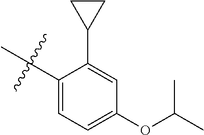 [SMILES: *c1ccc(OC(C)C)cc1C1CC1] | 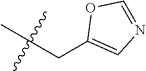 [SMILES: *Cc1cnco1] | 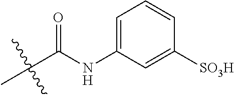 [SMILES: *C(=O)Nc1cccc(S(=O)(=O)O)c1] |
| 125 | 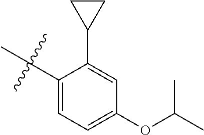 [SMILES: *c1ccc(OC(C)C)cc1C1CC1] | 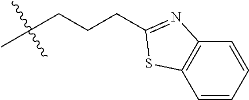 [SMILES: *CCCc1nc2ccccc2s1] | 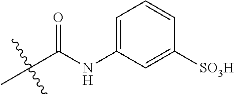 [SMILES: *C(=O)Nc1cccc(S(=O)(=O)O)c1] |
| 126 | 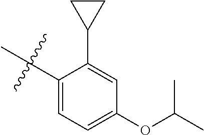 [SMILES: *c1ccc(OC(C)C)cc1C1CC1] | 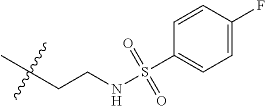 [SMILES: *CCNS(=O)(=O)c1ccc(F)cc1] | 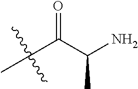 [SMILES: *C(=O)[C@H](C)N] |
| 127 | 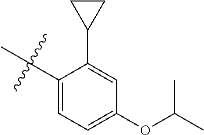 [SMILES: *c1ccc(OC(C)C)cc1C1CC1] | 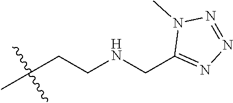 [SMILES: *CCNCc1nnnn1C] | 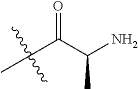 [SMILES: *C(=O)[C@H](C)N] |
| 128 | 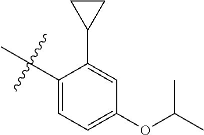 [SMILES: *c1ccc(OC(C)C)cc1C1CC1] | 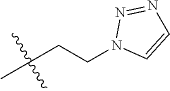 [SMILES: *CCn1ccnn1] | 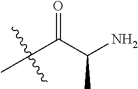 [SMILES: *C(=O)[C@H](C)N] |
| 129 | 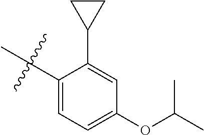 [SMILES: *c1ccc(OC(C)C)cc1C1CC1] | 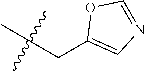 [SMILES: *Cc1cnco1] | 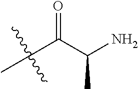 [SMILES: *C(=O)[C@H](C)N] |
| 130 | 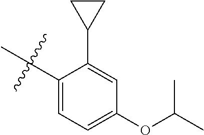 [SMILES: *c1ccc(OC(C)C)cc1C1CC1] | 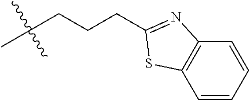 [SMILES: *CCCc1nc2ccccc2s1] | 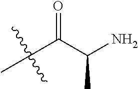 [SMILES: *C(=O)[C@H](C)N] |
| 131 | 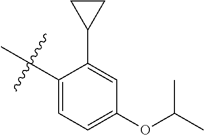 [SMILES: *c1ccc(OC(C)C)cc1C1CC1] | 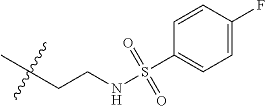 [SMILES: *CCNS(=O)(=O)c1ccc(F)cc1] | 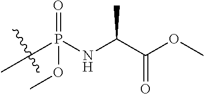 [SMILES: *P(=O)(N[C@@H](C)C(=O)OC)OC] |
| 132 | 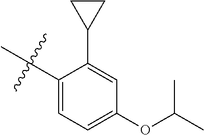 [SMILES: *c1ccc(OC(C)C)cc1C1CC1] | 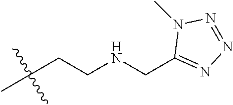 [SMILES: *CCNCc1nnnn1C] | 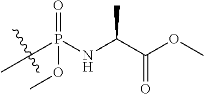 [SMILES: *P(=O)(N[C@@H](C)C(=O)OC)OC] |
| 133 | 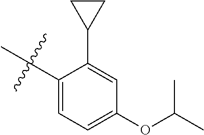 [SMILES: *c1ccc(OC(C)C)cc1C1CC1] | 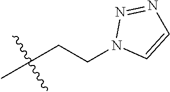 [SMILES: *CCn1ccnn1] | 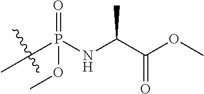 [SMILES: *P(=O)(N[C@@H](C)C(=O)OC)OC] |
| 134 | 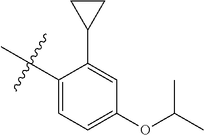 [SMILES: *c1ccc(OC(C)C)cc1C1CC1] | 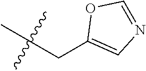 [SMILES: *Cc1cnco1] | 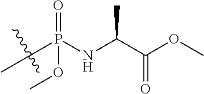 [SMILES: *P(=O)(N[C@@H](C)C(=O)OC)OC] |
| 135 | 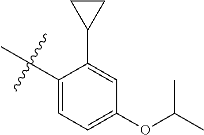 [SMILES: *c1ccc(OC(C)C)cc1C1CC1] | 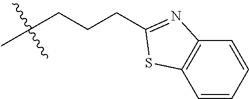 [SMILES: *CCCc1nc2ccccc2s1] | 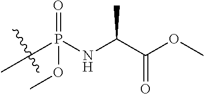 [SMILES: *P(=O)(N[C@@H](C)C(=O)OC)OC] |
| 136 | 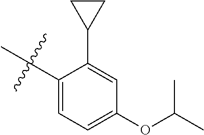 [SMILES: *c1ccc(OC(C)C)cc1C1CC1] | 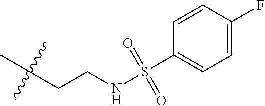 [SMILES: *CCNS(=O)(=O)c1ccc(F)cc1] | 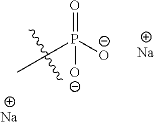 [SMILES: *P(=O)([O-])[O-] ; [Na+] ; [Na+]] |
| 137 | 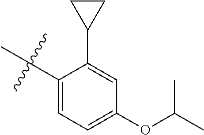 [SMILES: *c1ccc(OC(C)C)cc1C1CC1] | 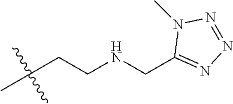 [SMILES: *CCNCc1nnnn1C] | 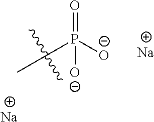 [SMILES: *P(=O)([O-])[O-] ; [Na+] ; [Na+]] |
| 138 | 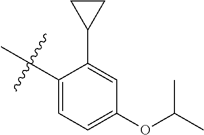 [SMILES: *c1ccc(OC(C)C)cc1C1CC1] | 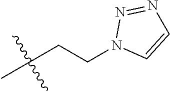 [SMILES: *CCn1ccnn1] | 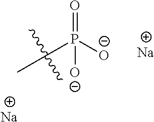 [SMILES: *P(=O)([O-])[O-] ; [Na+] ; [Na+]] |
| 139 | 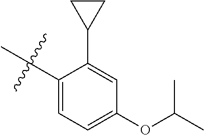 [SMILES: *c1ccc(OC(C)C)cc1C1CC1] | 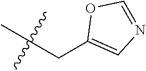 [SMILES: *Cc1cnco1] | 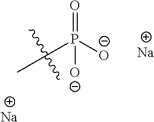 [SMILES: *P(=O)([O-])[O-] ; [Na+] ; [Na+]] |
| 140 | 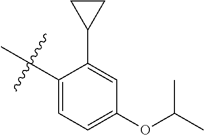 [SMILES: *c1ccc(OC(C)C)cc1C1CC1] | 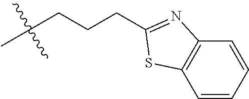 [SMILES: *CCCc1nc2ccccc2s1] | 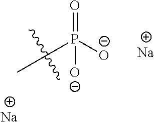 [SMILES: *P(=O)([O-])[O-] ; [Na+] ; [Na+]] |
| 141 | 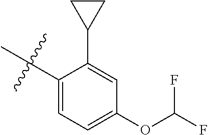 [SMILES: *c1ccc(OC(F)F)cc1C1CC1] | 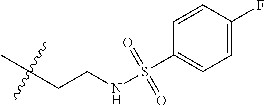 [SMILES: *CCNS(=O)(=O)c1ccc(F)cc1] | 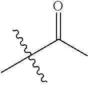 [SMILES: *C(C)=O] |
| 142 | 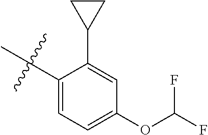 [SMILES: *c1ccc(OC(F)F)cc1C1CC1] | 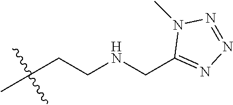 [SMILES: *CCNCc1nnnn1C] | 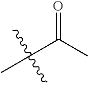 [SMILES: *C(C)=O] |
| 143 | 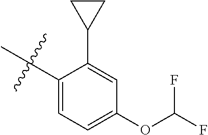 [SMILES: *c1ccc(OC(F)F)cc1C1CC1] | 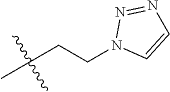 [SMILES: *CCn1ccnn1] | 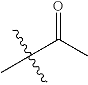 [SMILES: *C(C)=O] |
| 144 | 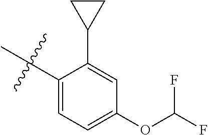 [SMILES: *c1ccc(OC(F)F)cc1C1CC1] | 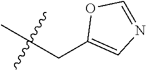 [SMILES: *Cc1cnco1] | 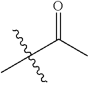 [SMILES: *C(C)=O] |
| 145 | 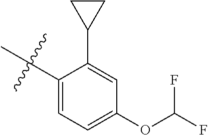 [SMILES: *c1ccc(OC(F)F)cc1C1CC1] | 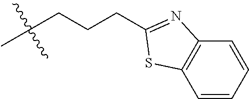 [SMILES: *CCCc1nc2ccccc2s1] | 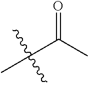 [SMILES: *C(C)=O] |
| 146 | 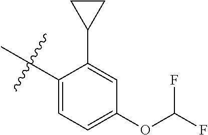 [SMILES: *c1ccc(OC(F)F)cc1C1CC1] | 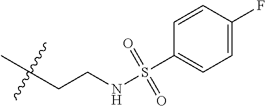 [SMILES: *CCNS(=O)(=O)c1ccc(F)cc1] | 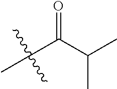 [SMILES: *C(=O)C(C)C] |
| 147 | 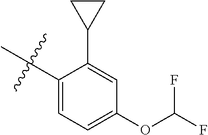 [SMILES: *c1ccc(OC(F)F)cc1C1CC1] | 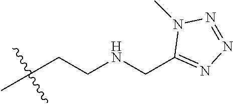 [SMILES: *CCNCc1nnnn1C] | 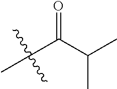 [SMILES: *C(=O)C(C)C] |
| 148 | 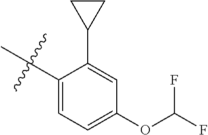 [SMILES: *c1ccc(OC(F)F)cc1C1CC1] | 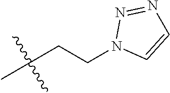 [SMILES: *CCn1ccnn1] | 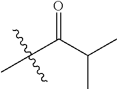 [SMILES: *C(=O)C(C)C] |
| 149 | 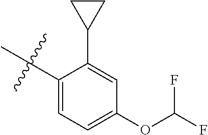 [SMILES: *c1ccc(OC(F)F)cc1C1CC1] | 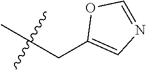 [SMILES: *Cc1cnco1] | 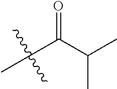 [SMILES: *C(=O)C(C)C] |
| 150 | 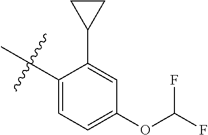 [SMILES: *c1ccc(OC(F)F)cc1C1CC1] | 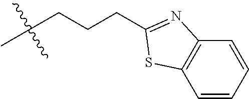 [SMILES: *CCCc1nc2ccccc2s1] | 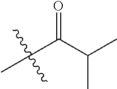 [SMILES: *C(=O)C(C)C] |
| 151 | 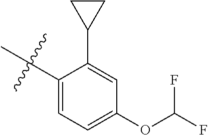 [SMILES: *c1ccc(OC(F)F)cc1C1CC1] | 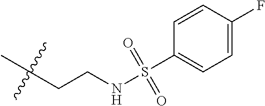 [SMILES: *CCNS(=O)(=O)c1ccc(F)cc1] | 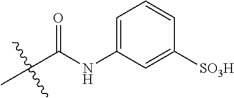 [SMILES: *C(=O)Nc1cccc(S(=O)(=O)O)c1] |
| 152 | 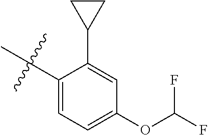 [SMILES: *c1ccc(OC(F)F)cc1C1CC1] | 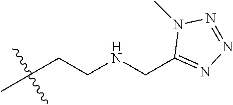 [SMILES: *CCNCc1nnnn1C] | 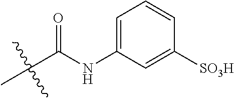 [SMILES: *C(=O)Nc1cccc(S(=O)(=O)O)c1] |
| 153 | 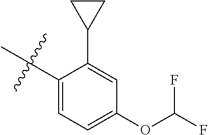 [SMILES: *c1ccc(OC(F)F)cc1C1CC1] | 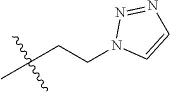 [SMILES: *CCn1ccnn1] | 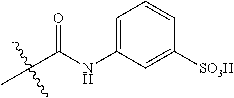 [SMILES: *C(=O)Nc1cccc(S(=O)(=O)O)c1] |
| 154 | 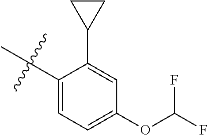 [SMILES: *c1ccc(OC(F)F)cc1C1CC1] | 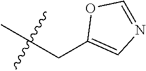 [SMILES: *Cc1cnco1] | 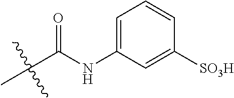 [SMILES: *C(=O)Nc1cccc(S(=O)(=O)O)c1] |
| 155 | 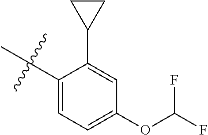 [SMILES: *c1ccc(OC(F)F)cc1C1CC1] | 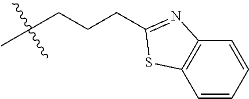 [SMILES: *CCCc1nc2ccccc2s1] | 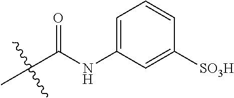 [SMILES: *C(=O)Nc1cccc(S(=O)(=O)O)c1] |
| 156 | 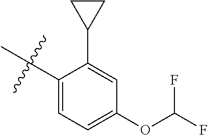 [SMILES: *c1ccc(OC(F)F)cc1C1CC1] | 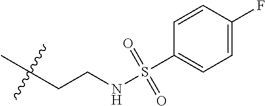 [SMILES: *CCNS(=O)(=O)c1ccc(F)cc1] | 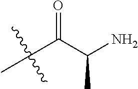 [SMILES: *C(=O)[C@H](C)N] |
| 157 | 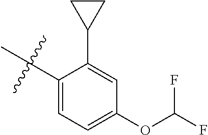 [SMILES: *c1ccc(OC(F)F)cc1C1CC1] | 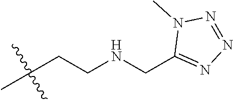 [SMILES: *CCNCc1nnnn1C] | 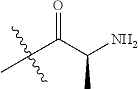 [SMILES: *C(=O)[C@H](C)N] |
| 158 | 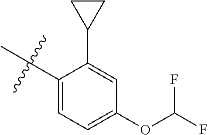 [SMILES: *c1ccc(OC(F)F)cc1C1CC1] | 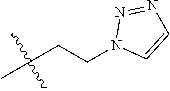 [SMILES: *CCn1ccnn1] | 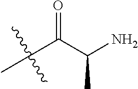 [SMILES: *C(=O)[C@H](C)N] |
| 159 | 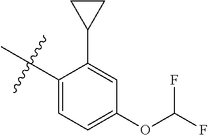 [SMILES: *c1ccc(OC(F)F)cc1C1CC1] | 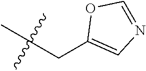 [SMILES: *Cc1cnco1] | 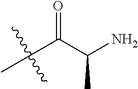 [SMILES: *C(=O)[C@H](C)N] |
| 160 | 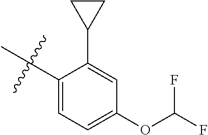 [SMILES: *c1ccc(OC(F)F)cc1C1CC1] | 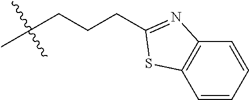 [SMILES: *CCCc1nc2ccccc2s1] | 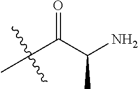 [SMILES: *C(=O)[C@H](C)N] |
| 161 | 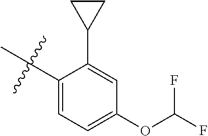 [SMILES: *c1ccc(OC(F)F)cc1C1CC1] | 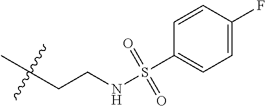 [SMILES: *CCNS(=O)(=O)c1ccc(F)cc1] | 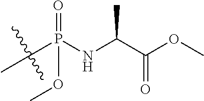 [SMILES: *P(=O)(N[C@@H](C)C(=O)OC)OC] |
| 162 | 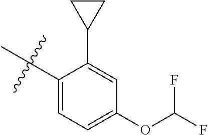 [SMILES: *c1ccc(OC(F)F)cc1C1CC1] | 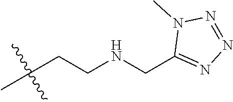 [SMILES: *CCNCc1nnnn1C] | 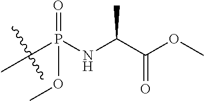 [SMILES: *P(=O)(N[C@@H](C)C(=O)OC)OC] |
| 163 | 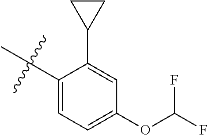 [SMILES: *c1ccc(OC(F)F)cc1C1CC1] | 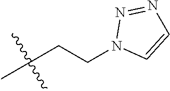 [SMILES: *CCn1ccnn1] | 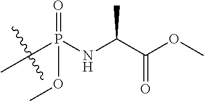 [SMILES: *P(=O)(N[C@@H](C)C(=O)OC)OC] |
| 164 | 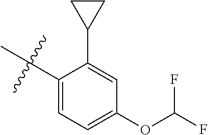 [SMILES: *c1ccc(OC(F)F)cc1C1CC1] | 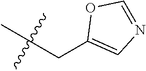 [SMILES: *Cc1cnco1] | 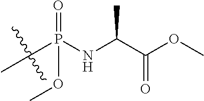 [SMILES: *P(=O)(N[C@@H](C)C(=O)OC)OC] |
| 165 | 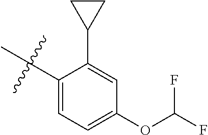 [SMILES: *c1ccc(OC(F)F)cc1C1CC1] | 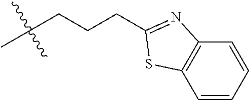 [SMILES: *CCCc1nc2ccccc2s1] | 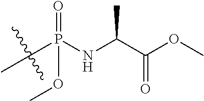 [SMILES: *P(=O)(N[C@@H](C)C(=O)OC)OC] |
| 166 | 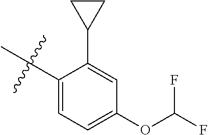 [SMILES: *c1ccc(OC(F)F)cc1C1CC1] | 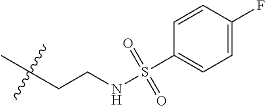 [SMILES: *CCNS(=O)(=O)c1ccc(F)cc1] | 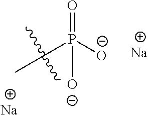 [SMILES: *P(=O)([O-])[O-] ; [Na+] ; [Na+]] |
| 167 | 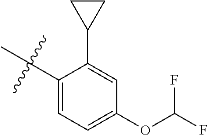 [SMILES: *c1ccc(OC(F)F)cc1C1CC1] | 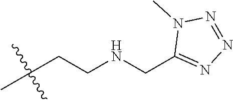 [SMILES: *CCNCc1nnnn1C] | 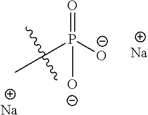 [SMILES: *P(=O)([O-])[O-] ; [Na+] ; [Na+]] |
| 168 | 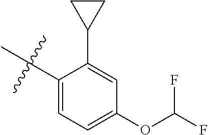 [SMILES: *c1ccc(OC(F)F)cc1C1CC1] | 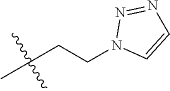 [SMILES: *CCn1ccnn1] | 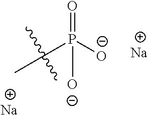 [SMILES: *P(=O)([O-])[O-] ; [Na+] ; [Na+]] |
| 169 | 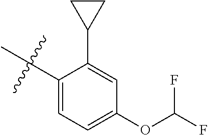 [SMILES: *c1ccc(OC(F)F)cc1C1CC1] | 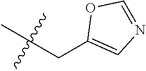 [SMILES: *Cc1cnco1] | 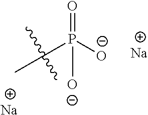 [SMILES: *P(=O)([O-])[O-] ; [Na+] ; [Na+]] |
| 170 | 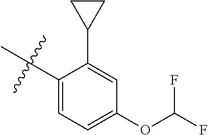 [SMILES: *c1ccc(OC(F)F)cc1C1CC1] | 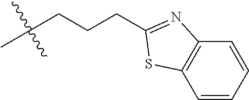 [SMILES: *CCCc1nc2ccccc2s1] | 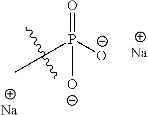 [SMILES: *P(=O)([O-])[O-] ; [Na+] ; [Na+]] |
| 171 | 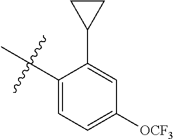 [SMILES: *c1ccc(OC(F)(F)F)cc1C1CC1] | 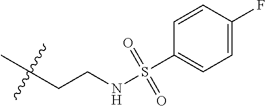 [SMILES: *CCNS(=O)(=O)c1ccc(F)cc1] | 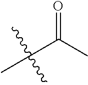 [SMILES: *C(C)=O] |
| 172 | 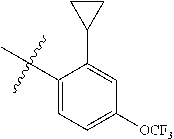 [SMILES: *c1ccc(OC(F)(F)F)cc1C1CC1] | 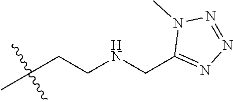 [SMILES: *CCNCc1nnnn1C] | 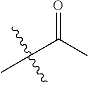 [SMILES: *C(C)=O] |
| 173 | 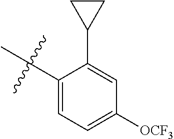 [SMILES: *c1ccc(OC(F)(F)F)cc1C1CC1] | 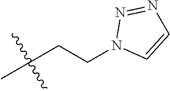 [SMILES: *CCn1ccnn1] | 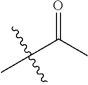 [SMILES: *C(C)=O] |
| 174 | 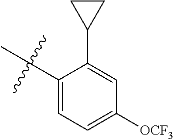 [SMILES: *c1ccc(OC(F)(F)F)cc1C1CC1] | 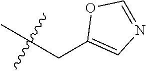 [SMILES: *Cc1cnco1] | 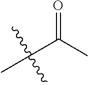 [SMILES: *C(C)=O] |
| 175 | 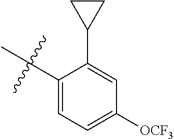 [SMILES: *c1ccc(OC(F)(F)F)cc1C1CC1] | 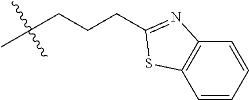 [SMILES: *CCCc1nc2ccccc2s1] | 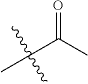 [SMILES: *C(C)=O] |
| 176 | 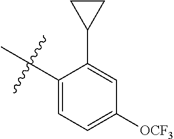 [SMILES: *c1ccc(OC(F)(F)F)cc1C1CC1] | 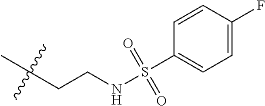 [SMILES: *CCNS(=O)(=O)c1ccc(F)cc1] | 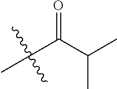 [SMILES: *C(=O)C(C)C] |
| 177 | 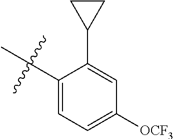 [SMILES: *c1ccc(OC(F)(F)F)cc1C1CC1] | 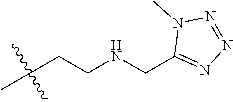 [SMILES: *CCNCc1nnnn1C] | 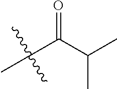 [SMILES: *C(=O)C(C)C] |
| 178 | 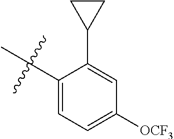 [SMILES: *c1ccc(OC(F)(F)F)cc1C1CC1] | 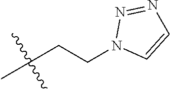 [SMILES: *CCn1ccnn1] | 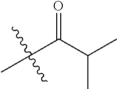 [SMILES: *C(=O)C(C)C] |
| 179 | 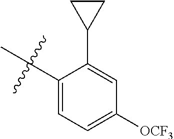 [SMILES: *c1ccc(OC(F)(F)F)cc1C1CC1] | 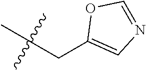 [SMILES: *Cc1cnco1] | 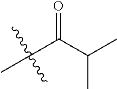 [SMILES: *C(=O)C(C)C] |
| 180 | 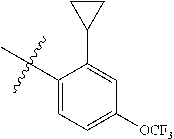 [SMILES: *c1ccc(OC(F)(F)F)cc1C1CC1] | 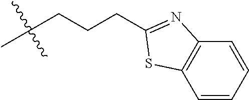 [SMILES: *CCCc1nc2ccccc2s1] | 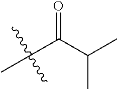 [SMILES: *C(=O)C(C)C] |
| 181 | 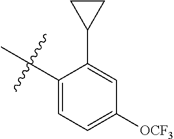 [SMILES: *c1ccc(OC(F)(F)F)cc1C1CC1] | 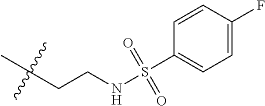 [SMILES: *CCNS(=O)(=O)c1ccc(F)cc1] | 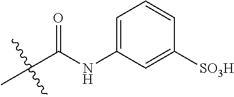 [SMILES: *C(=O)Nc1cccc(S(=O)(=O)O)c1] |
| 182 | 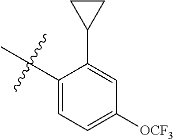 [SMILES: *c1ccc(OC(F)(F)F)cc1C1CC1] | 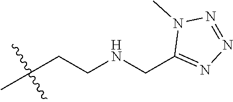 [SMILES: *CCNCc1nnnn1C] | 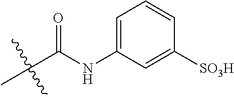 [SMILES: *C(=O)Nc1cccc(S(=O)(=O)O)c1] |
| 183 | 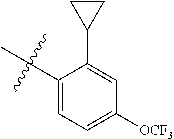 [SMILES: *c1ccc(OC(F)(F)F)cc1C1CC1] | 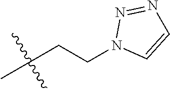 [SMILES: *CCn1ccnn1] | 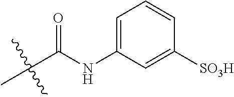 [SMILES: *C(=O)Nc1cccc(S(=O)(=O)O)c1] |
| 184 | 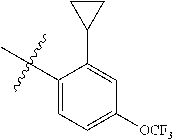 [SMILES: *c1ccc(OC(F)(F)F)cc1C1CC1] | 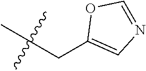 [SMILES: *Cc1cnco1] | 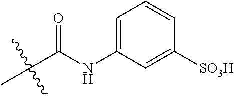 [SMILES: *C(=O)Nc1cccc(S(=O)(=O)O)c1] |
| 185 | 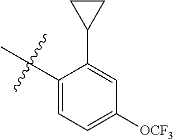 [SMILES: *c1ccc(OC(F)(F)F)cc1C1CC1] | 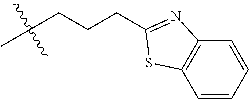 [SMILES: *CCCc1nc2ccccc2s1] | 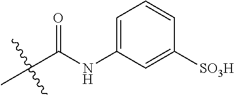 [SMILES: *C(=O)Nc1cccc(S(=O)(=O)O)c1] |
| 186 | 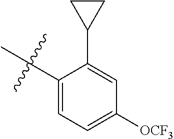 [SMILES: *c1ccc(OC(F)(F)F)cc1C1CC1] | 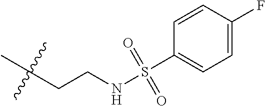 [SMILES: *CCNS(=O)(=O)c1ccc(F)cc1] | 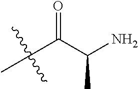 [SMILES: *C(=O)[C@H](C)N] |
| 187 | 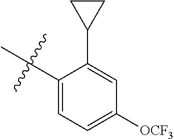 [SMILES: *c1ccc(OC(F)(F)F)cc1C1CC1] | 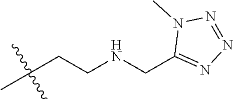 [SMILES: *CCNCc1nnnn1C] | 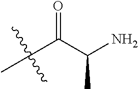 [SMILES: *C(=O)[C@H](C)N] |
| 188 | 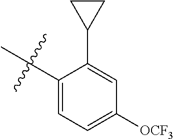 [SMILES: *c1ccc(OC(F)(F)F)cc1C1CC1] | 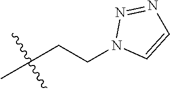 [SMILES: *CCn1ccnn1] | 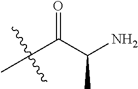 [SMILES: *C(=O)[C@H](C)N] |
| 189 | 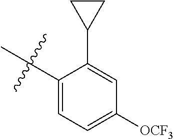 [SMILES: *c1ccc(OC(F)(F)F)cc1C1CC1] | 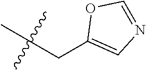 [SMILES: *Cc1cnco1] | 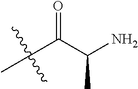 [SMILES: *C(=O)[C@H](C)N] |
| 190 | 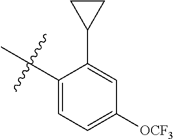 [SMILES: *c1ccc(OC(F)(F)F)cc1C1CC1] | 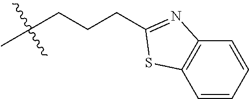 [SMILES: *CCCc1nc2ccccc2s1] | 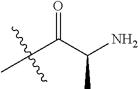 [SMILES: *C(=O)[C@H](C)N] |
| 191 | 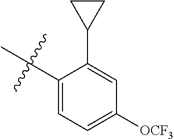 [SMILES: *c1ccc(OC(F)(F)F)cc1C1CC1] | 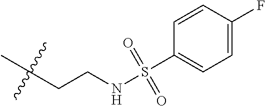 [SMILES: *CCNS(=O)(=O)c1ccc(F)cc1] | 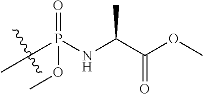 |
| 192 | 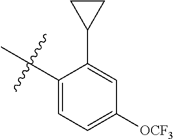 [SMILES: *c1ccc(OC(F)(F)F)cc1C1CC1] | 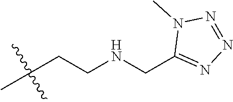 [SMILES: *CCNCc1nnnn1C] | 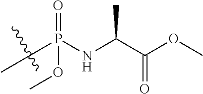 |
| 193 | 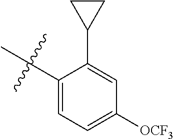 [SMILES: *c1ccc(OC(F)(F)F)cc1C1CC1] | 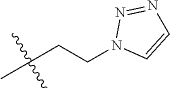 [SMILES: *CCn1ccnn1] | 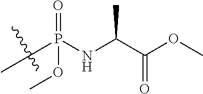 |
| 194 | 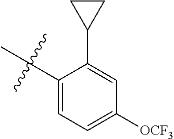 [SMILES: *c1ccc(OC(F)(F)F)cc1C1CC1] | 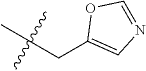 [SMILES: *Cc1cnco1] | 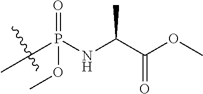 |
| 195 | 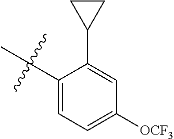 [SMILES: *c1ccc(OC(F)(F)F)cc1C1CC1] | 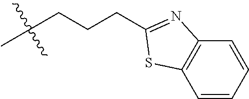 [SMILES: *CCCc1nc2ccccc2s1] | 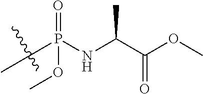 |
| 196 | 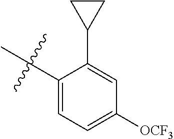 [SMILES: *c1ccc(OC(F)(F)F)cc1C1CC1] | 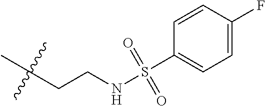 [SMILES: *CCNS(=O)(=O)c1ccc(F)cc1] | 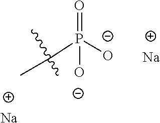 [SMILES: *P(=O)([O-])O ; [Na+] ; [Na+]] |
| 197 | 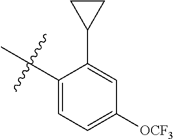 [SMILES: *c1ccc(OC(F)(F)F)cc1C1CC1] | 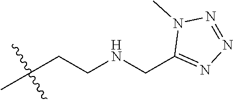 [SMILES: *CCNCc1nnnn1C] | 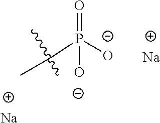 [SMILES: *P(=O)([O-])[O-] ; [Na+] ; [Na+]] |
| 198 | 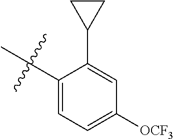 [SMILES: *c1ccc(OC(F)(F)F)cc1C1CC1] | 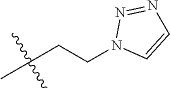 [SMILES: *CCn1ccnn1] | 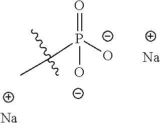 [SMILES: *P(=O)([O-])[O-] ; [Na+] ; [Na+]] |
| 199 | 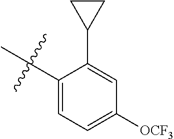 [SMILES: *c1ccc(OC(F)(F)F)cc1C1CC1] | 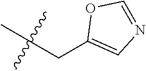 [SMILES: *Cc1cnco1] | 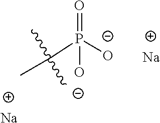 [SMILES: *P(=O)([O-])O ; [Na+] ; [Na+]] |
| 200 | 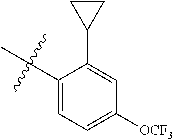 [SMILES: *c1ccc(OC(F)(F)F)cc1C1CC1] | 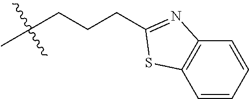 [SMILES: *CCCc1nc2ccccc2s1] | 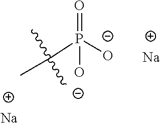 [SMILES: *P(=O)([O-])O ; [Na+] ; [Na+]] |
| 201 | 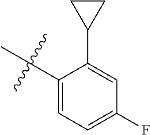 [SMILES: *c1ccc(F)cc1C1CC1] | 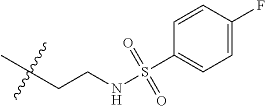 [SMILES: *CCNS(=O)(=O)c1ccc(F)cc1] | 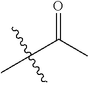 [SMILES: *C(C)=O] |
| 202 | 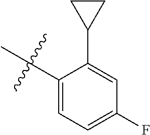 [SMILES: *c1ccc(F)cc1C1CC1] | 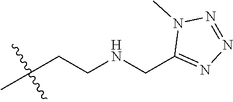 [SMILES: *CCNCc1nnnn1C] | 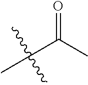 [SMILES: *C(C)=O] |
| 203 | 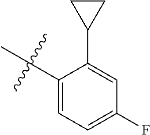 [SMILES: *c1ccc(F)cc1C1CC1] | 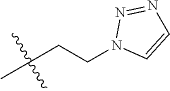 [SMILES: *CCn1ccnn1] | 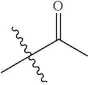 [SMILES: *C(C)=O] |
| 204 | 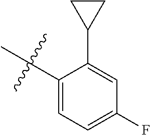 [SMILES: *c1ccc(F)cc1C1CC1] | 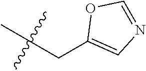 [SMILES: *Cc1cnco1] | 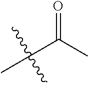 [SMILES: *C(C)=O] |
| 205 | 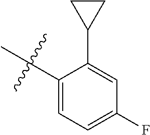 [SMILES: *c1ccc(F)cc1C1CC1] | 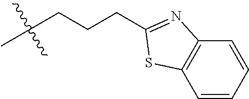 [SMILES: *CCCc1nc2ccccc2s1] | 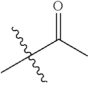 [SMILES: *C(C)=O] |
| 206 | 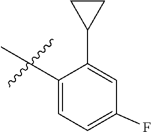 [SMILES: *c1ccc(F)cc1C1CC1] | 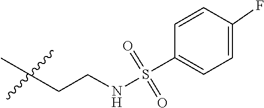 [SMILES: *CCNS(=O)(=O)c1ccc(F)cc1] | 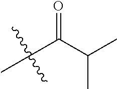 [SMILES: *C(=O)C(C)C] |
| 207 | 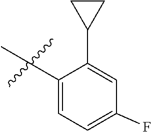 [SMILES: *c1ccc(F)cc1C1CC1] | 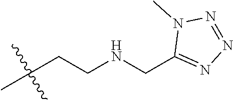 [SMILES: *CCNCc1nnnn1C] | 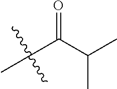 [SMILES: *C(=O)C(C)C] |
| 208 | 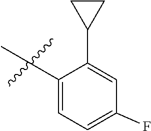 [SMILES: *c1ccc(F)cc1C1CC1] | 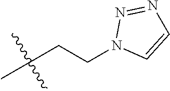 [SMILES: *CCn1ccnn1] | 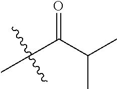 [SMILES: *C(=O)C(C)C] |
| 209 | 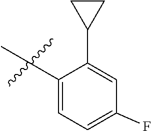 [SMILES: *c1ccc(F)cc1C1CC1] | 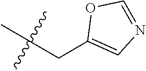 [SMILES: *Cc1cnco1] | 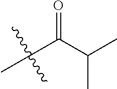 [SMILES: *C(=O)C(C)C] |
| 210 | 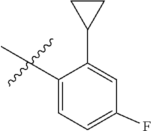 [SMILES: *c1ccc(F)cc1C1CC1] | 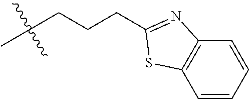 [SMILES: *CCCc1nc2ccccc2s1] | 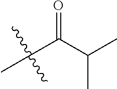 [SMILES: *C(=O)C(C)C] |
| 211 | 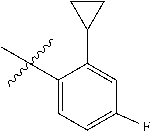 [SMILES: *c1ccc(F)cc1C1CC1] | 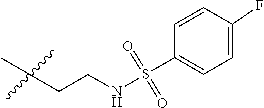 [SMILES: *CCNS(=O)(=O)c1ccc(F)cc1] | 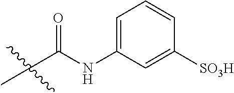 [SMILES: *C(=O)Nc1cccc(S(=O)(=O)O)c1] |
| 212 | 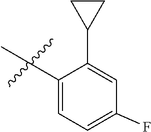 [SMILES: *c1ccc(F)cc1C1CC1] | 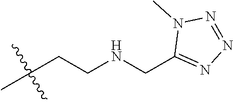 [SMILES: *CCNCc1nnnn1C] | 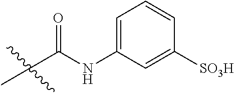 [SMILES: *C(=O)Nc1cccc(S(=O)(=O)O)c1] |
| 213 | 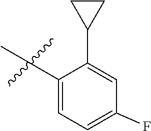 [SMILES: *c1ccc(F)cc1C1CC1] | 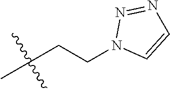 [SMILES: *CCn1ccnn1] | 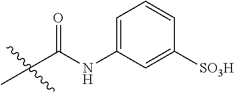 [SMILES: *C(=O)Nc1cccc(S(=O)(=O)O)c1] |
| 214 | 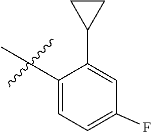 [SMILES: *c1ccc(F)cc1C1CC1] | 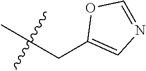 [SMILES: *Cc1cnco1] | 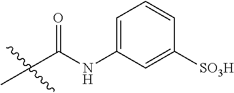 [SMILES: *C(=O)Nc1cccc(S(=O)(=O)O)c1] |
| 215 | 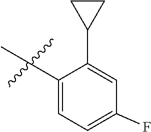 [SMILES: *c1ccc(F)cc1C1CC1] | 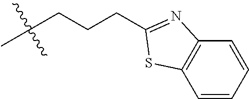 [SMILES: *CCCc1nc2ccccc2s1] | 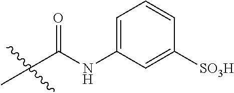 [SMILES: *C(=O)Nc1cccc(S(=O)(=O)O)c1] |
| 216 | 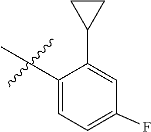 [SMILES: *c1ccc(F)cc1C1CC1] | 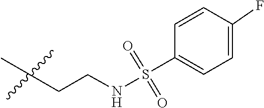 [SMILES: *CCNS(=O)(=O)c1ccc(F)cc1] | 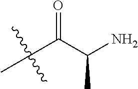 [SMILES: *C(=O)[C@H](C)N] |
| 217 | 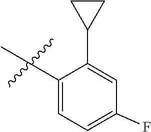 [SMILES: *c1ccc(F)cc1C1CC1] | 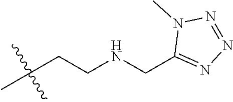 [SMILES: *CCNCc1nnnn1C] | 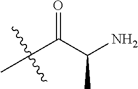 [SMILES: *C(=O)[C@H](C)N] |
| 218 | 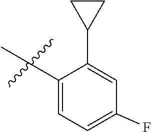 [SMILES: *c1ccc(F)cc1C1CC1] | 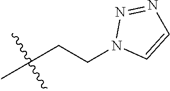 [SMILES: *CCn1ccnn1] | 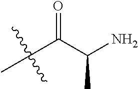 [SMILES: *C(=O)[C@H](C)N] |
| 219 | 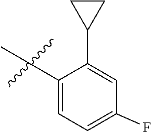 [SMILES: *c1ccc(F)cc1C1CC1] | 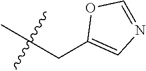 [SMILES: *Cc1cnco1] | 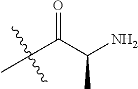 [SMILES: *C(=O)[C@H](C)N] |
| 220 | 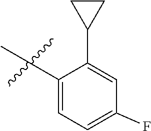 [SMILES: *c1ccc(F)cc1C1CC1] | 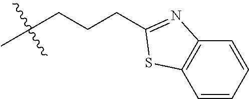 [SMILES: *CCCc1nc2ccccc2s1] | 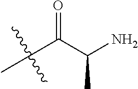 [SMILES: *C(=O)[C@H](C)N] |
| 221 | 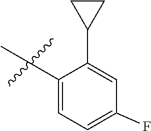 [SMILES: *c1ccc(F)cc1C1CC1] | 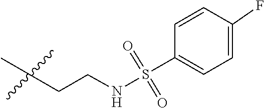 [SMILES: *CCNS(=O)(=O)c1ccc(F)cc1] | 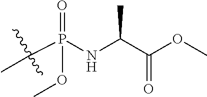 [SMILES: *P(=O)(N[C@@H](C)C(=O)OC)OC] |
| 222 | 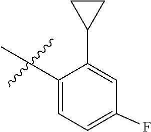 [SMILES: *c1ccc(F)cc1C1CC1] | 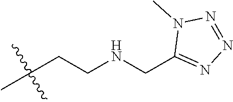 [SMILES: *CCNCc1nnnn1C] | 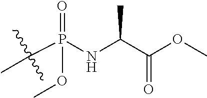 [SMILES: *P(=O)(N[C@@H](C)C(=O)OC)OC] |
| 223 | 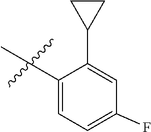 [SMILES: *c1ccc(F)cc1C1CC1] | 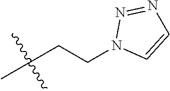 [SMILES: *CCn1ccnn1] | 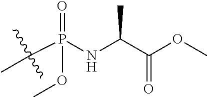 [SMILES: *P(=O)(N[C@@H](C)C(=O)OC)OC] |
| 224 | 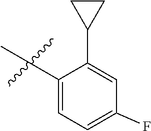 [SMILES: *c1ccc(F)cc1C1CC1] | 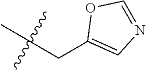 [SMILES: *Cc1cnco1] | 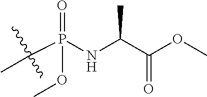 [SMILES: *P(=O)(N[C@@H](C)C(=O)OC)OC] |
| 225 | 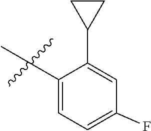 [SMILES: *c1ccc(F)cc1C1CC1] | 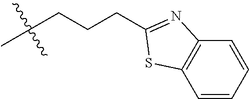 [SMILES: *CCCc1nc2ccccc2s1] | 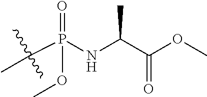 [SMILES: *P(=O)(N[C@@H](C)C(=O)OC)OC] |
| 226 | 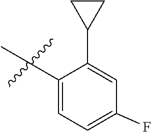 [SMILES: *c1ccc(F)cc1C1CC1] | 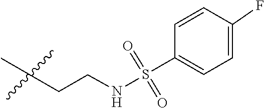 [SMILES: *CCNS(=O)(=O)c1ccc(F)cc1] | 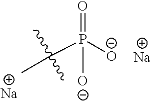 [SMILES: *P(=O)([O-])[O-] ; [Na+] ; [Na+]] |
| 227 | 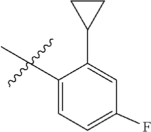 [SMILES: *c1ccc(F)cc1C1CC1] | 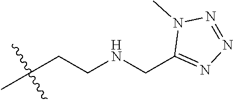 [SMILES: *CCNCc1nnnn1C] | 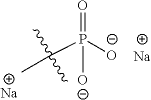 [SMILES: *P(=O)([O-])[O-] ; [Na+] ; [Na+]] |
| 228 | 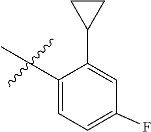 [SMILES: *c1ccc(F)cc1C1CC1] | 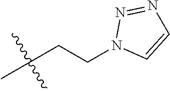 [SMILES: *CCn1ccnn1] | 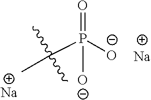 [SMILES: *P(=O)([O-])[O-] ; [Na+] ; [Na+]] |
| 229 | 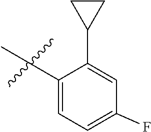 [SMILES: *c1ccc(F)cc1C1CC1] | 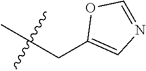 [SMILES: *Cc1cnco1] | 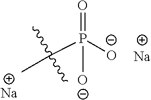 [SMILES: *P(=O)([O-])[O-] ; [Na+] ; [Na+]] |
| 230 | 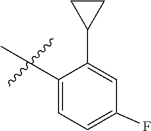 [SMILES: *c1ccc(F)cc1C1CC1] | 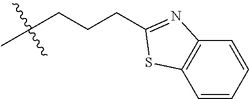 [SMILES: *CCCc1nc2ccccc2s1] | 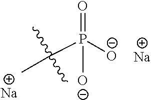 [SMILES: *P(=O)([O-])[O-] ; [Na+] ; [Na+]] |

In certain embodiments, the present invention provides compounds represented by Formula (XII) or (XIII), or a pharmaceutically acceptable salt or ester thereof:

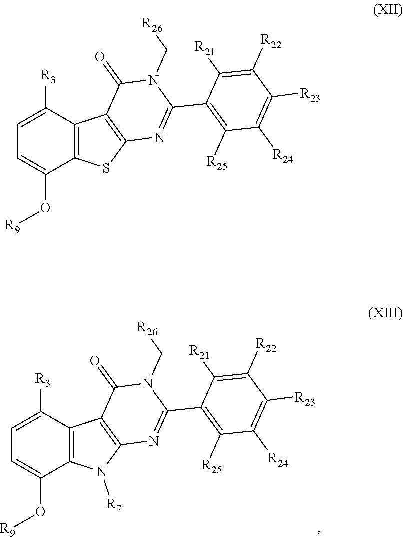

wherein each R₂₁, R₂₂, R₂₃, R₂₄, or R₂₅ is independently hydrogen, halogen, optionally substituted —C₁-C₆ alkyl, optionally substituted —C₁-C₆ alkoxyl, or optionally substituted —C₃-C₈-cycloalkyl; R₂₆ is optionally substituted aryl, optionally substituted heteroaryl, optionally substituted heteroarylalkyl, optionally substituted —C₃-C₈-cycloalkyl, optionally substituted —C₁-C₆ alkyl, —NR₇R₈, —CH₂NR₇R₈, —CH₂NR₇C(O)R₈, or

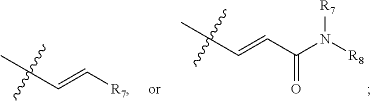

and R₃, R₇, R₈, and R₉ are as previously defined.

Alternatively, R₂₁ and R₂₂ are taken together with the carbon atoms to which they are attached to form an optionally substituted carbocyclic or heterocyclic ring which is fused with phenyl.

Alternatively, R₂₂ and R₂₃ are taken together with the carbon atoms to which they are attached to form an optionally substituted carbocyclic or heterocyclic ring which is fused with phenyl.

Alternatively, R₇ and R₅ are taken together with the nitrogen atom to which they are attached to form an optionally substituted heterocyclic ring.

In certain embodiments, the present invention provides a pharmaceutical composition comprising a therapeutically effective amount of a compound or combination of compounds of the present invention, or a pharmaceutically acceptable salt, ester or combination thereof, in combination with a pharmaceutically acceptable carrier or excipient.

In certain embodiments, the present invention provides a method for the prevention or treatment of an 17β-HSD13 mediated disease or condition. The method comprises administering a therapeutically effective amount of a compound of Formula (I) or a pharmaceutically acceptable salt thereof. The present invention also provides the use of a compound of Formula (I), or a pharmaceutically acceptable salt thereof, in the manufacture of a medicament for the prevention or treatment of a 17β-HSD13 mediated disease or condition including, but not limited to: nonalcoholic fatty liver disease (NAFLD), nonalcoholic steatohepatitis (NASH), liver cirrhosis, liver fibrosis, hepatocellular carcinoma (HCC), and metabolic disorders.

It will be appreciated that the description of the present invention herein should be construed in congruity with the laws and principles of chemical bonding. In some instances, it may be necessary to remove a hydrogen atom in order to accommodate a substituent at any given location.

It will yet be appreciated that the compounds of the present invention may contain one or more asymmetric carbon atoms and may exist in racemic, diastereoisomeric, and optically active forms. It will still be appreciated that certain compounds of the present invention may exist in different tautomeric forms. All tautomers are contemplated to be within the scope of the present invention.

It should be understood that the compounds encompassed by the present invention are those that are suitably stable for use as pharmaceutical agent.

### Definitions

Listed below are definitions of various terms used to describe this invention. These definitions apply to the terms as they are used throughout this specification and claims, unless otherwise limited in specific instances, either individually or as part of a larger group.

The term “aryl,” as used herein, refers to a mono- or polycyclic carbocyclic ring system comprising at least one aromatic ring. Preferred aryl groups are C₆-C₁₂-aryl groups, including, but not limited to, phenyl, naphthyl, tetrahydronaphthyl, indanyl, and indenyl. A polycyclic aryl is a polycyclic ring system that comprises at least one aromatic ring. Polycyclic aryls can comprise fused rings, covalently attached rings or a combination thereof.

The term “heteroaryl,” as used herein, refers to a mono- or polycyclic aromatic radical having one or more ring atom selected from S, O and N; and the remaining ring atoms are carbon, wherein any N or S contained within the ring may be optionally oxidized. In certain embodiments, a heteroaryl group is a 5- to 10-membered heteroaryl, such as a 5- or 6-membered monocyclic heteroaryl or an 8- to 10-membered bicyclic heteroaryl. Heteroaryl groups include, but are not limited to, pyridinyl, pyrazinyl, pyrimidinyl, pyrrolyl, pyrazolyl, imidazolyl, thiazolyl, oxazolyl, isooxazolyl, thiadiazolyl, oxadiazolyl, thiophenyl, furanyl, quinolinyl, isoquinolinyl, benzimidazolyl, benzoxazolyl, quinoxalinyl. A polycyclic heteroaryl can comprise fused rings, covalently attached rings or a combination thereof. A heteroaryl group can be C-attached or N-attached where possible.

In accordance with the invention, aryl and heteroaryl groups can be substituted or unsubstituted.

The term “bicyclic aryl” or “bicyclic heteroaryl” refers to a ring system consisting of two rings wherein at least one ring is aromatic; and the two rings can be fused or covalently attached.

The term “alkyl” as used herein, refers to saturated, straight- or branched-chain hydrocarbon radicals. “C₁-C₄ alkyl,” “C₁-C₆ alkyl,” “C₁-C₈ alkyl,” “C₁-C₁₂ alkyl,” “C₂-C₄ alkyl,” and “C₃-C₆ alkyl,” refer to alkyl groups containing from 1 to 4, 1 to 6, 1 to 8, 1 to 12, 2 to 4 and 3 to 6 carbon atoms respectively. Examples of alkyl groups include, but are not limited to, methyl, ethyl, propyl, isopropyl, n-butyl, sec-butyl, tert-butyl, n-pentyl, neopentyl, n-hexyl, n-heptyl and n-octyl radicals.

The term “alkenyl” as used herein, refers to straight- or branched-chain hydrocarbon radicals having at least one carbon-carbon double bond. “C₂-C₈ alkenyl,” “C₂-C₁₂ alkenyl,” “C₂—C₄ alkenyl,” “C₃-C₄ alkenyl,” and “C₃-C₆ alkenyl,” refer to alkenyl groups containing from 2 to 8, 2 to 12, 2 to 4, 3 to 4 or 3 to 6 carbon atoms respectively. Alkenyl groups include, but are not limited to, ethenyl, propenyl, butenyl, 2-methyl-2-buten-2-yl, heptenyl, octenyl, and the like.

The term “alkynyl” as used herein, refers to straight- or branched-chain hydrocarbon radicals having at least one carbon-carbon triple bond. “C₂-C₈ alkynyl,” “C₂-C₁₂ alkynyl,” “C₂-C₄ alkynyl,” “C₃-C₄ alkynyl,” and “C₃-C₆ alkynyl,” refer to alkynyl groups containing from 2 to 8, 2 to 12, 2 to 4, 3 to 4 or 3 to 6 carbon atoms respectively. Representative alkynyl groups include, but are not limited to, ethynyl, 2-propynyl, 2-butynyl, heptynyl, octynyl, and the like.

The term “cycloalkyl”, as used herein, refers to a monocyclic or polycyclic saturated carbocyclic ring, such as a bi- or tri-cyclic fused, bridged or spiro system. The ring carbon atoms are optionally oxo-substituted or optionally substituted with an exocyclic olefinic double bond. Preferred cycloalkyl groups include C₃-C₁₂ cycloalkyl, C₃-C₆ cycloalkyl, C₃-C₈ cycloalkyl and C₄-C₇ cycloalkyl. Examples of cycloalkyl include, but are not limited to, cyclopropyl, cyclobutyl, cyclopentyl, cyclohexyl, cyclopentyl, cyclooctyl, 4-methylene-cyclohexyl, bicyclo[2.2.1]heptyl, bicyclo[3.1.0]hexyl, spiro[2.5]octyl, 3-methylenebicyclo[3.2.1]octyl, spiro[4.4]nonanyl, and the like.

The term “cycloalkenyl”, as used herein, refers to monocyclic or polycyclic carbocyclic ring, such as a bi- or tri-cyclic fused, bridged or spiro system having at least one carbon-carbon double bond. The ring carbon atoms are optionally oxo-substituted or optionally substituted with an exocyclic olefinic double bond. Preferred cycloalkenyl groups include C₃-C₁₂ cycloalkenyl, C₄-C₁₂-cycloalkenyl, C₃-C₈ cycloalkenyl, C₄-C₈ cycloalkenyl and C₅-C₇ cycloalkenyl groups. Examples of cycloalkenyl include, but are not limited to, cyclopropenyl, cyclobutenyl, cyclopentenyl, cyclohexenyl, cycloheptenyl, cyclooctenyl, bicyclo[2.2.1]hept-2-enyl, bicyclo[3.1.0]hex-2-enyl, spiro[2.5]oct-4-enyl, spiro[4.4]non-2-enyl, bicyclo[4.2.1]non-3-en-12-yl, and the like.

As used herein, the term “arylalkyl” means a functional group wherein an alkylene chain is attached to an aryl group, e.g., —(CH₂)ₙ-phenyl, where n is 1 to 12, preferably 1 to 6 and more preferably 1 or 2. The term “substituted arylalkyl” means an arylalkyl functional group in which the aryl group is substituted. Similarly, the term “heteroarylalkyl” means a functional group wherein an alkylene chain, is attached to a heteroaryl group, e.g., —(CH₂)ₙ-heteroaryl, where n is 1 to 12, preferably 1 to 6 and more preferably 1 or 2. The term “substituted heteroarylalkyl” means a heteroarylalkyl functional group in which the heteroaryl group is substituted.

As used herein, the term “alkoxy” refers to a radical in which an alkyl group having the designated number of carbon atoms is connected to the rest of the molecule via an oxygen atom. Alkoxy groups include C₁-C₁₂-alkoxy, C₁-C₈-alkoxy, C₁-C₆-alkoxy, C₁-C₄-alkoxy and C₁-C₃-alkoxy groups. Examples of alkoxy groups includes, but are not limited to, methoxy, ethoxy, n-propoxy, 2-propoxy (isopropoxy) and the higher homologs and isomers. Preferred alkoxy is C₁-C₃alkoxy.

An “aliphatic” group is a non-aromatic moiety comprised of any combination of carbon atoms, hydrogen atoms, halogen atoms, oxygen, nitrogen or other atoms, and optionally contains one or more units of unsaturation, e.g., double and/or triple bonds. Examples of aliphatic groups are functional groups, such as alkyl, alkenyl, alkynyl, O, OH, NH, NH₂, C(O), S(O)₂, C(O)O, C(O)NH, OC(O)O, OC(O)NH, OC(O)NH₂, S(O)₂NH, S(O)₂NH₂, NHC(O)NH₂, NHC(O)C(O)NH, NHS(O)₂NH, NHS(O)₂NH₂, C(O)NHS(O)₂, C(O)NHS(O)₂NH or C(O)NHS(O)₂NH₂, and the like, groups comprising one or more functional groups, non-aromatic hydrocarbons (optionally substituted), and groups wherein one or more carbons of a non-aromatic hydrocarbon (optionally substituted) is replaced by a functional group. Carbon atoms of an aliphatic group can be optionally oxo-substituted. An aliphatic group may be straight chained, branched, cyclic, or a combination thereof and preferably contains between about 1 and about 24 carbon atoms, more typically between about 1 and about 12 carbon atoms. In addition to aliphatic hydrocarbon groups, as used herein, aliphatic groups expressly include, for example, alkoxyalkyls, polyalkoxyalkyls, such as polyalkylene glycols, polyamines, and polyimines, for example. Aliphatic groups may be optionally substituted.

The terms “heterocyclic” and “heterocycloalkyl” can be used interchangeably and refer to a non-aromatic ring or a polycyclic ring system, such as a bi- or tri-cyclic fused, bridged or spiro system, where (i) each ring system contains at least one heteroatom independently selected from oxygen, sulfur and nitrogen, (ii) each ring system can be saturated or unsaturated (iii) the nitrogen and sulfur heteroatoms may optionally be oxidized, (iv) the nitrogen heteroatom may optionally be quaternized, (v) any of the above rings may be fused to an aromatic ring, and (vi) the remaining ring atoms are carbon atoms which may be optionally oxo-substituted or optionally substituted with exocyclic olefinic double bond. Representative heterocycloalkyl groups include, but are not limited to, 1,3-dioxolane, pyrrolidinyl, pyrazolinyl, pyrazolidinyl, imidazolinyl, imidazolidinyl, piperidinyl, piperazinyl, oxazolidinyl, isoxazolidinyl, morpholinyl, thiazolidinyl, isothiazolidinyl, quinoxalinyl, pyridazinonyl, 2-azabicyclo[2.2.1]-heptyl, 8-azabicyclo[3.2.1]octyl, 5-azaspiro[2.5]octyl, 2-oxa-7-azaspiro[4.4]nonanyl, 7-oxooxepan-4-yl, and tetrahydrofuryl. Such heterocyclic or heterocycloalkyl groups may be further substituted. A heterocycloalkyl or heterocyclic group can be C-attached or N-attached where possible.

It is understood that any alkyl, alkenyl, alkynyl, alicyclic, cycloalkyl, cycloalkenyl, aryl, heteroaryl, heterocyclic, aliphatic moiety or the like described herein can also be a divalent or multivalent group when used as a linkage to connect two or more groups or substituents, which can be at the same or different atom(s). One of skill in the art can readily determine the valence of any such group from the context in which it occurs.

The term “substituted” refers to substitution by independent replacement of one, two, or three or more of the hydrogen atoms with substituents including, but not limited to, —F, —Cl, —Br, —I, —OH, C₁-C₁₂-alkyl; C₂-C₁₂-alkenyl, C₂-C₁₂-alkynyl, —C₃-C₁₂-cycloalkyl, protected hydroxy, —NO₂, —N₃, —CN, —NH₂, protected amino, oxo, thioxo, —NH—C₂-C₈-alkenyl, —NH—C₂-C₈-alkynyl, —NH—C₃-C₁₂-cycloalkyl, —NH-aryl, —NH-heteroaryl, —NH-heterocycloalkyl, -dialkylamino, -diarylamino, -diheteroarylamino, —O—C₁-C₁₂-alkyl, —O—C₂-C₈-alkenyl, —O—C₂-C₈-alkynyl, —O—C₃-C₁₂-cycloalkyl, —O-aryl, —O-heteroaryl, —O-heterocycloalkyl, —C(O)—C₂-C₈-alkenyl, —C(O)—C₂-C₈-alkynyl, —C(O)—C₃-C₁₂-cycloalkyl, —C(O)-aryl, —C(O)— heteroaryl, —C(O)-heterocycloalkyl, —CONH₂, —CONH—C₁-C₁₂-alkyl, —CONH—C₂-C₈-alkenyl, —CONH—C₂-C₈-alkynyl, —CONH—C₃-C₁₂-cycloalkyl, —CONH-aryl, —CONH-heteroaryl, —CONH— heterocycloalkyl, —OCO₂—C₁-C₁₂-alkyl, —OCO₂—C₂-C₈-alkenyl, —OCO₂—C₂-C₈-alkynyl, —OCO₂—C₃-C₁₂-cycloalkyl, —OCO₂-aryl, —OCO₂-heteroaryl, —OCO₂-heterocycloalkyl, —OCO₂—C₁-C₁₂ alkyl, —CO₂—C₂-C₈ alkenyl, —CO₂—C₂-C₈ alkynyl, —CO₂—C₃-C₁₂-cycloalkyl, —CO₂-aryl, —CO₂-heteroaryl, —CO₂-heterocyloalkyl, —OCONH₂, —OCONH—C₁-C₁₂-alkyl, —OCONH—C₂-C₈-alkenyl, —OCONH—C₂-C₈-alkynyl, —OCONH—C₃-C₁₂-cycloalkyl, —OCONH-aryl, —OCONH-heteroaryl, —OCONH-heterocycloalkyl, —NHC(O)H, —NHC(O)—C₁-C₁₂-alkyl, —NHC(O)—C₂-C₈-alkenyl, —NHC(O)—C₂-C₈-alkynyl, —NHC(O)—C₃-C₁₂-cycloalkyl, —NHC(O)-aryl, —NHC(O)-heteroaryl, —NHC(O)— heterocycloalkyl, —NHCO₂—C₁-C₁₂-alkyl, —NHCO₂—C₂-C₈-alkenyl, —NHCO₂—C₂-C₈-alkynyl, —NHCO₂—C₃-C₁₂-cycloalkyl, —NHCO₂-aryl, —NHCO₂-heteroaryl, —NHCO₂-heterocycloalkyl, —NHC(O)NH₂, —NHC(O)NH—C₁-C₁₂-alkyl, —NHC(O)NH—C₂-C₈-alkenyl, —NHC(O)NH—C₂-C₈-alkynyl, —NHC(O)NH—C₃-C₁₂-cycloalkyl, —NHC(O)NH-aryl, —NHC(O)NH-heteroaryl, —NHC(O)NH-heterocycloalkyl, —NHC(S)NH₂, —NHC(S)NH—C₁-C₁₂-alkyl, —NHC(S)NH—C₂-C₈-alkenyl, —NHC(S)NH—C₂-C₈-alkynyl, —NHC(S)NH—C₃-C₁₂-cycloalkyl, —NHC(S)NH-aryl, —NHC(S)NH-heteroaryl, —NHC(S)NH-heterocycloalkyl, —NHC(NH)NH₂, —NHC(NH)NH—C₁-C₁₂-alkyl, —NHC(NH)NH—C₂-C₈-alkenyl, —NHC(NH)NH—C₂-C₈-alkynyl, —NHC(NH)NH—C₃-C₁₂-cycloalkyl, —NHC(NH)NH-aryl, —NHC(NH)NH-heteroaryl, —NHC(NH)NH-heterocycloalkyl, —NHC(NH)—C₁-C₁₂-alkyl, —NHC(NH)—C₂-C₈-alkenyl, —NHC(NH)—C₂-C₈-alkynyl, —NHC(NH)—C₃-C₁₂-cycloalkyl, —NHC(NH)-aryl, —NHC(NH)-heteroaryl, —NHC(NH)-heterocycloalkyl, —C(NH)NH₂, —C(NH)NH—C₂-C₈-alkenyl, —C(NH)NH—C₂-C₈-alkynyl, —C(NH)NH—C₃-C₁₂-cycloalkyl, —C(NH)NH-aryl, —C(NH)NH-heteroaryl, —C(NH)NH-heterocycloalkyl, —S(O)—C₁-C₁₂-alkyl, —S(O)—C₂-C₈-alkenyl, —S(O)—C₂-C₈-alkynyl, —S(O)—C₃-C₁₂-cycloalkyl, —S(O)-aryl, —S(O)-heteroaryl, —S(O)-heterocycloalkyl, —SO₂NH₂, —SO₂NH—C₁-C₁₂-alkyl, —SO₂NH—C₂-C₈-alkenyl, —SO₂NH—C₂-C₈-alkynyl, —SO₂—C₂-C₈-alkenyl, —SO₂—C₂-C₈-alkynyl, —SO₂—C₃-C₁₂-cycloalkyl, —SO₂-aryl, —SO₂-heteroaryl, —SO₂-heterocycloalkyl, —SO₂NH—C₃-C₁₂-cycloalkyl, —SO₂NH-aryl, —SO₂NH-heteroaryl, —SO₂NH-heterocycloalkyl, —NHSO₂—C₁-C₁₂-alkyl, —NHSO₂—C₂-C₈-alkenyl, —NHSO₂—C₂-C₈-alkynyl, —NHSO₂—C₃-C₁₂-cycloalkyl, —NHSO₂-aryl, —NHSO₂-heteroaryl, —NHSO₂-heterocycloalkyl, —CH₂NH₂, —CH₂SO₂CH₃, -aryl, -arylalkyl, -heteroaryl, -heteroarylalkyl, -heterocycloalkyl, —C₃-C₁₂-cycloalkyl, polyalkoxyalkyl, polyalkoxy, -methoxymethoxy, -methoxyethoxy, —SH, alkyl, —S—C₂-C₈-alkenyl, —S—C₂-C₈-alkynyl, —S—C₃-C₁₂-cycloalkyl, —S-aryl, —S-heteroaryl, -5-heterocycloalkyl, or methylthio-methyl. In certain embodiments, the substituents are independently selected from halo, preferably Cl and F; C₁-C₄-alkyl, preferably methyl and ethyl; halo-C₁-C₄-alkyl, such as fluoromethyl, difluoromethyl, and trifluoromethyl; C₂-C₄-alkenyl; halo-C₂-C₄-alkenyl; C₃-C₆-cycloalkyl, such as cyclopropyl; C₁-C₄-alkoxy, such as methoxy and ethoxy; halo-C₁-C₄-alkoxy, such as fluoromethoxy, difluoromethoxy, and trifluoromethoxy; —CN; —OH; NH₂; C₁-C₄-alkylamino; di(C₁-C₄-alkyl)amino; and NO₂. It is understood that an aryl, heteroaryl, alkyl, alkenyl, alkynyl, cycloalkyl, or heterocycloalkyl in a substituent can be further substituted. In certain embodiments, a substituent in a substituted moiety is additionally optionally substituted with one or more groups, each group being independently selected from C₁-C₄-alkyl; —CF₃, —OCH₃, —OCF₃, —F, —Cl, —Br, —I, —OH, —NO₂, —CN, and —NH₂. Preferably, a substituted alkyl group is substituted with one or more halogen atoms, more preferably one or more fluorine or chlorine atoms.

The term “halo” or halogen” alone or as part of another substituent, as used herein, refers to a fluorine, chlorine, bromine, or iodine atom.

The term “optionally substituted”, as used herein, means that the referenced group may be substituted or unsubstituted. In one embodiment, the referenced group is optionally substituted with zero substituents, i.e., the referenced group is unsubstituted. In certain embodiments, the referenced group is optionally substituted with one or more additional group(s) individually and independently selected from groups described herein.

The term “hydrogen” includes hydrogen and deuterium. In addition, the recitation of an element includes all isotopes of that element so long as the resulting compound is pharmaceutically acceptable. In certain embodiments, the isotopes of an element are present at a particular position according to their natural abundance. In other embodiments, one or more isotopes of an element at a particular position are enriched beyond their natural abundance.

The term “hydroxy activating group,” as used herein, refers to a labile chemical moiety which is known in the art to activate a hydroxyl group so that it will depart during synthetic procedures such as in a substitution or an elimination reaction. Examples of hydroxyl activating group include, but not limited to, mesylate, tosylate, triflate, p-nitrobenzoate, phosphonate and the like.

The term “activated hydroxyl,” as used herein, refers to a hydroxy group activated with a hydroxyl activating group, as defined above, including, but not limited to mesylate, tosylate, triflate, p-nitrobenzoate, phosphonate groups.

The term “hydroxy protecting group,” as used herein, refers to a labile chemical moiety which is known in the art to protect a hydroxyl group against undesired reactions during synthetic procedures. After said synthetic procedure(s) the hydroxy protecting group as described herein may be selectively removed. Hydroxy protecting groups as known in the art are described generally in P. G. M. Wuts, Greene's Protective Groups in Organic Synthesis, 5th edition, John Wiley & Sons, Hoboken, NJ (2014). Examples of hydroxyl protecting groups include, but are not limited to, benzyloxycarbonyl, 4-methoxybenzyloxycarbonyl, tert-butoxycarbonyl, isopropoxycarbonyl, diphenylmethoxycarbonyl, 2,2,2-trichloroethoxycarbonyl, allyloxycarbonyl, acetyl, formyl, chloroacetyl, trifluoroacetyl, methoxyacetyl, phenoxyacetyl, benzoyl, methyl, t-butyl, 2,2,2-trichloroethyl, 2-trimethylsilyl ethyl, allyl, benzyl, triphenyl-methyl (trityl), methoxymethyl, methylthiomethyl, benzyloxymethyl, 2-(trimethylsilyl)-ethoxymethyl, methanesulfonyl, trimethylsilyl, triisopropylsilyl, and the like.

The term “protected hydroxy,” as used herein, refers to a hydroxy group protected with a hydroxy protecting group, as defined above, including but not limited to, benzoyl, acetyl, trimethylsilyl, triethylsilyl, methoxymethyl groups, for example.

The term “hydroxy prodrug group,” as used herein, refers to a promoiety group which is known in the art to change the physicochemical, and hence the biological properties of a parent drug in a transient manner by covering or masking the hydroxy group. After said synthetic procedure(s), the hydroxy prodrug group as described herein must be capable of reverting back to hydroxy group in vivo. Hydroxy prodrug groups as known in the art are described generally in Kenneth B. Sloan, Prodrugs, Topical and Ocular Drug Delivery, (Drugs and the Pharmaceutical Sciences; Volume 53), Marcel Dekker, Inc., New York (1992).

The term “amino protecting group,” as used herein, refers to a labile chemical moiety which is known in the art to protect an amino group against undesired reactions during synthetic procedures. After said synthetic procedure(s) the amino protecting group as described herein may be selectively removed. Amino protecting groups as known in the art are described generally in P. G. M. Wuts, Greene's Protective Groups in Organic Synthesis, 5th edition, John Wiley & Sons, Hoboken, NJ (2014). Examples of amino protecting groups include, but are not limited to, methoxycarbonyl, t-butoxycarbonyl, 12-fluorenyl-methoxycarbonyl, benzyloxycarbonyl, and the like.

The term “protected amino,” as used herein, refers to an amino group protected with an amino protecting group as defined above.

The term “leaving group” means a functional group or atom which can be displaced by another functional group or atom in a substitution reaction, such as a nucleophilic substitution reaction. By way of example, representative leaving groups include chloro, bromo and iodo groups; sulfonic ester groups, such as mesylate, tosylate, brosylate, nosylate and the like; and acyloxy groups, such as acetoxy, trifluoroacetoxy and the like.

The term “aprotic solvent,” as used herein, refers to a solvent that is relatively inert to proton activity, i.e., not acting as a proton-donor. Examples include, but are not limited to, hydrocarbons, such as hexane and toluene, for example, halogenated hydrocarbons, such as, for example, methylene chloride, ethylene chloride, chloroform, and the like, heterocyclic compounds, such as, for example, tetrahydrofuran and N-methylpyrrolidinone, and ethers such as diethyl ether, bis-methoxymethyl ether. Such compounds are well known to those skilled in the art, and it will be obvious to those skilled in the art that individual solvents or mixtures thereof may be preferred for specific compounds and reaction conditions, depending upon such factors as the solubility of reagents, reactivity of reagents and preferred temperature ranges, for example. Further discussions of aprotic solvents may be found in organic chemistry textbooks or in specialized monographs, for example: Organic Solvents Physical Properties and Methods of Purification, 4^{th} ed., edited by John A. Riddick et al., Vol. II, in the Techniques of Chemistry Series, John Wiley & Sons, N Y, 1986.

The term “protic solvent,” as used herein, refers to a solvent that tends to provide protons, such as an alcohol, for example, methanol, ethanol, propanol, isopropanol, butanol, t-butanol, and the like. Such solvents are well known to those skilled in the art, and it will be obvious to those skilled in the art that individual solvents or mixtures thereof may be preferred for specific compounds and reaction conditions, depending upon such factors as the solubility of reagents, reactivity of reagents and preferred temperature ranges, for example. Further discussions of protogenic solvents may be found in organic chemistry textbooks or in specialized monographs, for example: Organic Solvents Physical Properties and Methods of Purification, 4th ed., edited by John A. Riddick et al., Vol. II, in the Techniques of Chemistry Series, John Wiley & Sons, N Y, 1986.

Combinations of substituents and variables envisioned by this invention are only those that result in the formation of stable compounds. The term “stable,” as used herein, refers to compounds which possess stability sufficient to allow manufacture and which maintains the integrity of the compound for a sufficient period of time to be useful for the purposes detailed herein (e.g., therapeutic or prophylactic administration to a subject).

The synthesized compounds can be separated from a reaction mixture and further purified by a method such as column chromatography, high pressure liquid chromatography, or recrystallization. As can be appreciated by the skilled artisan, further methods of synthesizing the compounds of the Formula herein will be evident to those of ordinary skill in the art. Additionally, the various synthetic steps may be performed in an alternate sequence or order to give the desired compounds. Synthetic chemistry transformations and protecting group methodologies (protection and deprotection) useful in synthesizing the compounds described herein are known in the art and include, for example, those such as described in R. Larock, Comprehensive Organic Transformations, 2^{nd} Ed. Wiley-VCH (1999); P. G. M. Wuts, Greene's Protective Groups in Organic Synthesis, 5th edition, John Wiley & Sons, Hoboken, N J (2014); L. Fieser and M. Fieser, Fieser and Fieser's Reagents for Organic Synthesis, John Wiley and Sons (1994); and L. Paquette, ed., Encyclopedia of Reagents for Organic Synthesis, John Wiley and Sons (1995), and subsequent editions thereof.

The term “subject,” as used herein, refers to an animal. Preferably, the animal is a mammal. More preferably, the mammal is a human. A subject also refers to, for example, a dog, cat, horse, cow, pig, guinea pig, fish, bird and the like.

The compounds of this invention may be modified by appending appropriate functionalities to enhance selective biological properties. Such modifications are known in the art and may include those which increase biological penetration into a given biological system (e.g., blood, lymphatic system, central nervous system), increase oral availability, increase solubility to allow administration by injection, alter metabolism and alter rate of excretion.

The compounds described herein contain one or more asymmetric centers and thus give rise to enantiomers, diastereomers, and other stereoisomeric forms that may be defined, in terms of absolute stereochemistry, as (R)- or (S)-, or as (D)- or (L)- for amino acids. The present invention is meant to include all such possible isomers, as well as their racemic and optically pure forms. Optical isomers may be prepared from their respective optically active precursors by the procedures described above, or by resolving the racemic mixtures. The resolution can be carried out in the presence of a resolving agent, by chromatography or by repeated crystallization or by some combination of these techniques which are known to those skilled in the art. Further details regarding resolutions can be found in Jacques, et al., Enantiomers, Racemates, and Resolutions (John Wiley & Sons, 1981). When the compounds described herein contain olefinic double bonds, other unsaturation, or other centers of geometric asymmetry, and unless specified otherwise, it is intended that the compounds include both E and Z geometric isomers or cis- and trans-isomers. Likewise, all tautomeric forms are also intended to be included. Tautomers may be in cyclic or acyclic. The configuration of any carbon-carbon double bond appearing herein is selected for convenience only and is not intended to designate a particular configuration unless the text so states; thus a carbon-carbon double bond or carbon-heteroatom double bond depicted arbitrarily herein as trans may be cis, trans, or a mixture of the two in any proportion.

Certain compounds of the present invention may also exist in different stable conformational forms which may be separable. Torsional asymmetry due to restricted rotation about an asymmetric single bond, for example because of steric hindrance or ring strain, may permit separation of different conformers. The present invention includes each conformational isomer of these compounds and mixtures thereof.

As used herein, the term “pharmaceutically acceptable salt,” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, S. M. Berge, et al. describes pharmaceutically acceptable salts in detail in J. Pharmaceutical Sciences, 66: 2-19 (1977). The salts can be prepared in situ during the final isolation and purification of the compounds of the invention, or separately by reacting the free base function with a suitable organic acid. Examples of pharmaceutically acceptable salts include, but are not limited to, nontoxic acid addition salts are salts of an amino group formed with inorganic acids such as hydrochloric acid, hydrobromic acid, phosphoric acid, sulfuric acid and perchloric acid or with organic acids such as acetic acid, maleic acid, tartaric acid, citric acid, succinic acid or malonic acid or by using other methods used in the art such as ion exchange. Other pharmaceutically acceptable salts include, but are not limited to, adipate, alginate, ascorbate, aspartate, benzenesulfonate, benzoate, bisulfate, borate, butyrate, camphorate, camphorsulfonate, citrate, cyclopentane-propionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, alkyl having from 1 to 6 carbon atoms, sulfonate and aryl sulfonate.

As used herein, the term “pharmaceutically acceptable ester” refers to esters which hydrolyze in vivo and include those that break down readily in the human body to leave the parent compound or a salt thereof. Suitable ester groups include, for example, those derived from pharmaceutically acceptable aliphatic carboxylic acids, particularly alkanoic, alkenoic, cycloalkanoic and alkanedioic acids, in which each alkyl or alkenyl moiety advantageously has not more than 6 carbon atoms. Examples of particular esters include, but are not limited to, formates, acetates, propionates, butyrates, acrylates and ethylsuccinates.

### Pharmaceutical Compositions

The pharmaceutical compositions of the present invention comprise a therapeutically effective amount of a compound of the present invention formulated together with one or more pharmaceutically acceptable carriers or excipients.

As used herein, the term “pharmaceutically acceptable carrier or excipient” means a non-toxic, inert solid, semi-solid or liquid filler, diluent, encapsulating material or formulation auxiliary of any type. Some examples of materials which can serve as pharmaceutically acceptable carriers are sugars such as lactose, glucose and sucrose; starches such as corn starch and potato starch; cellulose and its derivatives such as sodium carboxymethyl cellulose, ethyl cellulose and cellulose acetate; powdered tragacanth; malt; gelatin; talc; excipients such as cocoa butter and suppository waxes; oils such as peanut oil, cottonseed oil, safflower oil, sesame oil, olive oil, corn oil and soybean oil; glycols such as propylene glycol; esters such as ethyl oleate and ethyl laurate; agar; buffering agents such as magnesium hydroxide and aluminum hydroxide; alginic acid; pyrogen-free water; isotonic saline; Ringer's solution; ethyl alcohol, and phosphate buffer solutions, as well as other non-toxic compatible lubricants such as sodium lauryl sulfate and magnesium stearate, as well as coloring agents, releasing agents, coating agents, sweetening, flavoring and perfuming agents, preservatives and antioxidants can also be present in the composition, according to the judgment of the formulator.

The pharmaceutical compositions of this invention may be administered orally, parenterally, by inhalation spray, topically, rectally, nasally, buccally, vaginally or via an implanted reservoir, preferably by oral administration or administration by injection. The pharmaceutical compositions of this invention may contain any conventional non-toxic pharmaceutically-acceptable carriers, adjuvants or vehicles. In some cases, the pH of the formulation may be adjusted with pharmaceutically acceptable acids, bases or buffers to enhance the stability of the formulated compound or its delivery form. The term parenteral as used herein includes subcutaneous, intracutaneous, intravenous, intramuscular, intraarticular, intra-arterial, intrasynovial, intrasternal, intrathecal, intralesional and intracranial injection or infusion techniques.

Liquid dosage forms for oral administration include pharmaceutically acceptable emulsions, microemulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Injectable preparations, for example, sterile injectable aqueous or oleaginous suspensions, may be formulated according to the known art using suitable dispersing or wetting agents and suspending agents. The sterile injectable preparation may also be a sterile injectable solution, suspension or emulsion in a nontoxic parenterally acceptable diluent or solvent, for example, as a solution in 1,3-butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose, any bland fixed oil can be employed including synthetic mono- or diglycerides. In addition, fatty acids such as oleic acid are used in the preparation of injectable.

The injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

In order to prolong the effect of a drug, it is often desirable to slow the absorption of the drug from subcutaneous or intramuscular injection. This may be accomplished by the use of a liquid suspension of crystalline or amorphous material with poor water solubility. The rate of absorption of the drug then depends upon its rate of dissolution, which, in turn, may depend upon crystal size and crystalline form. Alternatively, delayed absorption of a parenterally administered drug form is accomplished by dissolving or suspending the drug in an oil vehicle. Injectable depot forms are made by forming microencapsule matrices of the drug in biodegradable polymers such as polylactide-polyglycolide. Depending upon the ratio of drug to polymer and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions that are compatible with body tissues.

Compositions for rectal or vaginal administration are preferably suppositories which can be prepared by mixing the compounds of this invention with suitable non-irritating excipients or carriers such as cocoa butter, polyethylene glycol or a suppository wax which are solid at ambient temperature but liquid at body temperature and therefore melt in the rectum or vaginal cavity and release the active compound.

Solid dosage forms for oral administration include capsules, tablets, pills, powders, and granules. In such solid dosage forms, the active compound is mixed with at least one inert, pharmaceutically acceptable excipient or carrier such as sodium citrate or dicalcium phosphate and/or: a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may also comprise buffering agents.

Solid compositions of a similar type may also be employed as fillers in soft and hard-filled gelatin capsules using such excipients as lactose or milk sugar as well as high molecular weight polyethylene glycols and the like.

The solid dosage forms of tablets, dragees, capsules, pills, and granules can be prepared with coatings and shells such as enteric coatings and other coatings well known in the pharmaceutical formulating art. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes.

Dosage forms for topical or transdermal administration of a compound of this invention include ointments, pastes, creams, lotions, gels, powders, solutions, sprays, inhalants or patches. The active component is admixed under sterile conditions with a pharmaceutically acceptable carrier and any needed preservatives or buffers as may be required. Ophthalmic formulations, ear drops, eye ointments, powders and solutions are also contemplated as being within the scope of this invention.

The ointments, pastes, creams and gels may contain, in addition to an active compound of this invention, excipients such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to the compounds of this invention, excipients such as lactose, talc, silicic acid, aluminum hydroxide, calcium silicates and polyamide powder, or mixtures of these substances. Sprays can additionally contain customary propellants such as chlorofluorohydrocarbons.

Transdermal patches have the added advantage of providing controlled delivery of a compound to the body. Such dosage forms can be made by dissolving or dispensing the compound in the proper medium. Absorption enhancers can also be used to increase the flux of the compound across the skin. The rate can be controlled by either providing a rate controlling membrane or by dispersing the compound in a polymer matrix or gel.

For pulmonary delivery, a therapeutic composition of the invention is formulated and administered to the patient in solid or liquid particulate form by direct administration e.g., inhalation into the respiratory system. Solid or liquid particulate forms of the active compound prepared for practicing the present invention include particles of respirable size: that is, particles of a size sufficiently small to pass through the mouth and larynx upon inhalation and into the bronchi and alveoli of the lungs. Delivery of aerosolized therapeutics, particularly aerosolized antibiotics, is known in the art (see, for example U.S. Pat. No. 5,767,068 to Van Devanter et al., U.S. Pat. No. 5,508,269 to Smith et al., and WO 98/43650 by Montgomery, all of which are incorporated herein by reference).

### Combination and Alternation Therapy

The compounds of the present invention may be used in combination with one or more antiviral therapeutic agents or anti-inflammatory agents useful in the prevention or treatment of viral diseases or associated pathophysiology. Thus, the compounds of the present invention and their salts, solvates, or other pharmaceutically acceptable derivatives thereof, may be employed alone or in combination with other antiviral or anti-inflammatory therapeutic agents. The compounds herein and pharmaceutically acceptable salts thereof may be used in combination with one or more other agents which may be useful in the prevention or treatment of respiratory disease, inflammatory disease, autoimmune disease, for example; anti-histamines, corticosteroids, (e.g., fluticasone propionate, fluticasone furoate, beclomethasone dipropionate, budesonide, ciclesonide, mometasone furoate, triamcinolone, flunisolide), NSAIDs, leukotriene modulators (e.g., montelukast, zafirlukast.pranlukast), tryptase inhibitors, IKK2 inhibitors, p38 inhibitors, Syk inhibitors, protease inhibitors such as elastase inhibitors, integrin antagonists (e.g., beta-2 integrin antagonists), adenosine A2a agonists, mediator release inhibitors such as sodium chromoglycate, 5-lipoxygenase inhibitors (zyflo), DP1 antagonists, DP2 antagonists, PI3K delta inhibitors, ITK inhibitors, LP (Iysophosphatidic) inhibitors or FLAP (5-lipoxygenase activating protein) inhibitors (e.g., sodium 3-(3-(tert-butylthio)-1-(4-(6-ethoxypyridin-3-yl)benzyl)-5-((5-ethylpyridin-2-yl)methoxy)-1H-indol-2-yl)-2,2-dimethylpropanoate), bronchodilators (e.g., muscarinic antagonists, beta-2 agonists), methotrexate, and similar agents; monoclonal antibody therapy such as anti-IgE, anti-TNF, anti-IL-5, anti-IL-6, anti-IL-12, anti-IL-1 and similar agents; cytokine receptor therapies e.g. etanercept and similar agents; antigen non-specific immunotherapies (e.g. interferon or other cytokines/chemokines, chemokine receptor modulators such as CCR3, CCR4 or CXCR2 antagonists, other cytokine/chemokine agonists or antagonists, TLR agonists and similar agents), suitable anti-infective agents including antibiotic agents, antifungal agents, antheimintic agents, antimalarial agents, antiprotozoal agents, antitubercuiosis agents, and antiviral agents, including those listed at https://www.drugs.com/drug-class/anti-infectives.html.

The compounds of the present invention and any other pharmaceutically active agent(s) may be administered together or separately and, when administered separately, administration may occur simultaneously or sequentially, in any order. The amounts of the compounds of the present invention and the other pharmaceutically active agent(s) and the relative timings of administration will be selected in order to achieve the desired combined therapeutic effect. The administration in combination of a compound of the present invention and salts, solvates, or other pharmaceutically acceptable derivatives thereof with other treatment agents may be achieved by concomitant administration in: (1) a unitary pharmaceutical composition including both compounds; or (2) separate pharmaceutical compositions each including one of the compounds.

In certain embodiments of the combination therapy, the additional therapeutic agent is administered at a lower dose and/or dosing frequency as compared to dose and/or dosing frequency of the additional therapeutic agent required to achieve similar results in treating or preventing of an 17β-HSD13 mediated disease or condition.

Although the invention has been described with respect to various preferred embodiments, it is not intended to be limited thereto, but rather those skilled in the art will recognize that variations and modifications may be made therein which are within the spirit of the invention and the scope of the appended claims.

### Therapeutic Activity

A therapeutically effective amount or dose of the compounds of the present invention may range from about 0.01 mg/Kg to about 500 mg/Kg, alternatively from about 1 to about 50 mg/Kg. Therapeutically effective amounts or doses will also vary depending on route of administration, as well as the possibility of co-usage with other agents.

According to the methods of treatment of the present invention, viral infections are treated or prevented in a patient such as a human or another animal by administering to the patient a therapeutically effective amount of a compound of the invention, in such amounts and for such time as is necessary to achieve the desired result.

By a “therapeutically effective amount” of a compound of the invention is meant an amount of the compound which confers a therapeutic effect on the treated subject, at a reasonable benefit/risk ratio applicable to any medical treatment. The therapeutic effect may be objective (i.e., measurable by some test or marker) or subjective (i.e., subject gives an indication of or feels an effect). An effective amount of the compound described above may range from about 0.1 mg/Kg to about 500 mg/Kg, preferably from about 1 to about 50 mg/Kg. Effective doses will also vary depending on route of administration, as well as the possibility of co-usage with other agents. It will be understood, however, that the total daily usage of the compounds and compositions of the present invention will be decided by the attending physician within the scope of sound medical judgment. The specific therapeutically effective dose level for any particular patient will depend upon a variety of factors including the disorder being treated and the severity of the disorder; the activity of the specific compound employed; the specific composition employed; the age, body weight, general health, sex and diet of the patient; the time of administration, route of administration, and rate of excretion of the specific compound employed; the duration of the treatment; drugs used in combination or contemporaneously with the specific compound employed; and like factors well known in the medical arts.

The total daily dose of the compounds of this invention administered to a human or other animal in single or in divided doses can be in amounts, for example, from 0.01 to 50 mg/kg body weight or more usually from 0.1 to 25 mg/kg body weight. Single dose compositions may contain such amounts or submultiples thereof to make up the daily dose. In general, treatment regimens according to the present invention comprise administration to a patient in need of such treatment from about 10 mg to about 1000 mg of the compound(s) of this invention per day in single or multiple doses.

The compounds of the present invention described herein can, for example, be administered by injection, intravenously, intra-arterial, subdermally, intraperitoneally, intramuscularly, or subcutaneously; or orally, buccally, nasally, transmucosally, topically, in an ophthalmic preparation, or by inhalation, with a dosage ranging from about 0.1 to about 500 mg/kg of body weight, alternatively dosages between 1 mg and 1000 mg/dose, every 4 to 120 hours, or according to the requirements of the particular drug. The methods herein contemplate administration of an effective amount of compound or compound composition to achieve the desired or stated effect. Typically, the pharmaceutical compositions of this invention will be administered from about 1 to about 6 times per day or alternatively, as a continuous infusion. Such administration can be used as a chronic or acute therapy. The amount of active ingredient that may be combined with pharmaceutically excipients or carriers to produce a single dosage form will vary depending upon the host treated and the particular mode of administration. A typical preparation will contain from about 5% to about 95% active compound (w/w). Alternatively, such preparations may contain from about 20% to about 80% active compound.

Lower or higher doses than those recited above may be required. Specific dosage and treatment regimens for any particular patient will depend upon a variety of factors, including the activity of the specific compound employed, the age, body weight, general health status, sex, diet, time of administration, rate of excretion, drug combination, the severity and course of the disease, condition or symptoms, the patient's disposition to the disease, condition or symptoms, and the judgment of the treating physician.

Upon improvement of a patient's condition, a maintenance dose of a compound, composition or combination of this invention may be administered, if necessary. Subsequently, the dosage or frequency of administration, or both, may be reduced, as a function of the symptoms, to a level at which the improved condition is retained when the symptoms have been alleviated to the desired level. Patients may, however, require intermittent treatment on a long-term basis upon any recurrence of disease symptoms.

When the compositions of this invention comprise a combination of a compound of Formula (I) described herein and one or more additional therapeutic or prophylactic agents, both the compound and the additional agent should be present at dosage levels of between about 1 to 100%, and more preferably between about 5 to 95% of the dosage normally administered in a monotherapy regimen. The additional agents may be administered separately, as part of a multiple dose regimen, from the compounds of this invention. Alternatively, those agents may be part of a single dosage form, mixed together with the compounds of this invention in a single composition.

The “additional therapeutic or prophylactic agents” include but are not limited to, immune therapies (e.g. interferon), therapeutic vaccines, antifibrotic agents, anti-inflammatory agents such as corticosteroids or NSAIDs, bronchodilators such as beta-2 adrenergic agonists and xanthines (e.g. theophylline), mucolytic agents, anti-muscarinics, anti-leukotrienes, inhibitors of cell adhesion (e.g. ICAM antagonists), anti-oxidants (e.g. N-acetylcysteine), cytokine agonists, cytokine antagonists, lung surfactants and/or antimicrobial and anti-viral agents (e.g. ribavirin and amantidine). The compositions according to the invention may also be used in combination with gene replacement therapy.

### ABBREVIATIONS

Abbreviations which have been used in the descriptions of the schemes and the examples that follow are:
Alloc for allyloxycarbonyl;
Alloc-Cl for allyl chloroformate;
ASK1 for apoptosis signal-regulating kinase 1;
ATP for adenosine triphosphate;
Boc for tert-butyloxycarbonyl;
BOP-Cl for bis(2-oxo-3-oxazolidinyl)phosphinic chloride;
Cbz for benzyloxycarbonyl;
Cbz-Cl for benzyl chloroformate;
CDI for carbonyldiimidazole;
(COCl)₂ for oxalyl chloride;
DBU for 1,8-diazabicycloundec-7-ene;
DCC for N,N-dicyclohexylcarbodiimide;
1,2-DCE for 1,2-dichloroethane;
DCM for dichloromethane;
DIPEA or Hunig's base or i-Pr₂NEt for N,N-diisopropylethylamine;
DMAc for N,N-dimethylacetamide;
DMAP for N,N-dimethylaminopyridine;
DMF for N,N-dimethyl formamide;
EDC for 1-(3-diethylaminopropyl)-3-ethylcarbodiimide hydrochloride;
EGTA for ethylene glycol-bis(2-aminoethylether)-N,N,N′,N′-tetraacetic acid;
ESI for electrospray ionization;
Et₃N or TEA for triethylamine;
Et₂O for diethylether;
EtOAc for ethyl acetate;
Ghosez's Reagent for 1-chloro-N,N,2-trimethyl-1-propenylamine;
HATU for 1-[bis(dimethylamino)methylene]-1H-1,2,3-triazolo[4,5-b]pyridinium 3-oxid hexafluorophosphate;
HEPES for 4-(2-Hydroxyethyl)piperazine-1-ethanesulfonic acid, N-(2-Hydroxyethyl)piperazine-N′-(2-ethanesulfonic acid);
IC₅₀ for half maximal inhibitory concentration;
KOt-Bu for potassium tert-butoxide;
LCMS for liquid chromatography-mass spectrometry;
MeCN for acetonitrile;
MTBE for methyl tert-butyl ether;
m/z for mass-to-charge ratio;
NaOt-Bu for sodium tert-butoxide;
NMP for 1-methyl-2-pyrrolidinone;
NMR for nuclear magnetic resonance spectroscopy;
OMs or mesylate for methanesulfonate;
OTf or triflate for trifluoromethanesulfonate;
OTs or tosylate for para-toluenesulfonate;
Pd₂(dba)₃ for tris(dibenzylideneacetone)dipalladium(0);
P(o-tolyl)₃ for tri(o-tolyl)phosphine;
PyAOP for 7-azabenzotriazol-1-yloxy)tripyrrolidinophosphonium hexafluorophosphate;
PyBOP for benzotriazol-1-yl-oxytripyrrolidinophosphonium hexafluorophosphate;
STK3 for serine/threonine-protein kinase 3
TEA for triethylamine;
THF for tetrahydrofuran.

### Synthetic Methods

All references cited herein, whether in print, electronic, computer readable storage media or other form, are expressly incorporated by reference in their entirety, including but not limited to, abstracts, articles, journals, publications, texts, treatises, internet web sites, databases, patents, and patent publications.

Various changes and modifications to the disclosed embodiments will be apparent to those skilled in the art and such changes and modifications including, without limitation, those relating to the chemical structures, substituents, derivatives, formulations and/or methods of the invention may be made without departing from the spirit of the invention and the scope of the appended claims.

Although the invention has been described with respect to various preferred embodiments, it is not intended to be limited thereto, but rather those skilled in the art will recognize that variations and modifications may be made therein which are within the spirit of the invention and the scope of the appended claims.

The compounds and processes of the present invention will be better understood in connection with the following synthetic schemes that illustrate the methods by which the compounds of the invention may be prepared, which are intended as an illustration only and not to limit the scope of the invention.

As shown in Scheme 1, in a one-pot fashion, the compound of Formula (I) can be prepared from condensation reaction of the amino ester compound (1) prepared according to literature (New inhibitors of 17β-hydroxysteroid dehydrogenase type 1, Molecular and Cellular Endocrinology 2006, 248, 192-198, Josef Messinger, Leena Hirvela, Pasi Koskimies, Bettina Husen, Lauri Kangas, Olli Pentikainen, Pauli Saarenketo and Hubert Thole) and the amide compound (2) in the presence of POCl₃, SOCl₂ or PCl₅, wherein, R₁, R₂, R₃, R₄, R₅, R₆ and M are as previously defined. Thus, a mixture of amino ester compound (1) and amide compound (2) in an aprotic solvent is treated with POCl₃, SOCl₂ or PCl₅ to form compound of Formula (I). The aprotic solvent can be, such as, but not limited to, THF, DCE and DMF. The reaction temperature is from 0° C. to 140° C.

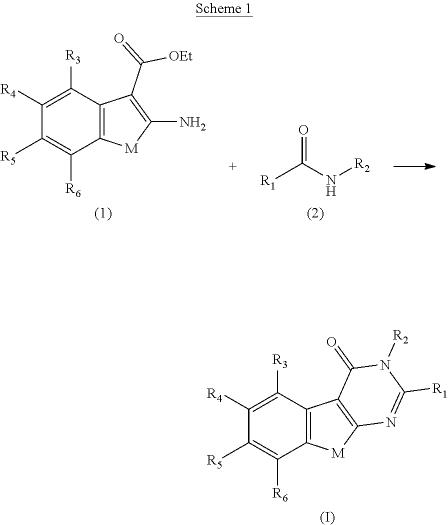

Alternatively, as shown in Scheme 2, the compound of Formula (I) can also be prepared via a stepwise fashion, wherein, R₁, R₂, R₃, R₄, R₅, R₆, and M are as previously defined. The amide compound (2) can react with POCl₃, SOCl₂ or PCl₅ in an aprotic solvent such as DCM, DCE, THF, or DMF to form imine chloride (3) at 0° C.˜80° C. followed by reacting with the amino ester compound (1) to give the cyclized compound (I). The reaction temperature is from 0° C. to 140° C.

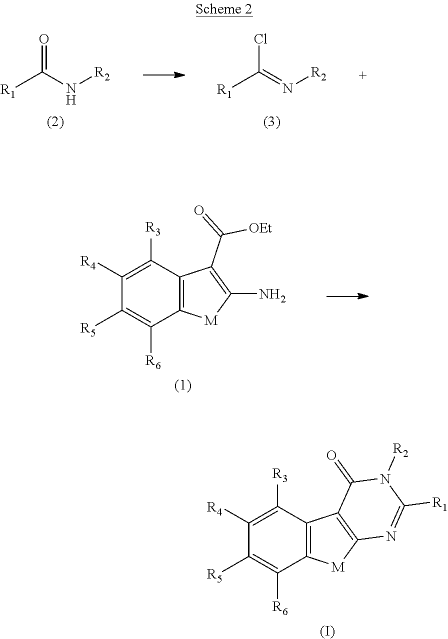

Alternatively, as shown in Scheme 3, the compound of Formula (I) can also be prepared by a transition metal/phosphine ligand complex catalyzed coupling reaction between the chloropyrimidone compound (7) and an organometallic reagent such as a boronic acid or related boron reagent partner (8), wherein, R₁, R₂, R₃, R₄, R₅, R₆ and M are as previously defined. The catalyst used in this reaction can be, but not limited to bis(triphenylphosphine)palladium(II) chloride. The base used in this reaction can be, but is not limited to, cesium carbonate. Compound (1) in an aprotic solvent is first treated with amine compound (4) in the presence of suitable coupling reagent and organic base to afford the urea compound (5). The suitable coupling reagent can be, such as, but not limited to, CDI, triphosgene or ethyl chloroformate and the organic base can be, such as, but not limited to, DBU, DIPEA or TEA. The aprotic solvent can be, such as, but not limited to, MeCN, THF, DCE or DMF. The reaction temperature is from 0° C. to 80° C. Then urea compound (5) is treated with suitable inorganic base in alcohol solvents at elevated temperature to provide the pyrimidinedione compound (6). The inorganic bases can be, such as, but not limited to, NaOMe, NaOEt, or NaOtBu. The alcohol solvent can be, such as, but not limited to, MeOH, EtOH or tBuOH. The temperature is from 40° C. to 80° C. The chloropyrimidone compound (7) is prepared from pyrimidinedione compound (6) in the presence of suitable chlorinating reagent in an aprotic solvent at elevated temperature. The suitable chlorinating reagent can be such as, but not limited to POCl₃. The aprotic solvent can be, such as, but not limited to, DCE. The reaction temperature is from 80° C. to 140° C. The chloropyrimidone compound (7) reacts with a boronic acid partner or related boron reagents (8) catalyzed by a transition metal/phosphine ligand complex in mixed solvents mixture. The solvent in this coupling reaction can be, but not limited to DME/H₂O, dioxane/H₂O, toluene/H₂O, etc. The reaction temperature is from 0° C. to 140° C.

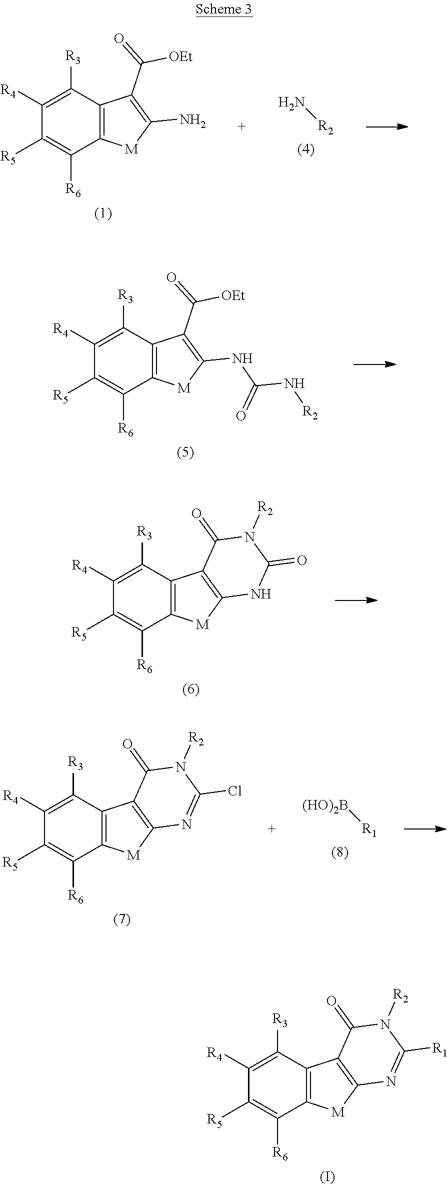

As shown in Scheme 4, the compound of Formula (IV) could be synthesized by substitution reaction between compound (9) and R₉-LG in aprotic solvent and in the presence of base, wherein, R₁, R₂, R₃, R₄, R₅, R₆ and M are as previously defined. The base can be organic bases or inorganic bases, but not limited to pyridine, DIPEA, DMAP, Cs₂CO₃, NaH, etc. The aprotic solvent can be, such as, but not limited to, pyridine, MeCN, THF, DCE or DMF. The reaction temperature is from 0° C. to 80° C.

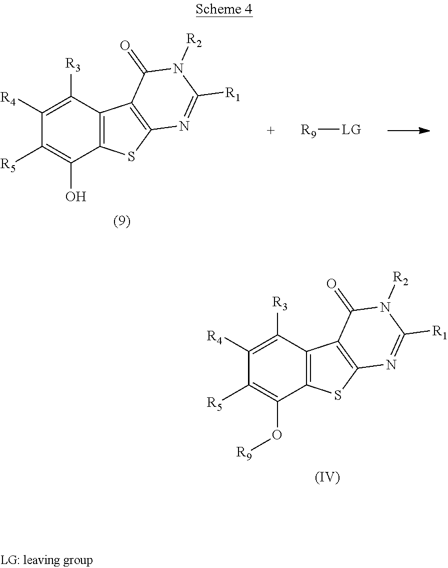

### EXAMPLES

The compounds and processes of the present invention will be better understood in connection with the following examples, which are intended as an illustration only and not limiting of the scope of the invention. Starting materials were either available from a commercial vendor or produced by methods well known to those skilled in the art.

### Synthesis of 2-(2-cyclopropyl-4-methoxyphenyl)-3-(oxazol-5-ylmethyl)-4-oxo-3,4-dihydrobenzo[4,5]thieno[2,3-d]pyrimidin-8-yl acetate (Example 1)

### Example 1

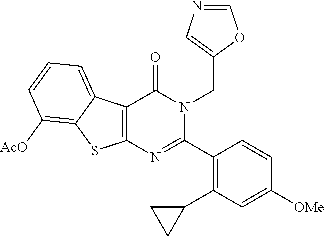 [SMILES: COc1ccc(-c2nc3sc4c(OC(C)=O)cccc4c3c(=O)n2Cc2cnco2)c(C2CC2)c1]

### Step 1

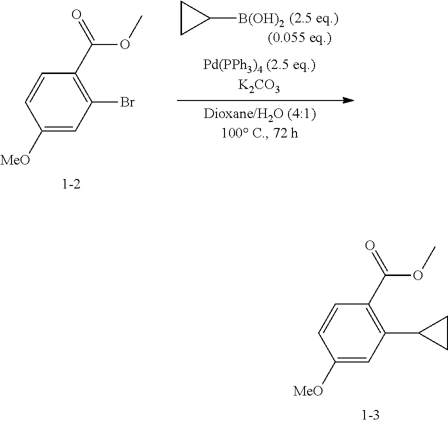

To a 250 mL round-bottomed flask were added compound 1-2 (5.0 g, 20.4 mmol), cyclopropylboronic acid (5.3 g, 61.2 mmol), tetrakis(triphenylphosphine)palladium (2.4 g, 2.0 mmol), potassium carbonate (8.5 g, 61.2 mmol) respectively under N₂ atmosphere followed by addition of dioxane (82 mL) and water (20 mL). The suspension was heated at 100° C. for 48 h. Cooled to rt, diluted with EtOAc, washed with water and brine respectively. Dried (Na₂SO₄), filtered, concentrated, and purified by CombiFlash (120 g SiO₂, EA/c-Hex: 0˜20%) to give compound 1-3 as a colorless liquid 2.4 g, 57.0% yield. LC-MS observed [M+H], 207.10. ¹H NMR (400 MHz, Chloroform-d) δ 7.86 (d, J=8.7 Hz, 1H), 6.70 (dd, J=8.7, 2.6 Hz, 1H), 6.51 (d, J=2.6 Hz, 1H), 3.88 (s, 3H), 3.82 (s, 3H), 2.90-2.69 (m, 1H), 1.11-0.90 (m, 2H), 0.77-0.60 (m, 2H).

### Step 2

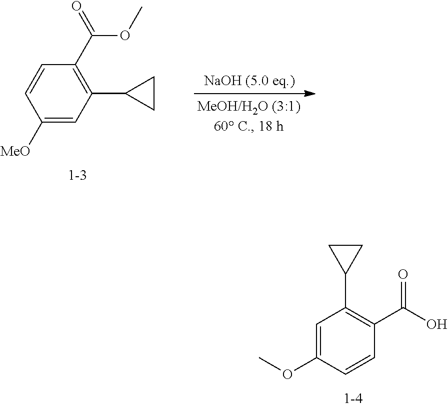

To a 250 mL round-bottomed flask containing compound 1-3 (2.4 g, 11.6 mmol) were added MeOH (87 mL), Water (29 mL), and sodium hydroxide (2.3 g, 58.2 mmol) respectively and the mixture was stirred at 60° C. overnight. The reaction was complete by TLC (70% EA/Hex). Cooled to rt, concentrated to remove half of the solvents. Water (10 mL) was added and the white suspension was cooled to 0° C. Acidified with 3N HCl (23.3 mL, 69.8 mmol) dropwise to pH˜1. The precipitates were filtered via a Buchi funnel and dried in vacuo to give compound 1-4 as a white powder, 2.17 g, 97% yield. LC-MS observed [M+H], 193.0. ¹H NMR (400 MHz, DMSO-d₆) δ 12.52 (s, 1H), 7.77 (d, J=8.7 Hz, 1H), 6.78 (dd, J=8.7, 2.6 Hz, 1H), 6.43 (d, J=2.6 Hz, 1H), 3.78 (s, 3H), 3.00-2.79 (m, 1H), 1.06-0.85 (m, 2H), 0.80-0.59 (m, 2H).

### Step 3

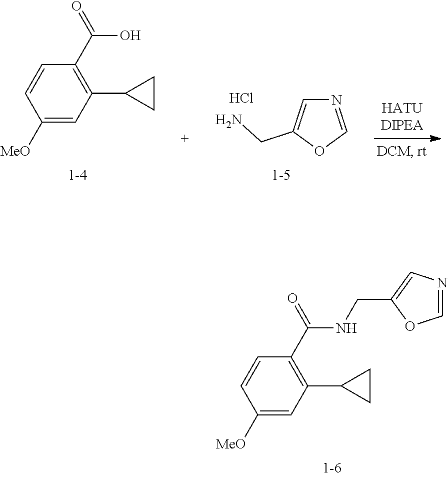

To a 25 mL round-bottomed flask were added 1-4 (0.45 g, 2.34 mmol), 1-5 (0.32 g, 2.34 mmol), HATU (1.42 g, 3.75 mmol), CH₂Cl₂ (7.5 mL), and DIPEA (1.31 mL, 7.49 mmol) respectively and the resulting mixture was stirred at rt for 25 h. Diluted with DCM, washed with Sat. NaHCO₃ and brine. Dried (Na₂SO₄), filtered, concentrated, and purified by CombiFlash (24 g SiO₂, EtOAc/c-Hex: 0˜100%) to give compound 1-6 as a white solid, 0.41 g, 63.8% yield. LC-MS observed [M+H], 273.12. ¹H NMR (400 MHz, Chloroform-d) δ 7.86 (s, 1H), 7.46 (d, J=8.5 Hz, 1H), 7.06 (s, 1H), 6.71 (dd, J=8.7, 2.5 Hz, 1H), 6.49 (d, J=2.5 Hz, 1H), 6.45 (s, 1H), 4.71 (d, J=5.7 Hz, 2H), 3.79 (s, 3H), 2.25 (m, 1H), 1.11-0.85 (m, 2H), 0.85-0.62 (m, 2H).

### Step 4

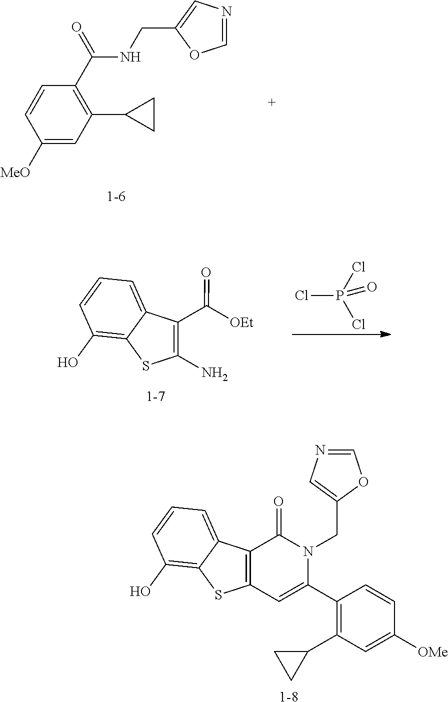

To a 25 mL round-bottomed flask were added compound 1-7 (0.52 g, 2.203 mmol) which was prepared according to literature method (Molecular and Cellular Endocrinology 2006, 248, 192-198), compound 1-6 (0.30 mg, 1.102 mmol), DCE (6.12 mL), and phosphoryl trichloride (154 μL, 1.65 mmol) respectively and the suspension was stirred at 80° C. for 18 h. The mixture was diluted with DCM and the insoluable brown solid was collected off by a Buchi funnel. The filtrate was washed thoroughly with Sat. NaHCO₃ and then washed with brine. The organic layer was dried (Na₂SO₄), filtered, concentrated, and purified by CombiFlash (40 g SiO₂, EtOAc/Cyclohexane: 0100%) to give compound 1-8 as a pale-yellow solid, 0.14 g, 28.5% yield. LC-MS observed [M+H], 446.12. ¹H NMR (400 MHz, DMSO-d₆) δ 10.68 (s, 1H), 8.22 (s, 1H), 8.02 (dd, J=7.9, 0.9 Hz, 1H), 7.43 (t, J=7.9 Hz, 1H), 7.37 (d, J=8.5 Hz, 1H), 6.98 (dd, J=7.9, 1.0 Hz, 1H), 6.88 (dd, J=8.5, 2.5 Hz, 1H), 6.85 (s, 1H), 6.48 (d, J=2.5 Hz, 1H), 5.40 (d, J=15.9 Hz, 1H), 5.09 (dd, J=15.9, 1.1 Hz, 1H), 3.82 (s, 3H), 1.57 (m, J=8.0, 7.5, 4.5 Hz, 1H), 0.83 (m, J=7.8, 4.7 Hz, 3H), 0.63 (m, J=4.2 Hz, 1H).

### Step 5

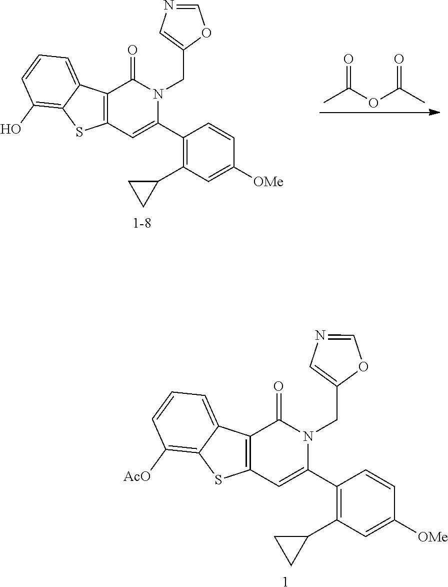

To a 2-dram vial were added compound 1-8 (0.1 g, 0.22 mmol), and acetic anhydride (106 μl, 1.12 mmol) respectively and the reaction was stirred at rt for 16 h. Diluted with DCM, washed with Sat. NaHCO₃. Dried (Na₂SO₄), filtered, concentrated, purified by CombiFlash (12 g SiO₂, EA/Hex: 0˜100%) to give compound 1 as a white foam, 0.1 g, 91% yield. LC-MS observed [M+H], 488.13. ¹H NMR (400 MHz, Chloroform-d) δ 8.56 (dd, J=7.9, 1.0 Hz, 1H), 7.74 (s, 1H), 7.57 (t, J=8.0 Hz, 1H), 7.31 (dd, J=8.0, 1.0 Hz, 1H), 7.27 (s, OH), 7.24 (d, J=8.4 Hz, 1H), 6.89-6.78 (m, 2H), 6.49 (d, J=2.5 Hz, 1H), 5.61 (d, J=15.4 Hz, 1H), 5.09 (dd, J=15.3, Hz, 1H), 3.85 (s, 3H), 2.42 (s, 3H), 1.56 (m, 1H), 1.01-0.76 (m, 3H), 0.74-0.56 (m, 1H).

### Synthesis of 2-(2-cyclopropyl-4-methoxyphenyl)-3-(oxazol-5-ylmethyl)-4-oxo-3,4-dihydrobenzo[4,5]thieno[2,3-d]pyrimidin-8-yl propionate (Example 2)

### Example 2

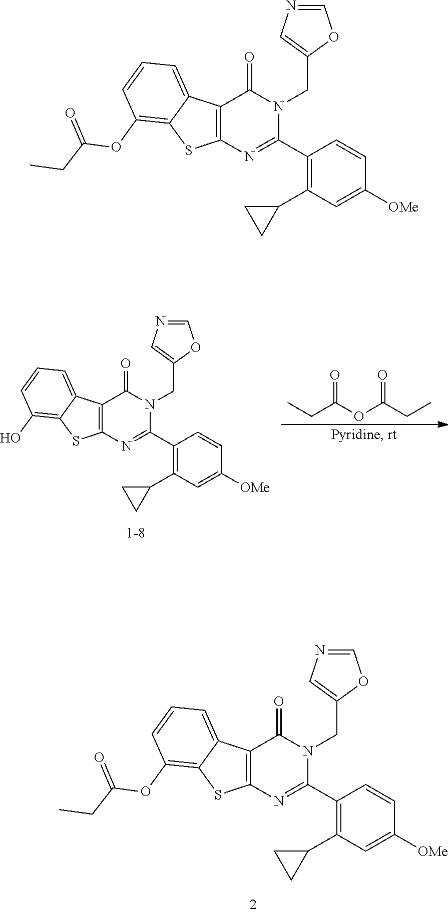

Example 2 was prepared by using similar procedure as described for compound 1. LC-MS observed [M+H], 502.14. ¹H NMR (400 MHz, Chloroform-d) δ 8.55 (dd, J=8.0, 1.0 Hz, 1H), 7.75 (s, 1H), 7.57 (t, J=8.0 Hz, 1H), 7.32 (dd, J=8.0, 1.0 Hz, 1H), 7.24 (d, J=8.5 Hz, 1H), 6.88-6.79 (m, 2H), 6.49 (d, J=2.5 Hz, 1H), 5.69-5.49 (m, 1H), 5.09 (dd, J=15.4, 0.9 Hz, 1H), 3.85 (s, 3H), 2.72 (q, J=7.5 Hz, 2H), 1.56 (m, 1H), 1.34 (t, J=7.5 Hz, 3H), 0.97-0.76 (m, 3H), 0.66 (m, 1H).

### Synthesis of 2-(2-cyclopropyl-4-methoxyphenyl)-3-(oxazol-5-ylmethyl)-4-oxo-3,4-dihydrobenzo[4,5]thieno[2,3-d]pyrimidin-8-yl benzoate (Example 3)

### Example 3

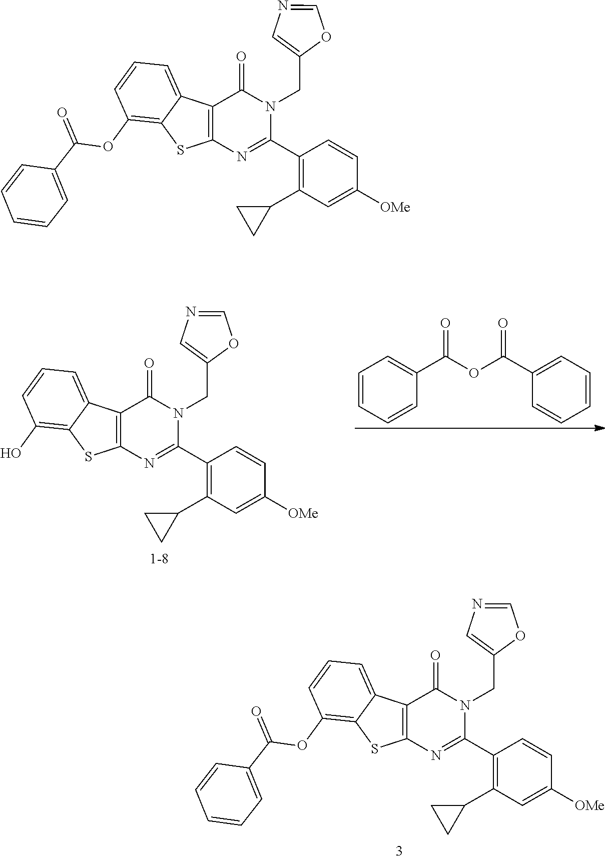

Example 3 was prepared by using similar procedure as described for compound 1. LC-MS observed [M+H], 282.08. ¹H NMR (400 MHz, DMSO-d₆) δ 8.05 (s, 2H), 7.69 (d, J=8.1 Hz, 1H), 7.26 (t, J=8.0 Hz, 1H), 6.88 (d, J=8.1 Hz, 1H), 5.37 (s, 2H), 4.36 (q, J=7.0 Hz, 2H), 3.47 (s, 3H), 1.41 (t, J=7.1 Hz, 3H).

The following examples were prepared by using procedures similar to those described above. Compounds 17-24 were isolated as trifluoroacetate salts.

| | | LC-MS | |
|---|---|---|---|
| Example | Structure | (M + H)⁺ | ¹H NMR |
| 4 | 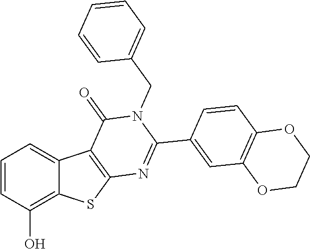 [SMILES: O=c1c2c(nc(-c3ccc4c(c3)OCCO4)n1Cc1ccccc1)sc1c(O)cccc12] | 443.05 | ¹H NMR (400 MHz, DMSO-d₆) δ 10.68 (s, 1H), 7.97 (d, J = 7.6 Hz, 1H), 7.38 (t, J = 7.6 Hz, 1H), 7.32 − 7.20 (m, 3H), 7.12 − 6.90 (m, 6H), 5.30 (s, 2H), 4.35 − 4.21 (m, 4H). |
| 5 | 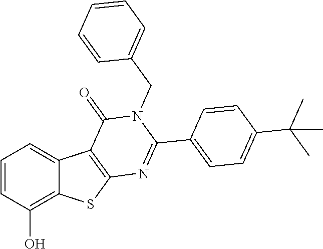 [SMILES: CC(C)(C)c1ccc(-c2nc3sc4c(O)cccc4c3c(=O)n2Cc2ccccc2)cc1] | 441.25 | ¹H NMR (400 MHz, DMSO-d₆) δ 10.67 (s, 1H), 7.99 (d, J = 8.0 Hz, 1H), 7.48 (s, 4H), 7.41 (t, J = 7.6 Hz, 1H), 7.30 − 7.18 (m, 3H), 7.05. 6.95 (m, 3H), 5.30 (s, 2H), 1.31 (s, 9H). |
| 6 | 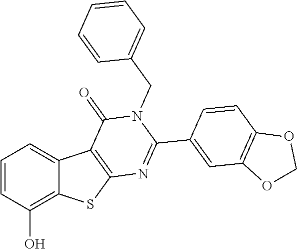 [SMILES: O=c1c2c(nc(-c3ccc4c(c3)OCO4)n1Cc1ccccc1)sc1c(O)cccc12] | 429.05 | ¹H NMR (400 MHz, DMSO-d₆) δ 10.68 (s, 1H), 7.99 (d, J = 7.2 Hz, 1H), 7.41 (t, J = 8.0 Hz, 1H), 7.32 − 7.21 (m, 3H), 7.12 (d, J = 1.6 Hz, 1H), 7.07 − 6.94 (m, 5H), 6.11 (s, 2H), 5.31 (s, 2H). |
| 7 | 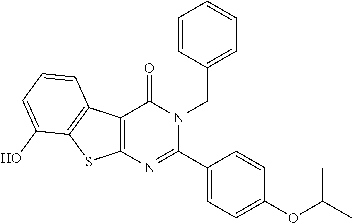 [SMILES: CC(C)Oc1ccc(-c2nc3sc4c(O)cccc4c3c(=O)n2Cc2ccccc2)cc1] | 443.25 | ¹H NMR (400 MHz, DMSO-d₆) δ 10.65 (s, 1H), 8.00 (d, J = 7.6 Hz, 1H), 7.50 − 7.38 (m, 3H), 7.30- 7.20 (m, 3H), 7.05 − 6.94 (m, 5H), 5.33 (s, 2H), 4.77 − 4.65 (m, 1H), 1.29 (d, J = 6.0 Hz, 1H), 1.25 (d, J = 3.2 Hz, 1H). |
| 8 | 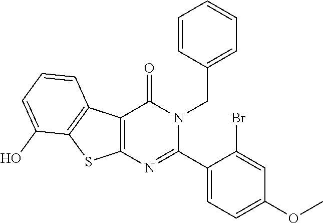 [SMILES: COc1ccc(-c2nc3sc4c(O)cccc4c3c(=O)n2Cc2ccccc2)c(Br)c1] | 493.15 | ¹H NMR (400 MHz, Chloroform-d) δ 8.37 (d, J = 7.2 Hz, 1H), 7.45 (t, J = 8.0 Hz, 1H), 7.25 − 7.19 (m, 4H), 7.05 − 6.91 (m, 4H), 6.82 (dd, J = 8.4, 2.4 Hz, 1H), 5.89 (d, J = 15.2 Hz, 1H), 4.76 (d, J = 15.2 Hz, 1H), 3.87 (s, 3H), 1.29 (d, J = 6.0 Hz, 1H), 1.25 (d, J = 3.2 Hz, 1H). |
| 9 | 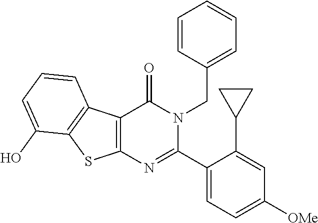 [SMILES: COc1ccc(-c2nc3sc4c(O)cccc4c3c(=O)n2Cc2ccccc2)c(C2CC2)c1] | 455.13 | ¹H NMR (500 MHz, DMSO-d₆) δ 10.66 (s, 1H), 8.01 (d, J = 8.2 Hz, 1H), 7.41 (t, J = 8.0 Hz, 1H), 7.26 − 7.19 (m, 4H), 6.99 − 6.93 (m, 3H), 6.77 (dd, J = 8.5, 2.6 Hz, 1H), 6.44 (d, J = 2.5 Hz, 1H), 5.41 (d, J = 15.5 Hz, 1H), 5.05 (d, J = 15.1 Hz, 1H), 3.78 (s, 3H), 1.60 − 1.52 (m, 1H), 0.86 − 0.72 (m, 3H), 0.58 − 0.50 (m, 1H). |
| 10 | 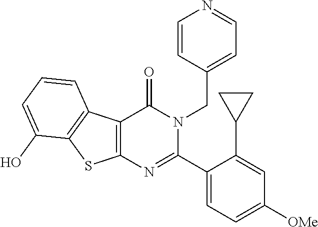 [SMILES: COc1ccc(-c2nc3sc4c(O)cccc4c3c(=O)n2Cc2ccncc2)c(C2CC2)c1] | 456.13 | ¹H NMR (400 MHz, DMSO-d₆) δ 10.77 (s, 1H), 8.41 (dd, J = 4.4, 1.6 Hz, 2H), 7.97 (d, J = 8.6 Hz, 1H), 7.41 (t, J = 7.8 Hz, 1H), 7.26 (d, J = 8.5 Hz, 1H), 7.03 (d, J = 5.8 Hz, 2H), 6.96 (d, J = 8.0 Hz, 1H), 6.76 (dd, J = 8.4, 2.4 Hz, 1H), 6.42 (d, J = 2.8 Hz, 1H), 5.37 (d, J = 16.4 Hz, 1H), 5.03 (d, J = 16.0 Hz, 1H), 3.77 (s, 3H), 1.65 − 1.54 (m, 1H), 0.90 − 0.68 (m, 3H), 0.61 − 0.49 (m, 1H). |
| 11 | 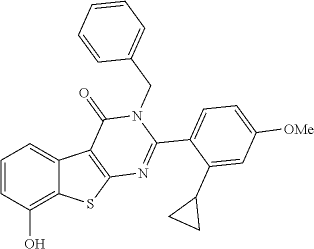 [SMILES: COc1ccc(-c2nc3sc4c(O)cccc4c3c(=O)n2Cc2ccccc2)c(C2CC2)c1] | 456.25 | ¹H NMR (400 MHz, DMSO-d₆) δ 10.72 (s, 1H), 8.54 (d, J = 4.8 Hz, 1H), 8.27 (s, 1H), 8.01 (d, J = 7.2 Hz, 1H), 7.69 (d, J = 8.0 Hz, 1H), 7.52 − 7.36 (m, 2H), 7.30 (t, J = 8.4 Hz, 1H), 6.97 (d, J = 7.6 Hz, 1H), 6.80 (dd, J = 8.8, 2.4 Hz, 1H), 6.40 (d, J = 2.4 Hz, 1H), 5.37 (d, J = 16.0 Hz, 1H), 5.17 (d, J = 15.6 Hz, 1H), 3.78 (s, 3H), 1.60 − 1.48 (m, 1H), 0.90 − 0.64 (m, 3H), 0.56 − 0.46 (m, 1H). |
| 12 | 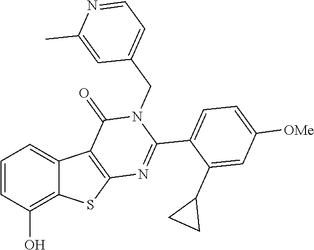 [SMILES: COc1ccc(-c2nc3sc4c(O)cccc4c3c(=O)n2Cc2ccnc(C)c2)c(C2CC2)c1] | 470.25 | ¹H NMR (400 MHz, DMSO-d₆) δ 10.80 (s, 1H), 8.27 (d, J = 4.8 Hz, 1H), 8.00 (d, J = 7.2 Hz, 1H), 7.42 (t, J = 8.0 Hz, 1H), 7.30 (t, J = 8.4 Hz, 1H), 7.26 (d, J = 8.8 Hz, 1H), 6.98 (d, J = 7.6 Hz, 1H), 6.88 − 6.78 (m, 2H), 6.76 (dd, J = 8.4, 2.4 Hz, 1H), 6.41 (d, J = 2.4 Hz, 1H), 5.32 (d, J = 16.0 Hz, 1H), 5.00 (d, J = 16.4 Hz, 1H), 3.77 (s, 3H), 2.35 (d, J = 6.4 Hz, 3H), 1.65 − 1.56 (m, 1H), 0.90 − 0.70 (m, 3H), 0.59 − 0.48 (m, 1H). |
| 13 | 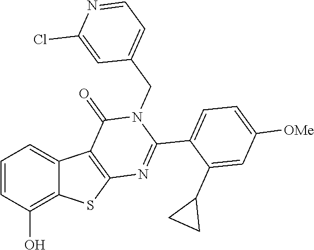 [SMILES: COc1ccc(-c2nc3sc4c(O)cccc4c3c(=O)n2Cc2ccnc(Cl)c2)c(C2CC2)c1] | 490.25 | ¹H NMR (400 MHz, DMSO-d₆) δ 10.73 (s, 1H), 8.24 (d, J = 4.8 Hz, 1H), 8.00 (dd, J = 7.6, 0.6 Hz, 1H), 7.42 (t, J = 8.0 Hz, 1H), 7.28 (d, J = 8.8 Hz, 1H), 7.15 (s, 1H), 7.10 (d, J = 1.2 Hz, 1H), 6.98 (dd, J = 7.6, 0.6 Hz, 1H), 6.79 (dd, J = 8.8, 2.6 Hz, 1H), 6.39 (d, J = 2.4 Hz, 1H), 5.30 (d, J = 16.4 Hz, 1H), 5.08 (d, J = 16.8 Hz, 1H), 3.77 (s, 3H), 1.65 − 1.55 (m, 1H), 0.87 − 0.67 (m, 3H), 0.58 − 0.46 (m, 1H). |
| 14 | 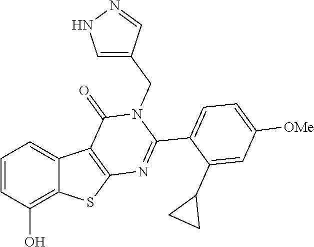 [SMILES: COc1ccc(-c2nc3sc4c(O)cccc4c3c(=O)n2Cc2cn[nH]c2)c(C2CC2)c1] | 445.25 | ¹H NMR (400 MHz, DMSO-d₆) δ 12.70 (s, 1H), 11.02 (s, 1H), 8.02 (d, J = 7.6 Hz, 1H), 7.41 (t, J = 8.0 Hz, 1H), 7.32 (t, J = 8.0 Hz, 1H), 7.30 − 7.10 (m, 2H), 6.96 (d, J = 7.6 Hz, 1H), 6.90 (dd, J = 8.4, 2.4 Hz, 1H), 6.79 (dd, J = 8.8, 2.6 Hz, 1H), 6.51 (d, J = 2.4 Hz, 1H), 5.23 (d, J = 14.4 Hz, 1H), 4.83 (d, J = 14.8 Hz, 1H), 3.83 (s, 3H), 1.60 − 1.51 (m, 1H), 0.90 − 0.72 (m, 3H), 0.70 − 0.62 (m, 1H). |
| 15 | 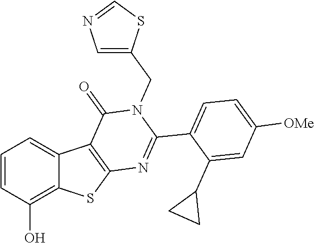 [SMILES: COc1ccc(-c2nc3sc4c(O)cccc4c3c(=O)n2Cc2cncs2)c(C2CC2)c1] | 462.25 | ¹H NMR (400 MHz, DMSO-d₆) δ 10.76 (s, 1H), 8.96 (d, J = 0.4 Hz, 1H), 8.05 (dd, J = 7.6, 0.6 Hz, 1H), 7.44 (t, J = 8.0 Hz, 1H), 7.33 (d, J = 8.4 Hz, 1H), 7.23 (s, 1H), 6.98 (dd, J = 8.0, 0.8 Hz, 1H), 6.91 (dd, J = 8.4, 2.4 Hz, 1H), 6.51 (d, J = 2.4 Hz, 1H), 5.48 (d, J = 15.2 Hz, 1H), 5.23 (d, J = 15.2 Hz, 1H), 3.84 (s, 3H), 1.55 − 1.42 (m, 1H), 0.88 − 0.75 (m, 2H), 0.75 − 0.63 (m, 1H), 0.63 − 0.52 (m, 1H). |
| 16 | 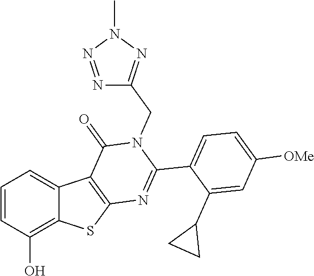 [SMILES: COc1ccc(-c2nc3sc4c(O)cccc4c3c(=O)n2Cc2nnn(C)n2)c(C2CC2)c1] | 461.05 | ¹H NMR (400 MHz, DMSO-d₆) δ 10.72 (s, 1H), 7.96 (d, J = 7.6 Hz, 1H), 7.42 (t, J = 7.6 Hz, 1H), 6.98 (d, J = 8.4 Hz, 1H), 6.79 (dd, J = 8.4, 2.4 Hz, 1H), 6.45 (d, J = 2.4 Hz, 1H), 5.61 (d, J = 16.0 Hz, 1H), 5.18 (d, J = 16.4 Hz, 1H), 4.28 (s, 3H), 3.77 (s, 3H), 1.69 − 1.58 (m, 1H), 0.90 − 0.75 (m, 3H), 0.65 − 0.56 (m, 1H). |
| 17 | 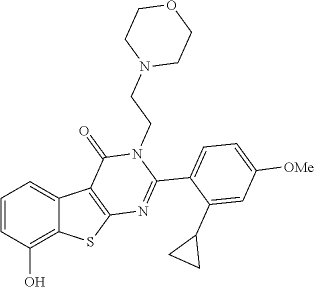 [SMILES: COc1ccc(-c2nc3sc4c(O)cccc4c3c(=O)n2CCN2CCOCC2)c(C2CC2)c1] | 478.20 | ¹H NMR (400 MHz, Methanol-d₄) δ 8.15 (dd, J = 8.0, 0.8 Hz, 1H), 7.47 − 7.39 (m, 2H), 7.03 − 6.95 (m, 2H), 6.62 (d, J = 2.4 Hz, 1H), 4.75 − 4.66 (m, 1H), 4.28 − 4.17 (m, 1H), 4.10 − 3.80 (m, 4H), 3.88 (s, 3H), 3.45 − 3.20 (m, 6H), 1.73 − 1.64 (m, 1H), 1.04 − 0.86 (m, 3H), 0.83 − 0.75 (m, 1H). |
| 18 | 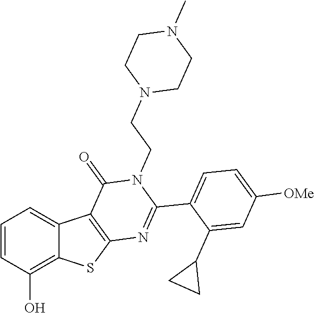 [SMILES: COc1ccc(-c2nc3sc4c(O)cccc4c3c(=O)n2CCN2CCN(C)CC2)c(C2CC2)c1] | 491.25 | ¹H NMR (400 MHz, Methanol-d₄) δ 8.11 (d, J = 8.0 Hz, 1H), 7.49 − 7.37 (m, 2H), 6.99 − 6.92 (m, 2H), 6.59 (d, J = 2.4 Hz, 1H), 4.50 (t, J = 7.2 Hz, 1H), 4.04 (t, J = 6.8 Hz, 1H), 3.87 (s, 3H), 3.53 − 2.95 (m, 6H), 2.85 (s, 3H), 2.70 (dd, J = 10.8, 6.4 Hz, 2H), 2.60 − 2.21 (m, 2H), 1.72 − 1.62 (m, 1H), 1.05 − 0.90 (m, 2H), 0.90 − 0.78 (m, 2H). |
| 19 | 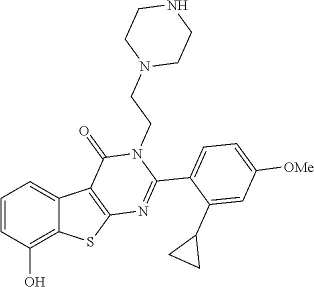 [SMILES: COc1ccc(-c2nc3sc4c(O)cccc4c3c(=O)n2CCN2CCNCC2)c(C2CC2)c1] | 477.20 | ¹H NMR (400 MHz, Methanol-d₄) δ 8.12 (dd, J = 8.0, 0.8 Hz, 1H), 7.50 − 7.38 (m, 2H), 6.99 − 6.93 (m, 2H), 6.59 (d, J = 2.4 Hz, 1H), 4.51 (t, J = 6.8 Hz, 1H), 4.04 (t, J = 6.8 Hz, 1H), 3.87 (s, 3H), 3.15 (t, J = 2.4 Hz, 4H), 2.71 (t, J = 6.8 Hz, 2H), 2.63 (s, 4H), 1.73 − 1.64 (m, 1H), 1.07 − 0.90 (m, 2H), 0.90 − 0.77 (m, 2H). |
| 20 | 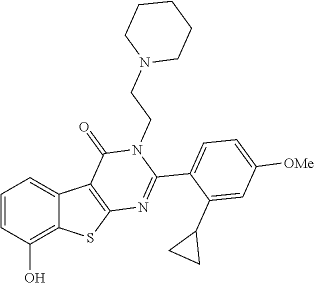 [SMILES: COc1ccc(-c2nc3sc4c(O)cccc4c3c(=O)n2CCN2CCCCC2)c(C2CC2)c1] | 476.15 | ¹H NMR (400 MHz, Methanol-d₄) δ 8.13 (dd, J = 8.0, 0.8 Hz, 1H), 7.48 − 7.39 (m, 2H), 7.02 − 6.92 (m, 2H), 6.62 (d, J = 2.4 Hz, 1H), 4.68 (t, J = 7.2 Hz, 1H), 4.21 (t, J = 7.2 Hz, 1H), 3.88 (s, 3H), 3.70 − 3.52 (m, 2H), 3.45 − 3.25 (m, 2H), 3.05 − 2.85 (m, 2H), 2.03 − 1.45 (m, 7H), 1.03 − 0.88 (m, 3H), 0.83 − 0.77 (m, 1H). |
| 21 | 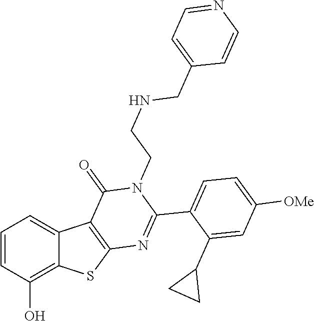 [SMILES: COc1ccc(-c2nc3sc4c(O)cccc4c3c(=O)n2CCNCc2ccncc2)c(C2CC2)c1] | 499.20 | ¹H NMR (400 MHz, Methanol-d₄) δ 8.83 − 8.65 (m, 2H), 8.15 (dd, J = 7.6, 1.2 Hz, 1H), 7.75 − 7.65 (m, 2H), 7.44 (dd, J = 8.0, 2.0 Hz, 2H), 6.99 − 6.92 (m, 2H), 6.60 (s, 1H), 4.78 − 4.65 (m, 1H), 4.45 − 4.35 (m, 2H), 4.28 − 4.20 (m, 1H), 3.88 (s, 3H), 3.45 − 3.28 (m, 2H), 1.70 − 1.61 (m, 1H), 1.03 − 0.86 (m, 3H), 0.80 − 0.72 (m, 1H). |
| 22 | 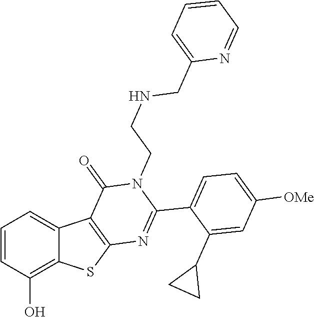 [SMILES: COc1ccc(-c2nc3sc4c(O)cccc4c3c(=O)n2CCNCc2ccccn2)c(C2CC2)c1] | 499.15 | ¹H NMR (400 MHz, Methanol-d₄) δ 8.56 (d, J = 4.8 Hz, 1H), 8.15 (d, J = 8.0 Hz, 1H), 7.87 (td, J = 7.6, 1.6 Hz, 1H), 7.46 − 7.36 (m, 4H), 6.99 − 6.92 (m, 2H), 6.59 (d, J = 8.0 Hz, 2H), 4.78 − 4.66 (m, 1H), 4.38 (s, 2H), 4.32 − 4.24 (m, 1H), 3.88 (s, 3H), 3.42 − 3.35 (m, 2H), 1.67 − 1.60 (m, 1H), 0.99 − 0.86 (m, 3H), 0.77 − 0.68 (m, 1H). |
| 23 | 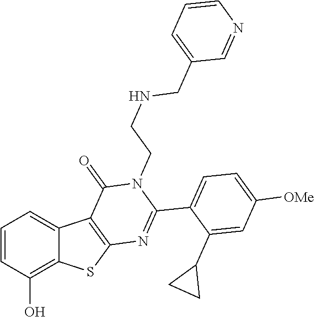 [SMILES: COc1ccc(-c2nc3sc4c(O)cccc4c3c(=O)n2CCNCc2cccnc2)c(C2CC2)c1] | 499.15 | ¹H NMR (400 MHz, Methanol-d₄) δ 8.75 (d, J = 2.0 Hz, 1H), 8.71 (dd, J = 5.2, 1.6 Hz, 1H), 8.14 (dd, J = 8.0, 0.8 Hz, 1H), 8.12 (t, J = 2.5 Hz, 1H), 7.65 (dd, J = 7.6, 4.8 Hz, 1H), 7.43 (t, J = 8.0 Hz, 1H), 7.39 (d, J = 8.4 Hz, 1H), 7.02 − 6.92 (m, 2H), 6.60 (d, J = 2.4 Hz, 1H), 4.75 − 4.66 (m, 1H), 4.38 (d, J = 4.4 Hz, 2H), 4.29 − 4.20 (m, 1H), 3.88 (s, 3H), 3.42 − 3.30 (m, 2H), 1.70 − 1.62 (m, 1H), 1.02 − 0.87 (m, 3H), 0.79 − 0.70 (m, 1H). |
| 24 | 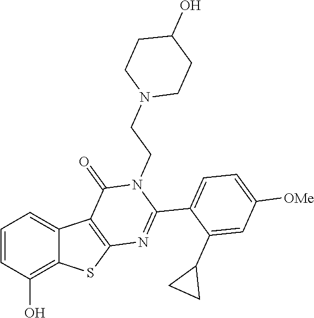 [SMILES: COc1ccc(-c2nc3sc4c(O)cccc4c3c(=O)n2CCN2CCC(O)CC2)c(C2CC2)c1] | 492.20 | ¹H NMR (400 MHz, Methanol-d₄) δ 8.13 (d, J = 7.6 Hz, 1H), 7.50 − 7.41 (m, 2H), 7.05 − 6.93 (m, 2H), 6.62 (d, J = 2.4 Hz, 1H), 4.75 − 4.64 (m, 1H), 4.28 − 4.18 (m, 1H), 4.15 − 4.05 (m, 1H), 3.88 (s, 3H), 3.75 − 2.95 (m, 6H), 2.22 − 1.60 (m, 4H), 1.73 − 1.63 (m, 1H), 1.06 − 0.87 (m, 3H), 0.84 − 0.77 (m, 1H). |
| 25 | 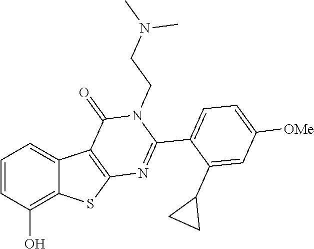 [SMILES: COc1ccc(-c2nc3sc4c(O)cccc4c3c(=O)n2CCN(C)C)c(C2CC2)c1] | 436.15 | ¹H NMR (400 MHz, Methanol-d₄) δ 8.12 (dd, J = 8.0, 0.8 Hz, 1H), 7.47 − 7.38 (m, 2H), 6.99 − 6.92 (m, 2H), 6.58 (d, J = 2.4 Hz, 1H), 4.48 − 4.38 (m, 1H), 4.08 − 3.98 (m, 1H), 4.15 − 4.05 (m, 1H), 3.87 (s, 3H), 2.69 (t, J = 4.8 Hz, 1H), 2.53 (t, J = 4.8 Hz, 1H), 2.13 (s, 6H), 1.71 − 1.61 (m, 1H), 1.05 − 0.85 (m, 3H), 0.84 − 0.75 (m, 1H). |
| 26 | 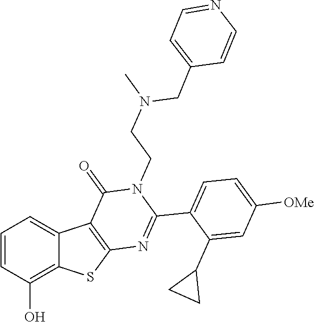 [SMILES: COc1ccc(-c2nc3sc4c(O)cccc4c3c(=O)n2CCN(C)Cc2ccncc2)c(C2CC2)c1] | 513.20 | ¹H NMR (400 MHz, DMSO-d₆) δ 10.65 (s, 1H), 8.27 (d, J = 4.0 Hz, 2H), 7.97 (d, J = 8.0 Hz, 1H), 7.40 (t, J = 8.0 Hz, 1H), 7.30 (d, J = 8.8 Hz, 1H), 7.05 (d, J = 4.4 Hz, 2H), 6.97 (dd, J = 8.0, 0.8 Hz, 1H), 6.82 (dd, J = 8.4, 2.4 Hz, 1H), 6.44 (d, J = 2.4 Hz, 1H), 4.38 − 4.28 (m, 1H), 3.91 − 3.82 (m, 1H), 3.81 (s, 3H), 3.46 − 3.30 (m, 2H), 2.50 − 2.40 (m, 2H), 2.00 (s, 3H), 1.62 − 1.52 (m, 1H), 0.95 − 0.78 (m, 3H), 0.67 − 0.59 (m, 1H). |
| 27 | 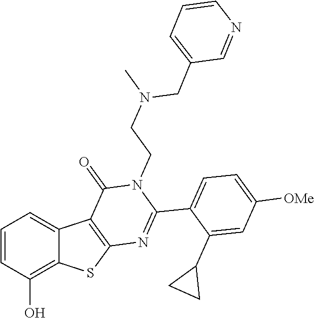 [SMILES: COc1ccc(-c2nc3sc4c(O)cccc4c3c(=O)n2CCN(C)Cc2cccnc2)c(C2CC2)c1] | 513.20 | ¹H NMR (400 MHz, DMSO-d₆) δ 10.64 (s, 1H), 8.29 (d, J = 1.6 Hz, 1H), 8.25 (dd, J = 4.8, 1.6 Hz, 1H), 7.96 (dd, J = 7.6, 0.8 Hz, 1H), 7.47 − 7.40 (m, 2H), 7.30 (d, J = 8.4 Hz, 1H), 7.06 (dd, J = 7.6, 4.8 Hz, 1H), 6.97 (d, J = 8.0 Hz, 1H), 6.82 (dd, J = 8.4, 2.4 Hz, 1H), 6.44 (d, J = 2.4 Hz, 1H), 4.35 − 4.25 (m, 1H), 3.88 − 3.80 (m, 1H), 3.80 (s, 3H), 3.45 − 3.25 (m, 2H), 2.50 − 2.40 (m, 2H), 1.96 (s, 3H), 1.62 − 1.51 (m, 1H), 0.95 − 0.78 (m, 3H), 0.72 − 0.62 (m, 1H). |
| 28 | 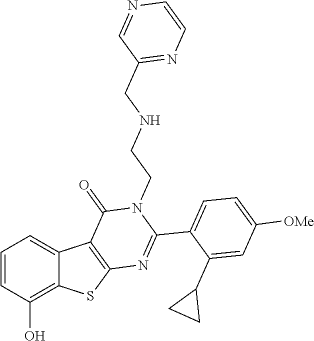 [SMILES: COc1ccc(-c2nc3sc4c(O)cccc4c3c(=O)n2CCNCc2cnccn2)c(C2CC2)c1] | 500.15 | ¹H NMR (400 MHz, DMSO-d₆) δ 10.65 (s, 1H), 8.55 − 8.46 (m, 3H), 8.02 (d, J = 1.8 Hz, 1H), 7.96 (dd, J = 7.6, 0.8 Hz, 1H), 7.45 − 7.34 (m, 2H), 6.96 (d, J = 8.0 Hz, 1H), 6.82 (dd, J = 8.4, 2.4 Hz, 1H), 6.44 (d, J = 2.4 Hz, 1H), 4.34 − 4.22 (m, 1H), 3.92 − 3.80 (m, 1H), 3.81 (s, 3H), 3.65 (s, 2H), 2.80 − 2.66 (m, 2H), 1.96 (s, 3H), 1.65 − 1.55 (m, 1H), 0.91 − 0.77 (m, 3H), 0.68 − 0.58 (m, 1H). |
| 29 | 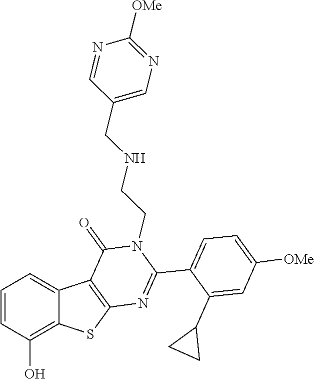 [SMILES: COc1ccc(-c2nc3sc4c(O)cccc4c3c(=O)n2CCNCc2cnc(OC)nc2)c(C2CC2)c1] | 530.05 | ¹H NMR (400 MHz, Methanol-d₄) δ 8.47 (s, 2H), 8.11 (d, J = 7.6 Hz, 1H), 7.41 (t, J = 7.6 Hz, 1H), 7.37 (d, J = 8.8 Hz, 1H), 6.94 (d, J = 7.6 Hz, 1H), 6.91 (dd, J = 8.4, 2.4 Hz, 1H), 6.56 (d, J = 2.4 Hz, 1H), 4.55 − 4.43 (m, 1H), 4.10 − 3.96 (m, 1H), 4.02 (s, 3H), 3.87 (s, 3H), 3.75 (s, 2H), 3.04 − 2.85 (m, 2H), 1.68 − 1.58 (m, 1H), 0.99 − 0.82 (m, 3H), 0.78 − 0.66 (m, 1H). |
| 30 | 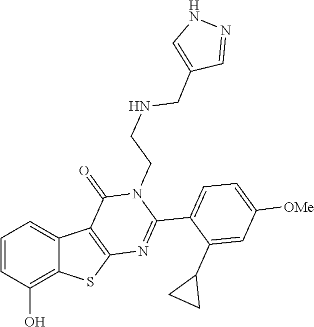 [SMILES: COc1ccc(-c2nc3sc4c(O)cccc4c3c(=O)n2CCNCc2cn[nH]c2)c(C2CC2)c1] | 488.05 | ¹H NMR (400 MHz, DMSO-d₆) δ 10.70 (s, 1H), 8.21 (s, 1H), 8.03 (d, J = 8.0 Hz, 1H), 7.47 − 7.34 (m, 4H), 6.96 (d, J = 7.2 Hz, 1H), 6.86 (dd, J = 8.8, 2.4 Hz, 1H), 6.47 (d, J = 2.4 Hz, 1H), 4.32 − 4.22 (m, 1H), 3.90 − 3.80 (m, 1H), 3.81 (s, 3H), 3.42 (s, 2H), 2.80 − 2.64 (m, 2H), 1.62 − 1.53 (m, 1H), 0.93 − 0.77 (m, 3H), 0.72 − 0.63 (m, 1H). |
| 31 | 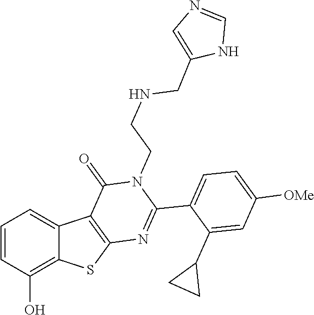 [SMILES: COc1ccc(-c2nc3sc4c(O)cccc4c3c(=O)n2CCNCc2cnc[nH]2)c(C2CC2)c1] | 487.57 | ¹H NMR (400 MHz, Methanol-d₄) δ 8.50 (s, 1H), 8.12 (d, J = 8.0 Hz, 1H), 7.77 (s, 1H), 7.41 (t, J = 8.0 Hz, 1H), 7.35 (d, J = 8.4 Hz, 1H), 7.20 (s, 1H), 6.99 − 6.89 (m, 2H), 6.57 (d, J = 2.4 Hz, 1H), 4.63 − 4.52 (m, 1H), 4.18 − 4.10 (m, 1H), 4.03 (s, 2H), 3.87 (s, 3H), 3.28 − 3.08 (s, 2H), 1.68 − 1.58 (m, 1H), 0.99 − 0.84 (m, 3H), 0.77 − 0.66 (m, 1H). |
| 32 | 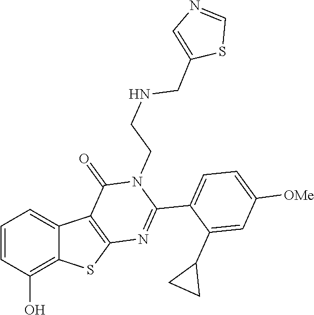 [SMILES: COc1ccc(-c2nc3sc4c(O)cccc4c3c(=O)n2CCNCc2cncs2)c(C2CC2)c1] | 505.00 | ¹H NMR (400 MHz, DMSO-d₆) δ 10.69 (s, 1H), 8.89 (d, J = 0.8 Hz, 1H), 8.02 (dd, J = 8.0, 0.8 Hz, 1H), 7.59 (s, 1H), 7.41 (t, J = 7.6 Hz, 1H), 7.36 (d, J = 8.4 Hz, 1H), 6.96 (dd, J = 8.0, 0.8 Hz, 1H), 6.84 (dd, J = 8.4, 2.4 Hz, 1H), 6.45 (d, J = 2.4 Hz, 1H), 4.30 − 4.20 (m, 1H), 3.90 − 3.80 (m, 1H), 3.80 (s, 3H), 3.72 (s, 2H), 2.75 − 2.62 (m, 2H), 1.62 − 1.53 (m, 1H), 0.92 − 0.77 (m, 3H), 0.67 − 0.58 (m, 1H). |
| 33 | 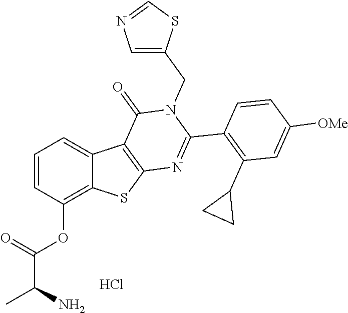 [SMILES: COc1ccc(-c2nc3sc4c(OC(=O)[C@H](C)N)cccc4c3c(=O)n2Cc2cncs2)c(C2CC2)c1 ; Cl] | 533.13 | ¹H NMR (500 MHz, DMSO-d₆) δ 8.98 (s, 1H), 8.55 (d, J = 8.0 Hz, 1H), 7.76 (t, J = 8.0 Hz, 1H), 7.50 (d, J = 8.1 Hz, 1H), 7.35 (d, J = 8.4 Hz, 1H), 7.27 (s, 1H), 6.94 (dd, J = 8.5, 2.5 Hz, 1H), 6.53 (d, J = 2.5 Hz, 1H), 5.52 (d, J = 15.5 Hz, 1H), 5.29 (d, J = 15.4 Hz, 1H), 4.63 (s, 1H), 1.68 (d, J = 7.2 Hz, 3H), 1.53 − 1.44 (m, 1H), 0.83 − 0.75 (m, 2H), 0.73 − 0.65 (m, 1H), 0.63 − 0.57 (m, 1H). |
| 34 | 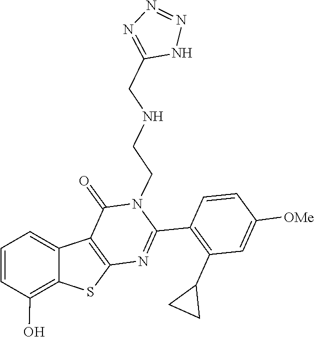 [SMILES: COc1ccc(-c2nc3sc4c(O)cccc4c3c(=O)n2CCNCc2nnn[nH]2)c(C2CC2)c1] | 490.10 | ¹H NMR (400 MHz, DMSO-d₆) δ 10.75 (s, 1H), 8.03 (dd, J = 8.0, 0.8 Hz, 1H), 7.43 (t, J = 7.6 Hz, 1H), 7.39 (d, J = 8.4 Hz, 1H), 6.97 (dd, J = 7.6, 0.4 Hz, 1H), 6.87 (dd, J = 8.4, 2.4 Hz, 1H), 6.48 (d, J = 2.4 Hz, 1H), 4.45 − 4.34 (m, 1H), 4.13 − 4.01 (m, 3H), 3.82 (s, 3H), 3.05 (t, J = 6.0 Hz, 2H), 1.69 − 1.58 (m, 1H), 0.95 − 0.78 (m, 3H), 0.73 − 0.60 (m, 1H). |
| 35 | 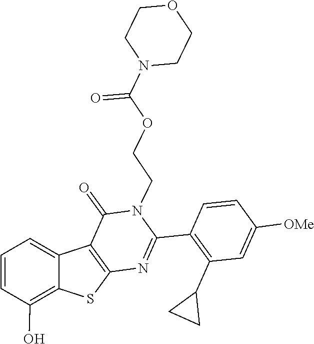 [SMILES: COc1ccc(-c2nc3sc4c(O)cccc4c3c(=O)n2CCOC(=O)N2CCOCC2)c(C2CC2)c1] | 522.20 | ¹H NMR (400 MHz, DMSO-d₆) δ 10.67 (s, 1H), 8.02 (d, J = 8.0 Hz, 1H), 7.47 − 7.36 (m, 2H), 6.97 (d, J = 7.6 Hz, 1H), 6.91 (dd, J = 8.4, 2.0 Hz, 1H), 6.49 (d, J = 2.0 Hz, 1H), 4.60 − 4.46 (m, 1H), 4.39 − 4.13 (m, 2H), 4.08 − 3.92 (m, 1H), 3.82 (s, 3H), 3.43 − 3.25 (m, 4H), 3.20 (s, 4H), 1.65 − 1.55 (m, 1H), 0.98 − 0.90 (m, 1H), 0.89 − 0.79 (m, 2H), 0.78 − 0.68 (m, 1H). |
| 36 | 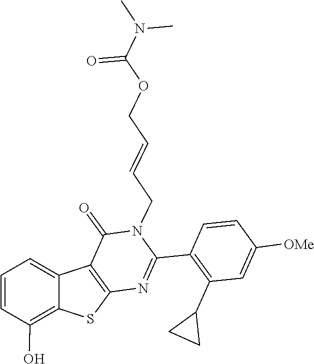 [SMILES: COc1ccc(-c2nc3sc4c(O)cccc4c3c(=O)n2C/C=C/COC(=O)N(C)C)c(C2CC2)c1] | 506.20 | ¹H NMR (400 MHz, DMSO-d₆) δ 10.71 (s, 1H), 8.02 (d, J = 8.0 Hz, 1H), 7.42 (t, J = 8.0 Hz, 1H), 7.32 (d, J = 8.4 Hz, 1H), 6.97 (d, J = 7.6 Hz, 1H), 6.83 (dd, J = 8.4, 2.4 Hz, 1H), 6.49 (d, J = 2.4 Hz, 1H), 5.74 − 5.62 (m, 1H), 5.41 − 5.30 (m, 1H), 4.81 (dd, J = 15.6, 5.2 Hz, 1H), 4.39 (d, J = 4.8 Hz, 1H), 4.33 (dd, J = 16.0, 5.2 Hz, 1H), 3.81 (d, J = 4.8 Hz, 3H), 2.80 (dd, J = 10.0, 6.8 Hz, 6H), 1.65 − 1.56 (m, 1H), 0.92 − 0.78 (m, 3H), 0.73 − 0.63 (m, 1H). |
| 37 | 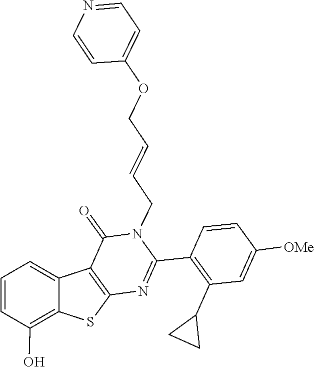 [SMILES: COc1ccc(-c2nc3sc4c(O)cccc4c3c(=O)n2C/C=C/COc2ccncc2)c(C2CC2)c1] | 512.20 | ¹H NMR (400 MHz, DMSO-d₆) δ 10.86 (s, 1H), 8.02 (d, J = 7.6 Hz, 1H), 7.52 (d, J = 7.6 Hz, 2H), 7.42 (t, J = 7.6 Hz, 1H), 7.19 (d, J = 8.8 Hz, 1H), 6.97 (d, J = 7.6 Hz, 1H), 6.68 (dd, J = 8.4, 2.4 Hz, 1H), 6.48 (d, J = 2.4 Hz, 1H), 6.08 (d, J = 7.6 Hz, 2H), 5.75 − 5.62 (m, 1H), 5.43 − 5.32 (m, 1H), 4.91 (dd, J = 16.0, 4.4 Hz, 1H), 4.36 (d, J = 6.0 Hz, 2H), 4.19 (dd, J = 15.6, 5.2 Hz, 1H), 3.81 (d, J = 4.8 Hz, 3H), 1.69 − 1.56 (m, 1H), 0.95 − 0.78 (m, 3H), 0.75 − 0.62 (m, 1H). |
| 38 | 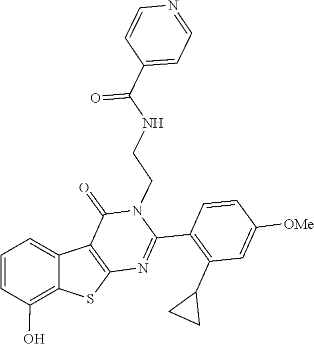 [SMILES: COc1ccc(-c2nc3sc4c(O)cccc4c3c(=O)n2CCNC(=O)c2ccncc2)c(C2CC2)c1] | 513.15 | ¹H NMR (400 MHz, Methanol-d₄) δ 10.86 (s, 1H), 8.61 (d, J = 5.6 Hz, 2H), 8.07 (d, J = 8.0 Hz, 1H), 7.60 (d, J = 5.2 Hz, 2H), 7.39 (t, J = 7.6 Hz, 1H), 7.35 (d, J = 8.4 Hz, 1H), 6.93 (d, J = 8.0 Hz, 1H), 6.69 (dd, J = 8.4, 2.0 Hz, 1H), 6.56 (d, J = 2.0 Hz, 1H), 4.80 − 4.72 (m, 1H), 4.15 − 4.07 (m, 1H), 3.84 (s, 3H), 3.78 − 3.69 (m, 1H), 3.68 − 3.59 (m, 1H), 1.70 − 1.61 (m, 1H), 1.03 − 0.73 (m, 4H). |
| 39 | 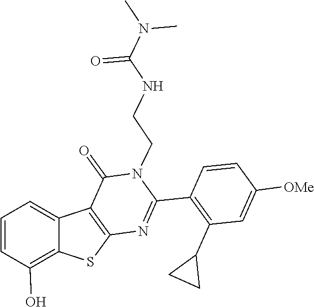 [SMILES: COc1ccc(-c2nc3sc4c(O)cccc4c3c(=O)n2CCNC(=O)N(C)C)c(C2CC2)c1] | 480.15 | ¹H NMR (400 MHz, DMSO-d₆) δ 10.67 (s, 1H), 8.02 (d, J = 8.0 Hz, 1H), 7.43 (t, J = 8.0 Hz, 1H), 7.35 (d, J = 8.8 Hz, 1H), 6.97 (d, J = 8.0 Hz, 1H), 6.90 (dd, J = 8.4, 2.4 Hz, 1H), 6.48 (d, J = 2.4 Hz, 1H), 4.55 − 4.47 (m, 1H), 4.27 − 4.19 (m, 1H), 4.19 − 4.07 (m, 1H), 4.07 − 3.95 (m, 1H), 3.81 (s, 3H), 2.70 (s, 3H), 2.68 (s, 3H), 1.63 − 1.51 (m, 1H), 1.01 − 0.67 (m, 4H). |
| 40 | 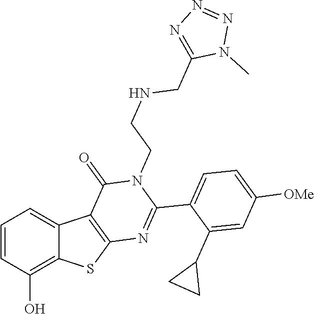 [SMILES: COc1ccc(-c2nc3sc4c(O)cccc4c3c(=O)n2CCNCc2nnnn2C)c(C2CC2)c1] | 504.30 | ¹H NMR (400 MHz, Methanol-d₄) δ 8.12 (dd, J = 8.0, 0.8 Hz, 1H), 7.42 (t, J = 8.0 Hz, 1H), 7.40 (d, J = 8.4 Hz, 1H), 6.99 − 6.90 (m, 2H), 6.59 (d, J = 2.4 Hz, 1H), 4.65 − 4.52 (m, 1H), 4.35 (s, 3H), 4.29 (s, 3H), 4.20 − 4.12 (m, 1H), 3.87 (s, 3H), 3.30 − 3.16 (m, 2H), 1.69 − 1.58 (m, 1H), 1.02 − 0.85 (m, 3H), 0.79 − 0.70 (m, 1H). |
| 41 | 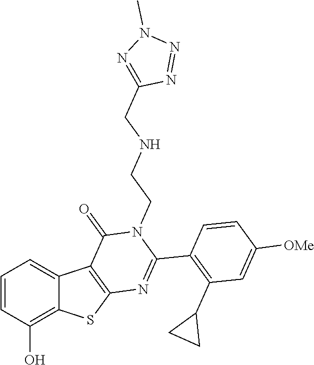 [SMILES: COc1ccc(-c2nc3sc4c(O)cccc4c3c(=O)n2CCNCc2nnn(C)n2)c(C2CC2)c1] | 504.30 | ¹H NMR (400 MHz, Methanol-d₄) δ 8.10 (d, J = 8.0 Hz, 1H), 7.41 (t, J = 8.0 Hz, 1H), 7.33 (d, J = 8.4 Hz, 1H), 6.93 (d, J = 8.4 Hz, 1H), 6.88 (dd, J = 8.4, 2.0 Hz, 1H), 6.55 (d, J = 2.4 Hz, 1H), 4.56 − 4.45 (m, 1H), 4.14 (s, 2H), 4.09 − 3.98 (m, 1H), 3.98 (s, 3H), 3.87 (s, 3H), 3.08 − 2.91 (m, 2H), 1.68 − 1.56 (m, 1H), 1.01 − 0.80 (m, 3H), 0.79 − 0.68 (m, 1H). |
| 42 | 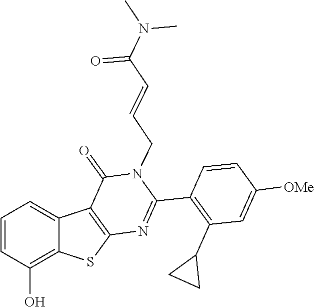 [SMILES: COc1ccc(-c2nc3sc4c(O)cccc4c3c(=O)n2C/C=C/C(=O)N(C)C)c(C2CC2)c1] | 476.15 | ¹H NMR (400 MHz, DMSO-d₆) δ 10.61 (s, 1H), 8.02 (d, J = 8.0 Hz, 1H), 7.42 (t, J = 8.0 Hz, 1H), 7.37 (d, J = 8.4 Hz, 1H), 6.97 (d, J = 8.0 Hz, 1H), 6.87 (dd, J = 8.4, 2.4 Hz, 1H), 6.55 − 6.45 (m, 2H), 6.22 (d, J = 15.6 Hz, 1H), 4.89 (dd, J = 16.8, 5.2 Hz, 1H), 4.48 (dd, J = 16.0, 4.4 Hz, 1H), 4.07 − 3.95 (m, 1H), 3.81 (d, J = 5.2 Hz, 3H), 2.91 (s, 3H), 2.82 (s, 3H), 1.68 − 1.58 (m, 1H), 0.93 − 0.79 (m, 3H), 0.71 − 0.60 (m, 1H). |
| 43 | 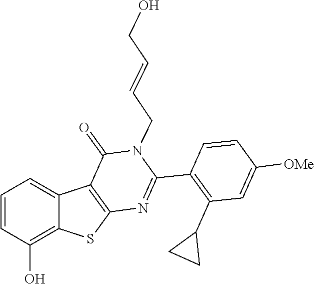 [SMILES: COc1ccc(-c2nc3sc4c(O)cccc4c3c(=O)n2C/C=C/CO)c(C2CC2)c1] | 435.10 | ¹H NMR (400 MHz, DMSO-d₆) δ 10.66 (s, 1H), 8.02 (dd, J = 8.0, 0.8 Hz, 1H), 7.42 (t, J = 8.0 Hz, 1H), 7.34 (d, J = 8.4 Hz, 1H), 6.97 (dd, J = 8.0, 0.8 Hz, 1H), 6.87 (dd, J = 8.4, 2.4 Hz, 1H), 6.50 (d, J = 1.4 Hz, 1H), 5.63 − 5.50 (m, 1H), 5.37 (d, J = 15.6 Hz, 1H), 4.82 − 4.72 (m, 1H), 4.67 (t, J = 5.6 Hz, 1H), 4.35 − 4.25 (m, 1H), 4.07 − 3.95 (m, 1H), 3.84 (d, J = 4.4 Hz, 2H), 3.81 (s, 3H), 1.65 − 1.55 (m, 1H), 0.95 − 0.78 (m, 3H), 0.73 − 0.65 (m, 1H). |
| 44 | 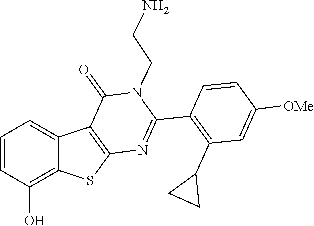 [SMILES: COc1ccc(-c2nc3sc4c(O)cccc4c3c(=O)n2CCN)c(C2CC2)c1] | 408.15 | ¹H NMR (400 MHz, Methanol-d₄) δ 8.14 (d, J = 7.6 Hz, 1H), 7.42 (t, J = 8.0 Hz, 1H), 7.41 (d, J = 8.4 Hz, 1H), 7.01 − 6.90 (m, 2H), 6.60 (d, J = 2.4 Hz, 1H), 4.52 − 4.42 (m, 1H), 4.13 − 4.02 (m, 1H), 3.87 (s, 3H), 3.13 − 2.95 (m, 2H), 1.71 − 1.62 (m, 1H), 1.04 − 0.85 (m, 3H), 0.83 − 0.72 (m, 1H). |
| 45 | 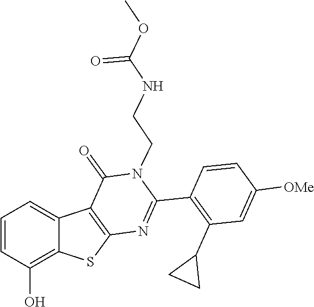 [SMILES: COC(=O)NCCn1c(-c2ccc(OC)cc2C2CC2)nc2sc3c(O)cccc3c2c1=O] | 466.05 | ¹H NMR (400 MHz, Methanol-d₄) δ 8.12 (d, J = 7.6 Hz, 1H), 7.45 − 7.35 (m, 2H), 6.98 − 6.89 (m, 2H), 6.57 (d, J = 2.0 Hz, 1H), 4.55 − 4.42 (m, 1H), 4.05 − 3.92 (m, 1H), 3.87 (s, 3H), 3.52 (s, 3H), 3.45 − 3.22 (m, 2H), 1.67 − 1.56 (m, 1H), 1.05 − 0.92 (m, 1H), 0.91 − 0.72 (m, 3H). |
| 46 | 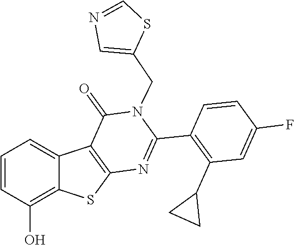 [SMILES: O=c1c2c(nc(-c3ccc(F)cc3C3CC3)n1Cc1cncs1)sc1c(O)cccc12] | 450.00 | ¹H NMR (400 MHz, DMSO-d₆) δ 10.73 (s, 1H), 8.98 (d, J = 0.4 Hz, 1H), 8.07 (dd, J = 7.6, 0.4 Hz, 1H), 7.55 − 7.42 (m, 2H), 7.24 (s, 1H), 7.23 − 7.15 (m, 1H), 7.00 (dd, J = 8.0, 0.8 Hz, 1H), 6.88 (dd, J = 10.8, 2.4 Hz, 1H), 5.44 (d, J = 15.6 Hz, 1H), 5.24 (d, J = 15.2 Hz, 1H), 1.52 − 1.38 (m, 1H), 0.95 − 0.76 (m, 2H), 0.75 − 0.52 (m, 2H). |
| 47 | 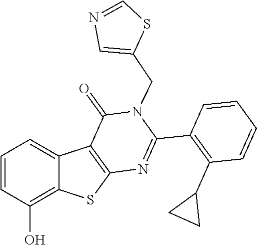 [SMILES: O=c1c2c(nc(-c3ccccc3C3CC3)n1Cc1cncs1)sc1c(O)cccc12] | 432.10 | ¹H NMR (400 MHz, DMSO-d₆) δ 10.72 (s, 1H), 8.98 (s, 1H), 8.07 (d, J = 7.6 Hz, 1H), 7.58 − 7.33 (m, 4H), 7.14 (s, 1H), 7.04 (d, J = 8.0 Hz, 1H), 6.99 (d, J = 7.6 Hz, 1H), 5.47 (d, J = 15.6 Hz, 1H), 5.22 (d, J = 15.6 Hz, 1H), 1.53 − 1.40 (m, 1H), 0.87 − 0.62 (m, 3H), 0.60 − 0.51 (m, 1H). |
| 48 | 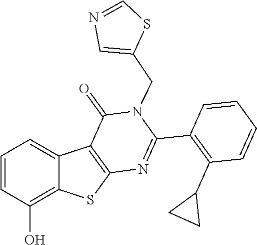 [SMILES: O=c1c2c(nc(-c3ccccc3C3CC3)n1Cc1cncs1)sc1c(O)cccc12] | 432.00 | ¹H NMR (400 MHz, DMSO-d₆) δ 10.72 (s, 1H), 8.99 (d, J = 0.8 Hz, 1H), 8.04 (dd, J = 8.0, 0.8 Hz, 1H), 7.50 − 7.32 (m, 5H), 7.23 (s, 1H), 6.98 (dd, J = 8.0, 0.8 Hz, 1H), 5.38 (s, 2H), 2.07 − 1.95 (m, 1H), 1.05 − 0.95 (m, 2H), 0.73 − 0.66 (m, 2H). |
| 49 | 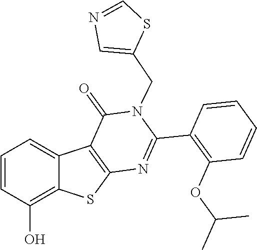 [SMILES: CC(C)Oc1ccccc1-c1nc2sc3c(O)cccc3c2c(=O)n1Cc1cncs1] | 450.10 | ¹H NMR (400 MHz, DMSO-d₆) δ 10.71 (s, 1H), 8.94 (s, 1H), 8.04 (d, J = 7.6 Hz, 1H), 7.65 − 7.56 (m, 1H), 7.50 − 7.35 (m, 2H), 7.27 (d, J = 8.4 Hz, 1H), 7.20 (d, J = 6.8 Hz, 1H), 7.11 (t, J = 7.2 Hz, 1H), 6.99 (d, J = 7.6 Hz, 1H), 5.64 (d, J = 15.6 Hz, 1H), 5.06 (d, J = 15.6 Hz, 1H), 4.68 (hept, J = 6.4 Hz, 1H), 1.15 (d, J = 6.0 Hz, 6H). |
| 50 | 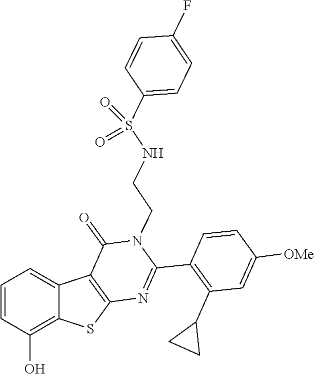 [SMILES: COc1ccc(-c2nc3sc4c(O)cccc4c3c(=O)n2CCNS(=O)(=O)c2ccc(F)cc2)c(C2CC2)c1] | 566.05 | ¹H NMR (400 MHz, Methanol-d₄) δ 8.08 (dd, J = 7.6, 0.8 Hz, 1H), 7.76 − 7.65 (m, 2H), 7.50 − 7.35 (m, 2H), 7.41 (dd, J = 8.4, 2.4 Hz, 2H), 7.18 −7.11 (m, 2H), 6.99 − 6.90 (m, 2H), 6.56 (d, J = 2.4 Hz, 1H), 4.43 − 4.33 (m, 1H), 3.95 − 3.87 (m, 1H), 3.89 (s, 3H), 3.26 − 3.10 (m, 2H), 1.62 − 1.52 (m, 1H), 0.97 − 0.78 (m, 3H), 0.72 − 0.63 (m, 1H). |
| 51 | 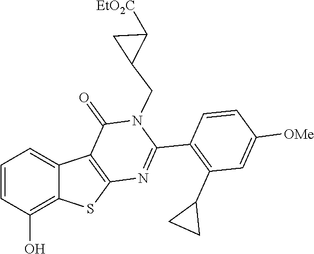 [SMILES: CCOC(=O)C1CC1Cn1c(-c2ccc(OC)cc2C2CC2)nc2sc3c(O)cccc3c2c1=O] | 491.30 | ¹H NMR (400 MHz, DMSO-d₆) δ 10.67 (s, 1H), 8.03 (d, J = 8.0 Hz, 1H), 7.48 − 7.38 (m, 2H), 6.97 (d, J = 8.0 Hz, 1H), 6.93 − 6.85 (m, 1H), 6.49 (d, J = 2.0 Hz, 1H), 4.22 − 4.12 (m, 1H), 4.05 − 3.90 (m, 2H), 3.80 (s, 3H), 3.80 − 3.70 (m, 1H), 1.69 − 1.42 (m, 3H), 1.14 (t, J = 7.2 Hz, 3H), 0.99 − 0.63 (m, 6H). |
| 52 | 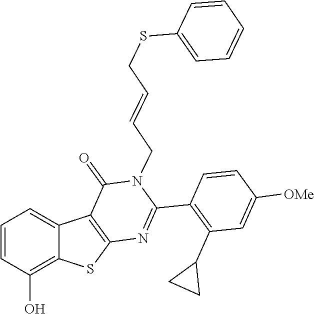 [SMILES: COc1ccc(-c2nc3sc4c(O)cccc4c3c(=O)n2C/C=C/CSc2ccccc2)c(C2CC2)c1] | 527.20 | ¹H NMR (400 MHz, DMSO-d₆) δ 10.66 (s, 1H), 8.03 (d, J = 7.6 Hz, 1H), 7.43 (t, J = 8.0 Hz, 1H), 7.24 (d, J = 7.6 Hz, 2H), 7.17 − 6.95 (m, 5H), 6.72 (dd, J = 8.8, 2.4 Hz, 1H), 6.47 (d, J = 2.4 Hz, 1H), 5.62 − 5.51 (m, 1H), 5.36 − 5.24 (m, 1H), 4.79 − 4.68 (m, 1H), 4.21 − 4.14 (m, 1H), 3.81 (s, 3H), 3.53 (d, J = 6.8 Hz, 2H), 1.56 − 1.46 (m, 1H), 0.92 − 0.78 (m, 3H), 0.72 − 0.64 (m, 1H). |
| 53 | 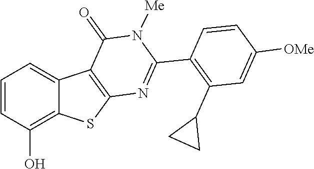 [SMILES: COc1ccc(-c2nc3sc4c(O)cccc4c3c(=O)n2C)c(C2CC2)c1] | 379.05 | ¹H NMR (400 MHz, DMSO-d₆) δ 10.63 (s, 1H), 8.03 (d, J = 8.0 Hz, 1H), 7.46 − 7.38 (m, 2H), 7.17 − 6.96 (d, J = 8.0 Hz, 1H), 6.91 (dd, J = 8.4, 2.4 Hz, 1H), 6.54 (d, J = 2.4 Hz, 1H), 3.82 (s, 3H), 3.36 (s, 3H), 1.70 − 1.61 (m, 1H), 0.95 − 0.78 (m, 3H), 0.72 − 0.62 (m, 1H). |
| 54 | 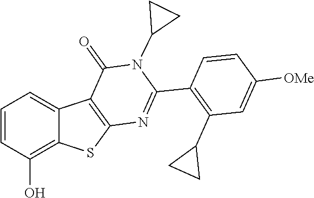 [SMILES: COc1ccc(-c2nc3sc4c(O)cccc4c3c(=O)n2C2CC2)c(C2CC2)c1] | 405.10 | ¹H NMR (400 MHz, DMSO-d₆) δ 10.60 (s, 1H), 8.02 (dd, J = 8.0, 0.8 Hz, 1H), 7.45 (d, J = 8.4 Hz, 1H), 7.40 (d, J = 8.0 Hz, 1H), 6.94 (dd, J = 8.0, 0.8 Hz, 1H), 6.86 (dd, J = 8.4, 2.4 Hz, 1H), 6.46 (d, J = 2.4 Hz, 1H), 3.81 (s, 3H), 3.12 − 3.02 (m, 1H), 1.88 − 1.80 (m, 1H), 1.02 − 0.51 (m, 8H). |
| 55 | 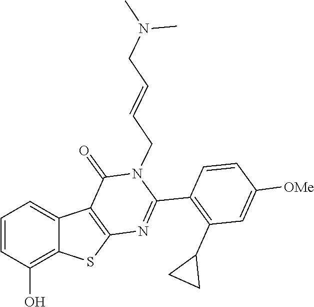 [SMILES: COc1ccc(-c2nc3sc4c(O)cccc4c3c(=O)n2C/C=C/CN(C)C)c(C2CC2)c1] | 462.30 | ¹H NMR (400 MHz, Chloroform-d) δ 8.15 (d, J = 8.0 Hz, 1H), 7.35 − 7.26 (m, 2H), 7.18 (d, J = 8.0 Hz, 1H), 6.82 (dd, J = 8.4, 2.8 Hz, 1H), 6.50 (d, J = 2.8 Hz, 1H), 5.92 − 5.82 (m, 1H), 5.15 (dd, J = 10.4, 1.2 Hz, 1H), 5.02 (dd, J = 15.2, 5.2 Hz, 1H), 4.94 (dd, J = 17.2, 1.2 Hz, 1H), 4.34 (dd, J = 15.2, 6.0 Hz, 1H), 3.88 (m, 1H), 3.86 (s, 3H), 2.44 (s, 6H), 1.70 − 1.61 (m, 1H), 0.95 − 0.80 (m, 3H), 0.71 − 0.60 (m, 1H). |
| 56 | 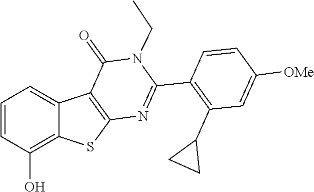 [SMILES: CCn1c(-c2ccc(OC)cc2C2CC2)nc2sc3c(O)cccc3c2c1=O] | 393.12 | ¹H NMR (400 MHz, DMSO-d₆) δ 10.56 (d, J = 1.0 Hz, 1H), 7.96 (d, J = 7.8 Hz, 1H), 7.40 − 7.30 (m, 2H), 6.89 (d, J = 7.9 Hz, 1H), 6.83 (dd, J = 8.5, 2.5 Hz, 1H), 6.43 (d, J = 2.5 Hz, 1H), 4.18 − 4.03 (m, 1H), 3.74 (d, J = 1.0 Hz, 3H), 3.72 − 3.59 (m, 1H), 1.57 − 1.46 (m, 1H), 1.04 (t, J = 7.0 Hz, 3H), 0.88 − 0.71 (m, 3H), 0.67 − 0.56 (m, 1H). |
| 57 | 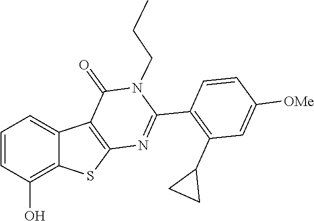 [SMILES: CCCn1c(-c2ccc(OC)cc2C2CC2)nc2sc3c(O)cccc3c2c1=O] | 407.14 | ¹H NMR (500 MHz, DMSO-d₆) δ 10.58 (s, 1H), 7.96 (d, J = 7.8 Hz, 1H), 7.37 − 7.31 (m, 2H), 6.89 (d, J = 7.9 Hz, 1H), 6.82 (dd, J = 8.5, 2.5 Hz, 1H), 6.42 (d, J = 2.5 Hz, 1H), 4.09 − 3.99 (m, 1H), 3.74 (s, 3H), 3.62 − 3.51 (m, 1H), 1.57 − 1.39 (m, 1H), 0.86 − 0.72 (m, 3H), 0.63 (t, J = 7.4 Hz, 4H). |
| 58 | 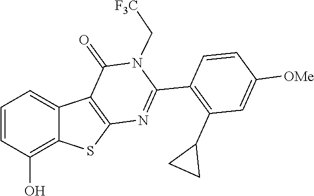 [SMILES: COc1ccc(-c2nc3sc4c(O)cccc4c3c(=O)n2CC(F)(F)F)c(C2CC2)c1] | 447.10 | ¹H NMR (500 MHz, DMSO-d₆) δ 10.68 (s, 1H), 7.93 (d, J = 7.9 Hz, 1H), 7.42 − 7.31 (m, 2H), 6.92 (d, J = 7.9 Hz, 1H), 6.84 (dd, J = 8.6, 2.5 Hz, 1H), 6.43 (d, J = 2.5 Hz, 1H), 5.16 − 5.01 (m, 1H), 4.60 − 4.45 (m, 1H), 3.75 (s, 3H), 1.62 − 1.50 (m, 1H), 0.91 − 0.81 (m, 1H), 0.80 − 0.72 (m, 2H), 0.72 − 0.60 (m, 1H). |
| 59 | 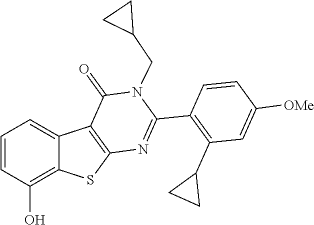 [SMILES: COc1ccc(-c2nc3sc4c(O)cccc4c3c(=O)n2CC2CC2)c(C2CC2)c1] | 419.14 | ¹H NMR (500 MHz, DMSO-d₆) δ 10.60 (s, 1H), 7.95 (d, J = 7.8 Hz, 1H), 7.40 − 7.30 (m, 2H), 6.88 (d, J = 7.9 Hz, 1H), 6.82 (dd, J = 8.5, 2.5 Hz, 1H), 6.40 (d, J = 2.6 Hz, 1H), 4.04 (dd, J = 13.9, 7.0 Hz, 1H), 3.74 (s, 3H), 3.61 (dd, J = 14.0, 7.0 Hz, 1H), 1.56 − 1.47 (m, 1H), 0.98 − 0.89 (m, 1H), 0.89 − 0.70 (m, 3H), 0.69 − 0.60 (m, 1H), 0.39 − 0.21 (m, 2H), 0.20 − 0.10 (m, 1H), 0.05 − 0.04 (m, 1H). |
| 60 | 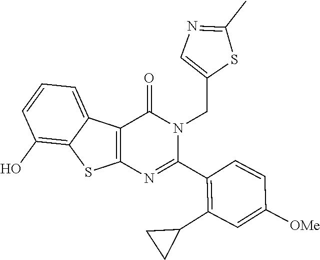 [SMILES: COc1ccc(-c2nc3sc4c(O)cccc4c3c(=O)n2Cc2cnc(C)s2)c(C2CC2)c1] | 476.11 | ¹H NMR (400 MHz, DMSO-d₆) δ 10.77 (s, 1H), 8.04 (d, J = 7.8 Hz, 1H), 7.44 (t, J = 7.9 Hz, 1H), 7.33 (d, J = 8.5 Hz, 1H), 6.99 (d, J = 7.9 Hz, 1H), 6.95 − 6.91 (m, 1H), 6.91 (s, 1H), 6.51 (d, J = 2.5 Hz, 1H), 5.41 (d, J = 15.3 Hz, 1H), 5.15 (d, J = 15.2 Hz, 1H), 3.84 (s, 3H), 2.53 (s, 3H), 1.56 − 1.42 (m, 1H), 0.92 − 0.67 (m, 3H), 0.66 − 0.54 (m, 1H). |
| 61 | 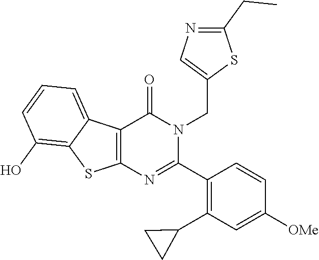 [SMILES: CCc1ncc(Cn2c(-c3ccc(OC)cc3C3CC3)nc3sc4c(O)cccc4c3c2=O)s1] | 490.12 | ¹H NMR (400 MHz, Methanol-d₄) δ 8.13 (dd, J = 8.0, 0.9 Hz, 1H), 7.42 (t, J = 7.9 Hz, 1H), 7.29 (d, J = 8.5 Hz, 1H), 6.99 − 6.90 (m, 3H), 6.58 (d, J = 2.5 Hz, 1H), 5.50 (d, J = 15.1 Hz, 1H), 5.33 (d, J = 15.1 Hz, 1H), 3.89 (s, 3H), 2.94 (q, J = 7.6 Hz, 2H), 1.52 − 1.40 (m, 1H), 1.31 (t, J = 8.0 Hz, 1H), 0.94 − 0.83 (m, 1H), 0.83 − 0.69 (m, 3H), 0.69 − 0.55 (m, 1H). |
| 62 | 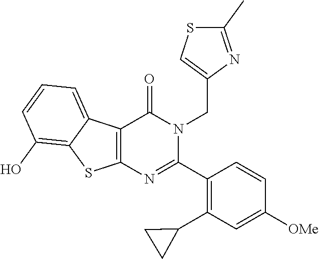 [SMILES: COc1ccc(-c2nc3sc4c(O)cccc4c3c(=O)n2Cc2csc(C)n2)c(C2CC2)c1] | 476.12 | ¹H NMR (400 MHz, Methanol-d₄) δ 8.12 (d, J = 7.9 Hz, 1H), 7.42 (t, J = 8.0 Hz, 1H), 7.21 (d, J = 8.5 Hz, 1H), 6.95 (dd, J = 7.8, 1.0 Hz, 1H), 6.90 (d, J = 1.2 Hz, 1H), 6.78 (dd, J = 8.5, 2.5 Hz, 1H), 6.52 (d, J = 2.4 Hz, 1H), 5.61 (d, J = 15.1 Hz, 1H), 5.13 (d, J = 15.1 Hz, 1H), 3.83 (s, 3H), 2.61 (s, 3H), 1.69 − 1.59 (m, 1H), 0.95 − 0.73 (m, 3H), 0.71 − 0.62 (m, 1H). |
| 63 | 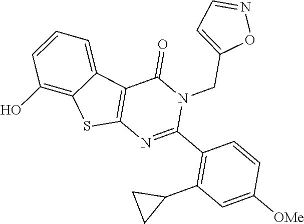 [SMILES: COc1ccc(-c2nc3sc4c(O)cccc4c3c(=O)n2Cc2ccno2)c(C2CC2)c1] | 446.12 | ¹H NMR (400 MHz, Chloroform-d) δ 8.17 (d, J = 8.0 Hz, 1H), 8.14 (d, J = 4.0 Hz, 1H), 7.98 (s, 1H), 7.32 (t, J = 8.0 Hz, 1H), 7.27 (d, J = 8.0 Hz, 1H), 6.83 (dd, J = 8.0, 1.0 Hz, 1H), 6.77 (dd, J = 8.0, 4.0 Hz, 1H), 6.45 (d, J = 2.5 Hz, 1H), 6.21 (d, J = 1.8 Hz, 1H), 5.69 (d, J = 15.0 Hz, 1H), 5.12 (d, J = 15.0 Hz, 1H), 3.80 (s, 3H), 1.66 − 1.54 (m, 1H), 0.96 − 0.75 (m, 3H), 0.70 − 0.59 (m, 1H). |
| 64 | 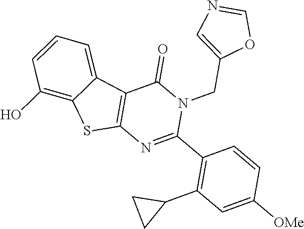 [SMILES: COc1ccc(-c2nc3sc4c(O)cccc4c3c(=O)n2Cc2cnco2)c(C2CC2)c1] | 445.90 | ¹H NMR (400 MHz, Methanol-d₄) δ 8.15 (dd, J = 8.0, 0.9 Hz, 1H), 7.90 (s, 1H), 7.42 (t, J = 7.9 Hz, 1H), 7.29 (d, J = 8.5 Hz, 1H), 6.95 (d, J = 8.0 Hz, 1H), 6.90 (s, 1H), 6.88 (dd, J = 8.5, 2.5 Hz, 1H), 6.52 (d, J = 15.0 Hz, 1H), 5.60 (d, J = 2.5 Hz, 1H), 5.13 (d, J = 15.0 Hz, 1H), 3.88 (s, 1H), 1.62 − 1.51 (m, 3H), 0.99 − 0.80 (m, 3H), 0.75 − 0.64 (m, 1H). |
| 65 | 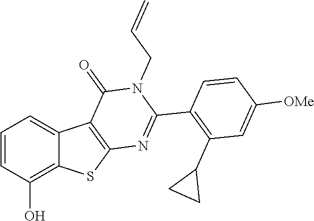 [SMILES: C=CCn1c(-c2ccc(OC)cc2C2CC2)nc2sc3c(O)cccc3c2c1=O] | 405.25 | ¹H NMR (400 MHz, DMSO-d₆) δ 10.67 (s, 1H), 8.02 (d, J = 8.0 Hz, 1H), 7.42 (t, J = 8.0 Hz, 1H), 7.34 (t, J = 8.4 Hz, 1H), 6.97 (d, J = 8.0 Hz, 1H), 6.86 (dd, J = 8.4, 2.4 Hz, 1H), 6.49 (d, J = 2.0 Hz, 1H), 5.84 − 5.72 (m, 1H), 5.07 (d, J = 10.4 Hz, 1H), 4.90 − 4.78 (m, 2H), 4.28 (dd, J = 16.0, 4.8 Hz, 1H), 3.81 (s, 3H), 1.67 − 1.56 (m, 1H), 1.01 − 0.80 (m, 3H), 0.75 − 0.62 (m, 1H). |
| 66 | 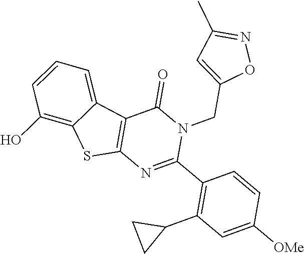 [SMILES: COc1ccc(-c2nc3sc4c(O)cccc4c3c(=O)n2Cc2cc(C)no2)c(C2CC2)c1] | 460.13 | ¹H NMR (400 MHz, Chloroform-d) δ 8.21 (d, J = 7.9 Hz, 1H), 7.36 (t, J = 7.9 Hz, 1H), 7.28 (d, J = 8.5 Hz, 1H), 7.00 (s, 1H), 6.87 (d, J = 7.8 Hz, 1H), 6.79 (dd, J = 8.5, 2.5 Hz, 1H), 6.47 (d, J = 2.5 Hz, 1H), 6.04 (s, 1H), 5.61 (d, J = 15.5 Hz, 1H), 5.03 (d, J = 15.5 Hz, 1H), 3.82 (s, 3H), 2.22 (s, 3H), 1.65 − 1.55 (m, 1H), 0.99 − 0.78 (m, 3H), 0.70 − 0.57 (m, 1H). |
| 67 | 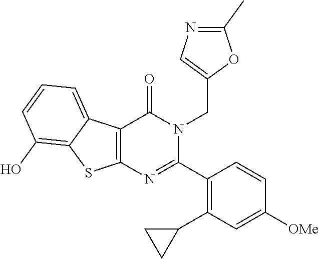 [SMILES: COc1ccc(-c2nc3sc4c(O)cccc4c3c(=O)n2Cc2cnc(C)o2)c(C2CC2)c1] | 460.12 | ¹H NMR (400 MHz, Chloroform-d) δ 8.23 (dd, J = 8.0, 0.9 Hz, 1H), 7.36 (t, J = 7.9 Hz, 1H), 7.26 (d, J = 8.6 Hz, 1H), 6.85 (dd, J = 8.0, 0.9 Hz, 1H), 6.82 (dd, J = 8.0, 4.0 Hz, 1H), 6.69 (s, 1H), 6.47 (d, J = 2.5 Hz, 1H), 5.59 (d, J = 15.4 Hz, 1H), 5.03 (d, J = 15.4 Hz, 1H), 3.83 (s, 3H), 2.39 (s, 3H), 1.62 − 1.51 (m, 1H), 0.97 − 0.74 (m, 3H), 0.72 − 0.61 (m, 1H). |
| 68 | 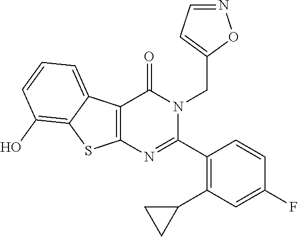 [SMILES: O=c1c2c(nc(-c3ccc(F)cc3C3CC3)n1Cc1ccno1)sc1c(O)cccc12] | 434.08 | ¹H NMR (400 MHz, Chloroform-d) δ 8.21 (d, J = 8.0 Hz, 1H), 8.15 (d, J = 4.0 Hz, 1H), 7.38 (t, J = 8.0 Hz, 1H), 7.34 (dd, J = 8.0, 4.0 Hz, 1H), 6.98 (td, J = 8.2, 2.6 Hz, 1H), 6.91 (d, J = 7.8 Hz, 1H), 6.65 (dd, J = 10.2, 2.5 Hz, 1H), 6.22 (s, 1H), 5.68 (d, J = 16.0 Hz, 1H), 5.06 (d, J = 16.0 Hz, 1H), 1.67 − 1.55 (m, 1H), 1.03 − 0.90 (m, 2H), 0.88 − 0.78 (m, 1H), 0.73 − 0.60 (m, 1H). |
| 69 | 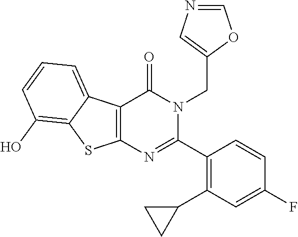 [SMILES: O=c1c2c(nc(-c3ccc(F)cc3C3CC3)n1Cc1cnco1)sc1c(O)cccc12] | 434.08 | ¹H NMR (400 MHz, Chloroform-d) δ 8.17 (d, J = 7.9 Hz, 1H), 7.72 (s, 1H), 7.32 (t, J = 7.9 Hz, 1H), 7.25 (dd, J = 8.5, 5.6 Hz, 1H), 6.94 (td, J = 8.2, 2.5 Hz, 1H), 6.89 − 6.79 (m, 2H), 6.58 (dd, J = 10.2, 2.5 Hz, 1H), 5.54 (d, J = 16.0 Hz, 1H), 4.97 (d, J = 16.0 Hz, 1H), 1.56 − 1.44 (m, 1H), 0.97 − 0.81 (m, 2H), 0.81 − 0.71 (m, 1H), 0.67 − 0.55 (m, 1H). |
| 70 | 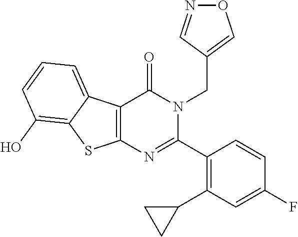 [SMILES: O=c1c2c(nc(-c3ccc(F)cc3C3CC3)n1Cc1cnoc1)sc1c(O)cccc12] | 434.08 | ¹H NMR (400 MHz, Chloroform-d) δ 8.28 (dd, J = 8.0, 0.9 Hz, 1H), 8.23 (s, 1H), 8.13 (s, 1H), 7.43 (t, J = 7.9 Hz, 1H), 7.25 (dd, J = 8.5, 5.7 Hz, 1H), 7.06 (td, J = 8.2, 2.5 Hz, 1H), 6.92 (dd, J = 7.9, 0.9 Hz, 1H), 6.68 (dd, J = 10.1, 2.5 Hz, 1H), 5.31 (d, J = 16.0 Hz, 1H), 4.88 (d, J = 14.7 Hz, 1H), 1.57 − 1.46 (m, 1H), 1.04 − 0.88 (m, 2H), 0.88 − 0.79 (m, 1H), 0.75 − 0.66 (m, 1H). |
| 71 | 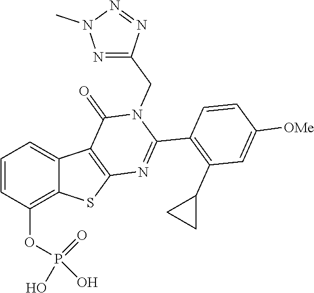 [SMILES: COc1ccc(-c2nc3sc4c(OP(=O)(O)O)cccc4c3c(=O)n2Cc2nnn(C)n2)c(C2CC2)c1] | 541.09 | ¹H NMR (400 MHz, DMSO-d₆) δ 8.26 (d, J = 7.8 Hz, 1H), 7.61 (t, J = 8.0 Hz, 1H), 7.50 (d, J = 8.1 Hz, 1H), 7.26 (d, J = 8.5 Hz, 1H), 6.80 (dd, J = 8.6, 2.5 Hz, 1H), 6.45 (d, J = 2.5 Hz, 1H), 5.63 (d, J = 16.0 Hz, 1H), 5.21 (d, J = 16.0 Hz, 1H), 4.29 (s, 3H), 3.78 (s, 3H), 1.70 − 1.61 (m, 1H), 0.90 − 0.73 (m, 3H), 0.67 − 0.56 (m, 1H). |
| 72 | 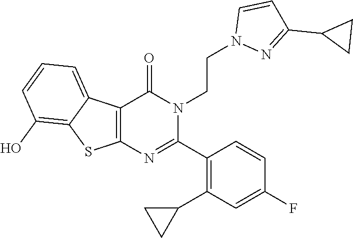 [SMILES: O=c1c2c(nc(-c3ccc(F)cc3C3CC3)n1CCn1ccc(C3CC3)n1)sc1c(O)cccc12] | 487.14 | ¹H NMR (400 MHz, Chloroform-d) δ 9.02 (s, 1H), 8.17 (d, J = 7.9 Hz, 1H), 7.35 (t, J = 7.9 Hz, 1H), 6.97 (d, J = 2.3 Hz, 1H), 6.85 (d, J = 8.0 Hz, 1H), 6.80 (td, J = 8.2, 2.5 Hz, 1H), 6.66 − 6.53 (m, 2H), 5.84 (d, J = 2.3 Hz, 1H), 4.84 − 4.67 (m, 2H), 4.37 − 4.26 (m, 1H), 4.12 − 4.02 (m, 1H), 1.99 − 1.87 (m, 1H), 1.50 − 1.38 (m, 1H), 1.03 − 0.56 (m, 8H). |
| 73 | 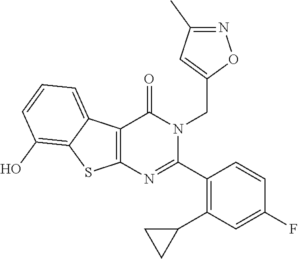 [SMILES: Cc1cc(Cn2c(-c3ccc(F)cc3C3CC3)nc3sc4c(O)cccc4c3c2=O)on1] | 448.09 | ¹H NMR (400 MHz, Chloroform-d) δ 8.20 (dd, J = 7.9, 1.1 Hz, 1H), 7.43 (s, 1H), 7.41 − 7.31 (m, 2H), 6.98 (td, J = 8.3, 2.5 Hz, 1H), 6.90 (dd, J = 7.8, 0.9 Hz, 1H), 6.64 (dd, J = 10.1, 2.5 Hz, 1H), 6.07 (s, 1H), 5.60 (d, J = 15.5 Hz, 1H), 4.99 (d, J = 15.6 Hz, 1H), 2.23 (s, 3H), 1.67 − 1.56 (m, 1H), 1.02 − 0.88 (m, 2H), 0.88 − 0.79 (m, 1H), 0.72 − 0.61 (m, 1H). |
| 74 | 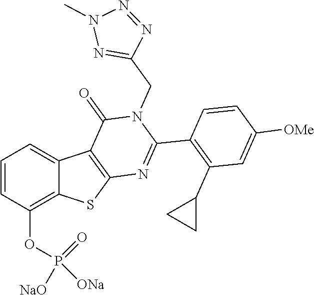 | 585.05 | ¹H NMR (500 MHz, DMSO-d₆) δ 8.02 (d, J = 7.8 Hz, 1H), 7.67 (d, J = 8.0 Hz, 1H), 7.43 (t, J = 8.0 Hz, 1H), 7.26 (d, J = 8.5 Hz, 1H), 7.20 (s, 1H), 6.78 (dd, J = 8.5, 2.5 Hz, 1H), 6.44 (d, J = 2.5 Hz, 1H), 5.62 (d, J = 16.5 Hz, 1H), 5.18 (d, J = 16.5 Hz, 1H), 4.29 (s, 3H), 3.78 (s, 3H), 1.69 − 1.61 (m, 1H), 0.92 − 0.72 (m, 3H), 0.64 − 0.56 (m, 1H). |
| 75 | 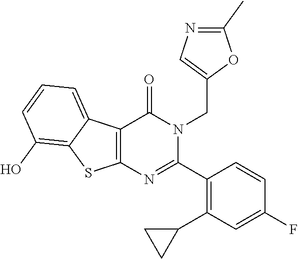 [SMILES: Cc1ncc(Cn2c(-c3ccc(F)cc3C3CC3)nc3sc4c(O)cccc4c3c2=O)o1] | 448.10 | ¹H NMR (400 MHz, Chloroform-d) δ 8.24 (dd, J = 8.0, 0.9 Hz, 1H), 7.39 (t, J = 8.0 Hz, 1H), 7.32 (td, J = 8.4, 5.6 Hz, 1H), 7.01 (td, J = 8.2, 2.5 Hz, 1H), 6.90 (dd, J = 7.9, 1.0 Hz, 1H), 6.73 (s, 1H), 6.65 (dd, J = 10.2, 2.5 Hz, 1H), 5.56 (d, J = 15.4 Hz, 1H), 4.98 (d, J = 15.4 Hz, 1H), 2.39 (s, 3H), 1.63 − 1.53 (m, 1H), 1.03 − 0.89 (m, 2H), 0.88 − 0.78 (m, 1H), 0.74 − 0.65 (m, 1H). |
| 76 | 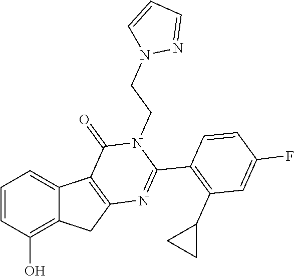 [SMILES: O=c1c2c(nc(-c3ccc(F)cc3C3CC3)n1CCn1cccn1)Cc1c(O)cccc1-2] | 447.10 | ¹H NMR (400 MHz, DMSO-d₆) δ 10.69 (s, 1H), 8.02 (d, J = 7.6 Hz, 1H), 7.67 (d, J = 8.0 Hz, 1H), 7.52 − 7.39 (m, 2H), 7.35 (s, 1H), 7.08 − 7.69 (m, 3H), 6.84 (dd, J = 10.8, 2.4 Hz, 1H), 6.22 (s, 1H), 4.60 − 4.35 (m, 3H), 4.05 − 3.92 (m, 1H), 1.65 − 1.56 (m, 1H), 1.02 − 0.79 (m, 3H), 0.77 − 0.57 (m, 1H). |
| 77 | 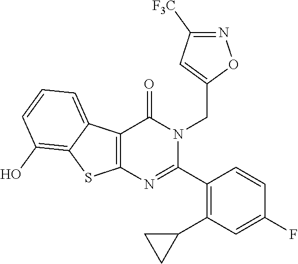 [SMILES: O=c1c2c(nc(-c3ccc(F)cc3C3CC3)n1Cc1cc(C(F)(F)F)no1)sc1c(O)cccc12] | 502.06 | ¹H NMR (400 MHz, Chloroform-d) δ 8.24 (dd, J = 8.0, 1.0 Hz, 1H), 7.42 (td, J = 8.0, 1.0 Hz, 1H), 7.33 (dd, J = 8.5, 5.6 Hz, 1H), 7.02 (td, J = 8.2, 2.5 Hz, 1H), 6.90 (dd, J = 7.9, 0.9 Hz, 1H), 6.65 (dd, J = 10.1, 2.5 Hz, 1H), 6.52 (s, 1H), 6.05 (s, 1H), 5.62 (d, J = 15.7 Hz, 1H), 5.17 (d, J = 15.7 Hz, 1H), 1.63 − 1.52 (m, 1H), 1.05 − 0.80 (m, 3H), 0.73 − 0.63 (m, 1H). |
| 78 | 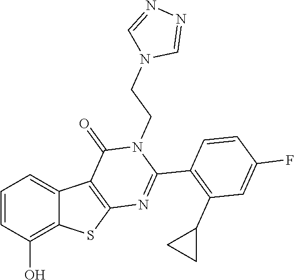 [SMILES: O=c1c2c(nc(-c3ccc(F)cc3C3CC3)n1CCn1cnnc1)sc1c(O)cccc12] | 448.10 | ¹H NMR (400 MHz, DMSO-d₆) δ 10.70 (s, 1H), 8.27 (s, 2H), 8.02 (dd, J = 8.0, 0.8 Hz, 1H), 7.44 (t, J = 7.6 Hz, 1H), 7.15 − 6.92 (m, 3H), 6.86 (dd, J = 10.8, 2.0 Hz, 1H), 4.61 (dd, J = 8.8, 5.2 Hz, 1H), 4.42 − 4.23 (m, 2H), 4.02 − 3.85 (m, 1H), 1.65 − 1.56 (m, 1H), 1.02 − 0.69 (m, 4H). |
| 79 | 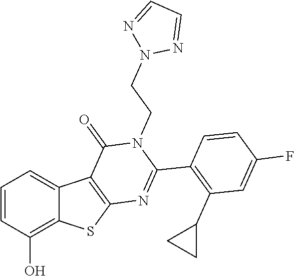 [SMILES: O=c1c2c(nc(-c3ccc(F)cc3C3CC3)n1CCn1nccn1)sc1c(O)cccc12] | 448.10 | ¹H NMR (400 MHz, DMSO-d₆) δ 10.72 (s, 1H), 8.03 (d, J = 8.0 Hz, 1H), 7.94 (d, J = 0.8 Hz, 1H), 7.68 (s, 1H), 7.45 (t, J = 8.0 Hz, 1H), 7.10 − 6.92 (m, 2H), 6.90 − 6.78 (m, 2H), 4.85 − 4.55 (m, 3H), 4.15 − 3.96 (m, 1H), 1.70 − 1.56 (m, 1H), 1.03 − 0.65 (m, 4H). |
| 80 | 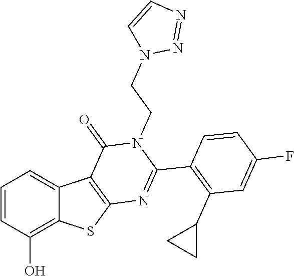 [SMILES: O=c1c2c(nc(-c3ccc(F)cc3C3CC3)n1CCn1ccnn1)sc1c(O)cccc12] | 448.10 | ¹H NMR (400 MHz, DMSO-d₆) δ 10.72 (s, 1H), 8.03 (d, J = 7.6 Hz, 1H), 7.72 (s, 1H), 7.45 (t, J = 8.0 Hz, 1H), 7.12 − 6.95 (m, 3H), 6.85 (dd, J = 10.8, 2.4 Hz, 1H), 4.85 − 4.55 (m, 3H), 4.20 − 4.05 (m, 1H), 1.70 − 1.58 (m, 1H), 1.05 − 0.68 (m, 4H). |
| 81 | 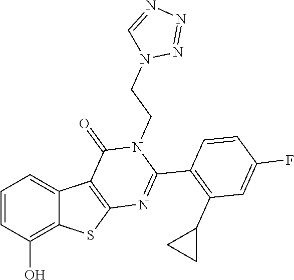 [SMILES: O=c1c2c(nc(-c3ccc(F)cc3C3CC3)n1CCn1cnnn1)sc1c(O)cccc12] | 449.10 | ¹H NMR (400 MHz, DMSO-d₆) δ 10.72 (s, 1H), 9.30 (s, 1H), 7.98 (d, J = 8.0 Hz, 1H), 7.44 (t, J = 8.0 Hz, 1H), 7.10 − 6.92 (m, 3H), 6.86 (dd, J = 10.4, 2.4 Hz, 1H), 4.85 − 4.62 (m, 3H), 4.19 − 4.05 (m, 1H), 1.69 − 1.58 (m, 1H), 1.02 − 0.68 (m, 4H). |
| 82 | 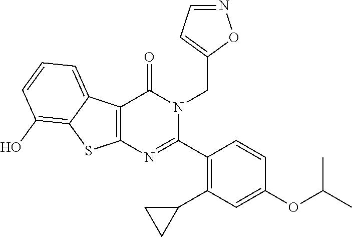 [SMILES: CC(C)Oc1ccc(-c2nc3sc4c(O)cccc4c3c(=O)n2Cc2ccno2)c(C2CC2)c1] | 474.13 | ¹H NMR (400 MHz, Chloroform-d) δ 8.21 (d, J = 7.9 Hz, 1H), 8.14 (d, J = 1.8 Hz, 1H), 7.36 (t, J = 7.9 Hz, 1H), 7.25 (d, J = 8.5 Hz, 1H), 6.85 (d, J = 7.8 Hz, 1H), 6.77 (dd, J = 8.5, 2.5 Hz, 1H), 6.45 (d, J = 2.4 Hz, 1H), 6.19 (d, J = 1.8 Hz, 1H), 5.70 (d, J = 15.5 Hz, 1H), 5.13 (d, J = 15.5 Hz, 1H), 4.57 (hept, J = 6.1 Hz, 1H), 1.63 − 1.54 (m, 1H), 1.34 (t, J = 5.6 Hz, 6H), 0.96 − 0.76 (m, 3H), 0.70 − 0.59 (m, 1H). |
| 83 | 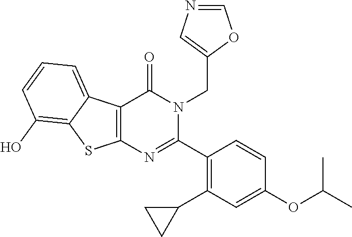 [SMILES: CC(C)Oc1ccc(-c2nc3sc4c(O)cccc4c3c(=O)n2Cc2cnco2)c(C2CC2)c1] | 474.13 | ¹H NMR (400 MHz, Chloroform-d) δ 8.26 (dd, J = 8.0, 0.9 Hz, 1H), 7.76 (s, 1H), 7.39 (t, J = 7.9 Hz, 1H), 7.22 (d, J = 8.4 Hz, 1H), 6.91 (dd, J = 7.9, 1.0 Hz, 1H), 6.84 (s, 1H), 6.81 (dd, J = 8.4, 2.4 Hz, 1H), 6.46 (d, J = 2.4 Hz, 1H), 5.64 (d, J = 15.4 Hz, 1H), 5.10 (d, J = 15.3 Hz, 1H), 4.60 (hept, J = 6.0 Hz, 1H), 1.61 − 1.50 (m, 1H), 1.36 (t, J = 6.0 Hz, 3H), 0.96 − 0.78 (m, 3H), 0.70 − 0.61 (m, 1H). |
| 84 | 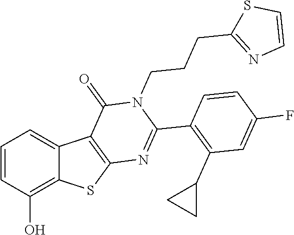 [SMILES: O=c1c2c(nc(-c3ccc(F)cc3C3CC3)n1CCCc1nccs1)sc1c(O)cccc12] | 478.05 | ¹H NMR (400 MHz, DMSO-d₆) δ 10.67 (s, 1H), 8.04 (d, J = 7.6 Hz, 1H), 7.60 − 7.49 (m, 2H), 7.49 (d, J = 3.6 Hz, 1H), 7.42 (t, J = 8.0 Hz, 1H), 7.09 (td, J = 8.4, 2.4 Hz, 1H), 6.97 (d, J = 7.6 Hz, 1H), 6.77 (dd, J = 10.8, 2.4 Hz, 1H), 4.18 (t, J = 5.2 Hz, 1H), 3.85 − 3.72 (m, 1H), 2.92 (td, J = 7.6, 2.4 Hz, 1H), 2.18 − 2.01 (m, 1H), 1.98 − 1.84 (m, 1H), 1.67 − 1.56 (m, 1H), 0.99 − 0.82 (m, 3H), 0.70 − 0.58 (m, 1H). |
| 85 | 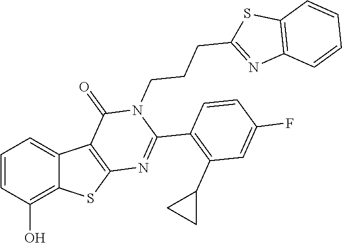 [SMILES: O=c1c2c(nc(-c3ccc(F)cc3C3CC3)n1CCCc1nc3ccccc3s1)sc1c(O)cccc12] | 528.15 | ¹H NMR (400 MHz, DMSO-d₆) δ 10.65 (s, 1H), 8.04 (d, J = 7.6 Hz, 1H), 7.96 (d, J = 7.6 Hz, 1H), 7.82 (d, J = 8.0 Hz, 1H), 7.54 (dd, J = 8.4, 6.0 Hz, 1H), 7.52 − 7.35 (m, 3H), 7.05 − 6.90 (m, 2H), 6.61 (dd, J = 10.4, 2.4 Hz, 1H), 4.32 − 4.21 (m, 1H), 3.92 − 3.78 (m, 1H), 3.06 (dd, J = 12.8, 6.4 Hz, 2H), 2.25 − 2.12 (m, 1H), 2.11 − 1.95 (m, 1H), 1.66 − 1.56 (m, 1H), 0.95 − 0.78 (m, 3H), 0.61 − 0.51 (m, 1H). |
| 86 | 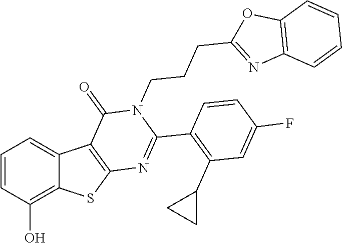 [SMILES: O=c1c2c(nc(-c3ccc(F)cc3C3CC3)n1CCCc1nc3ccccc3o1)sc1c(O)cccc12] | 512.10 | ¹H NMR (400 MHz, DMSO-d₆) δ 10.64 (s, 1H), 8.03 (d, J = 7.2 Hz, 1H), 7.65 − 7.51 (m, 3H), 7.42 (t, J = 8.0 Hz, 1H), 7.39 − 7.26 (m, 2H), 7.05 − 6.92 (m, 2H), 6.67 (dd, J = 10.8, 2.4 Hz, 1H), 4.36 − 4.22 (m, 1H), 3.94 − 3.83 (m, 1H), 2.97 − 2.81 (m, 2H), 2.23 − 1.95 (m, 2H), 1.67 − 1.56 (m, 1H), 0.95 − 0.78 (m, 3H), 0.67 − 0.56 (m, 1H). |
| 87 | 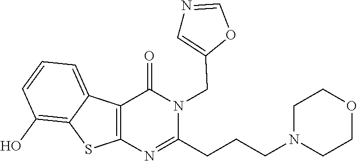 [SMILES: O=c1c2c(nc(CCCN3CCOCC3)n1Cc1cnco1)sc1c(O)cccc12] | 443.13 | ¹H NMR (400 MHz, DMSO-d₆) δ 10.64 (s, 1H), 9.05 (s, 1H), 8.08 (s, 1H), 7.99 (dd, J = 7.8, 1.0 Hz, 1H), 7.40 (t, J = 7.9 Hz, 1H), 6.95 (dd, J = 8.0, 1.0 Hz, 1H), 5.76 (s, 2H), 3.51 − 3.39 (m, 4H), 3.07 (t, J = 7.0 Hz, 2H), 2.47 − 2.17 (m, 6H), 2.03 − 1.90 (m, 2H). |
| 88 | 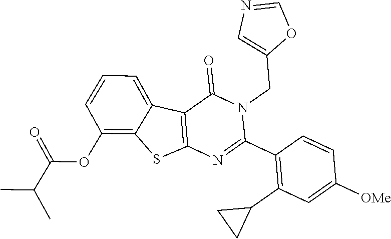 [SMILES: COc1ccc(-c2nc3sc4c(OC(=O)C(C)C)cccc4c3c(=O)n2Cc2cnco2)c(C2CC2)c1] | 516.16 | ¹H NMR (400 MHz, Chloroform-d) δ 8.55 (d, J = 7.9 Hz, 1H), 7.75 (s, 1H), 7.57 (t, J = 8.0 Hz, 1H), 7.27 (dd, J = 25.1, 8.2 Hz, 2H), 6.84 (d, J = 6.0 Hz, 2H), 6.49 (d, J = 2.5 Hz, 1H), 5.61 (d, J = 15.4 Hz, 1H), 5.09 (d, J = 15.4 Hz, 1H), 3.85 (s, 3H), 2.94 (hept, J = 7.0 Hz, 1H), 1.55 (m, 1H), 1.40 (d, J = 7.0 Hz, 6H), 1.05 − 0.76 (m, 3H), 0.66 (m, 1H). |
| 89 | 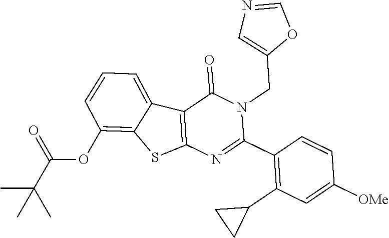 [SMILES: COc1ccc(-c2nc3sc4c(OC(=O)C(C)(C)C)cccc4c3c(=O)n2Cc2cnco2)c(C2CC2)c1] | 530.17 | ¹H NMR (400 MHz, Chloroform-d) δ 8.54 (dd, J = 8.0, 1.0 Hz, 1H), 7.75 (s, 1H), 7.57 (t, J = 8.0 Hz, 1H), 7.29 (dd, J = 7.9, 1.0 Hz, 1H), 7.24 (d, J = 8.5 Hz, 1H), 6.92 − 6.77 (m, 2H), 6.49 (d, J = 2.5 Hz, 1H), 5.60 (d, J = 15.3 Hz, 1H), 5.10 (dd, J = 15.3, 0.9 Hz, 1H), 3.85 (s, 3H), 1.55 (m, 1H), 1.44 (s, 9H), 0.99 − 0.76 (m, 3H), 0.73 − 0.61 (m, 1H). |
| 90 | 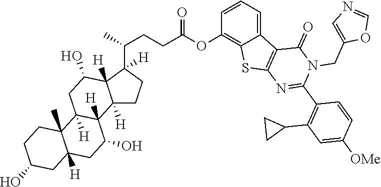 | 836.39 | ¹H NMR (400 MHz, Chloroform-d) δ 8.53 (d, J = 7.9 Hz, 1H), 7.75 (s, 1H), 7.55 (t, J = 8.0 Hz, 1H), 7.29 (d, J = 7.9 Hz, 1H), 7.24 (d, J = 8.5 Hz, 1H), 6.90 − 6.78 (m, 2H), 6.48 (d, J = 2.5 Hz, 1H), 5.59 (dd, J = 15.5, 1.9 Hz, 1H), 5.09 (d, J = 15.3 Hz, 1H), 3.97 (d, J = 3.1 Hz, 1H), 3.84 (s, 5H), 3.43 (m, 1H), 3.11 (s, 6H), 2.73 (m, 1H), 2.59 (m, 1H), 2.19 (m, 2H), 2.03 − 0.54 (m, 35H). |
| 91 | 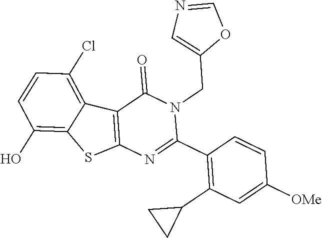 [SMILES: COc1ccc(-c2nc3sc4c(O)ccc(Cl)c4c3c(=O)n2Cc2cnco2)c(C2CC2)c1] | 480.0 | ¹H NMR (400 MHz, DMSO-d₆) δ 10.98 (s, 1H), 8.22 (s, 1H), 7.43 (d, J = 8.5 Hz, 1H), 7.38 (d, J = 8.5 Hz, 1H), 6.93 (d, J = 8.5 Hz, 1H), 6.90 − 6.85 (m, 2H), 6.48 (d, J = 2.5 Hz, 1H), 5.37 (d, J = 15.9 Hz, 1H), 5.04 (dd, J = 15.9, 1.1 Hz, 1H), 3.81 (s, 3H), 1.62 (m, 1H), 0.93 − 0.76 (m, 3H), 0.64 (m, 1H). |

### Assay

17β-HSD13 rapid-fire mass spectrometry assay (RF/MS assay). Recombinant human 17β-HSD13 was expressed and purified from sf9 cells at Charles River Labs (Saffron Walden, UK). Leukotriene B4 (Catalog #71160-24-2) and 12-oxoleukotriene B4 (Catalog #20140) were purchased from Cayman Chemicals (Ann Arbor, MI). NAD+ (Catalog #N8285), BSA (Catalog #A7030), DMSO (Catalog #D2650), and Tween-20 (Catalog #11332465001) were purchased from Sigma (St. Louis, MO). Formic acid (Catalog #28905) was from ThermoFisher Scientific and 384 deep well PP microplates (Catalog #784261) were from Greiner Bio-One. In a typical IC₅₀ assay performed in a 384w PP microplate, test compounds (0-100 μM) were incubated with HSD17B13 (80 nM), LTB4 (10 μM), and NAD⁺ (0.5 mM) in 10 μL assay buffer (20 mM Tris (pH 7.5), BSA (0.005%), and Tween-20 (0.01%)) at RT for 3 h. The assays were quenched by adding 20 μL of 0.15% aqueous formic acid and the plates were frozen at −80° C. RF/MS analysis was performed at PureHoney Technologies (Billerica, MA) on a RapidFire RF300 system (Agilent Technologies, Inc.) coupled to an API 4000 triple quadrupole mass spectrometer (Sciex) equipped with Agilent RapidFire cartridge type A (C4). The mobile phase was 0.09% formic acid and 0.01% trifluoracetic acid in water (Buffer A) and 0.09% formic acid and 0.01% trifluoracetic acid in 80% aqueous acetonitrile (Buffer B). The RapidFire method conditions were the following: 250 ms aspirate, 3000 ms load/desalt, 4000 ms elute, and 500 ms re-equilibrate. RF-MS/MS was performed in negative polarity (−4500 V), the source temperature was 650° C., and gas 1 and gas 2 settings for nitrogen were set to 50. The curtain gas and collision gas were also nitrogen and were set to 20 and 12, respectively. Leukotriene B4 (335.3) and 12-oxoLeukotriene B4 (333.3) SRM transitions were optimized with Discovery Quant software and extracted ion counts for these analytes were determined.

Data Analysis. 17β-HSD13 enzyme activity was measured as percent conversion of extracted ion counts and normalized to high and low controls to determine percent residual activity at various concentrations of test compounds. Data were fitted to normalized activity (variable slope) versus concentration fit in GraphPad Prism 7 to determine IC₅₀. All experiments were run in duplicates.

By using the above method, the inhibition of 17β-HSD13 was evaluated for the compounds of Formula (I). IC₅₀ ranges are as follows: A is <0.1 μM; B is 0.1 μM-1.0 μM; C is 1.0 μM-10 μM; and D is >10 μM.

| Example | IC₅₀ |
|---|---|
| 1 | B |
| 2 | A |
| 3 | B |
| 4 | B |
| 5 | D |
| 6 | D |
| 7 | D |
| 8 | B |
| 9 | A |
| 10 | A |
| 11 | A |
| 12 | A |
| 13 | A |
| 14 | A |
| 15 | A |
| 16 | A |
| 17 | A |
| 18 | A |
| 19 | A |
| 20 | A |
| 21 | A |
| 22 | A |
| 23 | A |
| 24 | A |
| 25 | A |
| 26 | A |
| 27 | A |
| 28 | A |
| 29 | A |
| 30 | A |
| 31 | A |
| 32 | A |
| 33 | A |
| 34 | A |
| 35 | A |
| 36 | A |
| 37 | A |
| 38 | B |
| 39 | A |
| 40 | A |
| 41 | A |
| 42 | A |
| 43 | A |
| 44 | B |
| 45 | B |
| 46 | A |
| 47 | A |
| 48 | A |
| 49 | A |
| 50 | B |
| 51 | A |
| 52 | B |
| 53 | A |
| 54 | B |
| 55 | D |
| 56 | A |
| 57 | B |
| 58 | B |
| 59 | B |
| 60 | A |
| 61 | B |
| 62 | A |
| 63 | A |
| 64 | A |
| 65 | A |
| 66 | A |
| 67 | A |
| 68 | A |
| 69 | A |
| 70 | A |
| 71 | C |
| 72 | B |
| 73 | C |
| 74 | — |
| 75 | A |
| 76 | B |
| 77 | A |
| 78 | B |
| 79 | A |
| 80 | A |
| 81 | A |
| 82 | A |
| 83 | A |
| 84 | A |
| 85 | A |
| 86 | A |
| 87 | B |
| 88 | C |
| 89 | D |
| 90 | B |
| 91 | A |

While this invention has been particularly shown and described with references to preferred embodiments thereof, it will be understood by those skilled in the art that various changes in form and details may be made therein without departing from the scope of the invention encompassed by the appended claims.

1. A compound represented by Formula I or a pharmaceutically acceptable salt, or ester thereof: 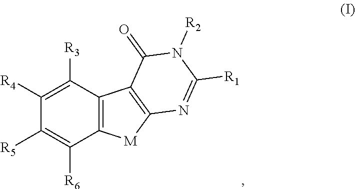
wherein
M is S, SO, SO₂, O or NR₇;
R₁ and R₂ are each independently selected from the group consisting of:
1) Hydrogen;
2) Optionally substituted —C₁-C₈ alkyl;
3) Optionally substituted —C₂-C₈ alkenyl;
4) Optionally substituted —C₂-C₈ alkynyl;
5) Optionally substituted —C₃-C₈ cycloalkyl;
6) Optionally substituted aryl;
7) Optionally substituted arylalkyl;
8) Optionally substituted 3- to 8-membered heterocycloalkyl;
9) Optionally substituted heteroaryl; and
10) Optionally substituted heteroarylalkyl;
R₃, R₄, R₅, and R₆ are each independently selected from the group consisting of hydrogen, halogen, —CN, —OR₉, —SR₉, —C(O)R₇, —C(O)OR₇, —NR₇R₈, —C(O)NR₇R₈, optionally substituted —C₁-C₈ alkyl, optionally substituted aryl, and optionally substituted heteroaryl;
alternatively, R₅ and R₆ are taken together with the carbon atoms to which they are attached to form an optionally substituted carbocyclic or heterocyclic ring;
alternatively, R₄ and R₅ are taken together with the carbon atoms to which they are attached to form an optionally substituted carbocyclic or heterocyclic ring;
alternatively, R₃ and R₄ are taken together with the carbon atoms to which they are attached to form an optionally substituted carbocyclic or heterocyclic ring;
each R₇ and R₈ is independently selected from the group consisting of:
1) Hydrogen;
2) Optionally substituted —C₁-C₈ alkyl;
3) Optionally substituted —C₂-C₈ alkenyl;
4) Optionally substituted —C₂-C₈ alkynyl;
5) Optionally substituted —C₃-C₈ cycloalkyl;
6) Optionally substituted 3- to 8-membered heterocycloalkyl;
7) Optionally substituted aryl;
8) Optionally substituted arylalkyl;
9) Optionally substituted heteroaryl; and
10) Optionally substituted heteroarylalkyl;
alternatively, R₇ and R₈ are taken together with the nitrogen atom to which they are attached to form an optionally substituted heterocyclic ring;
R₉ is selected from the group consisting of:
1) Hydrogen;
2) Optionally substituted —C₁-C₈ alkyl;
3) Optionally substituted —C₂-C₈ alkenyl;
4) Optionally substituted —C₂-C₈ alkynyl;
5) Optionally substituted —C₃-C₈ cycloalkyl;
6) Optionally substituted 3- to 8-membered heterocycloalkyl;
7) Optionally substituted aryl;
8) Optionally substituted arylalkyl;
9) Optionally substituted heteroaryl;
10) Optionally substituted heteroarylalkyl;
11) —C(O)R₁₁;
12) —C(O)NR₁₁R₁₂;
13) —C(O)OR₁₁;
14) —P(O)(OR₁₃)₂; and
15) —P(O)(OR₁₃)(NR₁₁R₁₂);
each R₁₁ and R₁₂ is independently selected from the group consisting of:
1) Hydrogen;
2) Optionally substituted —C₁-C₈ alkyl;
3) Optionally substituted —C₂-C₈ alkenyl;
4) Optionally substituted —C₂-C₈ alkynyl;
5) Optionally substituted —C₃-C₈ cycloalkyl;
6) Optionally substituted 3- to 8-membered heterocycloalkyl;
7) Optionally substituted aryl;
8) Optionally substituted arylalkyl;
9) Optionally substituted heteroaryl; and
10) Optionally substituted heteroarylalkyl;
and R₁₃ is hydrogen, optionally substituted —C₁-C₈ alkyl, or Na⁺.

2. The compound of claim 1, wherein M is S or NR 7, and R 7 is as defined in claim 1.

3. The compound of claim 1, wherein
R₁ is selected from the groups below, wherein each group is optionally substituted: 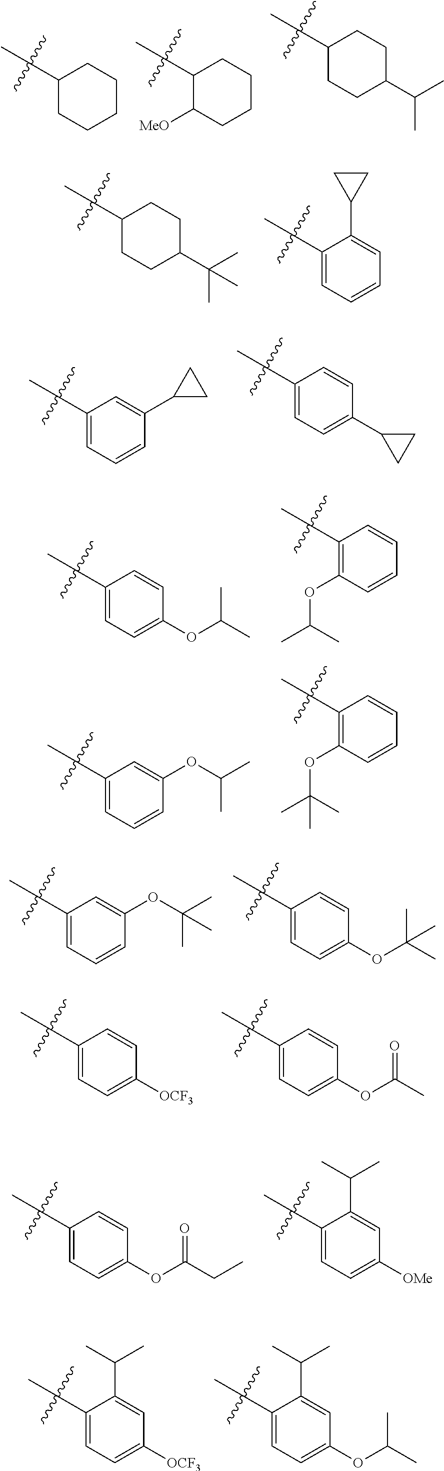 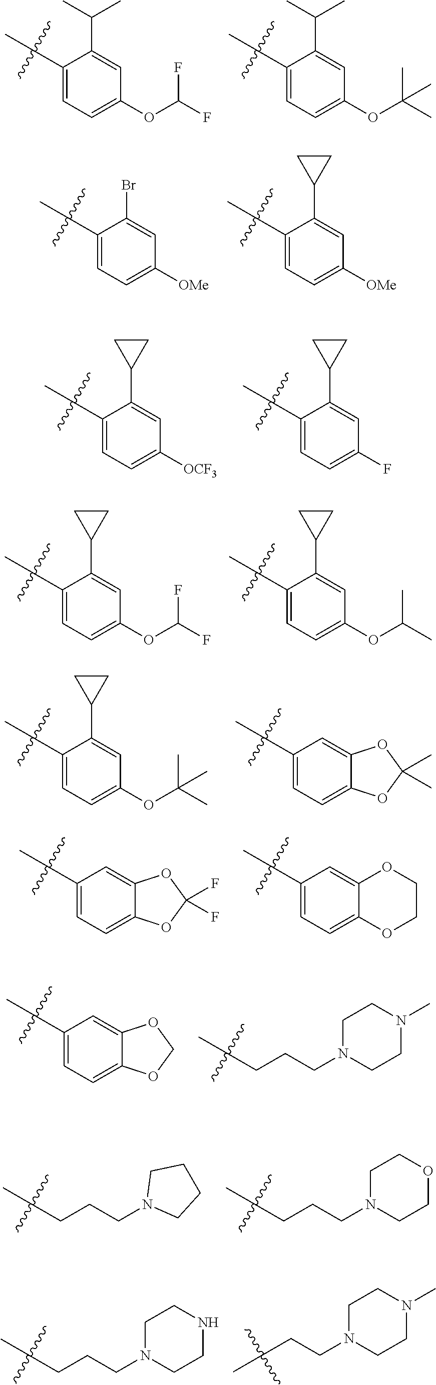 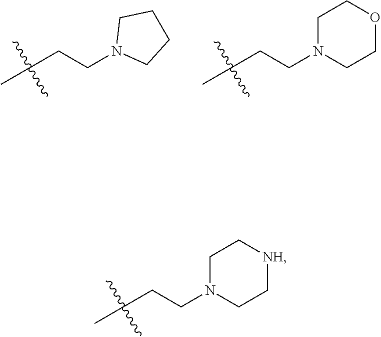 [SMILES: *CCN1CCCC1 ; *CCN1CCOCC1 ; *CCN1CCNCC1]
and
R₂ is selected from the group below, wherein each group is optionally substituted:

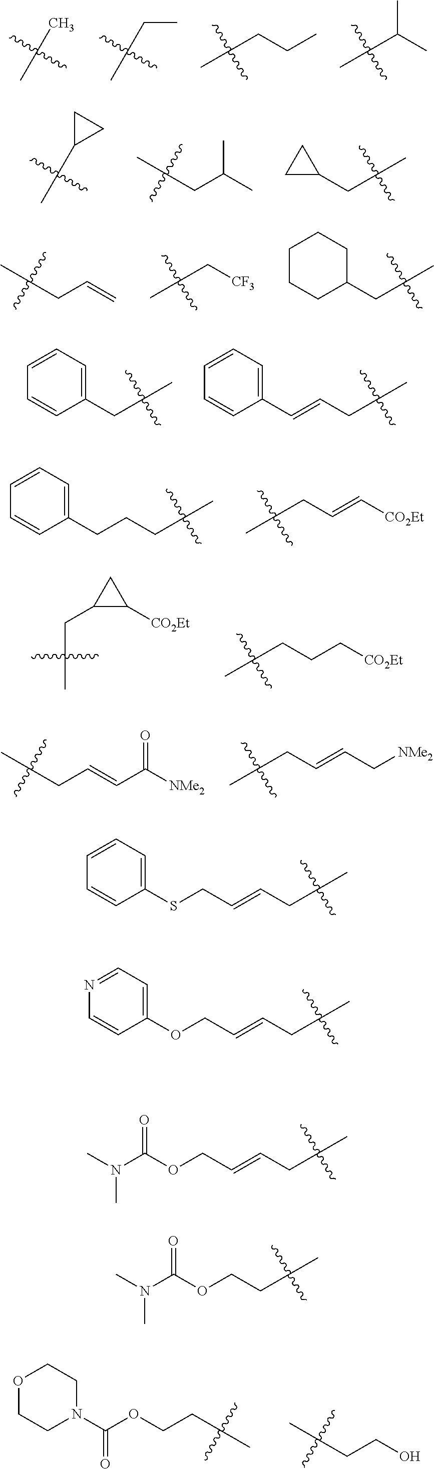

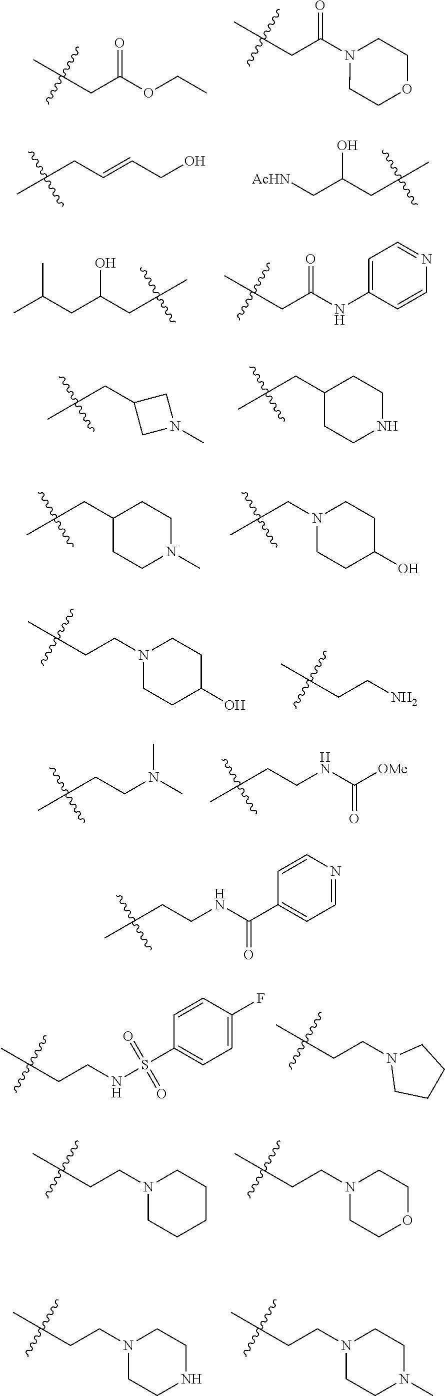

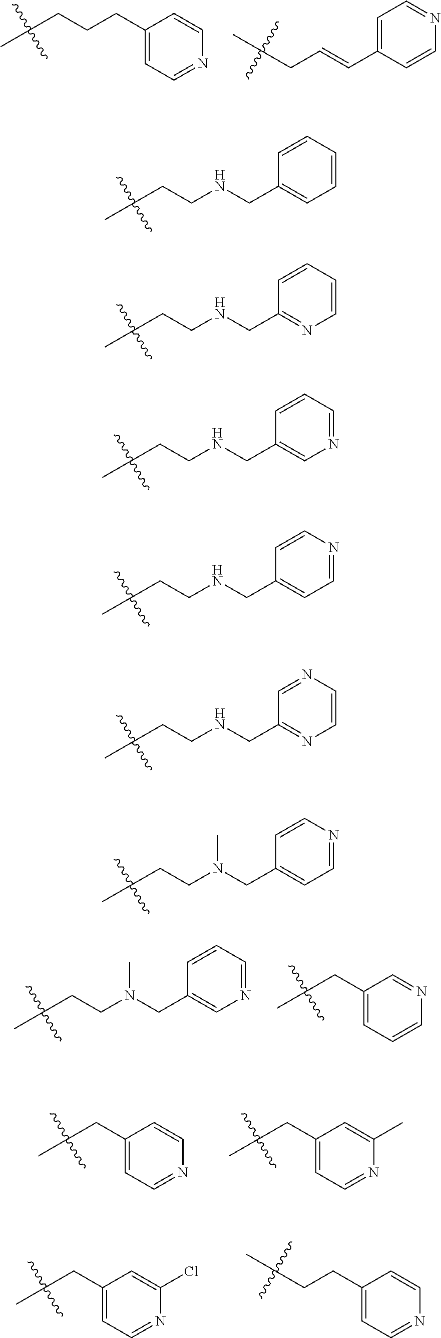

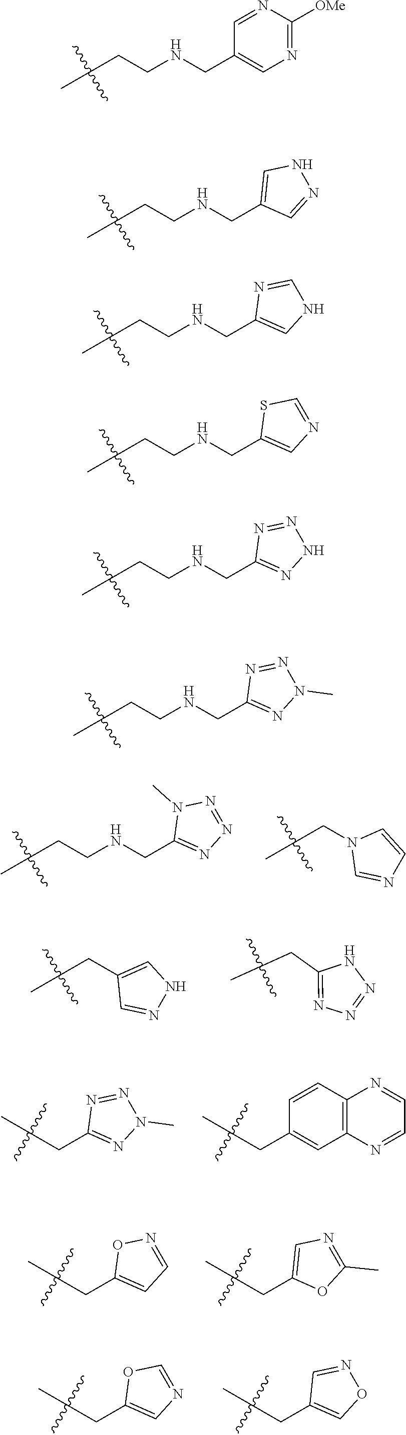

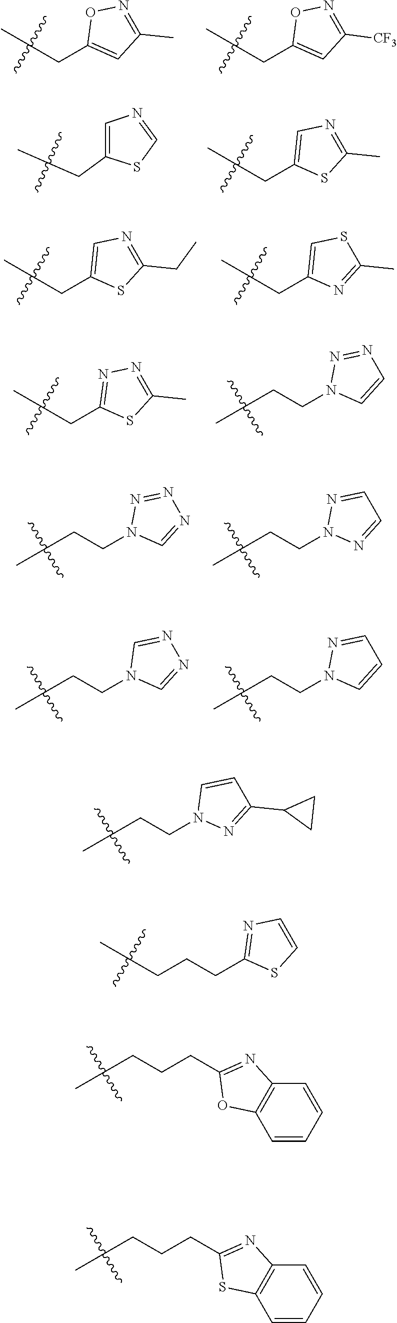

4. The compound of claim 1, represented by Formula (IV) or Formula (V), or a pharmaceutically acceptable salt thereof: 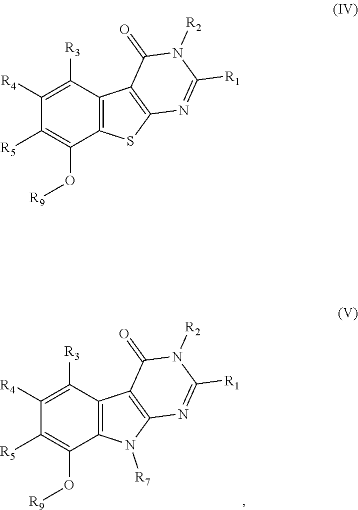
wherein R₁, R₂, R₃, R₄, R₅, R₉, and R₇ are as defined in claim 1.

5. The compound of claim 4, wherein R₉ is selected from the group below, wherein each group is optionally substituted:

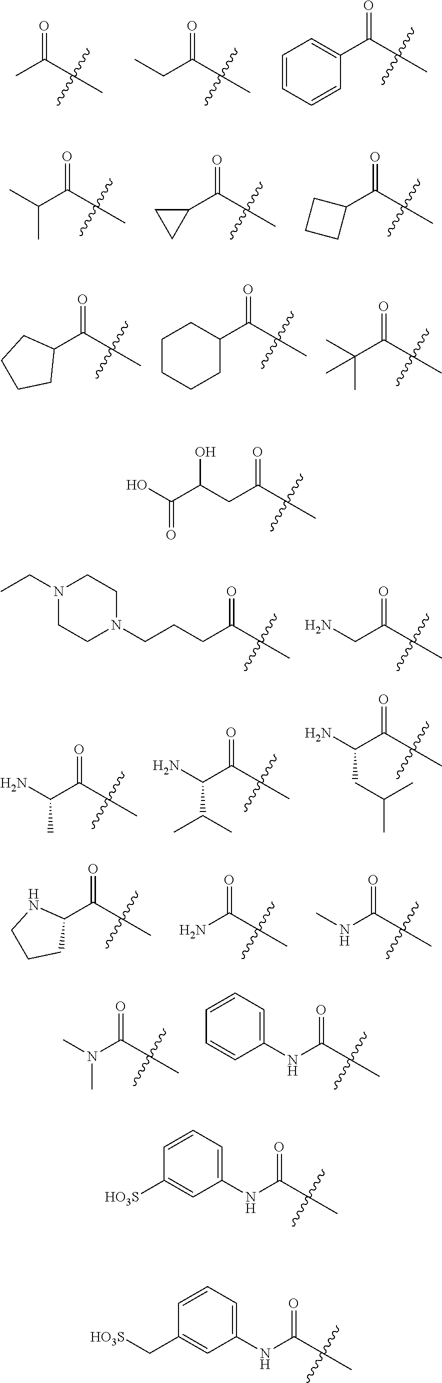

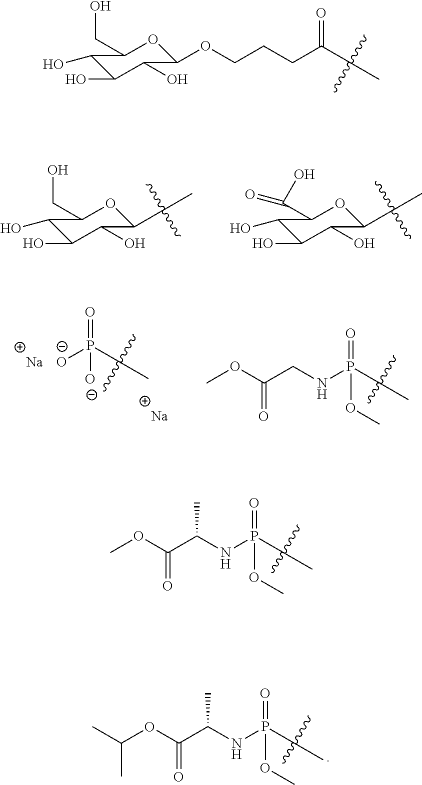

6. The compound of claim 1, represented by Formula (X) or Formula (XI), or a pharmaceutically acceptable salt thereof: 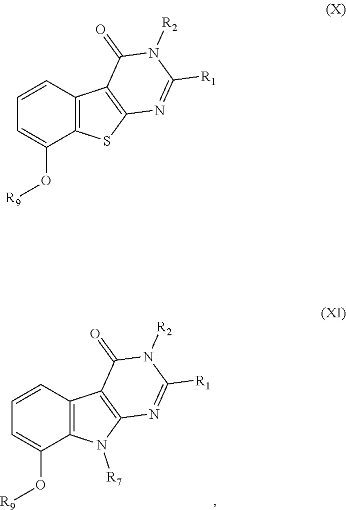
wherein, R₁, R₂, R₇, and R₉ are as defined in claim 1.

7. The compound of claim 1, selected from compounds represented by Formula (X) or a pharmaceutically acceptable salt thereof, 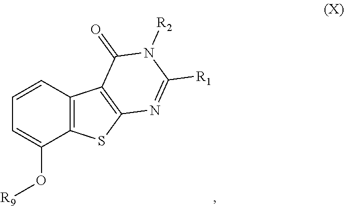
wherein R₉ is hydrogen, and R₁ and R₂ are delineated for each compound in the table below:

| Entry | R₁ | R₂ |
|---|---|---|
| 1 | 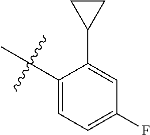 [SMILES: *c1ccc(F)cc1C1CC1] | 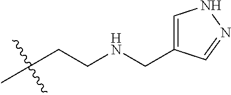 [SMILES: *CCNCc1cn[nH]c1] |
| 2 | 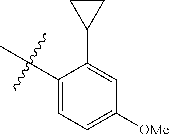 [SMILES: *c1ccc(OC)cc1C1CC1] | 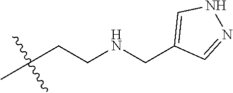 [SMILES: *CCNCc1cn[nH]c1] |
| 3 | 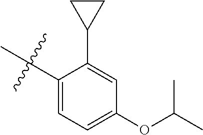 [SMILES: *c1ccc(OC(C)C)cc1C1CC1] | 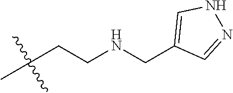 [SMILES: *CCNCc1cn[nH]c1] |
| 4 | 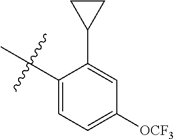 [SMILES: *c1ccc(OC(F)(F)F)cc1C1CC1] | 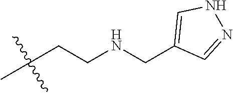 [SMILES: *CCNCc1cn[nH]c1] |
| 5 | 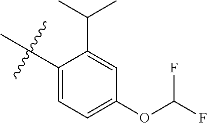 [SMILES: *c1ccc(OC(F)F)cc1C(C)C] | 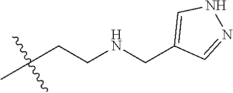 [SMILES: *CCNCc1cn[nH]c1] |
| 6 | 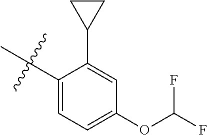 [SMILES: *c1ccc(OC(F)F)cc1C1CC1] | 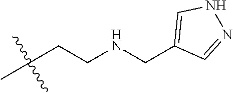 [SMILES: *CCNCc1cn[nH]c1] |
| 7 | 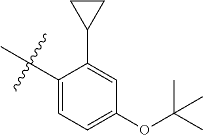 [SMILES: *c1ccc(OC(C)(C)C)cc1C1CC1] | 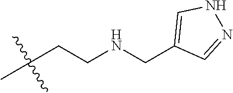 [SMILES: *CCNCc1cn[nH]c1] |
| 8 | 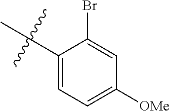 [SMILES: *c1ccc(OC)cc1Br] | 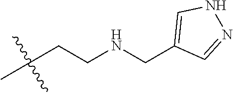 [SMILES: *CCNCc1cn[nH]c1] |
| 9 | 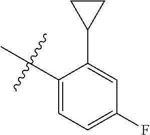 [SMILES: *c1ccc(F)cc1C1CC1] | 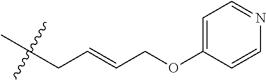 [SMILES: *C/C=C/COc1ccncc1] |
| 10 | 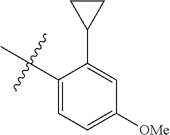 [SMILES: *c1ccc(OC)cc1C1CC1] | 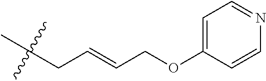 [SMILES: *C/C=C/COc1ccncc1] |
| 11 | 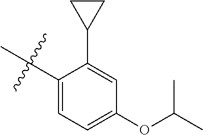 [SMILES: *c1ccc(OC(C)C)cc1C1CC1] | 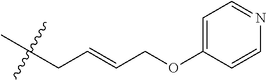 [SMILES: *C/C=C/COc1ccncc1] |
| 12 | 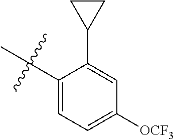 [SMILES: *c1ccc(OC(F)(F)F)cc1C1CC1] | 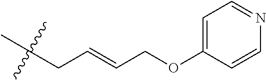 [SMILES: *C/C=C/COc1ccncc1] |
| 13 | 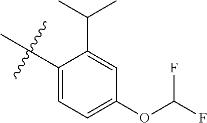 [SMILES: *c1ccc(OC(F)F)cc1C(C)C] | 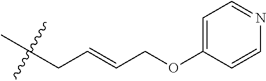 [SMILES: *C/C=C/COc1ccncc1] |
| 14 | 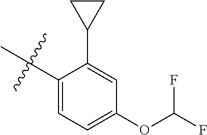 [SMILES: *c1ccc(OC(F)F)cc1C1CC1] | 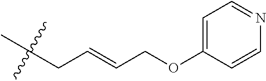 [SMILES: *C/C=C/COc1ccncc1] |
| 15 | 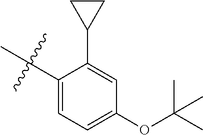 [SMILES: *c1ccc(OC(C)(C)C)cc1C1CC1] | 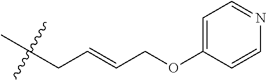 [SMILES: *C/C=C/COc1ccncc1] |
| 16 | 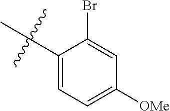 [SMILES: *c1ccc(OC)cc1Br] | 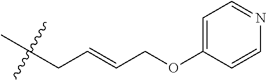 [SMILES: *C/C=C/COc1ccncc1] |
| 17 | 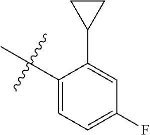 [SMILES: *c1ccc(F)cc1C1CC1] | 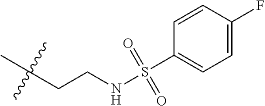 [SMILES: *CCNS(=O)(=O)c1ccc(F)cc1] |
| 18 | 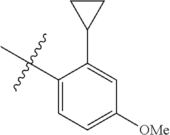 [SMILES: *c1ccc(OC)cc1C1CC1] | 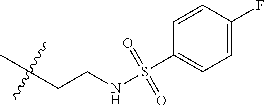 [SMILES: *CCNS(=O)(=O)c1ccc(F)cc1] |
| 19 | 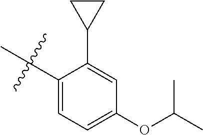 [SMILES: *c1ccc(OC(C)C)cc1C1CC1] | 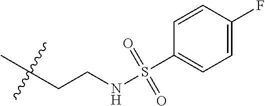 [SMILES: *CCNS(=O)(=O)c1ccc(F)cc1] |
| 20 | 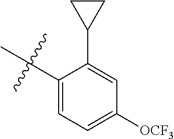 [SMILES: *c1ccc(OC(F)(F)F)cc1C1CC1] | 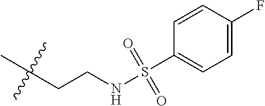 [SMILES: *CCNS(=O)(=O)c1ccc(F)cc1] |
| 21 | 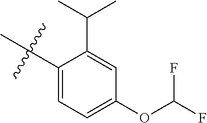 [SMILES: *c1ccc(OC(F)F)cc1C(C)C] | 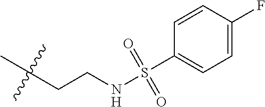 [SMILES: *CCNS(=O)(=O)c1ccc(F)cc1] |
| 22 | 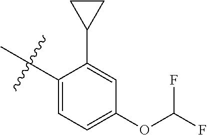 [SMILES: *c1ccc(OC(F)F)cc1C1CC1] | 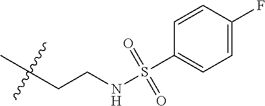 [SMILES: *CCNS(=O)(=O)c1ccc(F)cc1] |
| 23 | 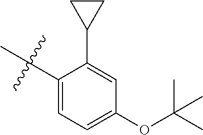 [SMILES: *c1ccc(OC(C)(C)C)cc1C1CC1] | 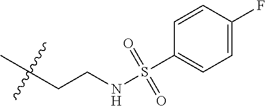 [SMILES: *CCNS(=O)(=O)c1ccc(F)cc1] |
| 24 | 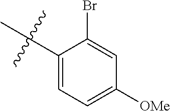 [SMILES: *c1ccc(OC)cc1Br] | 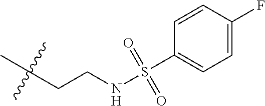 [SMILES: *CCNS(=O)(=O)c1ccc(F)cc1] |
| 25 | 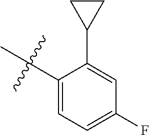 [SMILES: *c1ccc(F)cc1C1CC1] | 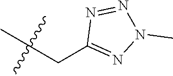 [SMILES: *Cc1nnn(C)n1] |
| 26 | 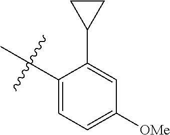 [SMILES: *c1ccc(OC)cc1C1CC1] | 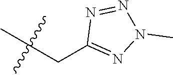 [SMILES: *Cc1nnn(C)n1] |
| 27 | 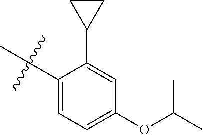 [SMILES: *c1ccc(OC(C)C)cc1C1CC1] | 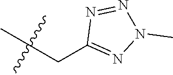 [SMILES: *Cc1nnn(C)n1] |
| 28 | 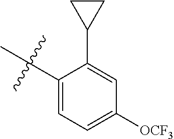 [SMILES: *c1ccc(OC(F)(F)F)cc1C1CC1] | 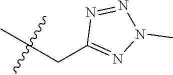 [SMILES: *Cc1nnn(C)n1] |
| 29 | 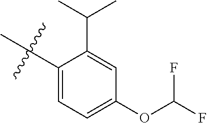 [SMILES: *c1ccc(OC(F)F)cc1C(C)C] | 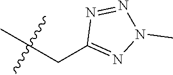 [SMILES: *Cc1nnn(C)n1] |
| 30 | 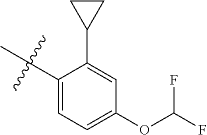 [SMILES: *c1ccc(OC(F)F)cc1C1CC1] | 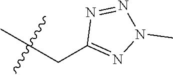 [SMILES: *Cc1nnn(C)n1] |
| 31 | 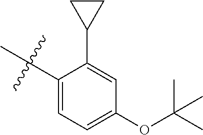 [SMILES: *c1ccc(OC(C)(C)C)cc1C1CC1] | 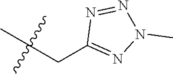 [SMILES: *Cc1nnn(C)n1] |
| 32 | 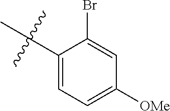 [SMILES: *c1ccc(OC)cc1Br] | 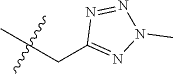 [SMILES: *Cc1nnn(C)n1] |
| 33 | 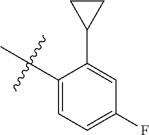 [SMILES: *c1ccc(F)cc1C1CC1] | 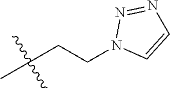 [SMILES: *CCn1ccnn1] |
| 34 | 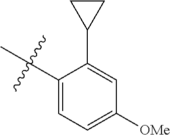 [SMILES: *c1ccc(OC)cc1C1CC1] | 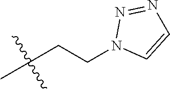 [SMILES: *CCn1ccnn1] |
| 35 | 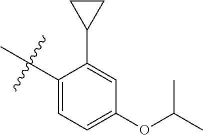 [SMILES: *c1ccc(OC(C)C)cc1C1CC1] | 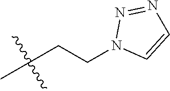 [SMILES: *CCn1ccnn1] |
| 36 | 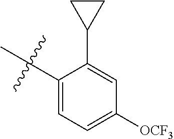 [SMILES: *c1ccc(OC(F)(F)F)cc1C1CC1] | 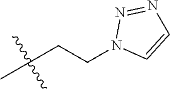 [SMILES: *CCn1ccnn1] |
| 37 | 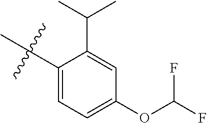 [SMILES: *c1ccc(OC(F)F)cc1C(C)C] | 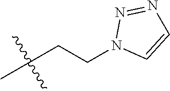 [SMILES: *CCn1ccnn1] |
| 38 | 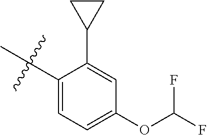 [SMILES: *c1ccc(OC(F)F)cc1C1CC1] | 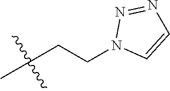 [SMILES: *CCn1ccnn1] |
| 39 | 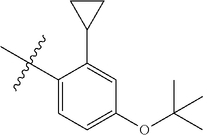 [SMILES: *c1ccc(OC(C)(C)C)cc1C1CC1] | 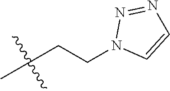 [SMILES: *CCn1ccnn1] |
| 40 | 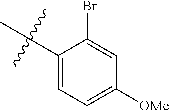 [SMILES: *c1ccc(OC)cc1Br] | 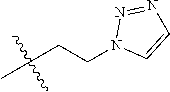 [SMILES: *CCn1ccnn1] |
| 41 | 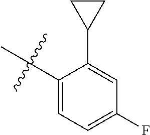 [SMILES: *c1ccc(F)cc1C1CC1] | 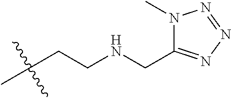 [SMILES: *CCNCc1nnnn1C] |
| 42 | 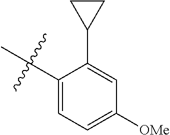 [SMILES: *c1ccc(OC)cc1C1CC1] | 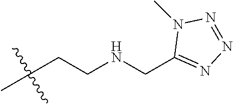 [SMILES: *CCNCc1nnnn1C] |
| 43 | 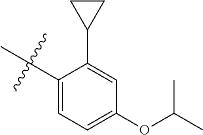 [SMILES: *c1ccc(OC(C)C)cc1C1CC1] | 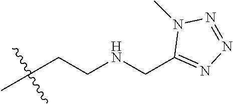 [SMILES: *CCNCc1nnnn1C] |
| 44 | 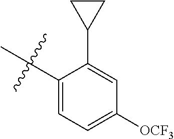 [SMILES: *c1ccc(OC(F)(F)F)cc1C1CC1] | 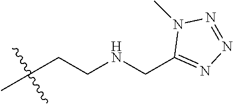 [SMILES: *CCNCc1nnnn1C] |
| 45 | 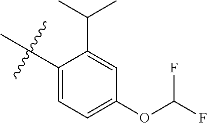 [SMILES: *c1ccc(OC(F)F)cc1C(C)C] | 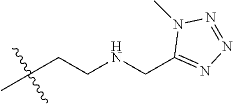 [SMILES: *CCNCc1nnnn1C] |
| 46 | 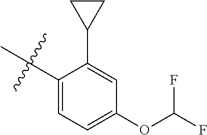 [SMILES: *c1ccc(OC(F)F)cc1C1CC1] | 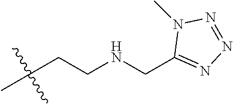 [SMILES: *CCNCc1nnnn1C] |
| 47 | 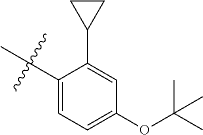 [SMILES: *c1ccc(OC(C)(C)C)cc1C1CC1] | 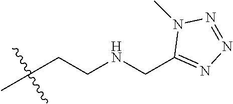 [SMILES: *CCNCc1nnnn1C] |
| 48 | 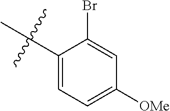 [SMILES: *c1ccc(OC)cc1Br] | 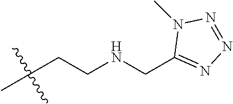 [SMILES: *CCNCc1nnnn1C] |
| 49 | 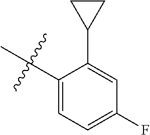 [SMILES: *c1ccc(F)cc1C1CC1] | 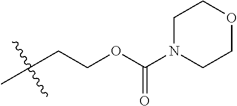 [SMILES: *CCOC(=O)N1CCOCC1] |
| 50 | 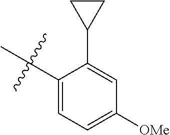 [SMILES: *c1ccc(OC)cc1C1CC1] | 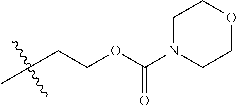 [SMILES: *CCOC(=O)N1CCOCC1] |
| 51 | 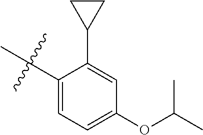 [SMILES: *c1ccc(OC(C)C)cc1C1CC1] | 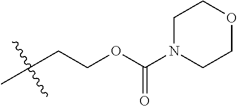 [SMILES: *CCOC(=O)N1CCOCC1] |
| 52 | 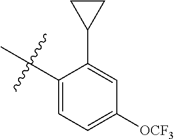 [SMILES: *c1ccc(OC(F)(F)F)cc1C1CC1] | 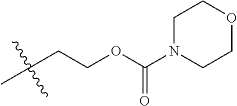 [SMILES: *CCOC(=O)N1CCOCC1] |
| 53 | 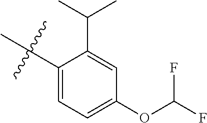 [SMILES: *c1ccc(OC(F)F)cc1C(C)C] | 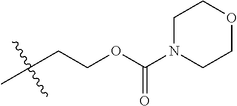 [SMILES: *CCOC(=O)N1CCOCC1] |
| 54 | 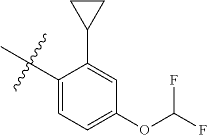 [SMILES: *c1ccc(OC(F)F)cc1C1CC1] | 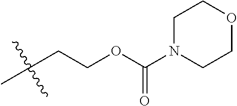 [SMILES: *CCOC(=O)N1CCOCC1] |
| 55 | 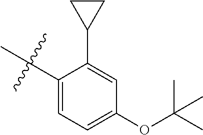 [SMILES: *c1ccc(OC(C)(C)C)cc1C1CC1] | 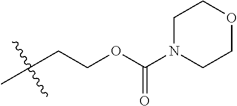 [SMILES: *CCOC(=O)N1CCOCC1] |
| 56 | 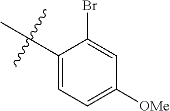 [SMILES: *c1ccc(OC)cc1Br] | 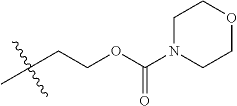 [SMILES: *CCOC(=O)N1CCOCC1] |
| 57 | 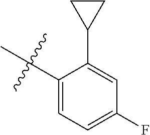 [SMILES: *c1ccc(F)cc1C1CC1] | 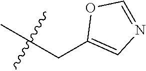 [SMILES: *Cc1cnco1] |
| 58 | 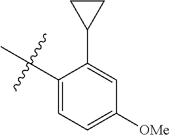 [SMILES: *c1ccc(OC)cc1C1CC1] | 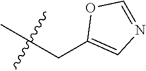 [SMILES: *Cc1cnco1] |
| 59 | 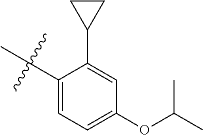 [SMILES: *c1ccc(OC(C)C)cc1C1CC1] | 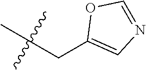 [SMILES: *Cc1cnco1] |
| 60 | 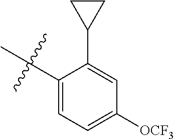 [SMILES: *c1ccc(OC(F)(F)F)cc1C1CC1] | 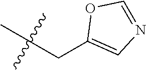 [SMILES: *Cc1cnco1] |
| 61 | 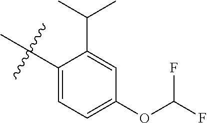 [SMILES: *c1ccc(OC(F)F)cc1C(C)C] | 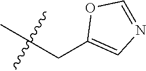 [SMILES: *Cc1cnco1] |
| 62 | 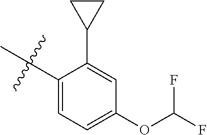 [SMILES: *c1ccc(OC(F)F)cc1C1CC1] | 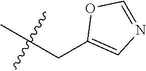 [SMILES: *Cc1cnco1] |
| 63 | 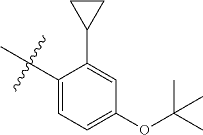 [SMILES: *c1ccc(OC(C)(C)C)cc1C1CC1] | 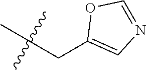 [SMILES: *Cc1cnco1] |
| 64 | 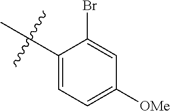 [SMILES: *c1ccc(OC)cc1Br] | 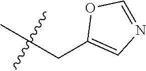 [SMILES: *Cc1cnco1] |
| 65 | 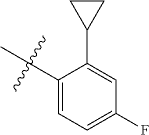 [SMILES: *c1ccc(F)cc1C1CC1] | 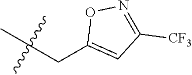 [SMILES: *Cc1cc(C(F)(F)F)no1] |
| 66 | 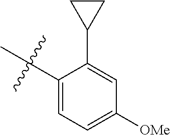 [SMILES: *c1ccc(OC)cc1C1CC1] | 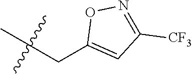 [SMILES: *Cc1cc(C(F)(F)F)no1] |
| 67 | 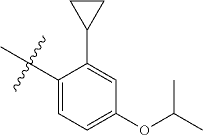 [SMILES: *c1ccc(OC(C)C)cc1C1CC1] | 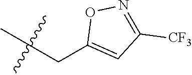 [SMILES: *Cc1cc(C(F)(F)F)no1] |
| 68 | 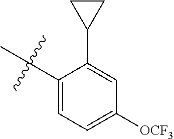 [SMILES: *c1ccc(OC(F)(F)F)cc1C1CC1] | 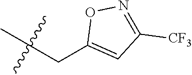 [SMILES: *Cc1cc(C(F)(F)F)no1] |
| 69 | 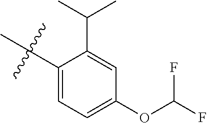 [SMILES: *c1ccc(OC(F)F)cc1C(C)C] | 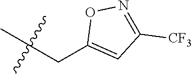 [SMILES: *Cc1cc(C(F)(F)F)no1] |
| 70 | 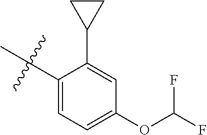 [SMILES: *c1ccc(OC(F)F)cc1C1CC1] | 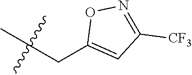 [SMILES: *Cc1cc(C(F)(F)F)no1] |
| 71 | 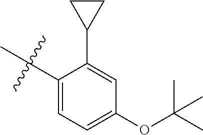 [SMILES: *c1ccc(OC(C)(C)C)cc1C1CC1] | 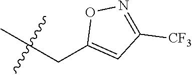 [SMILES: *Cc1cc(C(F)(F)F)no1] |
| 72 | 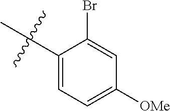 [SMILES: *c1ccc(OC)cc1Br] | 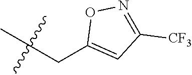 [SMILES: *Cc1cc(C(F)(F)F)no1] |
| 73 | 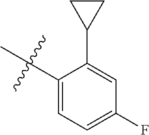 [SMILES: *c1ccc(F)cc1C1CC1] | 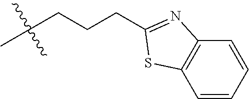 [SMILES: *CCCc1nc2ccccc2s1] |
| 74 | 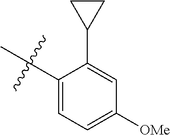 [SMILES: *c1ccc(OC)cc1C1CC1] | 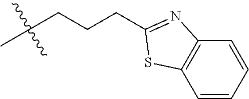 [SMILES: *CCCc1nc2ccccc2s1] |
| 75 | 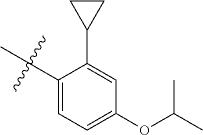 [SMILES: *c1ccc(OC(C)C)cc1C1CC1] | 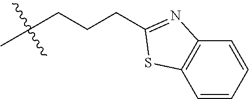 [SMILES: *CCCc1nc2ccccc2s1] |
| 76 | 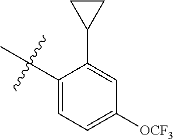 [SMILES: *c1ccc(OC(F)(F)F)cc1C1CC1] | 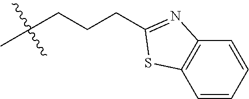 [SMILES: *CCCc1nc2ccccc2s1] |
| 77 | 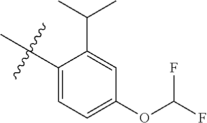 [SMILES: *c1ccc(OC(F)F)cc1C(C)C] | 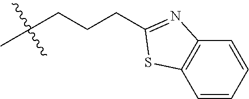 [SMILES: *CCCc1nc2ccccc2s1] |
| 78 | 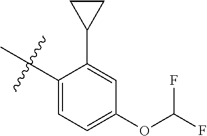 [SMILES: *c1ccc(OC(F)F)cc1C1CC1] | 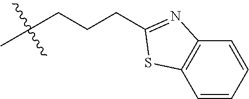 [SMILES: *CCCc1nc2ccccc2s1] |
| 79 | 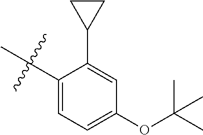 [SMILES: *c1ccc(OC(C)(C)C)cc1C1CC1] | 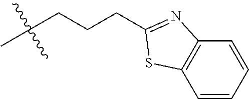 [SMILES: *CCCc1nc2ccccc2s1] |
| 80 | 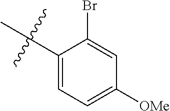 [SMILES: *c1ccc(OC)cc1Br] | 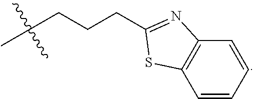 [SMILES: *CCCc1nc2ccccc2s1] |

8. The compound of claim 1, selected from compounds represented by Formula (X) or a pharmaceutically acceptable salt thereof, 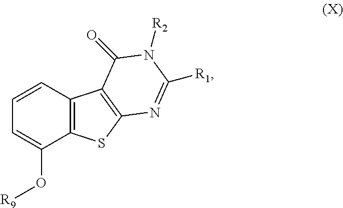
wherein R₁, R₂ and R₉ are delineated for each compound are set forth in the table below:

| Entry | R₁ | R₂ | R₉ |
|---|---|---|---|
| 81 | 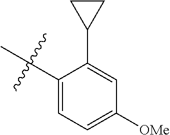 [SMILES: *c1ccc(OC)cc1C1CC1] | 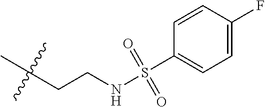 [SMILES: *CCNS(=O)(=O)c1ccc(F)cc1] | 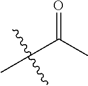 [SMILES: *C(C)=O] |
| 82 | 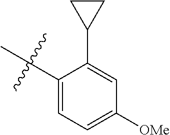 [SMILES: *c1ccc(OC)cc1C1CC1] | 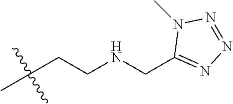 [SMILES: *CCNCc1nnnn1C] | 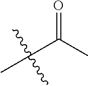 [SMILES: *C(C)=O] |
| 83 | 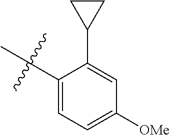 [SMILES: *c1ccc(OC)cc1C1CC1] | 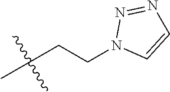 [SMILES: *CCn1ccnn1] | 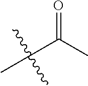 [SMILES: *C(C)=O] |
| 84 | 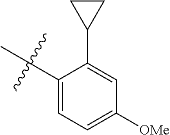 [SMILES: *c1ccc(OC)cc1C1CC1] | 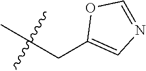 [SMILES: *Cc1cnco1] | 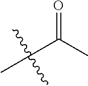 [SMILES: *C(C)=O] |
| 85 | 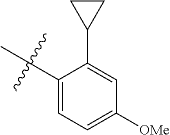 [SMILES: *c1ccc(OC)cc1C1CC1] | 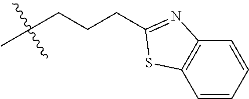 [SMILES: *CCCc1nc2ccccc2s1] | 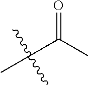 [SMILES: *C(C)=O] |
| 86 | 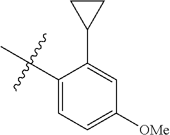 [SMILES: *c1ccc(OC)cc1C1CC1] | 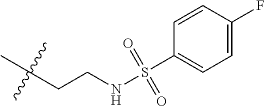 [SMILES: *CCNS(=O)(=O)c1ccc(F)cc1] | 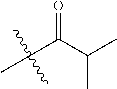 [SMILES: *C(=O)C(C)C] |
| 87 | 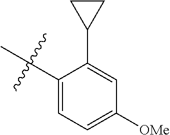 [SMILES: *c1ccc(OC)cc1C1CC1] | 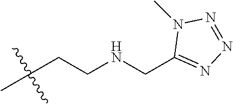 [SMILES: *CCNCc1nnnn1C] | 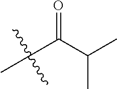 [SMILES: *C(=O)C(C)C] |
| 88 | 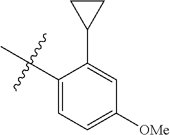 [SMILES: *c1ccc(OC)cc1C1CC1] | 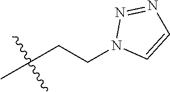 [SMILES: *CCn1ccnn1] | 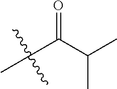 [SMILES: *C(=O)C(C)C] |
| 89 | 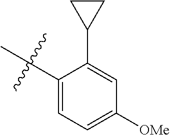 [SMILES: *c1ccc(OC)cc1C1CC1] | 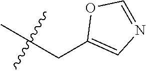 [SMILES: *Cc1cnco1] | 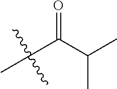 [SMILES: *C(=O)C(C)C] |
| 90 | 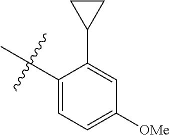 [SMILES: *c1ccc(OC)cc1C1CC1] | 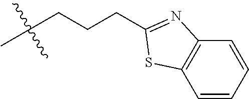 [SMILES: *CCCc1nc2ccccc2s1] | 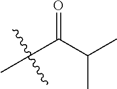 [SMILES: *C(=O)C(C)C] |
| 91 | 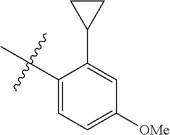 [SMILES: *c1ccc(OC)cc1C1CC1] | 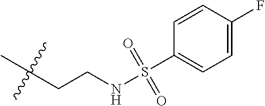 [SMILES: *CCNS(=O)(=O)c1ccc(F)cc1] | 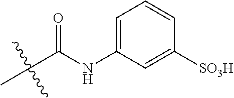 [SMILES: *C(=O)Nc1cccc(S(=O)(=O)O)c1] |
| 92 | 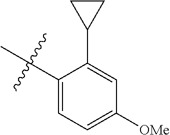 [SMILES: *c1ccc(OC)cc1C1CC1] | 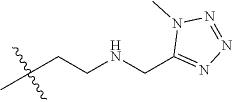 [SMILES: *CCNCc1nnnn1C] | 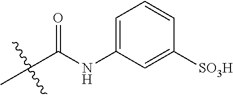 [SMILES: *C(=O)Nc1cccc(S(=O)(=O)O)c1] |
| 93 | 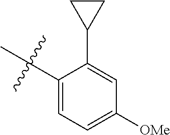 [SMILES: *c1ccc(OC)cc1C1CC1] | 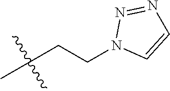 [SMILES: *CCn1ccnn1] | 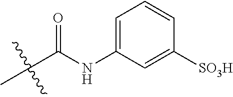 [SMILES: *C(=O)Nc1cccc(S(=O)(=O)O)c1] |
| 94 | 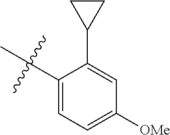 [SMILES: *c1ccc(OC)cc1C1CC1] | 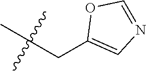 [SMILES: *Cc1cnco1] | 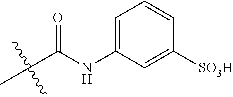 [SMILES: *C(=O)Nc1cccc(S(=O)(=O)O)c1] |
| 95 | 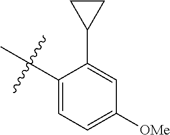 [SMILES: *c1ccc(OC)cc1C1CC1] | 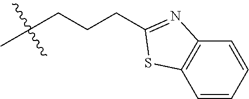 [SMILES: *CCCc1nc2ccccc2s1] | 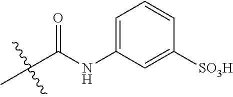 [SMILES: *C(=O)Nc1cccc(S(=O)(=O)O)c1] |
| 96 | 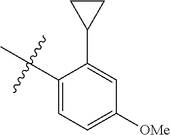 [SMILES: *c1ccc(OC)cc1C1CC1] | 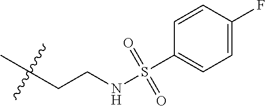 [SMILES: *CCNS(=O)(=O)c1ccc(F)cc1] | 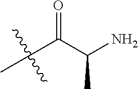 [SMILES: *C(=O)[C@H](C)N] |
| 97 | 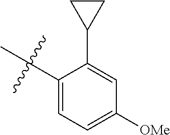 [SMILES: *c1ccc(OC)cc1C1CC1] | 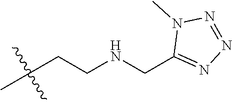 [SMILES: *CCNCc1nnnn1C] | 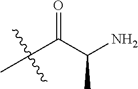 [SMILES: *C(=O)[C@H](C)N] |
| 98 | 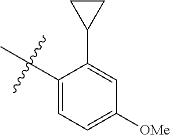 [SMILES: *c1ccc(OC)cc1C1CC1] | 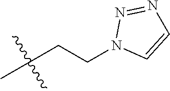 [SMILES: *CCn1ccnn1] | 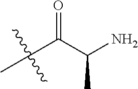 [SMILES: *C(=O)[C@H](C)N] |
| 99 | 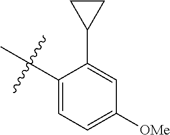 [SMILES: *c1ccc(OC)cc1C1CC1] | 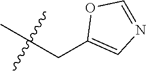 [SMILES: *Cc1cnco1] | 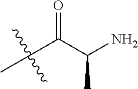 [SMILES: *C(=O)[C@H](C)N] |
| 100 | 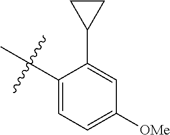 [SMILES: *c1ccc(OC)cc1C1CC1] | 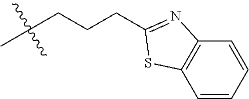 [SMILES: *CCCc1nc2ccccc2s1] | 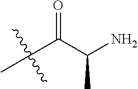 [SMILES: *C(=O)[C@H](C)N] |
| 101 | 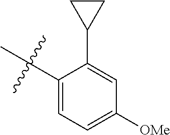 [SMILES: *c1ccc(OC)cc1C1CC1] | 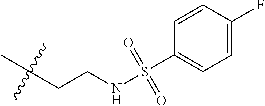 [SMILES: *CCNS(=O)(=O)c1ccc(F)cc1] | 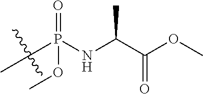 [SMILES: *P(=O)(N[C@@H](C)C(=O)OC)OC] |
| 102 | 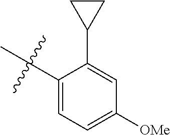 [SMILES: *c1ccc(OC)cc1C1CC1] | 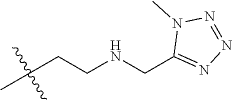 [SMILES: *CCNCc1nnnn1C] | 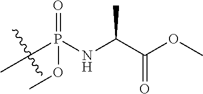 [SMILES: *P(=O)(N[C@@H](C)C(=O)OC)OC] |
| 103 | 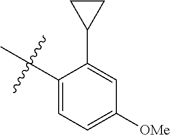 [SMILES: *c1ccc(OC)cc1C1CC1] | 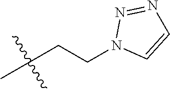 [SMILES: *CCn1ccnn1] | 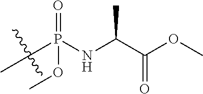 [SMILES: *P(=O)(N[C@@H](C)C(=O)OC)OC] |
| 104 | 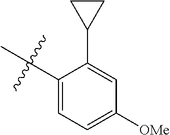 [SMILES: *c1ccc(OC)cc1C1CC1] | 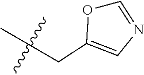 [SMILES: *Cc1cnco1] | 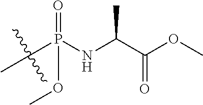 [SMILES: *P(=O)(N[C@@H](C)C(=O)OC)OC] |
| 105 | 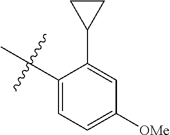 [SMILES: *c1ccc(OC)cc1C1CC1] | 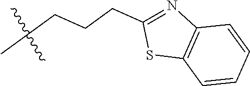 [SMILES: *CCCc1nc2ccccc2s1] | 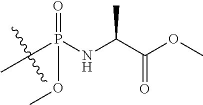 [SMILES: *P(=O)(N[C@@H](C)C(=O)OC)OC] |
| 106 | 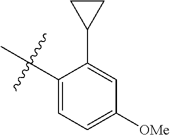 [SMILES: *c1ccc(OC)cc1C1CC1] | 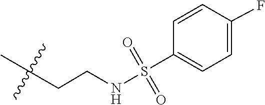 [SMILES: *CCNS(=O)(=O)c1ccc(F)cc1] | 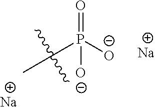 [SMILES: *P(=O)([O-])[O-] ; [Na+] ; [Na+]] |
| 107 | 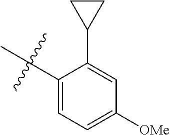 [SMILES: *c1ccc(OC)cc1C1CC1] | 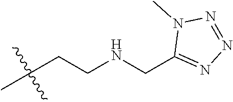 [SMILES: *CCNCc1nnnn1C] | 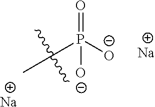 [SMILES: *P(=O)([O-])[O-] ; [Na+] ; [Na+]] |
| 108 | 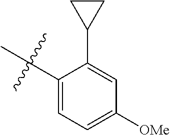 [SMILES: *c1ccc(OC)cc1C1CC1] | 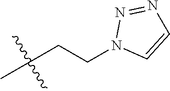 [SMILES: *CCn1ccnn1] | 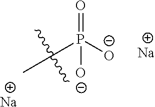 [SMILES: *P(=O)([O-])[O-] ; [Na+] ; [Na+]] |
| 109 | 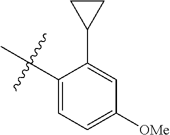 [SMILES: *c1ccc(OC)cc1C1CC1] | 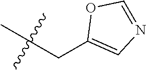 [SMILES: *Cc1cnco1] | 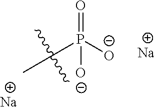 [SMILES: *P(=O)([O-])[O-] ; [Na+] ; [Na+]] |
| 110 | 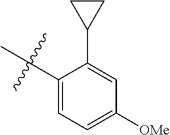 [SMILES: *c1ccc(OC)cc1C1CC1] | 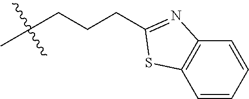 [SMILES: *CCCc1nc2ccccc2s1] | 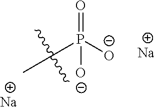 [SMILES: *P(=O)([O-])[O-] ; [Na+] ; [Na+]] |
| 111 | 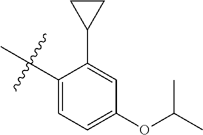 [SMILES: *c1ccc(OC(C)C)cc1C1CC1] | 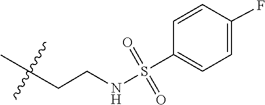 [SMILES: *CCNS(=O)(=O)c1ccc(F)cc1] | 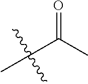 [SMILES: *C(C)=O] |
| 112 | 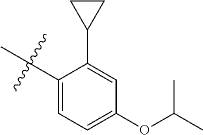 [SMILES: *c1ccc(OC(C)C)cc1C1CC1] | 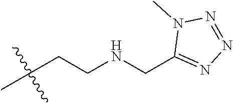 [SMILES: *CCNCc1nnnn1C] | 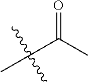 [SMILES: *C(C)=O] |
| 113 | 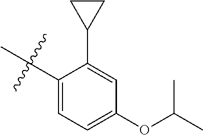 [SMILES: *c1ccc(OC(C)C)cc1C1CC1] | 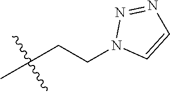 [SMILES: *CCn1ccnn1] | 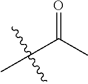 [SMILES: *C(C)=O] |
| 114 | 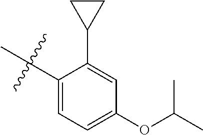 [SMILES: *c1ccc(OC(C)C)cc1C1CC1] | 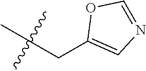 [SMILES: *Cc1cnco1] | 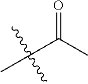 [SMILES: *C(C)=O] |
| 115 | 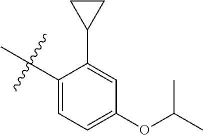 [SMILES: *c1ccc(OC(C)C)cc1C1CC1] | 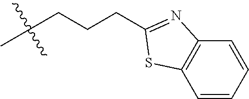 [SMILES: *CCCc1nc2ccccc2s1] | 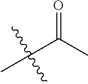 [SMILES: *C(C)=O] |
| 116 | 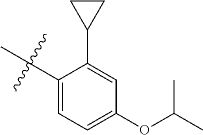 [SMILES: *c1ccc(OC(C)C)cc1C1CC1] | 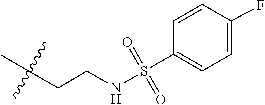 [SMILES: *CCNS(=O)(=O)c1ccc(F)cc1] | 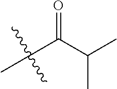 [SMILES: *C(=O)C(C)C] |
| 117 | 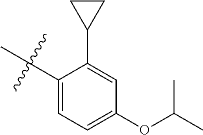 [SMILES: *c1ccc(OC(C)C)cc1C1CC1] | 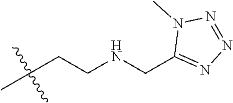 [SMILES: *CCNCc1nnnn1C] | 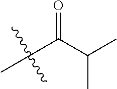 [SMILES: *C(=O)C(C)C] |
| 118 | 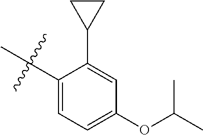 [SMILES: *c1ccc(OC(C)C)cc1C1CC1] | 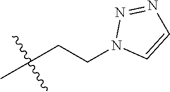 [SMILES: *CCn1ccnn1] | 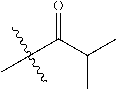 [SMILES: *C(=O)C(C)C] |
| 119 | 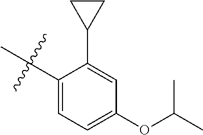 [SMILES: *c1ccc(OC(C)C)cc1C1CC1] | 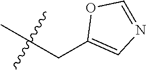 [SMILES: *Cc1cnco1] | 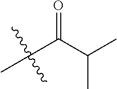 [SMILES: *C(=O)C(C)C] |
| 120 | 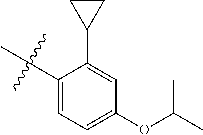 [SMILES: *c1ccc(OC(C)C)cc1C1CC1] | 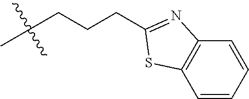 [SMILES: *CCCc1nc2ccccc2s1] | 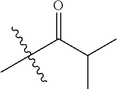 [SMILES: *C(=O)C(C)C] |
| 121 | 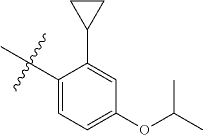 [SMILES: *c1ccc(OC(C)C)cc1C1CC1] | 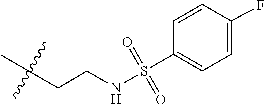 [SMILES: *CCNS(=O)(=O)c1ccc(F)cc1] | 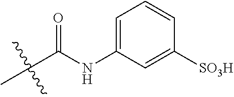 [SMILES: *C(=O)Nc1cccc(S(=O)(=O)O)c1] |
| 122 | 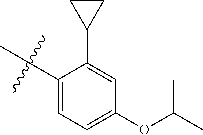 [SMILES: *c1ccc(OC(C)C)cc1C1CC1] | 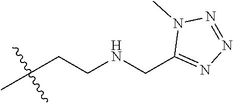 [SMILES: *CCNCc1nnnn1C] | 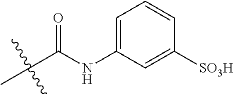 [SMILES: *C(=O)Nc1cccc(S(=O)(=O)O)c1] |
| 123 | 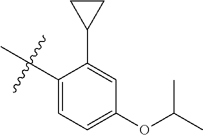 [SMILES: *c1ccc(OC(C)C)cc1C1CC1] | 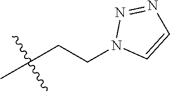 [SMILES: *CCn1ccnn1] | 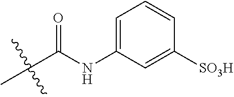 [SMILES: *C(=O)Nc1cccc(S(=O)(=O)O)c1] |
| 124 | 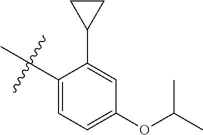 [SMILES: *c1ccc(OC(C)C)cc1C1CC1] | 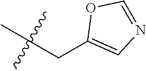 [SMILES: *Cc1cnco1] | 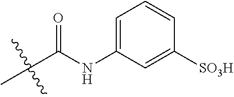 [SMILES: *C(=O)Nc1cccc(S(=O)(=O)O)c1] |
| 125 | 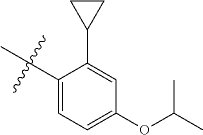 [SMILES: *c1ccc(OC(C)C)cc1C1CC1] | 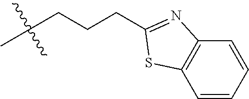 [SMILES: *CCCc1nc2ccccc2s1] | 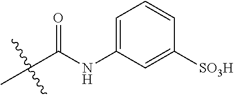 [SMILES: *C(=O)Nc1cccc(S(=O)(=O)O)c1] |
| 126 | 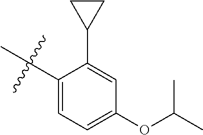 [SMILES: *c1ccc(OC(C)C)cc1C1CC1] | 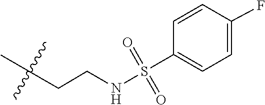 [SMILES: *CCNS(=O)(=O)c1ccc(F)cc1] | 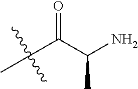 [SMILES: *C(=O)[C@H](C)N] |
| 127 | 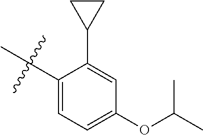 [SMILES: *c1ccc(OC(C)C)cc1C1CC1] | 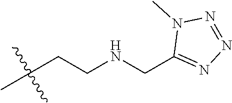 [SMILES: *CCNCc1nnnn1C] | 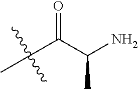 [SMILES: *C(=O)[C@H](C)N] |
| 128 | 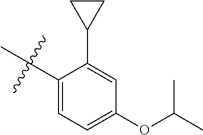 [SMILES: *c1ccc(OC(C)C)cc1C1CC1] | 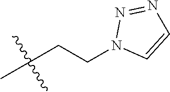 [SMILES: *CCn1ccnn1] | 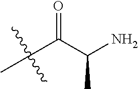 [SMILES: *C(=O)[C@H](C)N] |
| 129 | 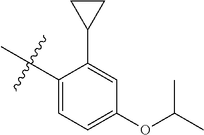 [SMILES: *c1ccc(OC(C)C)cc1C1CC1] | 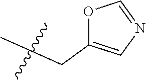 [SMILES: *Cc1cnco1] | 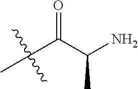 [SMILES: *C(=O)[C@H](C)N] |
| 130 | 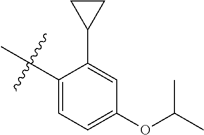 [SMILES: *c1ccc(OC(C)C)cc1C1CC1] | 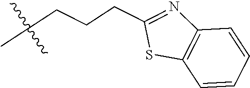 [SMILES: *CCCc1nc2ccccc2s1] | 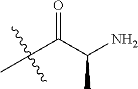 [SMILES: *C(=O)[C@H](C)N] |
| 131 | 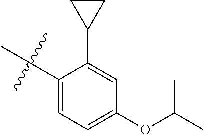 [SMILES: *c1ccc(OC(C)C)cc1C1CC1] | 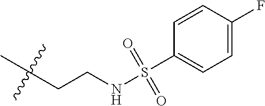 [SMILES: *CCNS(=O)(=O)c1ccc(F)cc1] | 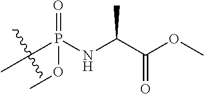 [SMILES: *P(=O)(N[C@@H](C)C(=O)OC)OC] |
| 132 | 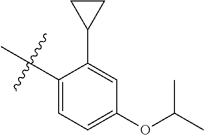 [SMILES: *c1ccc(OC(C)C)cc1C1CC1] | 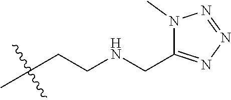 [SMILES: *CCNCc1nnnn1C] | 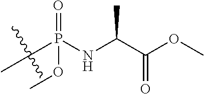 [SMILES: *P(=O)(N[C@@H](C)C(=O)OC)OC] |
| 133 | 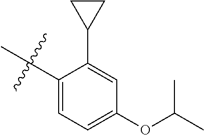 [SMILES: *c1ccc(OC(C)C)cc1C1CC1] | 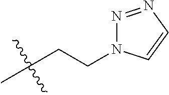 [SMILES: *CCn1ccnn1] | 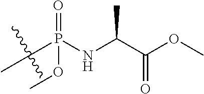 [SMILES: *P(=O)(N[C@@H](C)C(=O)OC)OC] |
| 134 | 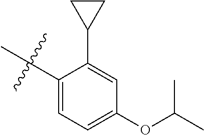 [SMILES: *c1ccc(OC(C)C)cc1C1CC1] | 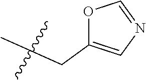 [SMILES: *Cc1cnco1] | 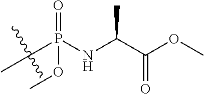 [SMILES: *P(=O)(N[C@@H](C)C(=O)OC)OC] |
| 135 | 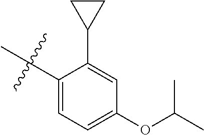 [SMILES: *c1ccc(OC(C)C)cc1C1CC1] | 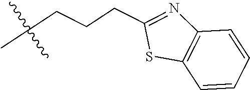 [SMILES: *CCCc1nc2ccccc2s1] | 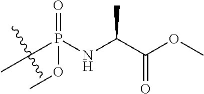 [SMILES: *P(=O)(N[C@@H](C)C(=O)OC)OC] |
| 136 | 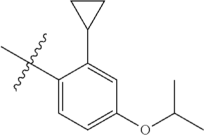 [SMILES: *c1ccc(OC(C)C)cc1C1CC1] | 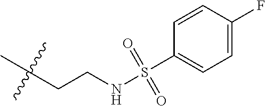 [SMILES: *CCNS(=O)(=O)c1ccc(F)cc1] | 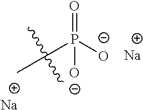 [SMILES: *P(=O)([O-])[O-] ; [Na+] ; [Na+]] |
| 137 | 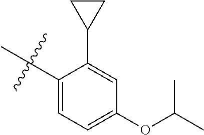 [SMILES: *c1ccc(OC(C)C)cc1C1CC1] | 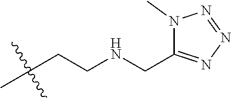 [SMILES: *CCNCc1nnnn1C] | 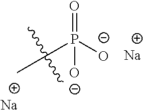 [SMILES: *P(=O)([O-])[O-] ; [Na+] ; [Na+]] |
| 138 | 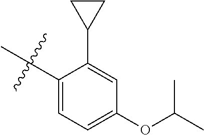 [SMILES: *c1ccc(OC(C)C)cc1C1CC1] | 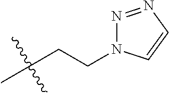 [SMILES: *CCn1ccnn1] | 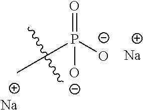 [SMILES: *P(=O)([O-])[O-] ; [Na+] ; [Na+]] |
| 139 | 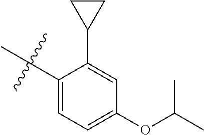 [SMILES: *c1ccc(OC(C)C)cc1C1CC1] | 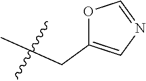 [SMILES: *Cc1cnco1] | 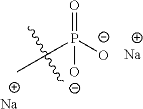 [SMILES: *P(=O)([O-])[O-] ; [Na+] ; [Na+]] |
| 140 | 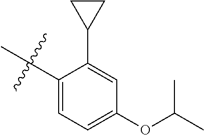 [SMILES: *c1ccc(OC(C)C)cc1C1CC1] | 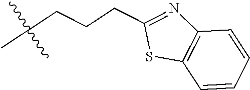 [SMILES: *CCCc1nc2ccccc2s1] | 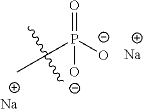 [SMILES: *P(=O)([O-])[O-] ; [Na+] ; [Na+]] |
| 141 | 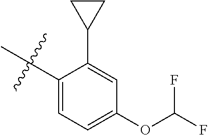 [SMILES: *c1ccc(OC(F)F)cc1C1CC1] | 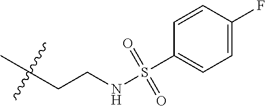 [SMILES: *CCNS(=O)(=O)c1ccc(F)cc1] | 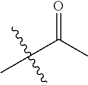 [SMILES: *C(C)=O] |
| 142 | 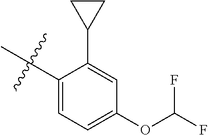 [SMILES: *c1ccc(OC(F)F)cc1C1CC1] | 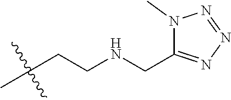 [SMILES: *CCNCc1nnnn1C] | 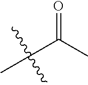 [SMILES: *C(C)=O] |
| 143 | 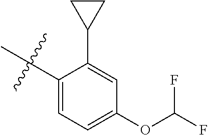 [SMILES: *c1ccc(OC(F)F)cc1C1CC1] | 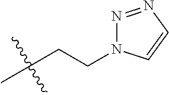 [SMILES: *CCn1ccnn1] | 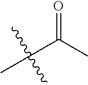 [SMILES: *C(C)=O] |
| 144 | 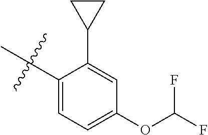 [SMILES: *c1ccc(OC(F)F)cc1C1CC1] | 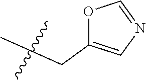 [SMILES: *Cc1cnco1] | 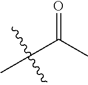 [SMILES: *C(C)=O] |
| 145 | 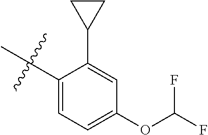 [SMILES: *c1ccc(OC(F)F)cc1C1CC1] | 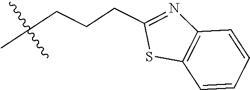 [SMILES: *CCCc1nc2ccccc2s1] | 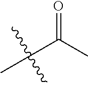 [SMILES: *C(C)=O] |
| 146 | 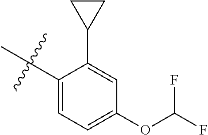 [SMILES: *c1ccc(OC(F)F)cc1C1CC1] | 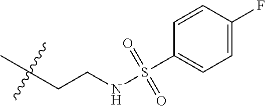 [SMILES: *CCNS(=O)(=O)c1ccc(F)cc1] | 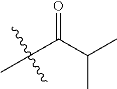 [SMILES: *C(=O)C(C)C] |
| 147 | 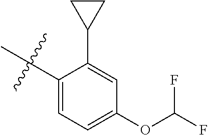 [SMILES: *c1ccc(OC(F)F)cc1C1CC1] | 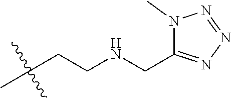 [SMILES: *CCNCc1nnnn1C] | 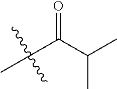 [SMILES: *C(=O)C(C)C] |
| 148 | 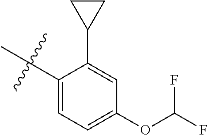 [SMILES: *c1ccc(OC(F)F)cc1C1CC1] | 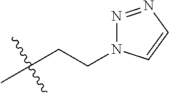 [SMILES: *CCn1ccnn1] | 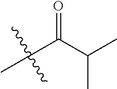 [SMILES: *C(=O)C(C)C] |
| 149 | 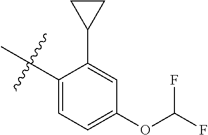 [SMILES: *c1ccc(OC(F)F)cc1C1CC1] | 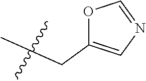 [SMILES: *Cc1cnco1] | 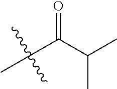 [SMILES: *C(=O)C(C)C] |
| 150 | 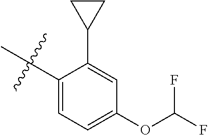 [SMILES: *c1ccc(OC(F)F)cc1C1CC1] | 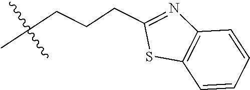 [SMILES: *CCCc1nc2ccccc2s1] | 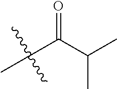 [SMILES: *C(=O)C(C)C] |
| 151 | 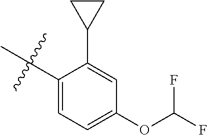 [SMILES: *c1ccc(OC(F)F)cc1C1CC1] | 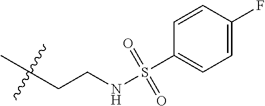 [SMILES: *CCNS(=O)(=O)c1ccc(F)cc1] | 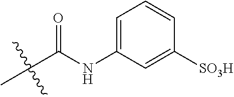 [SMILES: *C(=O)Nc1cccc(S(=O)(=O)O)c1] |
| 152 | 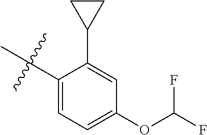 [SMILES: *c1ccc(OC(F)F)cc1C1CC1] | 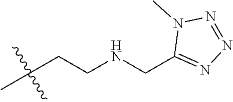 [SMILES: *CCNCc1nnnn1C] | 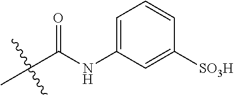 [SMILES: *C(=O)Nc1cccc(S(=O)(=O)O)c1] |
| 153 | 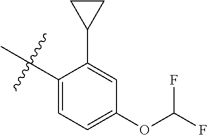 [SMILES: *c1ccc(OC(F)F)cc1C1CC1] | 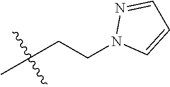 [SMILES: *CCn1cccn1] | 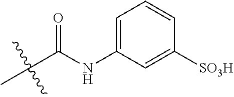 [SMILES: *C(=O)Nc1cccc(S(=O)(=O)O)c1] |
| 154 | 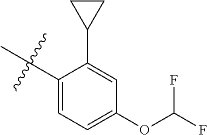 [SMILES: *c1ccc(OC(F)F)cc1C1CC1] | 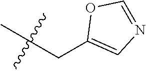 [SMILES: *Cc1cnco1] | 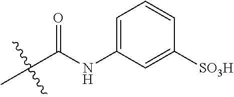 [SMILES: *C(=O)Nc1cccc(S(=O)(=O)O)c1] |
| 155 | 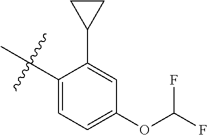 [SMILES: *c1ccc(OC(F)F)cc1C1CC1] | 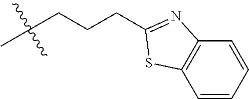 [SMILES: *CCCc1nc2ccccc2s1] | 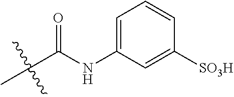 [SMILES: *C(=O)Nc1cccc(S(=O)(=O)O)c1] |
| 156 | 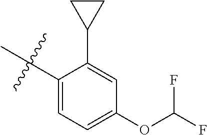 [SMILES: *c1ccc(OC(F)F)cc1C1CC1] | 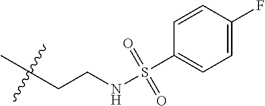 [SMILES: *CCNS(=O)(=O)c1ccc(F)cc1] | 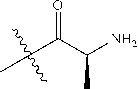 [SMILES: *C(=O)[C@H](C)N] |
| 157 | 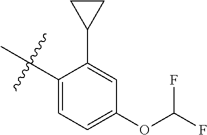 [SMILES: *c1ccc(OC(F)F)cc1C1CC1] | 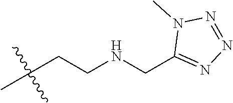 [SMILES: *CCNCc1nnnn1C] | 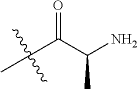 [SMILES: *C(=O)[C@H](C)N] |
| 158 | 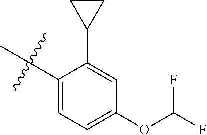 [SMILES: *c1ccc(OC(F)F)cc1C1CC1] | 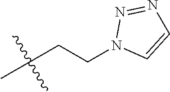 [SMILES: *CCn1ccnn1] | 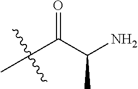 [SMILES: *C(=O)[C@H](C)N] |
| 159 | 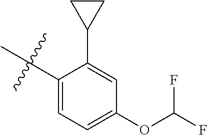 [SMILES: *c1ccc(OC(F)F)cc1C1CC1] | 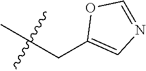 [SMILES: *Cc1cnco1] | 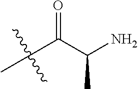 [SMILES: *C(=O)[C@H](C)N] |
| 160 | 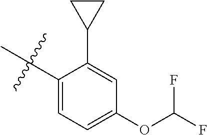 [SMILES: *c1ccc(OC(F)F)cc1C1CC1] | 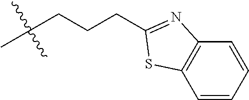 [SMILES: *CCCc1nc2ccccc2s1] | 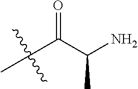 [SMILES: *C(=O)[C@H](C)N] |
| 161 | 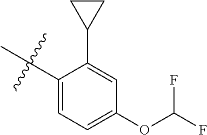 [SMILES: *c1ccc(OC(F)F)cc1C1CC1] | 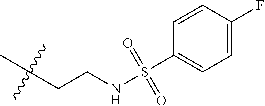 [SMILES: *CCNS(=O)(=O)c1ccc(F)cc1] | 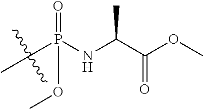 [SMILES: *P(=O)(N[C@@H](C)C(=O)OC)OC] |
| 162 | 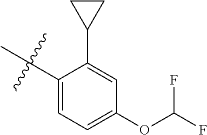 [SMILES: *c1ccc(OC(F)F)cc1C1CC1] | 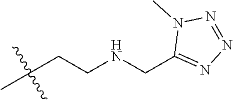 [SMILES: *CCNCc1nnnn1C] | 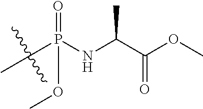 [SMILES: *P(=O)(N[C@@H](C)C(=O)OC)OC] |
| 163 | 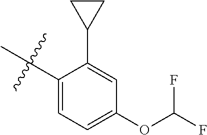 [SMILES: *c1ccc(OC(F)F)cc1C1CC1] | 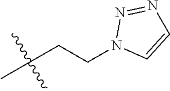 [SMILES: *CCn1ccnn1] | 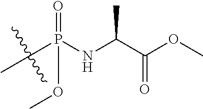 [SMILES: *P(=O)(N[C@@H](C)C(=O)OC)OC] |
| 164 | 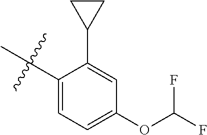 [SMILES: *c1ccc(OC(F)F)cc1C1CC1] | 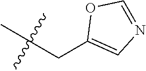 [SMILES: *Cc1cnco1] | 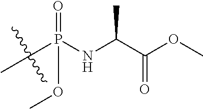 [SMILES: *P(=O)(N[C@@H](C)C(=O)OC)OC] |
| 165 | 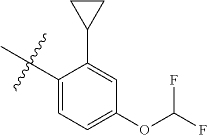 [SMILES: *c1ccc(OC(F)F)cc1C1CC1] | 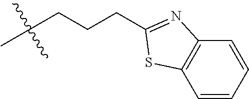 [SMILES: *CCCc1nc2ccccc2s1] | 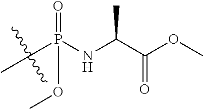 [SMILES: *P(=O)(N[C@@H](C)C(=O)OC)OC] |
| 166 | 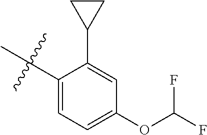 [SMILES: *c1ccc(OC(F)F)cc1C1CC1] | 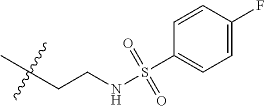 [SMILES: *CCNS(=O)(=O)c1ccc(F)cc1] | 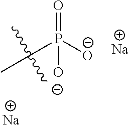 [SMILES: *P(=O)([O-])O ; [Na+] ; [Na+]] |
| 167 | 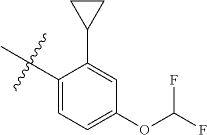 [SMILES: *c1ccc(OC(F)F)cc1C1CC1] | 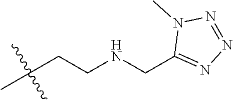 [SMILES: *CCNCc1nnnn1C] | 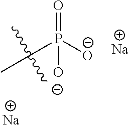 [SMILES: *P(=O)([O-])O ; [Na+] ; [Na+]] |
| 168 | 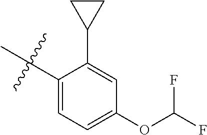 [SMILES: *c1ccc(OC(F)F)cc1C1CC1] | 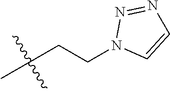 [SMILES: *CCn1ccnn1] | 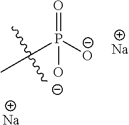 [SMILES: *P(=O)([O-])O ; [Na+] ; [Na+]] |
| 169 | 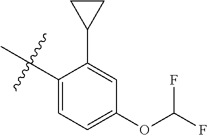 [SMILES: *c1ccc(OC(F)F)cc1C1CC1] | 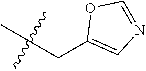 [SMILES: *Cc1cnco1] | 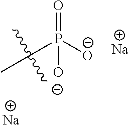 [SMILES: *P(=O)([O-])O ; [Na+] ; [Na+]] |
| 170 | 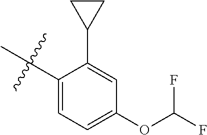 [SMILES: *c1ccc(OC(F)F)cc1C1CC1] | 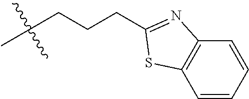 [SMILES: *CCCc1nc2ccccc2s1] | 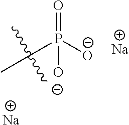 [SMILES: *P(=O)([O-])O ; [Na+] ; [Na+]] |
| 171 | 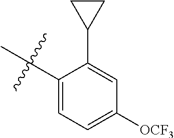 [SMILES: *c1ccc(OC(F)(F)F)cc1C1CC1] | 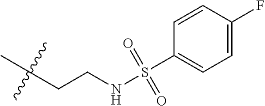 [SMILES: *CCNS(=O)(=O)c1ccc(F)cc1] | 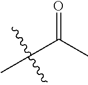 [SMILES: *C(C)=O] |
| 172 | 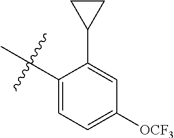 [SMILES: *c1ccc(OC(F)(F)F)cc1C1CC1] | 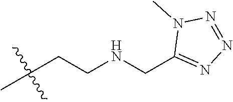 [SMILES: *CCNCc1nnnn1C] | 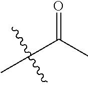 [SMILES: *C(C)=O] |
| 173 | 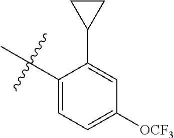 [SMILES: *c1ccc(OC(F)(F)F)cc1C1CC1] | 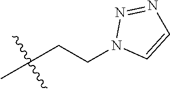 [SMILES: *CCn1ccnn1] | 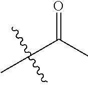 [SMILES: *C(C)=O] |
| 174 | 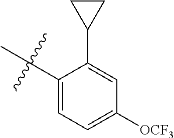 [SMILES: *c1ccc(OC(F)(F)F)cc1C1CC1] | 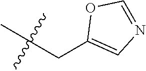 [SMILES: *Cc1cnco1] | 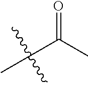 [SMILES: *C(C)=O] |
| 175 | 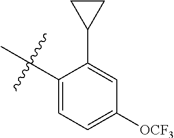 [SMILES: *c1ccc(OC(F)(F)F)cc1C1CC1] | 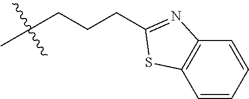 [SMILES: *CCCc1nc2ccccc2s1] | 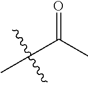 [SMILES: *C(C)=O] |
| 176 | 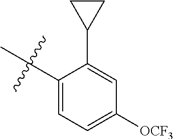 [SMILES: *c1ccc(OC(F)(F)F)cc1C1CC1] | 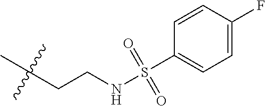 [SMILES: *CCNS(=O)(=O)c1ccc(F)cc1] | 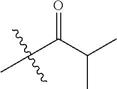 [SMILES: *C(=O)C(C)C] |
| 177 | 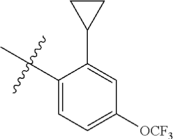 [SMILES: *c1ccc(OC(F)(F)F)cc1C1CC1] | 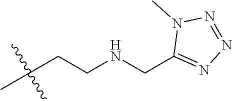 [SMILES: *CCNCc1nnnn1C] | 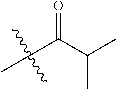 [SMILES: *C(=O)C(C)C] |
| 178 | 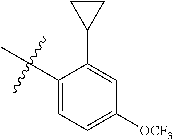 [SMILES: *c1ccc(OC(F)(F)F)cc1C1CC1] | 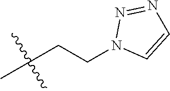 [SMILES: *CCn1ccnn1] | 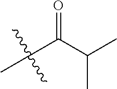 [SMILES: *C(=O)C(C)C] |
| 179 | 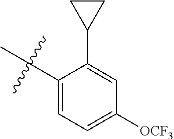 [SMILES: *c1ccc(OC(F)(F)F)cc1C1CC1] | 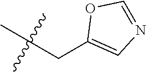 [SMILES: *Cc1cnco1] | 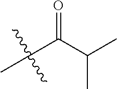 [SMILES: *C(=O)C(C)C] |
| 180 | 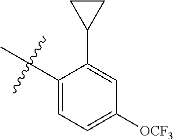 [SMILES: *c1ccc(OC(F)(F)F)cc1C1CC1] | 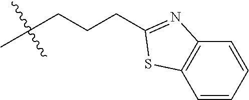 [SMILES: *CCCc1nc2ccccc2s1] | 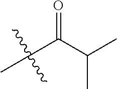 [SMILES: *C(=O)C(C)C] |
| 181 | 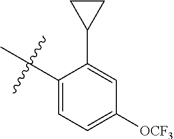 [SMILES: *c1ccc(OC(F)(F)F)cc1C1CC1] | 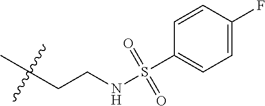 [SMILES: *CCNS(=O)(=O)c1ccc(F)cc1] | 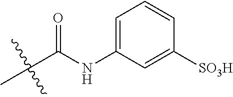 [SMILES: *C(=O)Nc1cccc(S(=O)(=O)O)c1] |
| 182 | 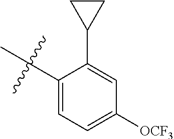 [SMILES: *c1ccc(OC(F)(F)F)cc1C1CC1] | 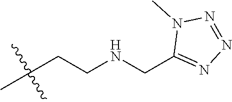 [SMILES: *CCNCc1nnnn1C] | 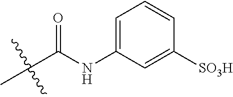 [SMILES: *C(=O)Nc1cccc(S(=O)(=O)O)c1] |
| 183 | 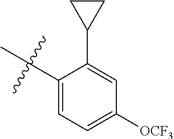 [SMILES: *c1ccc(OC(F)(F)F)cc1C1CC1] | 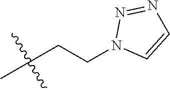 [SMILES: *CCn1ccnn1] | 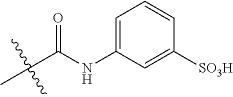 [SMILES: *C(=O)Nc1cccc(S(=O)(=O)O)c1] |
| 184 | 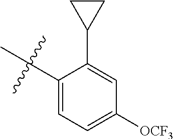 [SMILES: *c1ccc(OC(F)(F)F)cc1C1CC1] | 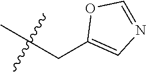 [SMILES: *Cc1cnco1] | 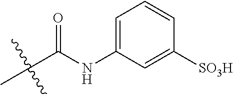 [SMILES: *C(=O)Nc1cccc(S(=O)(=O)O)c1] |
| 185 | 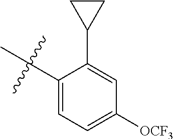 [SMILES: *c1ccc(OC(F)(F)F)cc1C1CC1] | 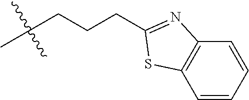 [SMILES: *CCCc1nc2ccccc2s1] | 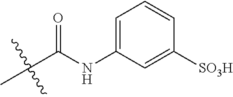 [SMILES: *C(=O)Nc1cccc(S(=O)(=O)O)c1] |
| 186 | 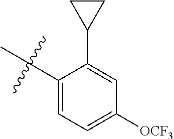 [SMILES: *c1ccc(OC(F)(F)F)cc1C1CC1] | 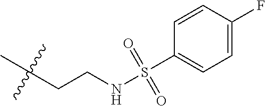 [SMILES: *CCNS(=O)(=O)c1ccc(F)cc1] | 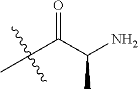 [SMILES: *C(=O)[C@H](C)N] |
| 187 | 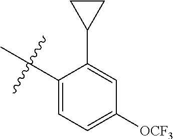 [SMILES: *c1ccc(OC(F)(F)F)cc1C1CC1] | 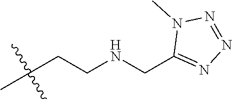 [SMILES: *CCNCc1nnnn1C] | 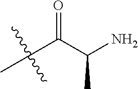 [SMILES: *C(=O)[C@H](C)N] |
| 188 | 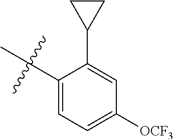 [SMILES: *c1ccc(OC(F)(F)F)cc1C1CC1] | 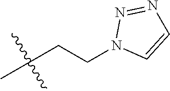 [SMILES: *CCn1ccnn1] | 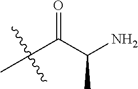 [SMILES: *C(=O)[C@H](C)N] |
| 189 | 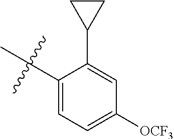 [SMILES: *c1ccc(OC(F)(F)F)cc1C1CC1] | 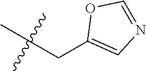 [SMILES: *Cc1cnco1] | 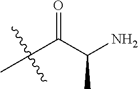 [SMILES: *C(=O)[C@H](C)N] |
| 190 | 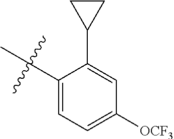 [SMILES: *c1ccc(OC(F)(F)F)cc1C1CC1] | 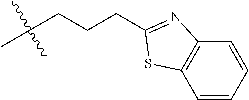 [SMILES: *CCCc1nc2ccccc2s1] | 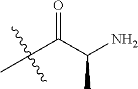 [SMILES: *C(=O)[C@H](C)N] |
| 191 | 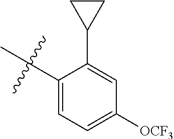 [SMILES: *c1ccc(OC(F)(F)F)cc1C1CC1] | 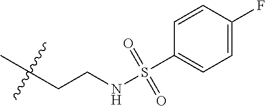 [SMILES: *CCNS(=O)(=O)c1ccc(F)cc1] | 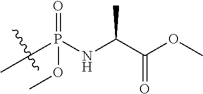 [SMILES: *P(=O)(N[C@@H](C)C(=O)OC)OC] |
| 192 | 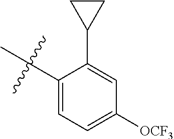 [SMILES: *c1ccc(OC(F)(F)F)cc1C1CC1] | 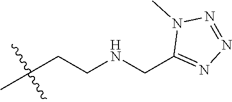 [SMILES: *CCNCc1nnnn1C] | 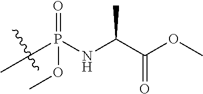 [SMILES: *P(=O)(N[C@@H](C)C(=O)OC)OC] |
| 193 | 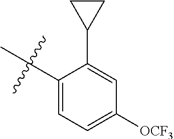 [SMILES: *c1ccc(OC(F)(F)F)cc1C1CC1] | 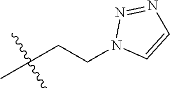 [SMILES: *CCn1ccnn1] | 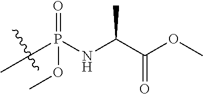 [SMILES: *P(=O)(N[C@@H](C)C(=O)OC)OC] |
| 194 | 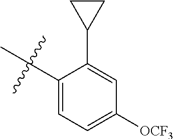 [SMILES: *c1ccc(OC(F)(F)F)cc1C1CC1] | 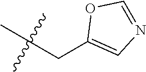 [SMILES: *Cc1cnco1] | 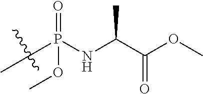 [SMILES: *P(=O)(N[C@@H](C)C(=O)OC)OC] |
| 195 | 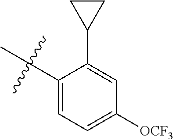 [SMILES: *c1ccc(OC(F)(F)F)cc1C1CC1] | 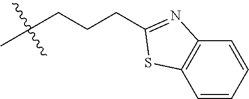 [SMILES: *CCCc1nc2ccccc2s1] | 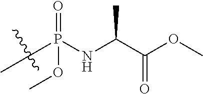 [SMILES: *P(=O)(N[C@@H](C)C(=O)OC)OC] |
| 196 | 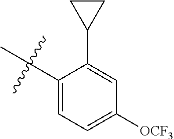 [SMILES: *c1ccc(OC(F)(F)F)cc1C1CC1] | 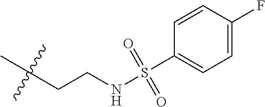 [SMILES: *CCNS(=O)(=O)c1ccc(F)cc1] | 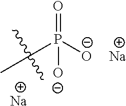 [SMILES: *P(=O)([O-])[O-] ; [Na+] ; [Na+]] |
| 197 | 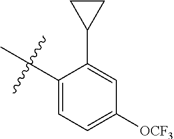 [SMILES: *c1ccc(OC(F)(F)F)cc1C1CC1] | 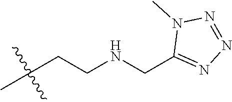 [SMILES: *CCNCc1nnnn1C] | 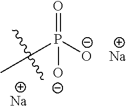 [SMILES: *P(=O)([O-])[O-] ; [Na+] ; [Na+]] |
| 198 | 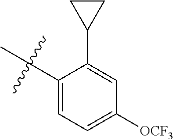 [SMILES: *c1ccc(OC(F)(F)F)cc1C1CC1] | 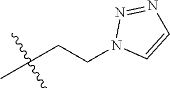 [SMILES: *CCn1ccnn1] | 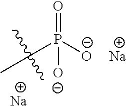 [SMILES: *P(=O)([O-])[O-] ; [Na+] ; [Na+]] |
| 199 | 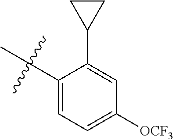 [SMILES: *c1ccc(OC(F)(F)F)cc1C1CC1] | 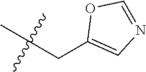 [SMILES: *Cc1cnco1] | 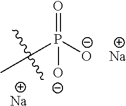 [SMILES: *P(=O)([O-])[O-] ; [Na+] ; [Na+]] |
| 200 | 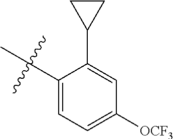 [SMILES: *c1ccc(OC(F)(F)F)cc1C1CC1] | 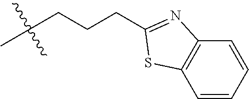 [SMILES: *CCCc1nc2ccccc2s1] | 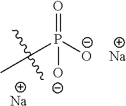 [SMILES: *P(=O)([O-])[O-] ; [Na+] ; [Na+]] |
| 201 | 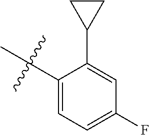 [SMILES: *c1ccc(F)cc1C1CC1] | 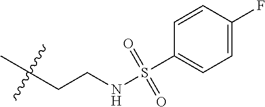 [SMILES: *CCNS(=O)(=O)c1ccc(F)cc1] | 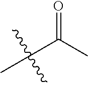 [SMILES: *C(C)=O] |
| 202 | 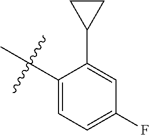 [SMILES: *c1ccc(F)cc1C1CC1] | 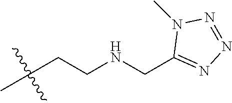 [SMILES: *CCNCc1nnnn1C] | 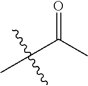 [SMILES: *C(C)=O] |
| 203 | 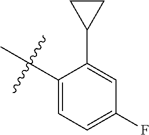 [SMILES: *c1ccc(F)cc1C1CC1] | 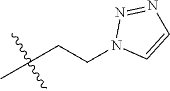 [SMILES: *CCn1ccnn1] | 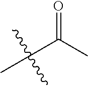 [SMILES: *C(C)=O] |
| 204 | 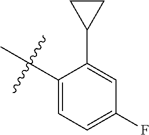 [SMILES: *c1ccc(F)cc1C1CC1] | 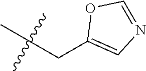 [SMILES: *Cc1cnco1] | 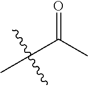 [SMILES: *C(C)=O] |
| 205 | 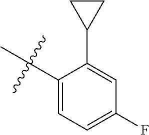 [SMILES: *c1ccc(F)cc1C1CC1] | 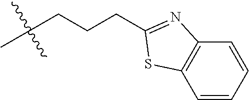 [SMILES: *CCCc1nc2ccccc2s1] | 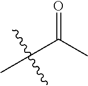 [SMILES: *C(C)=O] |
| 206 | 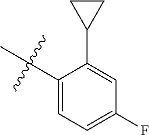 [SMILES: *c1ccc(F)cc1C1CC1] | 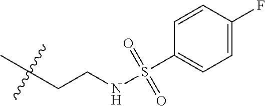 [SMILES: *CCNS(=O)(=O)c1ccc(F)cc1] | 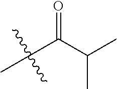 [SMILES: *C(=O)C(C)C] |
| 207 | 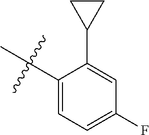 [SMILES: *c1ccc(F)cc1C1CC1] | 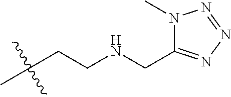 [SMILES: *CCNCc1nnnn1C] | 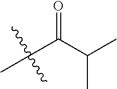 [SMILES: *C(=O)C(C)C] |
| 208 | 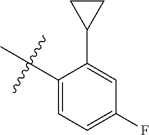 [SMILES: *c1ccc(F)cc1C1CC1] | 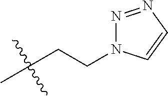 [SMILES: *CCn1ccnn1] | 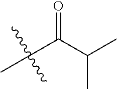 [SMILES: *C(=O)C(C)C] |
| 209 | 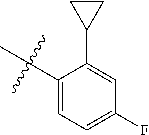 [SMILES: *c1ccc(F)cc1C1CC1] | 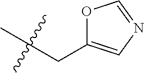 [SMILES: *Cc1cnco1] | 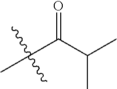 [SMILES: *C(=O)C(C)C] |
| 210 | 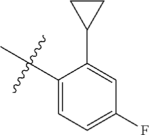 [SMILES: *c1ccc(F)cc1C1CC1] | 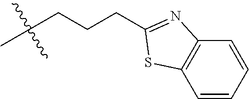 [SMILES: *CCCc1nc2ccccc2s1] | 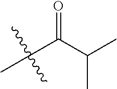 [SMILES: *C(=O)C(C)C] |
| 211 | 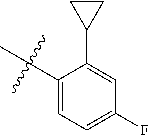 [SMILES: *c1ccc(F)cc1C1CC1] | 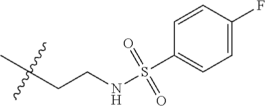 [SMILES: *CCNS(=O)(=O)c1ccc(F)cc1] | 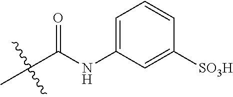 [SMILES: *C(=O)Nc1cccc(S(=O)(=O)O)c1] |
| 212 | 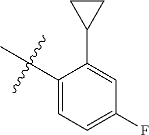 [SMILES: *c1ccc(F)cc1C1CC1] | 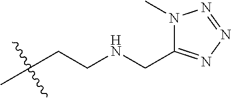 [SMILES: *CCNCc1nnnn1C] | 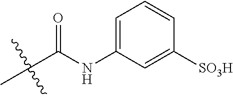 [SMILES: *C(=O)Nc1cccc(S(=O)(=O)O)c1] |
| 213 | 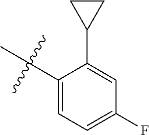 [SMILES: *c1ccc(F)cc1C1CC1] | 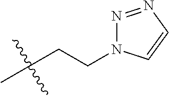 [SMILES: *CCn1ccnn1] | 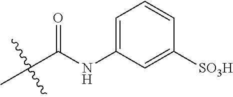 [SMILES: *C(=O)Nc1cccc(S(=O)(=O)O)c1] |
| 214 | 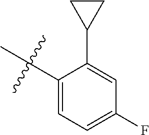 [SMILES: *c1ccc(F)cc1C1CC1] | 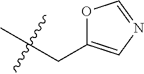 [SMILES: *Cc1cnco1] | 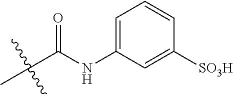 [SMILES: *C(=O)Nc1cccc(S(=O)(=O)O)c1] |
| 215 | 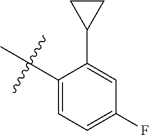 [SMILES: *c1ccc(F)cc1C1CC1] | 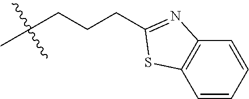 [SMILES: *CCCc1nc2ccccc2s1] | 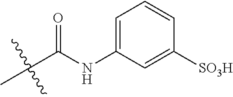 [SMILES: *C(=O)Nc1cccc(S(=O)(=O)O)c1] |
| 216 | 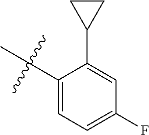 [SMILES: *c1ccc(F)cc1C1CC1] | 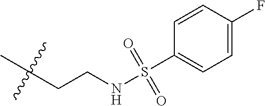 [SMILES: *CCNS(=O)(=O)c1ccc(F)cc1] | 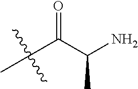 [SMILES: *C(=O)[C@H](C)N] |
| 217 | 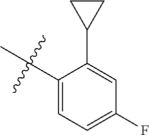 [SMILES: *c1ccc(F)cc1C1CC1] | 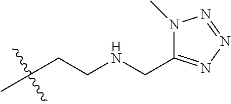 [SMILES: *CCNCc1nnnn1C] | 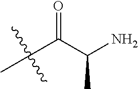 [SMILES: *C(=O)[C@H](C)N] |
| 218 | 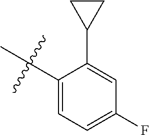 [SMILES: *c1ccc(F)cc1C1CC1] | 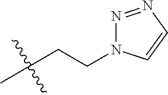 [SMILES: *CCn1ccnn1] | 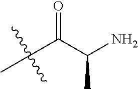 [SMILES: *C(=O)[C@H](C)N] |
| 219 | 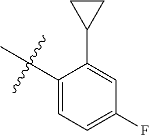 [SMILES: *c1ccc(F)cc1C1CC1] | 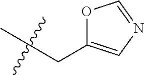 [SMILES: *Cc1cnco1] | 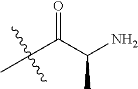 [SMILES: *C(=O)[C@H](C)N] |
| 220 | 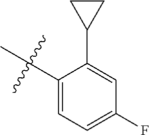 [SMILES: *c1ccc(F)cc1C1CC1] | 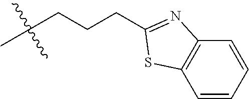 [SMILES: *CCCc1nc2ccccc2s1] | 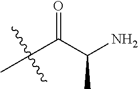 [SMILES: *C(=O)[C@H](C)N] |
| 221 | 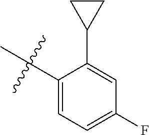 [SMILES: *c1ccc(F)cc1C1CC1] | 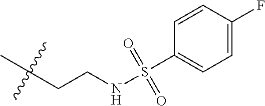 [SMILES: *CCNS(=O)(=O)c1ccc(F)cc1] | 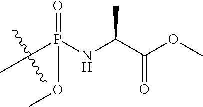 [SMILES: *P(=O)(N[C@@H](C)C(=O)OC)OC] |
| 222 | 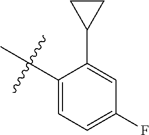 [SMILES: *c1ccc(F)cc1C1CC1] | 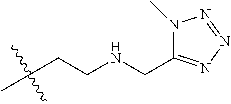 [SMILES: *CCNCc1nnnn1C] | 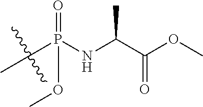 [SMILES: *P(=O)(N[C@@H](C)C(=O)OC)OC] |
| 223 | 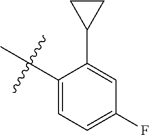 [SMILES: *c1ccc(F)cc1C1CC1] | 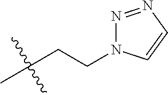 [SMILES: *CCn1ccnn1] | 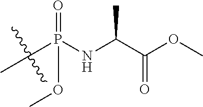 [SMILES: *P(=O)(N[C@@H](C)C(=O)OC)OC] |
| 224 | 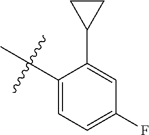 [SMILES: *c1ccc(F)cc1C1CC1] | 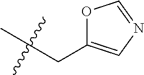 [SMILES: *Cc1cnco1] | 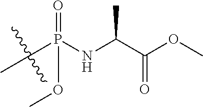 [SMILES: *P(=O)(N[C@@H](C)C(=O)OC)OC] |
| 225 | 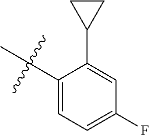 [SMILES: *c1ccc(F)cc1C1CC1] | 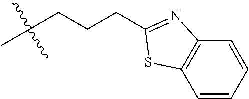 [SMILES: *CCCc1nc2ccccc2s1] | 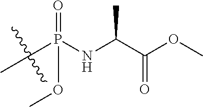 [SMILES: *P(=O)(N[C@@H](C)C(=O)OC)OC] |
| 226 | 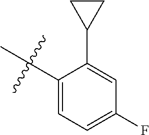 [SMILES: *c1ccc(F)cc1C1CC1] | 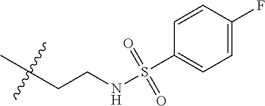 [SMILES: *CCNS(=O)(=O)c1ccc(F)cc1] | 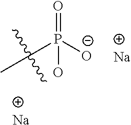 [SMILES: *P(=O)([O-])O ; [Na+] ; [Na+]] |
| 227 | 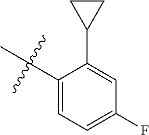 [SMILES: *c1ccc(F)cc1C1CC1] | 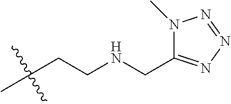 [SMILES: *CCNCc1nnnn1C] | 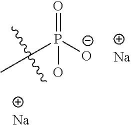 [SMILES: *P(=O)([O-])O ; [Na+] ; [Na+]] |
| 228 | 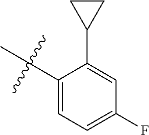 [SMILES: *c1ccc(F)cc1C1CC1] | 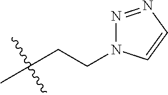 [SMILES: *CCn1ccnn1] | 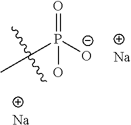 [SMILES: *P(=O)([O-])O ; [Na+] ; [Na+]] |
| 229 | 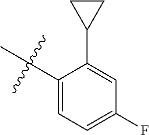 [SMILES: *c1ccc(F)cc1C1CC1] | 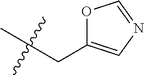 [SMILES: *Cc1cnco1] | 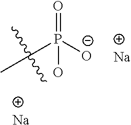 [SMILES: *P(=O)([O-])O ; [Na+] ; [Na+]] |
| 230 | 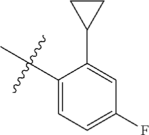 [SMILES: *c1ccc(F)cc1C1CC1] | 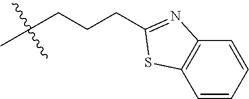 [SMILES: *CCCc1nc2ccccc2s1] | 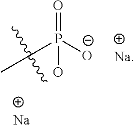 |

9. The compound of claim 1, selected from the compounds set forth below or a pharmaceutically acceptable salt thereof:

| Compound | Structure | Compound | Structure |
|---|---|---|---|
| 1 | 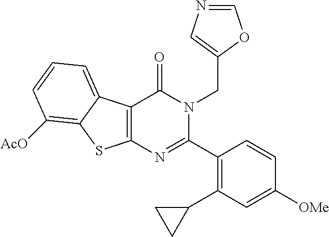 [SMILES: COc1ccc(-c2nc3sc4c(OC(C)=O)cccc4c3c(=O)n2Cc2cnco2)c(C2CC2)c1] | 2 | 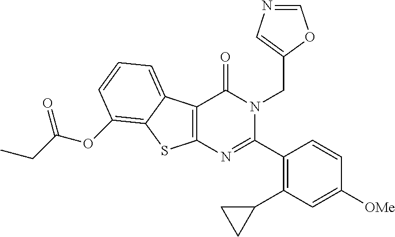 [SMILES: CCC(=O)Oc1cccc2c1sc1nc(-c3ccc(OC)cc3C3CC3)n(Cc3cnco3)c(=O)c12] |
| 3 | 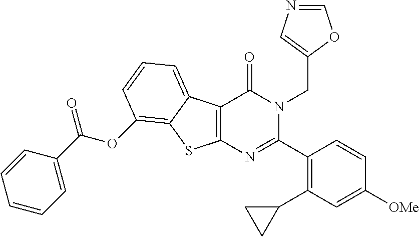 [SMILES: COc1ccc(-c2nc3sc4c(OC(=O)c5ccccc5)cccc4c3c(=O)n2Cc2cnco2)c(C2CC2)c1] | 4 | 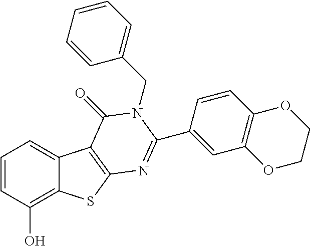 [SMILES: O=c1c2c(nc(-c3ccc4c(c3)OCCO4)n1Cc1ccccc1)sc1c(O)cccc12] |
| 5 | 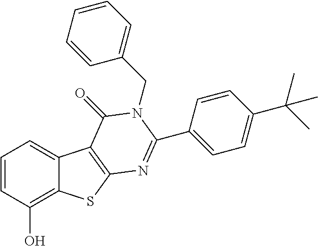 [SMILES: CC(C)(C)c1ccc(-c2nc3sc4c(O)cccc4c3c(=O)n2Cc2ccccc2)cc1] | 6 | 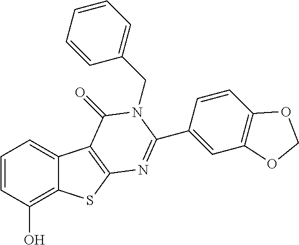 [SMILES: O=c1c2c(nc(-c3ccc4c(c3)OCO4)n1Cc1ccccc1)sc1c(O)cccc12] |
| 7 | 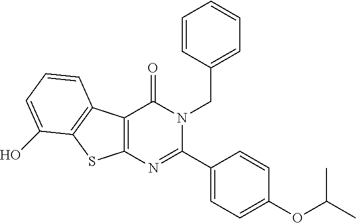 [SMILES: CC(C)Oc1ccc(-c2nc3sc4c(O)cccc4c3c(=O)n2Cc2ccccc2)cc1] | 8 | 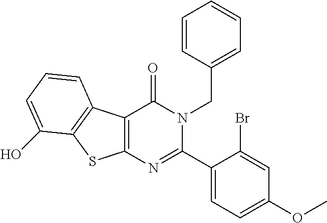 [SMILES: COc1ccc(-c2nc3sc4c(O)cccc4c3c(=O)n2Cc2ccccc2)c(Br)c1] |
| 9 | 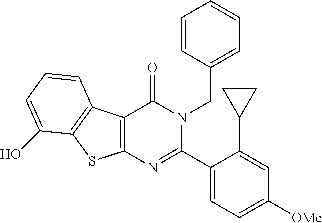 [SMILES: COc1ccc(-c2nc3sc4c(O)cccc4c3c(=O)n2Cc2ccccc2)c(C2CC2)c1] | 10 | 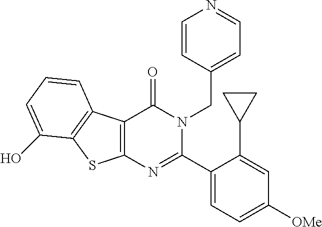 [SMILES: COc1ccc(-c2nc3sc4c(O)cccc4c3c(=O)n2Cc2ccncc2)c(C2CC2)c1] |
| 11 | 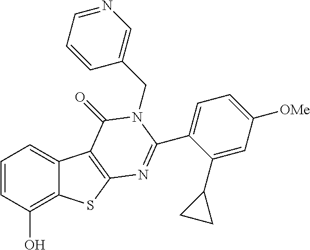 [SMILES: COc1ccc(-c2nc3sc4c(O)cccc4c3c(=O)n2Cc2cccnc2)c(C2CC2)c1] | 12 | 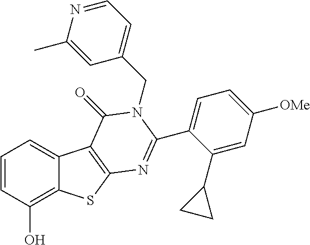 [SMILES: COc1ccc(-c2nc3sc4c(O)cccc4c3c(=O)n2Cc2ccnc(C)c2)c(C2CC2)c1] |
| 13 | 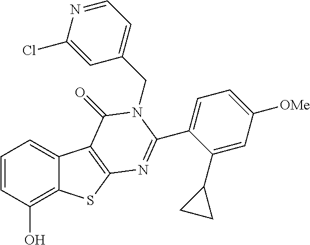 [SMILES: COc1ccc(-c2nc3sc4c(O)cccc4c3c(=O)n2Cc2ccnc(Cl)c2)c(C2CC2)c1] | 14 | 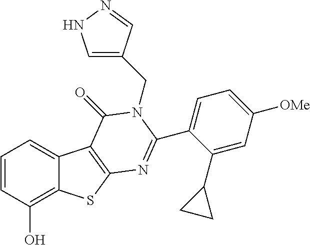 [SMILES: COc1ccc(-c2nc3sc4c(O)cccc4c3c(=O)n2Cc2cn[nH]c2)c(C2CC2)c1] |
| 15 | 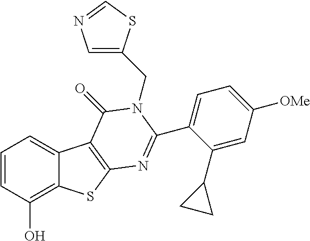 [SMILES: COc1ccc(-c2nc3sc4c(O)cccc4c3c(=O)n2Cc2cncs2)c(C2CC2)c1] | 16 | 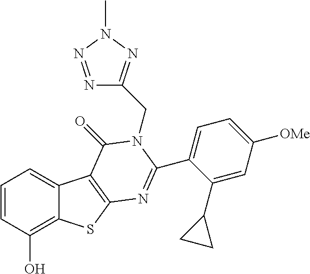 [SMILES: COc1ccc(-c2nc3sc4c(O)cccc4c3c(=O)n2Cc2nnn(C)n2)c(C2CC2)c1] |
| 17 | 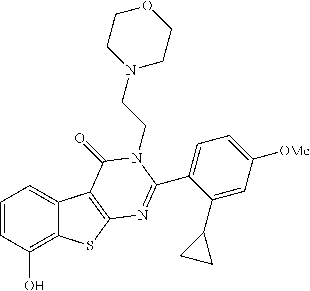 [SMILES: COc1ccc(-c2nc3sc4c(O)cccc4c3c(=O)n2CCN2CCOCC2)c(C2CC2)c1] | 18 | 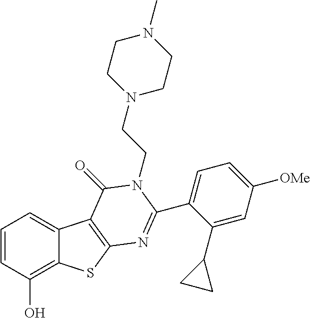 [SMILES: COc1ccc(-c2nc3sc4c(O)cccc4c3c(=O)n2CCN2CCN(C)CC2)c(C2CC2)c1] |
| 19 | 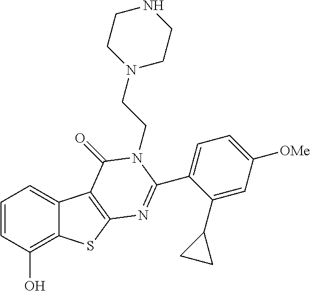 [SMILES: COc1ccc(-c2nc3sc4c(O)cccc4c3c(=O)n2CCN2CCNCC2)c(C2CC2)c1] | 20 | 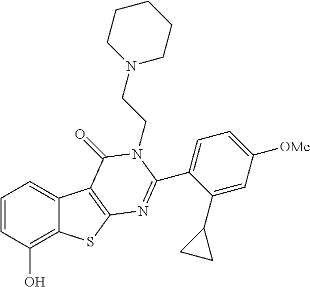 [SMILES: COc1ccc(-c2nc3sc4c(O)cccc4c3c(=O)n2CCN2CCCCC2)c(C2CC2)c1] |
| 21 | 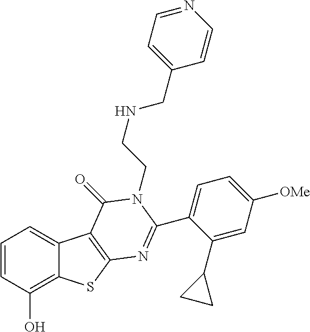 [SMILES: COc1ccc(-c2nc3sc4c(O)cccc4c3c(=O)n2CCNCc2ccncc2)c(C2CC2)c1] | 22 | 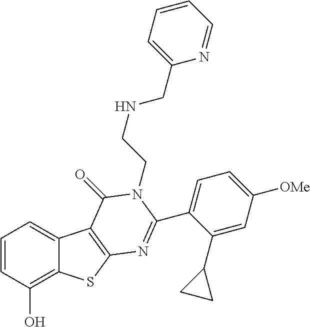 [SMILES: COc1ccc(-c2nc3sc4c(O)cccc4c3c(=O)n2CCNCc2ccccn2)c(C2CC2)c1] |
| 23 | 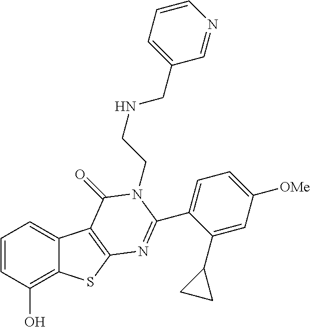 [SMILES: COc1ccc(-c2nc3sc4c(O)cccc4c3c(=O)n2CCNCc2cccnc2)c(C2CC2)c1] | 24 | 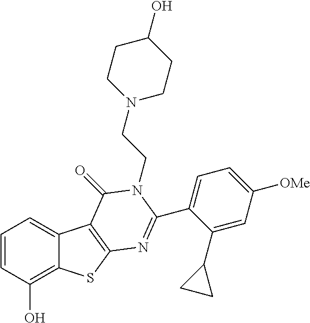 [SMILES: COc1ccc(-c2nc3sc4c(O)cccc4c3c(=O)n2CCN2CCC(O)CC2)c(C2CC2)c1] |
| 25 | 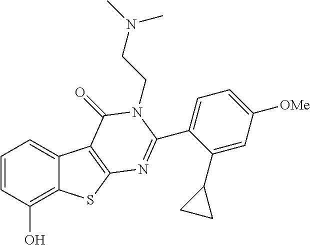 [SMILES: COc1ccc(-c2nc3sc4c(O)cccc4c3c(=O)n2CCN(C)C)c(C2CC2)c1] | 26 | 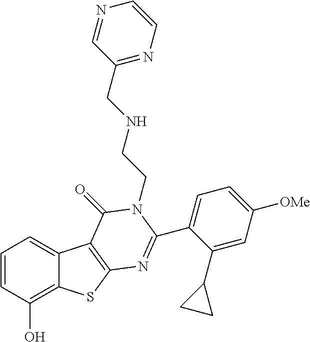 [SMILES: COc1ccc(-c2nc3sc4c(O)cccc4c3c(=O)n2CCNCc2cnccn2)c(C2CC2)c1] |
| 27 | 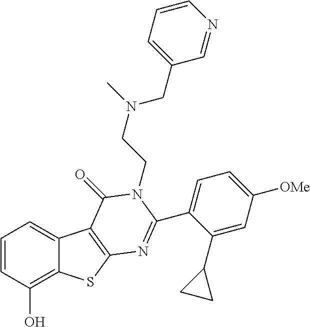 [SMILES: COc1ccc(-c2nc3sc4c(O)cccc4c3c(=O)n2CCN(C)Cc2cccnc2)c(C2CC2)c1] | 28 | 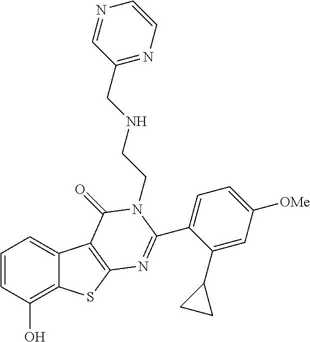 [SMILES: COc1ccc(-c2nc3sc4c(O)cccc4c3c(=O)n2CCNCc2cnccn2)c(C2CC2)c1] |
| 29 | 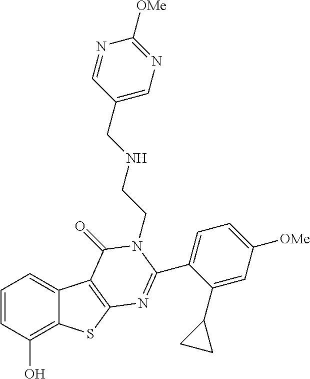 [SMILES: COc1ccc(-c2nc3sc4c(O)cccc4c3c(=O)n2CCNCc2cnc(OC)nc2)c(C2CC2)c1] | 30 | 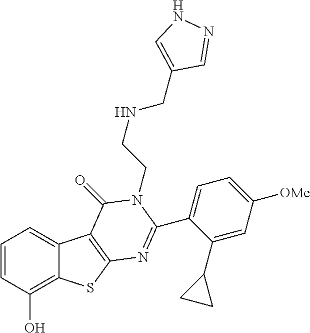 [SMILES: COc1ccc(-c2nc3sc4c(O)cccc4c3c(=O)n2CCNCc2cn[nH]c2)c(C2CC2)c1] |
| 31 | 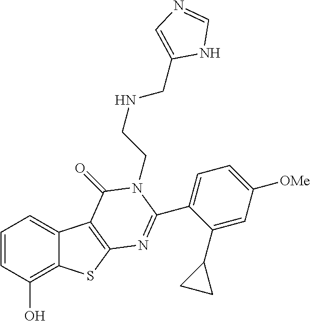 [SMILES: COc1ccc(-c2nc3sc4c(O)cccc4c3c(=O)n2CCNCc2cnc[nH]2)c(C2CC2)c1] | 32 | 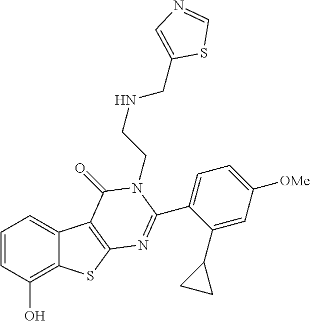 [SMILES: COc1ccc(-c2nc3sc4c(O)cccc4c3c(=O)n2CCNCc2cncs2)c(C2CC2)c1] |
| 33 | 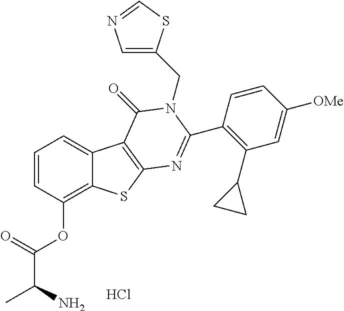 [SMILES: COc1ccc(-c2nc3sc4c(OC(=O)[C@H](C)N)cccc4c3c(=O)n2Cc2cncs2)c(C2CC2)c1 ; Cl] | 34 | 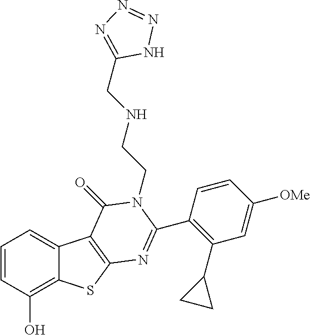 [SMILES: COc1ccc(-c2nc3sc4c(O)cccc4c3c(=O)n2CCNCc2nnn[nH]2)c(C2CC2)c1] |
| 35 | 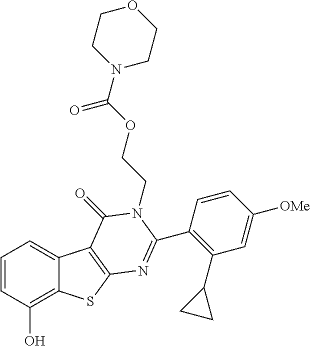 [SMILES: COc1ccc(-c2nc3sc4c(O)cccc4c3c(=O)n2CCOC(=O)N2CCOCC2)c(C2CC2)c1] | 36 | 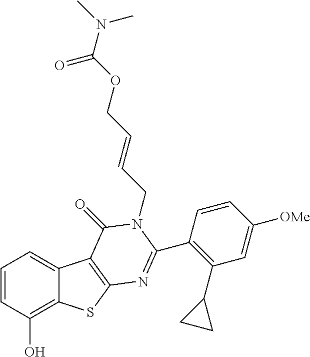 [SMILES: COc1ccc(-c2nc3sc4c(O)cccc4c3c(=O)n2C/C=C/COC(=O)N(C)C)c(C2CC2)c1] |
| 37 | 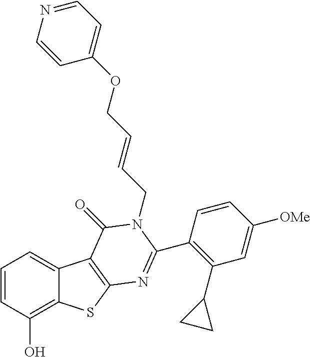 [SMILES: COc1ccc(-c2nc3sc4c(O)cccc4c3c(=O)n2C/C=C/COc2ccncc2)c(C2CC2)c1] | 38 | 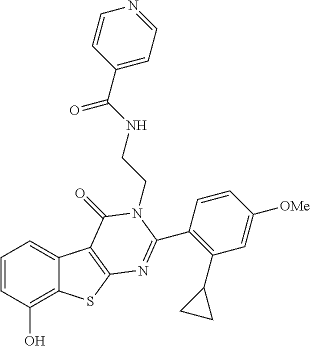 [SMILES: COc1ccc(-c2nc3sc4c(O)cccc4c3c(=O)n2CCNC(=O)c2ccncc2)c(C2CC2)c1] |
| 39 | 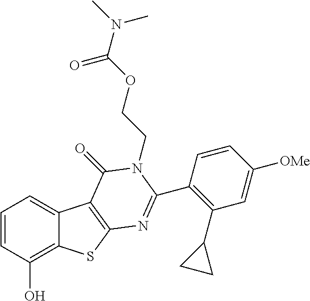 [SMILES: COc1ccc(-c2nc3sc4c(O)cccc4c3c(=O)n2CCOC(=O)N(C)C)c(C2CC2)c1] | 40 | 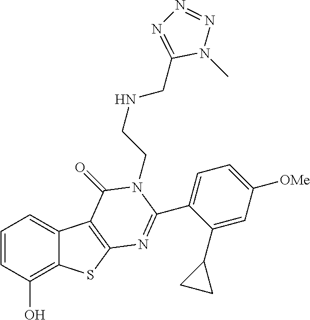 [SMILES: COc1ccc(-c2nc3sc4c(O)cccc4c3c(=O)n2CCNCc2nnnn2C)c(C2CC2)c1] |
| 41 | 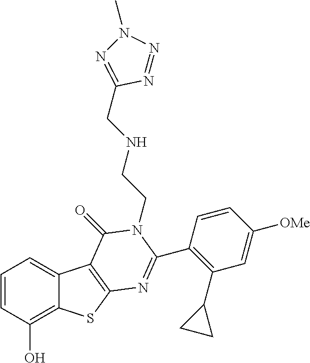 [SMILES: COc1ccc(-c2nc3sc4c(O)cccc4c3c(=O)n2CCNCc2nnn(C)n2)c(C2CC2)c1] | 42 | 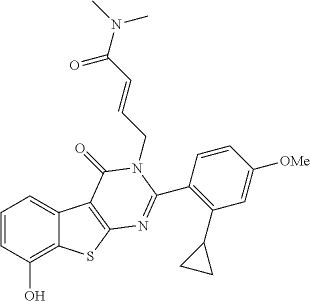 [SMILES: COc1ccc(-c2nc3sc4c(O)cccc4c3c(=O)n2C/C=C/C(=O)N(C)C)c(C2CC2)c1] |
| 43 | 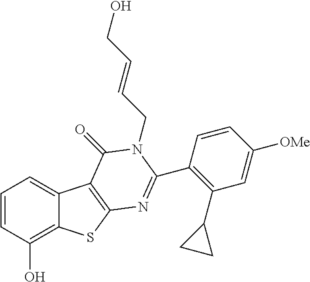 [SMILES: COc1ccc(-c2nc3sc4c(O)cccc4c3c(=O)n2C/C=C/CO)c(C2CC2)c1] | 44 | 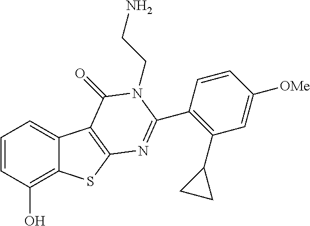 [SMILES: COc1ccc(-c2nc3sc4c(O)cccc4c3c(=O)n2CCN)c(C2CC2)c1] |
| 45 | 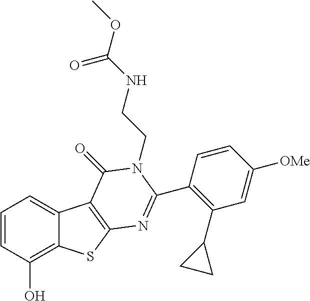 [SMILES: COC(=O)NCCn1c(-c2ccc(OC)cc2C2CC2)nc2sc3c(O)cccc3c2c1=O] | 46 | 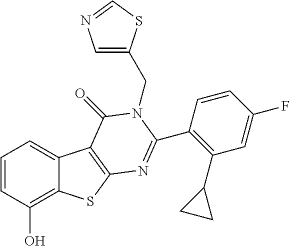 [SMILES: O=c1c2c(nc(-c3ccc(F)cc3C3CC3)n1Cc1cncs1)sc1c(O)cccc12] |
| 47 | 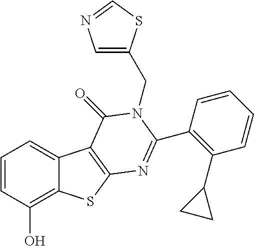 [SMILES: O=c1c2c(nc(-c3ccccc3C3CC3)n1Cc1cncs1)sc1c(O)cccc12] | 48 | 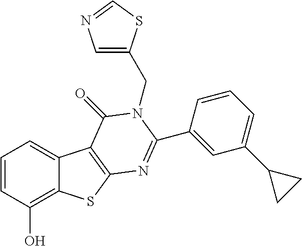 [SMILES: O=c1c2c(nc(-c3cccc(C4CC4)c3)n1Cc1cncs1)sc1c(O)cccc12] |
| 49 | 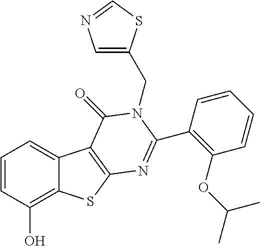 [SMILES: CC(C)Oc1ccccc1-c1nc2sc3c(O)cccc3c2c(=O)n1Cc1cncs1] | 50 | 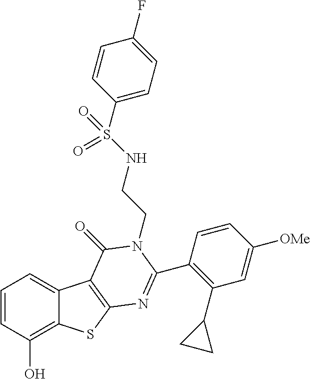 [SMILES: COc1ccc(-c2nc3sc4c(O)cccc4c3c(=O)n2CCNS(=O)(=O)c2ccc(F)cc2)c(C2CC2)c1] |
| 51 | 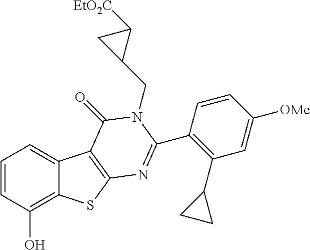 [SMILES: CCOC(=O)C1CC1Cn1c(-c2ccc(OC)cc2C2CC2)nc2sc3c(O)cccc3c2c1=O] | 52 | 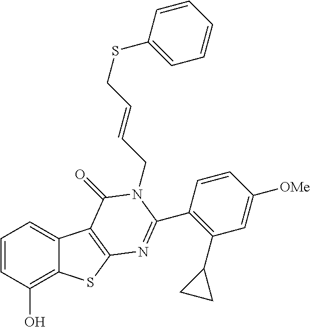 [SMILES: COc1ccc(-c2nc3sc4c(O)cccc4c3c(=O)n2C/C=C/CSc2ccccc2)c(C2CC2)c1] |
| 53 | 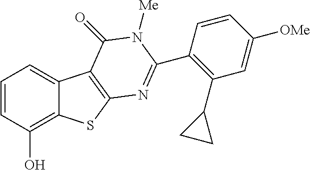 [SMILES: COc1ccc(-c2nc3sc4c(O)cccc4c3c(=O)n2C)c(C2CC2)c1] | 54 | 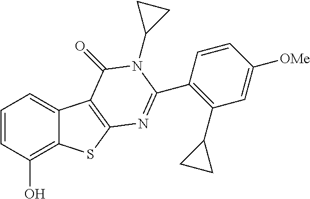 [SMILES: COc1ccc(-c2nc3sc4c(O)cccc4c3c(=O)n2C2CC2)c(C2CC2)c1] |
| 55 | 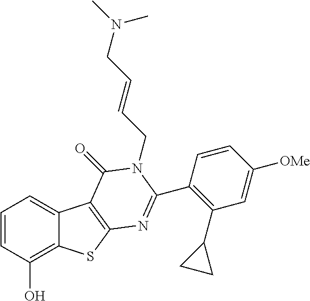 [SMILES: COc1ccc(-c2nc3sc4c(O)cccc4c3c(=O)n2C/C=C/CN(C)C)c(C2CC2)c1] | 56 | 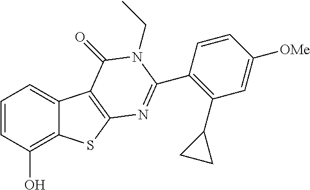 [SMILES: CCn1c(-c2ccc(OC)cc2C2CC2)nc2sc3c(O)cccc3c2c1=O] |
| 57 | 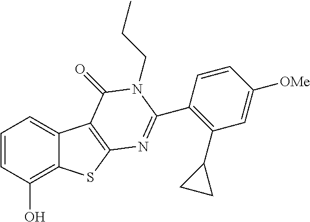 [SMILES: CCCn1c(-c2ccc(OC)cc2C2CC2)nc2sc3c(O)cccc3c2c1=O] | 58 | 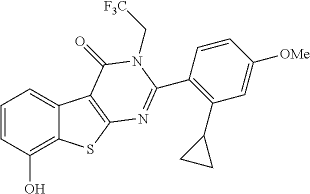 [SMILES: COc1ccc(-c2nc3sc4c(O)cccc4c3c(=O)n2CC(F)(F)F)c(C2CC2)c1] |
| 59 | 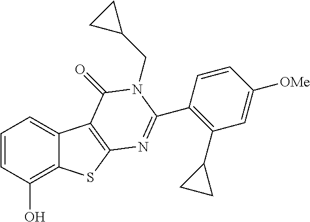 [SMILES: COc1ccc(-c2nc3sc4c(O)cccc4c3c(=O)n2CC2CC2)c(C2CC2)c1] | 60 | 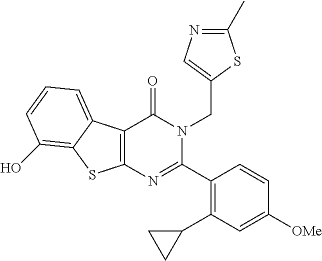 [SMILES: COc1ccc(-c2nc3sc4c(O)cccc4c3c(=O)n2Cc2cnc(C)s2)c(C2CC2)c1] |
| 61 | 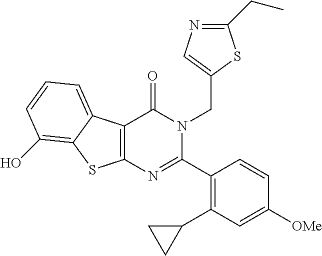 [SMILES: CCc1ncc(Cn2c(-c3ccc(OC)cc3C3CC3)nc3sc4c(O)cccc4c3c2=O)s1] | 62 | 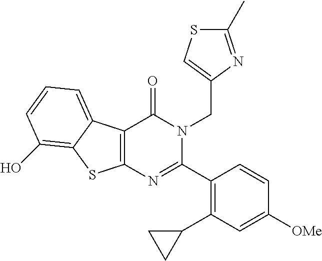 [SMILES: COc1ccc(-c2nc3sc4c(O)cccc4c3c(=O)n2Cc2csc(C)n2)c(C2CC2)c1] |
| 63 | 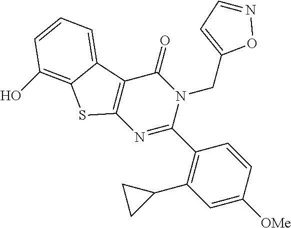 [SMILES: COc1ccc(-c2nc3sc4c(O)cccc4c3c(=O)n2Cc2ccno2)c(C2CC2)c1] | 64 | 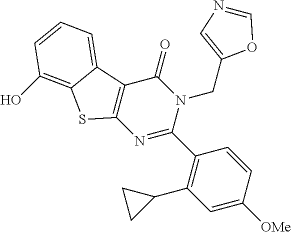 [SMILES: COc1ccc(-c2nc3sc4c(O)cccc4c3c(=O)n2Cc2cnco2)c(C2CC2)c1] |
| 65 | 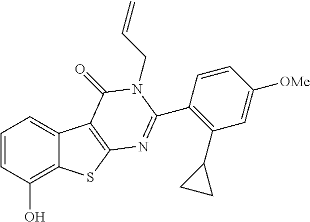 [SMILES: C=CCn1c(-c2ccc(OC)cc2C2CC2)nc2sc3c(O)cccc3c2c1=O] | 66 | 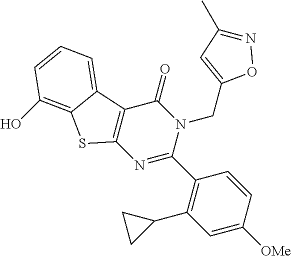 [SMILES: COc1ccc(-c2nc3sc4c(O)cccc4c3c(=O)n2Cc2cc(C)no2)c(C2CC2)c1] |
| 67 | 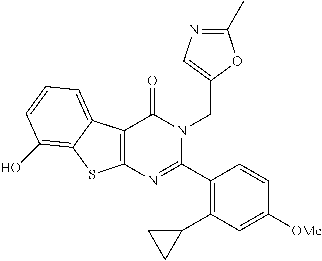 [SMILES: COc1ccc(-c2nc3sc4c(O)cccc4c3c(=O)n2Cc2cnc(C)o2)c(C2CC2)c1] | 68 | 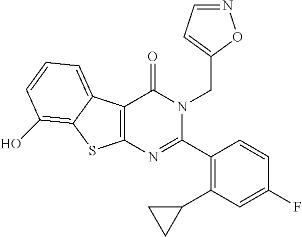 [SMILES: O=c1c2c(nc(-c3ccc(F)cc3C3CC3)n1Cc1ccno1)sc1c(O)cccc12] |
| 69 | 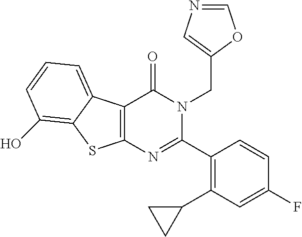 [SMILES: O=c1c2c(nc(-c3ccc(F)cc3C3CC3)n1Cc1cnco1)sc1c(O)cccc12] | 70 | 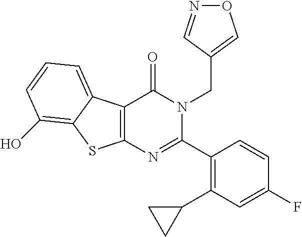 [SMILES: O=c1c2c(nc(-c3ccc(F)cc3C3CC3)n1Cc1cnoc1)sc1c(O)cccc12] |
| 71 | 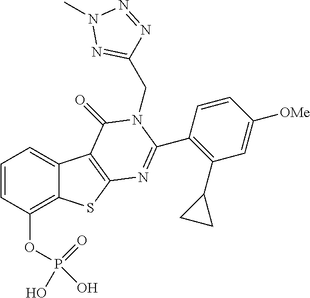 [SMILES: COc1ccc(-c2nc3sc4c(OP(=O)(O)O)cccc4c3c(=O)n2Cc2nnn(C)n2)c(C2CC2)c1] | 72 | 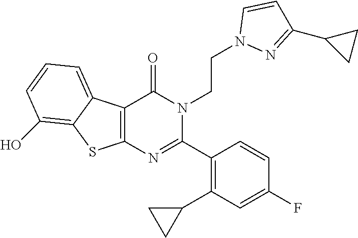 [SMILES: O=c1c2c(nc(-c3ccc(F)cc3C3CC3)n1CCn1ccc(C3CC3)n1)sc1c(O)cccc12] |
| 73 | 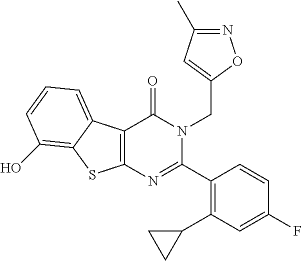 [SMILES: Cc1cc(Cn2c(-c3ccc(F)cc3C3CC3)nc3sc4c(O)cccc4c3c2=O)on1] | 74 | 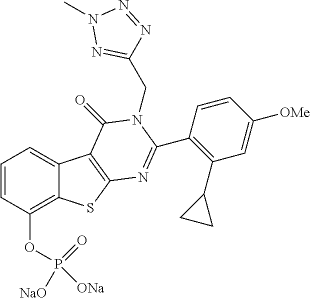 |
| 75 | 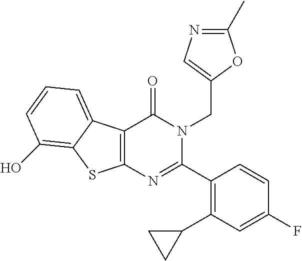 [SMILES: Cc1ncc(Cn2c(-c3ccc(F)cc3C3CC3)nc3sc4c(O)cccc4c3c2=O)o1] | 76 | 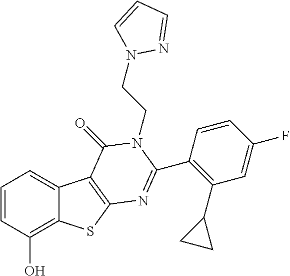 [SMILES: O=c1c2c(nc(-c3ccc(F)cc3C3CC3)n1CCn1cccn1)sc1c(O)cccc12] |
| 77 | 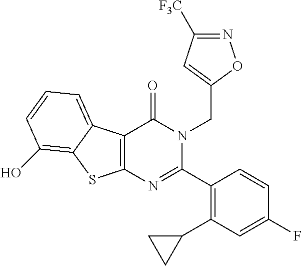 [SMILES: O=c1c2c(nc(-c3ccc(F)cc3C3CC3)n1Cc1cc(C(F)(F)F)no1)sc1c(O)cccc12] | 78 | 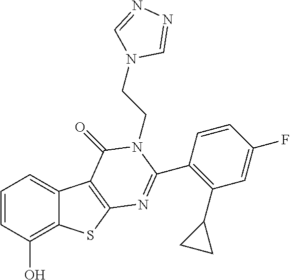 [SMILES: O=c1c2c(nc(-c3ccc(F)cc3C3CC3)n1CCn1cnnc1)sc1c(O)cccc12] |
| 79 | 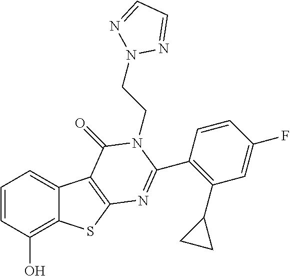 [SMILES: O=c1c2c(nc(-c3ccc(F)cc3C3CC3)n1CCn1nccn1)sc1c(O)cccc12] | 80 | 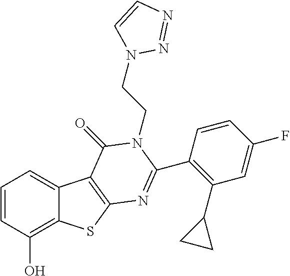 [SMILES: O=c1c2c(nc(-c3ccc(F)cc3C3CC3)n1CCn1ccnn1)sc1c(O)cccc12] |
| 81 | 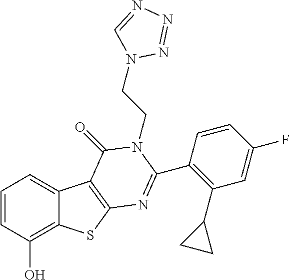 [SMILES: O=c1c2c(nc(-c3ccc(F)cc3C3CC3)n1CCn1cnnn1)sc1c(O)cccc12] | 82 | 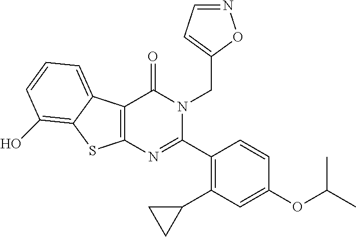 [SMILES: CC(C)Oc1ccc(-c2nc3sc4c(O)cccc4c3c(=O)n2Cc2ccno2)c(C2CC2)c1] |
| 83 | 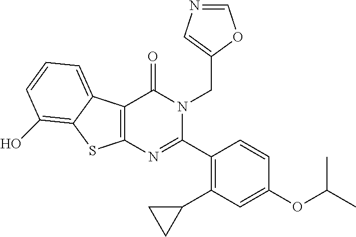 [SMILES: CC(C)Oc1ccc(-c2nc3sc4c(O)cccc4c3c(=O)n2Cc2cnco2)c(C2CC2)c1] | 84 | 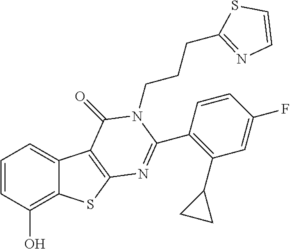 [SMILES: O=c1c2c(nc(-c3ccc(F)cc3C3CC3)n1CCCc1nccs1)sc1c(O)cccc12] |
| 85 | 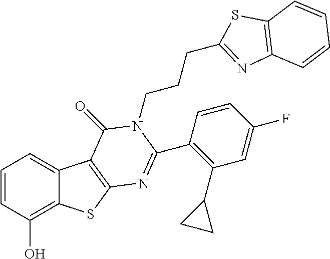 [SMILES: O=c1c2c(nc(-c3ccc(F)cc3C3CC3)n1CCCc1nc3ccccc3s1)sc1c(O)cccc12] | 86 | 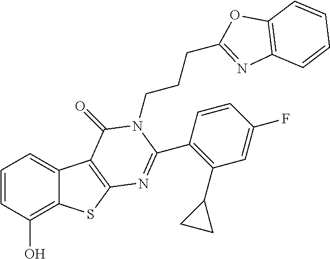 [SMILES: O=c1c2c(nc(-c3ccc(F)cc3C3CC3)n1CCCc1nc3ccccc3o1)sc1c(O)cccc12] |
| 87 | 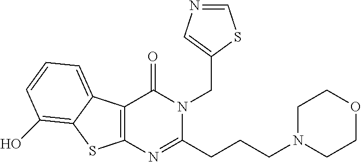 [SMILES: O=c1c2c(nc(CCCN3CCOCC3)n1Cc1cncs1)sc1c(O)cccc12] | 88 | 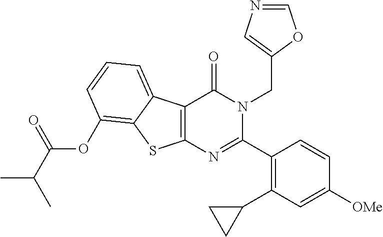 [SMILES: COc1ccc(-c2nc3sc4c(OC(=O)C(C)C)cccc4c3c(=O)n2Cc2cnco2)c(C2CC2)c1] |
| 89 | 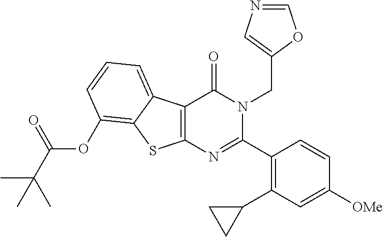 [SMILES: COc1ccc(-c2nc3sc4c(OC(=O)C(C)(C)C)cccc4c3c(=O)n2Cc2cnco2)c(C2CC2)c1] | 90 | 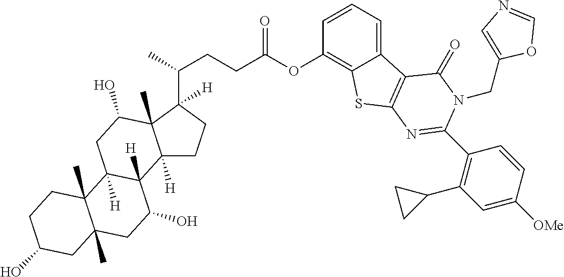 |
| 91 | 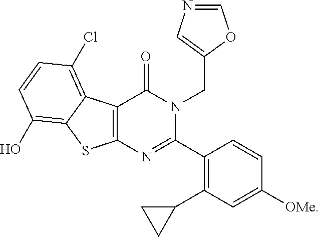 [SMILES: COc1ccc(-c2nc3sc4c(O)ccc(Cl)c4c3c(=O)n2Cc2cnco2)c(C2CC2)c1] | | |

10. A pharmaceutical composition comprising a compound according to claim 1 and a pharmaceutically acceptable carrier or excipient.

11. A method for preventing or treating a 17β-HSD13 mediated disease or condition in a subject in need thereof, comprising administering to the subject a therapeutically effective amount of a compound of claim 1.

12. The method of claim 11, wherein the 17β-HSD13 mediated disease or condition is selected from the group consisting of nonalcoholic fatty liver disease (NAFLD), nonalcoholic steatohepatitis (NASH), liver cirrhosis, liver fibrosis, and hepatocellular carcinoma (HCC).

13. Use of a compound of claim 1 in the manufacture of a medicament for treating or preventing a 17β-HSD13 mediated disease or condition.

14. The use of claim 13, wherein the 17β-HSD13 mediated disease or condition is selected from the group consisting of nonalcoholic fatty liver disease (NAFLD), nonalcoholic steatohepatitis (NASH), liver cirrhosis, liver fibrosis, and hepatocellular carcinoma (HCC).
